SEQUENCE LISTING (318 sequences)

SEQ ID NO: 1 - DNA, 45 bp, Artificial Sequence
  Description of Artificial Sequence Synthetic oligonucleotide
cccacctctccagggcgaacttcgacacacagcctgtccactgct

SEQ ID NO: 2 - DNA, 45 bp, Artificial Sequence
  Description of Artificial Sequence Synthetic oligonucleotide
cccacctctccagggcgaactccgacacacagcctgtccactgct

SEQ ID NO: 3 - DNA, 45 bp, Artificial Sequence
  Description of Artificial Sequence Synthetic oligonucleotide
cccacctctccagggcgaatttggacatacagcctgtccactgct

SEQ ID NO: 4 - DNA, 43 bp, Artificial Sequence
  Description of Artificial Sequence Synthetic oligonucleotide
cccacctctccgggacgaactcggacaacagttgtccactgct

SEQ ID NO: 5 - DNA, 43 bp, Artificial Sequence
  Description of Artificial Sequence Synthetic oligonucleotide
cccacctctccaggacgaactcggacaacagatgtccactgct

SEQ ID NO: 6 - DNA, 43 bp, Artificial Sequence
  Description of Artificial Sequence Synthetic oligonucleotide
cccacctctccggggcgaactcggacaacagttgtccactgct

SEQ ID NO: 7 - DNA, 45 bp, Artificial Sequence
  Description of Artificial Sequence Synthetic oligonucleotide
cccacctctccagggcgaatttggtcacacagcctgtccactgct

SEQ ID NO: 8 - DNA, 45 bp, Artificial Sequence
  Description of Artificial Sequence Synthetic oligonucleotide
cccacctctccagggcgaacttggacacacagcctgtccactgct

SEQ ID NO: 9 - DNA, 45 bp, Artificial Sequence
  Description of Artificial Sequence Synthetic oligonucleotide
cccaccgctccagggcgaacttggtcacacagcctgtccactgct

SEQ ID NO: 10 - DNA, 43 bp, Artificial Sequence
  Description of Artificial Sequence Synthetic oligonucleotide
cccacctctccgggacgaactcggacaacagatgtccactgct

SEQ ID NO: 11 - DNA, 45 bp, Artificial Sequence
  Description of Artificial Sequence Synthetic oligonucleotide
cccacctctccaggacgaactcggacacacaggctgtccactgct

SEQ ID NO: 12 - DNA, 45 bp, Artificial Sequence
  Description of Artificial Sequence Synthetic oligonucleotide
cccacctcttcagggcgatcttggacacacagcctgtccactgct

SEQ ID NO: 13 - DNA, 43 bp, Artificial Sequence
  Description of Artificial Sequence Synthetic oligonucleotide
cccacctctccaggacgaactcggacaacagctgtccactgct

SEQ ID NO: 14 - DNA, 45 bp, Artificial Sequence
  Description of Artificial Sequence Synthetic oligonucleotide
cccacctcttcagggcgaacttggacacacagcctgtccactgct

SEQ ID NO: 15 - DNA, 45 bp, Artificial Sequence
  Description of Artificial Sequence Synthetic oligonucleotide
cccaccgctccagggcgaacttggacatacagcctgtccactgct

SEQ ID NO: 16 - DNA, 43 bp, Artificial Sequence
  Description of Artificial Sequence Synthetic oligonucleotide
cccacctctccgggacgaactcggacaacagctgtccactgct

SEQ ID NO: 17 - DNA, 45 bp, Artificial Sequence
  Description of Artificial Sequence Synthetic oligonucleotide
cccaccgctccagggcgaatttggtcatacagcctgtccactgct

SEQ ID NO: 18 - DNA, 45 bp, Artificial Sequence
  Description of Artificial Sequence Synthetic oligonucleotide
cccaccgctccagggcgaatttggacatacagcctgtccactgct

SEQ ID NO: 19 - DNA, 45 bp, Artificial Sequence
  Description of Artificial Sequence Synthetic oligonucleotide
ccgacctcttcagggcgatcttggacacacagcctgtccactgct

SEQ ID NO: 20 - DNA, 45 bp, Artificial Sequence
  Description of Artificial Sequence Synthetic oligonucleotide
cccacctcttcagggcgatcttggacacacaacctgtccactgct

SEQ ID NO: 21 - DNA, 45 bp, Artificial Sequence
  Description of Artificial Sequence Synthetic oligonucleotide
cccacctctccagggcgaatttggtcatacagcctgtccactgct

SEQ ID NO: 22 - DNA, 43 bp, Artificial Sequence
  Description of Artificial Sequence Synthetic oligonucleotide
cccacctctccaggacgaactcggacaacagttgtccactgct

SEQ ID NO: 23 - DNA, 45 bp, Artificial Sequence
  Description of Artificial Sequence Synthetic oligonucleotide
ccgacctctccagggcgatcttggacacacagcctgtccactgct

SEQ ID NO: 24 - DNA, 45 bp, Artificial Sequence
  Description of Artificial Sequence Synthetic oligonucleotide
ccgacctcttcagggcgatcttggacacacaacctgtccactgct

SEQ ID NO: 25 - DNA, 45 bp, Artificial Sequence
  Description of Artificial Sequence Synthetic oligonucleotide
cccacctctccagggcgaacttggacatacagcctgtccactgct

SEQ ID NO: 26 - DNA, 45 bp, Artificial Sequence
  Description of Artificial Sequence Synthetic oligonucleotide
ccgacctcttcagggcgaactcggacacacagcctgtccactgct

SEQ ID NO: 27 - DNA, 45 bp, Artificial Sequence
  Description of Artificial Sequence Synthetic oligonucleotide
cccacctctccgggacgaactcggacacacaggctgtccactgct

SEQ ID NO: 28 - DNA, 43 bp, Artificial Sequence
  Description of Artificial Sequence Synthetic oligonucleotide
cccacctctccgggacgaactcggacaacaggtgtccactgct

SEQ ID NO: 29 - DNA, 45 bp, Artificial Sequence
  Description of Artificial Sequence Synthetic oligonucleotide
cccacctctccagggcgaatttggacacacagcctgtccactgct

SEQ ID NO: 30 - DNA, 46 bp, Artificial Sequence
  Description of Artificial Sequence Synthetic oligonucleotide
cccaccgctccagggcgaacttatcacatacagcctgtccactgct

SEQ ID NO: 31 - DNA, 45 bp, Artificial Sequence
  Description of Artificial Sequence Synthetic oligonucleotide
cccacctctccagggcgaacttggtcatacagcctgtccactgct

SEQ ID NO: 32 - DNA, 45 bp, Artificial Sequence
  Description of Artificial Sequence Synthetic oligonucleotide
cccaccgctccagggcgaatttggacacacagcctgtccactgct

SEQ ID NO: 33 - DNA, 45 bp, Artificial Sequence
  Description of Artificial Sequence Synthetic oligonucleotide
cccaccgctccagggcgaacttggtcatacagcctgtccactgct

SEQ ID NO: 34 - DNA, 45 bp, Artificial Sequence
  Description of Artificial Sequence Synthetic oligonucleotide
cccaccgctccagggcgaatttggtcacacagcctgtccactgct

SEQ ID NO: 35 - DNA, 45 bp, Artificial Sequence
  Description of Artificial Sequence Synthetic oligonucleotide
tacagcagtactgggtggtgccgtagtcagcaatctttgcaatga

SEQ ID NO: 36 - DNA, 45 bp, Artificial Sequence
  Description of Artificial Sequence Synthetic oligonucleotide
tacagcaggactgagcagtgccgtagtgtcgaatctttgcaatga

SEQ ID NO: 37 - DNA, 45 bp, Artificial Sequence
  Description of Artificial Sequence Synthetic oligonucleotide
taaagcaggactgagtagtgccgtagtcagcaatctttgcaatga

SEQ ID NO: 38 - DNA, 45 bp, Artificial Sequence
  Description of Artificial Sequence Synthetic oligonucleotide
tacagcagtactgagcagtgccgtagtgtcgtttctttgcaatga

SEQ ID NO: 39 - DNA, 45 bp, Artificial Sequence
  Description of Artificial Sequence Synthetic oligonucleotide
tacagcattactgagcagtgccgtagtgtcgtttctttgcaatga

SEQ ID NO: 40 - DNA, 45 bp, Artificial Sequence
  Description of Artificial Sequence Synthetic oligonucleotide
tacagcaggactgagtagtgccgtagtgtcgaatctttgcaatga

SEQ ID NO: 41 - DNA, 45 bp, Artificial Sequence
  Description of Artificial Sequence Synthetic oligonucleotide
tacagcaggactgagcggtgccgtagtcagcaatctttgcaatga

SEQ ID NO: 42 - DNA, 45 bp, Artificial Sequence
  Description of Artificial Sequence Synthetic oligonucleotide
tacagcagtactgagcggtgccgtagtcagcaatctttgcaatga

SEQ ID NO: 43 - DNA, 45 bp, Artificial Sequence
  Description of Artificial Sequence Synthetic oligonucleotide
tacagcaggactgggtgatgccgtagtcagcaatctttgcaatga

SEQ ID NO: 44 - DNA, 45 bp, Artificial Sequence
  Description of Artificial Sequence Synthetic oligonucleotide
taaagcagtactgagcggtgccgtagtcagcaatctttgcaatga

SEQ ID NO: 45 - DNA, 45 bp, Artificial Sequence
  Description of Artificial Sequence Synthetic oligonucleotide
tacagcagtcctgggtggtgccgtagtcagcaatctttgcaatga

SEQ ID NO: 46 - DNA, 45 bp, Artificial Sequence
  Description of Artificial Sequence Synthetic oligonucleotide
tacagcaggactgggtagtgccgtagtgtcgaatctttgcaatga

SEQ ID NO: 47 - DNA, 45 bp, Artificial Sequence
  Description of Artificial Sequence Synthetic oligonucleotide
tacagcaggactgagcagtgccgtagtcagcaatctttgcaatga

SEQ ID NO: 48 - DNA, 45 bp, Artificial Sequence
  Description of Artificial Sequence Synthetic oligonucleotide
taaagcagtactgagcagtgccgtagtcagcaatctttgcaatga

SEQ ID NO: 49 - DNA, 45 bp, Artificial Sequence
  Description of Artificial Sequence Synthetic oligonucleotide
taaagcaggcctgagcagtgccgtagtgtcgaatctttgcaatga

SEQ ID NO: 50 - DNA, 45 bp, Artificial Sequence
  Description of Artificial Sequence Synthetic oligonucleotide
taaagcaggactgggcagtgccgtagtgtcgaatctttgcaatga

SEQ ID NO: 51 - DNA, 45 bp, Artificial Sequence
  Description of Artificial Sequence Synthetic oligonucleotide
tacagcaggactgggcgatgccgtagtcagcaatctttgcaatga

SEQ ID NO: 52 - DNA, 45 bp, Artificial Sequence
  Description of Artificial Sequence Synthetic oligonucleotide
tacagcaggactgggcagtgccgtagtcagcaatctttgcaatga

SEQ ID NO: 53 - DNA, 45 bp, Artificial Sequence
  Description of Artificial Sequence Synthetic oligonucleotide
taaagcaggactgggcagtgccgtagtcagcaatctttgcaatga

SEQ ID NO: 54 - DNA, 45 bp, Artificial Sequence
  Description of Artificial Sequence Synthetic oligonucleotide
tacagcattacggagcagtgccgtagtgtcgtttctttgcaatga

SEQ ID NO: 55 - DNA, 45 bp, Artificial Sequence
  Description of Artificial Sequence Synthetic oligonucleotide
tacagcaggcctgagcagtgccgtagtgtcgaatctttgcaatga

SEQ ID NO: 56 - DNA, 45 bp, Artificial Sequence
  Description of Artificial Sequence Synthetic oligonucleotide
taaagcaggactgggtagtgccgtagtgtcgaatctttgcaatga

SEQ ID NO: 57 - DNA, 45 bp, Artificial Sequence
  Description of Artificial Sequence Synthetic oligonucleotide
tacagcagtactgagcagtgccgtagtgtcgaatctttgcaatga

SEQ ID NO: 58 - DNA, 45 bp, Artificial Sequence
  Description of Artificial Sequence Synthetic oligonucleotide
tacagcaataccgagcagtgccgtagtcagcaatctttgcaatga

SEQ ID NO: 59 - DNA, 45 bp, Artificial Sequence
  Description of Artificial Sequence Synthetic oligonucleotide
tacagcagtactgggcggtgccgtagtcagcaatctttgcaatga

SEQ ID NO: 60 - DNA, 45 bp, Artificial Sequence
  Description of Artificial Sequence Synthetic oligonucleotide
tacagcaggactgggtggtgccgtagtcagcaatctttgcaatga

SEQ ID NO: 61 - DNA, 45 bp, Artificial Sequence
  Description of Artificial Sequence Synthetic oligonucleotide
taaagcaggactgagcagtgccgtagtcagcaatctttgcaatga

SEQ ID NO: 62 - DNA, 45 bp, Artificial Sequence
  Description of Artificial Sequence Synthetic oligonucleotide
taaagcaggactgagtagtgccgtagtgtcgaatctttgcaatga

SEQ ID NO: 63 - DNA, 45 bp, Artificial Sequence
  Description of Artificial Sequence Synthetic oligonucleotide
tacagcagtactgagtggtgccgtagtcagcaatctttgcaatga

SEQ ID NO: 64 - DNA, 45 bp, Artificial Sequence
  Description of Artificial Sequence Synthetic oligonucleotide
tacagcaggactgggcggtgccgtagtcagcaatctttgcaatga

SEQ ID NO: 65 - DNA, 45 bp, Artificial Sequence
  Description of Artificial Sequence Synthetic oligonucleotide
tacagcagtcctgggcggtgccgtagtcagcaatctttgcaatga

SEQ ID NO: 66 - DNA, 45 bp, Artificial Sequence
  Description of Artificial Sequence Synthetic oligonucleotide
taaagcagtactgagcagtgccgtagtgtcgaatctttgcaatga

SEQ ID NO: 67 - DNA, 45 bp, Artificial Sequence
  Description of Artificial Sequence Synthetic oligonucleotide
taaagcaggactgagcagtgccgtagtgtcgaatctttgcaatga

SEQ ID NO: 68 - DNA, 45 bp, Artificial Sequence
  Description of Artificial Sequence Synthetic oligonucleotide
tacagcaggactgggcagtgccgtagtgtcgaatctttgcaatga

SEQ ID NO: 69 - DNA, 63 bp, Artificial Sequence
  Description of Artificial Sequence Synthetic oligonucleotide
gctaaatcgattctaaagcaggacggggtggtgccgtagtgtcgtatctttgcacttacagtc

SEQ ID NO: 70 - DNA, 63 bp, Artificial Sequence
  Description of Artificial Sequence Synthetic oligonucleotide
gctaaatcgattctaaagcaggactgggtggtgccgtagtgtcgtatctttgcacttacagtc

SEQ ID NO: 71 - DNA, 63 bp, Artificial Sequence
  Description of Artificial Sequence Synthetic oligonucleotide
gctaaatcgattctaaagcaggaccgggtggtgccgtagtgtcgtatctttgcacttacagtc

SEQ ID NO: 72 - DNA, 63 bp, Artificial Sequence
  Description of Artificial Sequence Synthetic oligonucleotide
gctaaatcgattctagagcaggactgggtggtgccgtagtgtcgtatctttgcacttacagtc

SEQ ID NO: 73 - DNA, 63 bp, Artificial Sequence
  Description of Artificial Sequence Synthetic oligonucleotide
gctaaatcgattctacagcaggactgggtggtgccgtagtgtcgtatctttgcacttacagtc

SEQ ID NO: 74 - DNA, 63 bp, Artificial Sequence
  Description of Artificial Sequence Synthetic oligonucleotide
gctaaatcgattctaaagcatgactgggtggtgccgtagtcagcaatctttgcacttacagtc

SEQ ID NO: 75 - DNA, 63 bp, Artificial Sequence
  Description of Artificial Sequence Synthetic oligonucleotide
gctaaatcgattctaaagcaggactgggtggtgccgtagtcagcaatctttgcacttacagtc

SEQ ID NO: 76 - DNA, 63 bp, Artificial Sequence
  Description of Artificial Sequence Synthetic oligonucleotide
gctaaatcgattctaaagcagtactgggtggtgccgtagtcagcaatctttgcacttacagtc

SEQ ID NO: 77 - DNA, 63 bp, Artificial Sequence
  Description of Artificial Sequence Synthetic oligonucleotide
gctaaatcgattctagagcatgactgggtggtgccgtagtcagcaatctttgcacttacagtc

SEQ ID NO: 78 - DNA, 63 bp, Artificial Sequence
  Description of Artificial Sequence Synthetic oligonucleotide
gctaaatcgattctagagcaggacggggtggtgccgtagtcagcaatctttgcacttacagtc

SEQ ID NO: 79 - DNA, 45 bp, Artificial Sequence
  Description of Artificial Sequence Synthetic oligonucleotide
attctacagcacgactgagcaatgccgtattcagcaatctttgca

SEQ ID NO: 80 - DNA, 45 bp, Artificial Sequence
  Description of Artificial Sequence Synthetic oligonucleotide
attctagagcagtactgagcaatgccgtggttaccaatctttgca

SEQ ID NO: 81 - DNA, 45 bp, Artificial Sequence
  Description of Artificial Sequence Synthetic oligonucleotide
attctacagcagtagtaagctttgccgaagtgagcaatctttaca

SEQ ID NO: 82 - DNA, 45 bp, Artificial Sequence
  Description of Artificial Sequence Synthetic oligonucleotide
attctacagcacgactgagcaaggccgtagtcagcgatctttgca

SEQ ID NO: 83 - DNA, 45 bp, Artificial Sequence
  Description of Artificial Sequence Synthetic oligonucleotide
attctacagcactacggggcaatgccgaagtctgcaatctttgca

SEQ ID NO: 84 - DNA, 45 bp, Artificial Sequence
  Description of Artificial Sequence Synthetic oligonucleotide
attctacggcggtactgaccaatcccgtagttagcaatctttgca

SEQ ID NO: 85 - DNA, 45 bp, Artificial Sequence
  Description of Artificial Sequence Synthetic oligonucleotide
attctacagcggtactcatcaatgccgtagtcgtcaatttttgca

SEQ ID NO: 86 - DNA, 45 bp, Artificial Sequence
  Description of Artificial Sequence Synthetic oligonucleotide
attctaaagccgtactaagcagtaccgtagtccccaatctttgca

SEQ ID NO: 87 - DNA, 45 bp, Artificial Sequence
  Description of Artificial Sequence Synthetic oligonucleotide
attctagagccatactgttcaatgccgaagtcagcaatctttgca

SEQ ID NO: 88 - DNA, 45 bp, Artificial Sequence
  Description of Artificial Sequence Synthetic oligonucleotide
attctacagcggtactgggcaatgccgtggtctgcaatctttgca

SEQ ID NO: 89 - DNA, 45 bp, Artificial Sequence
  Description of Artificial Sequence Synthetic oligonucleotide
attctacagctgtactgaggaatgccgtctttaccaatctttgca

SEQ ID NO: 90 - DNA, 45 bp, Artificial Sequence
  Description of Artificial Sequence Synthetic oligonucleotide
attctacagcagtacttatcagtgccgttgttagcaatctttgca

SEQ ID NO: 91 - DNA, 45 bp, Artificial Sequence
  Description of Artificial Sequence Synthetic oligonucleotide
attctatagctggactgagctctgccgtagtcgtcaatctttgca

SEQ ID NO: 92 - DNA, 45 bp, Artificial Sequence
  Description of Artificial Sequence Synthetic oligonucleotide
attctacagcaatactcagcagtgccgtagtcaccaatctttgca

SEQ ID NO: 93 - DNA, 45 bp, Artificial Sequence
  Description of Artificial Sequence Synthetic oligonucleotide
attctacaacagtactgcgcaccgccgtagtctgcaatctttgca

SEQ ID NO: 94 - DNA, 45 bp, Artificial Sequence
  Description of Artificial Sequence Synthetic oligonucleotide
attctagagccgtactgcgctttgccgtagtcatcaatctttgca

SEQ ID NO: 95 - DNA, 45 bp, Artificial Sequence
  Description of Artificial Sequence Synthetic oligonucleotide
attctacagccgtactgcccaatgccgaagtaaccaatctttgca

SEQ ID NO: 96 - DNA, 45 bp, Artificial Sequence
  Description of Artificial Sequence Synthetic oligonucleotide
attctaccgtgggactgagcagtcccgtagtctgcaatctttgca

SEQ ID NO: 97 - DNA, 45 bp, Artificial Sequence
  Description of Artificial Sequence Synthetic oligonucleotide
attctacagcagtactgttcagtgcgctagtaagcaatctttgca

SEQ ID NO: 98 - DNA, 45 bp, Artificial Sequence
  Description of Artificial Sequence Synthetic oligonucleotide
attctacaggtgtactgttctatgccgttgttagccatctttgca

SEQ ID NO: 99 - DNA, 45 bp, Artificial Sequence
  Description of Artificial Sequence Synthetic oligonucleotide
attctagggcagtactgagagatgccgatgtcagcaatctttgca

SEQ ID NO: 100 - DNA, 45 bp, Artificial Sequence
  Description of Artificial Sequence Synthetic oligonucleotide
attctacagcagtattgggcagtcccgtagtcagcaatctttgca

SEQ ID NO: 101 - DNA, 45 bp, Artificial Sequence
  Description of Artificial Sequence Synthetic oligonucleotide
attctacagcaggactgggcgatcccgtagtcatcaatctttgca

SEQ ID NO: 102 - DNA, 45 bp, Artificial Sequence
  Description of Artificial Sequence Synthetic oligonucleotide
attctacagcagtactaagctatggcgtacgcaccaatctttgca

SEQ ID NO: 103 - DNA, 45 bp, Artificial Sequence
  Description of Artificial Sequence Synthetic oligonucleotide
attctacagtaggactgagcactgccgagctgggcaatctttgca

SEQ ID NO: 104 - DNA, 45 bp, Artificial Sequence
  Description of Artificial Sequence Synthetic oligonucleotide
attctaccgcagtactgagcaaagccgaggtaaacaatctttgca

SEQ ID NO: 105 - DNA, 45 bp, Artificial Sequence
  Description of Artificial Sequence Synthetic oligonucleotide
attctacagcagtactgagcaattccgttaaccgcaatctttgca

SEQ ID NO: 106 - DNA, 45 bp, Artificial Sequence
  Description of Artificial Sequence Synthetic oligonucleotide
attctacagtagtccggagctattccgtagtccgcaatctttgca

SEQ ID NO: 107 - DNA, 45 bp, Artificial Sequence
  Description of Artificial Sequence Synthetic oligonucleotide
attctacagcaggactgagcaatggagtagtcggcaatctttgca

SEQ ID NO: 108 - DNA, 45 bp, Artificial Sequence
  Description of Artificial Sequence Synthetic oligonucleotide
attctaccgcagtactacccgatcccgtagtcagcaatctttgca

SEQ ID NO: 109 - DNA, 45 bp, Artificial Sequence
  Description of Artificial Sequence Synthetic oligonucleotide
attctacaggagttctgagcattcccgtcgtcatcaatctttgca

SEQ ID NO: 110 - DNA, 45 bp, Artificial Sequence
  Description of Artificial Sequence Synthetic oligonucleotide
attctacagcagtacagaggactgccgaggtcaccaatctttgca

SEQ ID NO: 111 - DNA, 45 bp, Artificial Sequence
  Description of Artificial Sequence Synthetic oligonucleotide
attctacagaaggaccgagcagtcccgtagtcagcaatctttgca

SEQ ID NO: 112 - DNA, 45 bp, Artificial Sequence
  Description of Artificial Sequence Synthetic oligonucleotide
attctacagcattactgagcaatcccgtattaagcaatctttgca

SEQ ID NO: 113 - DNA, 45 bp, Artificial Sequence
  Description of Artificial Sequence Synthetic oligonucleotide
attctacagcagtacgaagcaatcccgccgttagcaatctttgca

SEQ ID NO: 114 - DNA, 45 bp, Artificial Sequence
  Description of Artificial Sequence Synthetic oligonucleotide
attctacagcagtactgttcaattccgatgtaagcaatctttgca

SEQ ID NO: 115 - DNA, 45 bp, Artificial Sequence
  Description of Artificial Sequence Synthetic oligonucleotide
attctacaacagtactgagctatcccgaattcaacaatctttgca

SEQ ID NO: 116 - DNA, 45 bp, Artificial Sequence
  Description of Artificial Sequence Synthetic oligonucleotide
attctacggcagttcatagcaatcccgtagtcaacaatctttgcc

SEQ ID NO: 117 - DNA, 45 bp, Artificial Sequence
  Description of Artificial Sequence Synthetic oligonucleotide
cttctacagcagttcggaggaatcccgaggtcagcaatctttgca

SEQ ID NO: 118 - DNA, 45 bp, Artificial Sequence
  Description of Artificial Sequence Synthetic oligonucleotide
attctacagctgtactaggctatcccgtagtccgcaatctttgca

SEQ ID NO: 119 - DNA, 45 bp, Artificial Sequence
  Description of Artificial Sequence Synthetic oligonucleotide
attctacagccgtactgggaaatcccgtagtctgcaatctttgca

SEQ ID NO: 120 - DNA, 45 bp, Artificial Sequence
  Description of Artificial Sequence Synthetic oligonucleotide
attctacagaagtacagagacatcccgtagttaacaatcttcgca

SEQ ID NO: 121 - DNA, 45 bp, Artificial Sequence
  Description of Artificial Sequence Synthetic oligonucleotide
attctacagccgtactgagctatcccgtagtccgcaatctttgca

SEQ ID NO: 122 - DNA, 45 bp, Artificial Sequence
  Description of Artificial Sequence Synthetic oligonucleotide
attctactgcagcaccgagcaatcccgcattccccaatctttgca

SEQ ID NO: 123 - DNA, 45 bp, Artificial Sequence
  Description of Artificial Sequence Synthetic oligonucleotide
attctacagcagtcctgagcagtaccgtagtcagcaatctttgca

SEQ ID NO: 124 - DNA, 45 bp, Artificial Sequence
  Description of Artificial Sequence Synthetic oligonucleotide
attctaaggcagtactgagccgcgccgtagtcggcaatctttgca

SEQ ID NO: 125 - DNA, 45 bp, Artificial Sequence
  Description of Artificial Sequence Synthetic oligonucleotide
gaaagtactttgatgaatacatccacggctaatgaattcctttac

SEQ ID NO: 126 - DNA, 45 bp, Artificial Sequence
  Description of Artificial Sequence Synthetic oligonucleotide
gaaagtcctttcaagaatacatccacggctatactattcctttac

SEQ ID NO: 127 - DNA, 45 bp, Artificial Sequence
  Description of Artificial Sequence Synthetic oligonucleotide
gaaagtcctttgatgcatacatccacggctaatgaattcctttac

SEQ ID NO: 128 - DNA, 45 bp, Artificial Sequence
  Description of Artificial Sequence Synthetic oligonucleotide
gaaagtcctttgatgcatacatccacggctatactattcctttac

SEQ ID NO: 129 - DNA, 45 bp, Artificial Sequence
  Description of Artificial Sequence Synthetic oligonucleotide
gaaagtcctttgaggcatacatccacggctatactattcctttac

SEQ ID NO: 130 - DNA, 45 bp, Artificial Sequence
  Description of Artificial Sequence Synthetic oligonucleotide
gaaagtcctttgaggaatacatccacggctatactattcctttac

SEQ ID NO: 131 - DNA, 45 bp, Artificial Sequence
  Description of Artificial Sequence Synthetic oligonucleotide
gaaagtcctttgaagcatacatccacggctatactattcctttac

SEQ ID NO: 132 - DNA, 45 bp, Artificial Sequence
  Description of Artificial Sequence Synthetic oligonucleotide
gaaagtactttcacgaatacatccacggctatactattcctttac

SEQ ID NO: 133 - DNA, 45 bp, Artificial Sequence
  Description of Artificial Sequence Synthetic oligonucleotide
gaaagtcctttcacgcatacatccacggctatactattcctttac

SEQ ID NO: 134 - DNA, 45 bp, Artificial Sequence
  Description of Artificial Sequence Synthetic oligonucleotide
gaaagtcctttcaagcatacatccacggctatactattcctttac

SEQ ID NO: 135 - DNA, 43 bp, Artificial Sequence
  Description of Artificial Sequence Synthetic oligonucleotide
gccccagcagcttcagttccttaatcgtcgatgtagcacagcc

SEQ ID NO: 136 - DNA, 45 bp, Artificial Sequence
  Description of Artificial Sequence Synthetic oligonucleotide
attcccatggccccagcagcttcagtccttactcgtcgatggtca

SEQ ID NO: 137 - DNA, 45 bp, Artificial Sequence
  Description of Artificial Sequence Synthetic oligonucleotide
cctctaaaaacatggctccccacagcttcagtcctttctcgtcga

SEQ ID NO: 138 - DNA, 50 bp, Artificial Sequence
  Description of Artificial Sequence Synthetic oligonucleotide
atggcctgtgcagcttcagtcccttactcgtcgatggccgtcggagctca

SEQ ID NO: 139 - DNA, 42 bp, Artificial Sequence
  Description of Artificial Sequence Synthetic oligonucleotide
agtaaaacatgtacgccccagccttcagtcccttctcgtcga

SEQ ID NO: 140 - DNA, 45 bp, Artificial Sequence
  Description of Artificial Sequence Synthetic oligonucleotide
aaaacaatactacccagcagcttcagtccacctcgtcgatggtca

SEQ ID NO: 141 - DNA, 45 bp, Artificial Sequence
  Description of Artificial Sequence Synthetic oligonucleotide
ctaatccatggctaccccagcagcttgtccctttctcgtcgatgg

SEQ ID NO: 142 - DNA, 45 bp, Artificial Sequence
  Description of Artificial Sequence Synthetic oligonucleotide
tgctaaaaacatggtgcagcagcttcagtcctgactcgtcgctgg

SEQ ID NO: 143 - DNA, 45 bp, Artificial Sequence
  Description of Artificial Sequence Synthetic oligonucleotide
aaaaacatggccccagcagcttcagtccccaactcgtcgatggtc

SEQ ID NO: 144 - DNA, 45 bp, Artificial Sequence
  Description of Artificial Sequence Synthetic oligonucleotide
aaaacatggccccagcagcttcagtccctgactcgtcgatggtca

SEQ ID NO: 145 - DNA, 44 bp, Artificial Sequence
  Description of Artificial Sequence Synthetic oligonucleotide
ctaaaaacatggcacagcagcttcagtccctccattatcgatgg

SEQ ID NO: 146 - DNA, 45 bp, Artificial Sequence
  Description of Artificial Sequence Synthetic oligonucleotide
aactacaaggccccagcagcttcatccctttactcgtcgatgtgg

SEQ ID NO: 147 - DNA, 47 bp, Artificial Sequence
  Description of Artificial Sequence Synthetic oligonucleotide
ctaaaaacatggctcccagcagcttcatatcctttcctgcttcgagg

SEQ ID NO: 148 - DNA, 45 bp, Artificial Sequence
  Description of Artificial Sequence Synthetic oligonucleotide
taaaaacatggcccccagttcagtccctaattcttatcgaatggt

SEQ ID NO: 149 - DNA, 45 bp, Artificial Sequence
  Description of Artificial Sequence Synthetic oligonucleotide
ctaaaaacgccccagcagcttcagtcccacatttctcgtcgatgg

SEQ ID NO: 150 - DNA, 45 bp, Artificial Sequence
  Description of Artificial Sequence Synthetic oligonucleotide
gccccgtcagttcagtccctgttctcgtcgatgcacagcattgcc

SEQ ID NO: 151 - DNA, 45 bp, Artificial Sequence
  Description of Artificial Sequence Synthetic oligonucleotide
ctaaaaacccatggcgacagcagcttctccccttctcgtcgatgg

SEQ ID NO: 152 - DNA, 43 bp, Artificial Sequence
  Description of Artificial Sequence Synthetic oligonucleotide
aagctatggccccagcagcttcaccctttctcgtcgtggatcc

SEQ ID NO: 153 - DNA, 45 bp, Artificial Sequence
  Description of Artificial Sequence Synthetic oligonucleotide
aaaacatggcaccccagcagcttcagcctttctcgtcgaacgtca

SEQ ID NO: 154 - DNA, 48 bp, Artificial Sequence
  Description of Artificial Sequence Synthetic oligonucleotide
acatggccccagagcttcagtgccctttctcgtcgatggtcctaagca

SEQ ID NO: 155 - DNA, 44 bp, Artificial Sequence
  Description of Artificial Sequence Synthetic oligonucleotide
ctctaaaaacatgaccccagcagcttcagtcctccgttatcgat

SEQ ID NO: 156 - DNA, 43 bp, Artificial Sequence
  Description of Artificial Sequence Synthetic oligonucleotide
acatcaggccccacagcttcagtcccttctcgtcgatggacag

SEQ ID NO: 157 - DNA, 44 bp, Artificial Sequence
  Description of Artificial Sequence Synthetic oligonucleotide
ccccagcgtcttcagtccctttctcgtcgatggtcgaccagcct

SEQ ID NO: 158 - DNA, 45 bp, Artificial Sequence
  Description of Artificial Sequence Synthetic oligonucleotide
ggccccagcagcttcagtcccagactcgtcgatggtcagcacaac

SEQ ID NO: 159 - DNA, 45 bp, Artificial Sequence
  Description of Artificial Sequence Synthetic oligonucleotide
taaaaaacatggccccagcagtcagtccctttactcgtcgatggt

SEQ ID NO: 160 - DNA, 45 bp, Artificial Sequence
  Description of Artificial Sequence Synthetic oligonucleotide
ggccagcagcttcaccaagtttctcgtcgaatatcagcacagcct

SEQ ID NO: 161 - DNA, 47 bp, Artificial Sequence
  Description of Artificial Sequence Synthetic oligonucleotide
ctaaaaacatggccccagcagcttcagtcaacttctcgattcgatgg

SEQ ID NO: 162 - DNA, 49 bp, Artificial Sequence
  Description of Artificial Sequence Synthetic oligonucleotide
tggcgccccagcagcttcatcccttctcgtcgggatgctgtcagcacag

SEQ ID NO: 163 - DNA, 113 bp, Artificial Sequence
  Description of Artificial Sequence Synthetic polynucleotide
ccctggtgtgccctctgatgtttttatccccattctacagcggtactgagcaaatccgtggtcagcaatctttgcaatgatggcagcattgggatacagtgtgaagagcagca

SEQ ID NO: 164 - DNA, 113 bp, Artificial Sequence
  Description of Artificial Sequence Synthetic polynucleotide
ccctggtgtgccctctgatgttttttaggggattctacagcggtactgagcaaatccgtggtcagcaatcaaacgtatgatggcagcattgggatacagtgtgaagagcagca

SEQ ID NO: 165 - DNA, 113 bp, Artificial Sequence
  Description of Artificial Sequence Synthetic polynucleotide
ccctggtgtgccctctgatgtttttatccccattctacatctgtagtgagcaattccgtgctcagcaatctttgcaatgatggcagcattgggatacagtgtgaagagcagca

SEQ ID NO: 166 - DNA, 113 bp, Artificial Sequence
  Description of Artificial Sequence Synthetic polynucleotide
ccctggtgtgccctctgatgttttttaggggattctacatctgtagtgagcaattccgtgctcagcaatcaaacgtatgatggcagcattgggatacagtgtgaagagcagca

SEQ ID NO: 167 - DNA, 113 bp, Artificial Sequence
  Description of Artificial Sequence Synthetic polynucleotide
ccctggtgtgccctctgatgtttttatccccattctacacctggactgagaaatcccgtactcagcaatctttgcaatgatggcagcattgggatacagtgtgaagagcagca

SEQ ID NO: 168 - DNA, 113 bp, Artificial Sequence
  Description of Artificial Sequence Synthetic polynucleotide
ccctggtgtgccctctgatgttttttaggggattctacacctggactgagaaatcccgtactcagcaatcaaacgtatgatggcagcattgggatacagtgtgaagagcagca

SEQ ID NO: 169 - DNA, 113 bp, Artificial Sequence
  Description of Artificial Sequence Synthetic polynucleotide
ccctggtgtgccctctgatgtttttatccccattctacagcagtacggtgcagtgccgtggtcagcaatctttgcaatgatggcagcattgggatacagtgtgaagagcagca

SEQ ID NO: 170 - DNA, 113 bp, Artificial Sequence
  Description of Artificial Sequence Synthetic polynucleotide
ccctggtgtgccctctgatgttttttaggggattctacagcagtacggtgcagtgccgtggtcagcaatcaaacgtatgatggcagcattgggatacagtgtgaagagcagca

SEQ ID NO: 171 - DNA, 113 bp, Artificial Sequence
  Description of Artificial Sequence Synthetic polynucleotide
ccctggtgtgccctctgatgtttttatcccccttctacagcagtagtgagttttgccgtggtcagcaatctttgcaatgatggcagcattgggatacagtgtgaagagcagca

SEQ ID NO: 172 - DNA, 113 bp, Artificial Sequence
  Description of Artificial Sequence Synthetic polynucleotide
ccctggtgtgccctctgatgttttttaggggcttctacagcagtagtgagttttgccgtggtcagcaatcaaacgtatgatggcagcattgggatacagtgtgaagagcagca

SEQ ID NO: 173 - DNA, 113 bp, Artificial Sequence
  Description of Artificial Sequence Synthetic polynucleotide
ccctggtgtgccctctgatgtttttatccccattctacagcagtcctgtacagtgccgtgatcagcaatctttgcaatgatggcagcattgggatacagtgtgaagagcagca

SEQ ID NO: 174 - DNA, 113 bp, Artificial Sequence
  Description of Artificial Sequence Synthetic polynucleotide
ccctggtgtgccctctgatgttttttaggggattctacagcagtcctgtacagtgccgtgatcagcaatcaaacgtatgatggcagcattgggatacagtgtgaagagcagca

SEQ ID NO: 175 - DNA, 113 bp, Artificial Sequence
  Description of Artificial Sequence Synthetic polynucleotide
ccctggtgtgccctctgatgtttttatccccattcgcctgaggtactgaccaatcccgtagttagcctaggctgcaatgatggcagcattgggatacagtgtgaagagcagca

SEQ ID NO: 176 - DNA, 113 bp, Artificial Sequence
  Description of Artificial Sequence Synthetic polynucleotide
ccctggtgtgccctctgatgtttttctacagattctacagcagtacagaggactgccgaggtcaccaatctttgcaataacctgagcattgggatacagtgtgaagagcagca

SEQ ID NO: 177 - DNA, 113 bp, Artificial Sequence
  Description of Artificial Sequence Synthetic polynucleotide
ccctggtgtgccctctgatgttttttaggggattctacagcagtacagaggactgccgaggtcaccaatctttgcatactacgcagcattgggatacagtgtgaagagcagca

SEQ ID NO: 178 - DNA, 113 bp, Artificial Sequence
  Description of Artificial Sequence Synthetic polynucleotide
ccctggtgtgccctctgatgtttttcgggagattctacagcagtacagaggactgccgaggtcaccaatcttgtctaggatggcagcattgggatacagtgtgaagagcagca

SEQ ID NO: 179 - DNA, 113 bp, Artificial Sequence
  Description of Artificial Sequence Synthetic polynucleotide
ccctggtgtgccctctgatgtttttatggaaagtctacagcagtacagaggactgccgaggtcactttgatttgcaatgatggcagcattgggatacagtgtgaagagcagca

SEQ ID NO: 180 - DNA, 113 bp, Artificial Sequence
  Description of Artificial Sequence Synthetic polynucleotide
ccctggtgtgccctctgatgtttttcgaagaattctacagtaggactgagcactgccgagctgggcaatctttgcaatcaaaaaagcattgggatacagtgtgaagagcagca

SEQ ID NO: 181 - DNA, 113 bp, Artificial Sequence
  Description of Artificial Sequence Synthetic polynucleotide
ccctggtgtgccctctgatgttttttaggggattctacagtaggactgagcactgccgagctgggcaatctttgcatactacgcagcattgggatacagtgtgaagagcagca

SEQ ID NO: 182 - DNA, 113 bp, Artificial Sequence
  Description of Artificial Sequence Synthetic polynucleotide
ccctggtgtgccctctgatgtttttctacagattctacagtaggactgagcactgccgagctgggcaatcttcatttggatggcagcattgggatacagtgtgaagagcagca

SEQ ID NO: 183 - DNA, 113 bp, Artificial Sequence
  Description of Artificial Sequence Synthetic polynucleotide
ccctggtgtgccctctgatgtttttatggggtgtctacagtaggactgagcactgccgagctgggcaatcttgagtcggatggcagcattgggatacagtgtgaagagcagca

SEQ ID NO: 184 - DNA, 113 bp, Artificial Sequence
  Description of Artificial Sequence Synthetic polynucleotide
ccctggtgtgccctctgatgtttttatccgaaacatacagtaggactgagcactgccgagctgggcaatcttctcccagatggcagcattgggatacagtgtgaagagcagca

SEQ ID NO: 185 - DNA, 113 bp, Artificial Sequence
  Description of Artificial Sequence Synthetic polynucleotide
ccctggtgtgccctctgatgtttttcatgagattctacagtaggactgagcactgccgagctgggcctcaggtgcaatgatggcagcattgggatacagtgtgaagagcagca

SEQ ID NO: 186 - DNA, 113 bp, Artificial Sequence
  Description of Artificial Sequence Synthetic polynucleotide
ccctggtgtgccctctgatgttttttacagaattctacagtaggactgagcactgccgagctggggttcctttgcaatgatggcagcattgggatacagtgtgaagagcagca

SEQ ID NO: 187 - DNA, 113 bp, Artificial Sequence
  Description of Artificial Sequence Synthetic polynucleotide
ccctggtgtgccctctgatgttttttaggggattctaccgcagtactacccgatcccgtagtcagcaatctttgcatactacgcagcattgggatacagtgtgaagagcagca

SEQ ID NO: 188 - DNA, 113 bp, Artificial Sequence
  Description of Artificial Sequence Synthetic polynucleotide
ccctggtgtgccctctgatgtttttatccccattctagggcagtagggtgcactgccgtggtgggcaatctttgcaatgatggcagcattgggatacagtgtgaagagcagca

SEQ ID NO: 189 - DNA, 113 bp, Artificial Sequence
  Description of Artificial Sequence Synthetic polynucleotide
ccctggtgtgccctctgatgttttttaggggattctagggcagtagggtgcactgccgtggtgggcaatcaaacgtatgatggcagcattgggatacagtgtgaagagcagca

SEQ ID NO: 190 - DNA, 113 bp, Artificial Sequence
  Description of Artificial Sequence Synthetic polynucleotide
ccctggtgtgccctctgatgtttttatccccattctaccgctgtgctgggcaatcccgtggtcagcaatctttgcaatgatggcagcattgggatacagtgtgaagagcagca

SEQ ID NO: 191 - DNA, 113 bp, Artificial Sequence
  Description of Artificial Sequence Synthetic polynucleotide
ccctggtgtgccctctgatgttttttaggggattctaccgctgtgctgggcaatcccgtggtcagcaatctttgcatactacgcagcattgggatacagtgtgaagagcagca

SEQ ID NO: 192 - DNA, 113 bp, Artificial Sequence
  Description of Artificial Sequence Synthetic polynucleotide
ccctggtgtgccctctgatgttttttaggggattctaccgctgtgctgggcaatcccgtggtcagcaatcaaacgtatgatggcagcattgggatacagtgtgaagagcagca

SEQ ID NO: 193 - DNA, 113 bp, Artificial Sequence
  Description of Artificial Sequence Synthetic polynucleotide
ccctggtgtgccctctgatgtttttatccccattctacaacagtacggtgaagtgccgtggtcagcaatctttgcaatgatggcagcattgggatacagtgtgaagagcagca

SEQ ID NO: 194 - DNA, 113 bp, Artificial Sequence
  Description of Artificial Sequence Synthetic polynucleotide
ccctggtgtgccctctgatgttttttaggggattctacaacagtacggtgaagtgccgtggtcagcaatcaaacgtatgatggcagcattgggatacagtgtgaagagcagca

SEQ ID NO: 195 - DNA, 113 bp, Artificial Sequence
  Description of Artificial Sequence Synthetic polynucleotide
ccctggtgtgccctctgatgtttttctagatattctacggcagttcatagcaatcccgtagtcaacaatctttgccattatttgagcattgggatacagtgtgaagagcagca

SEQ ID NO: 196 - DNA, 113 bp, Artificial Sequence
  Description of Artificial Sequence Synthetic polynucleotide
ccctggtgtgccctctgatgtttttcggaggattctacggcagttcatagcaatcccgtagtcaacaagggtccccatgatggcagcattgggatacagtgtgaagagcagca

SEQ ID NO: 197 - DNA, 113 bp, Artificial Sequence
  Description of Artificial Sequence Synthetic polynucleotide
ccctggtgtgccctctgatgtttttatccccatcaccctgcagttcatagcaatcccgtagtcaacaacaatatccatgatggcagcattgggatacagtgtgaagagcagca

SEQ ID NO: 198 - DNA, 113 bp, Artificial Sequence
  Description of Artificial Sequence Synthetic polynucleotide
ccctggtgtgccctctgatgtttttctggtgattctacaacagtactgagctatcccgaattcaacaatctttgcaataagcgaagcattgggatacagtgtgaagagcagca

SEQ ID NO: 199 - DNA, 113 bp, Artificial Sequence
  Description of Artificial Sequence Synthetic polynucleotide
ccctggtgtgccctctgatgtttttatccccattcgaaaaaagtactgagctatcccgaattcaacaatctttgcaatcaatcaagcattgggatacagtgtgaagagcagca

SEQ ID NO: 200 - DNA, 113 bp, Artificial Sequence
  Description of Artificial Sequence Synthetic polynucleotide
ccctggtgtgccctctgatgtttttatccccattcgaataaagtactgagctatcccgaattcaacaatcttctattagatggcagcattgggatacagtgtgaagagcagca

SEQ ID NO: 201 - DNA, 113 bp, Artificial Sequence
  Description of Artificial Sequence Synthetic polynucleotide
ccctggtgtgccctctgatgtttttatccccattctacagcagtagtgtgcagtgccgtggtcatcaatctttgcaatgatggcagcattgggatacagtgtgaagagcagca

SEQ ID NO: 202 - DNA, 113 bp, Artificial Sequence
  Description of Artificial Sequence Synthetic polynucleotide
ccctggtgtgccctctgatgttttttaggggattctacagcagtagtgtgcagtgccgtggtcatcaatcaaacgtatgatggcagcattgggatacagtgtgaagagcagca

SEQ ID NO: 203 - DNA, 113 bp, Artificial Sequence
  Description of Artificial Sequence Synthetic polynucleotide
ccctggtgtgccctctgatgtttttcagtagcttctacagcagttcggaggaatcccgaggtcagcaatctttgcaataagagaagcattgggatacagtgtgaagagcagca

SEQ ID NO: 204 - DNA, 113 bp, Artificial Sequence
  Description of Artificial Sequence Synthetic polynucleotide
ccctggtgtgccctctgatgttttttaggggcttctacagcagttcggaggaatcccgaggtcagcaatctttgcatactacgcagcattgggatacagtgtgaagagcagca

SEQ ID NO: 205 - DNA, 113 bp, Artificial Sequence
  Description of Artificial Sequence Synthetic polynucleotide
ccctggtgtgccctctgatgtttttatggaacgtctacagcagttcggaggaatcccgaggtcagcaatctttggtagcttggcagcattgggatacagtgtgaagagcagca

SEQ ID NO: 206 - DNA, 113 bp, Artificial Sequence
  Description of Artificial Sequence Synthetic polynucleotide
ccctggtgtgccctctgatgtttttatccagcgggtacagcagttcggaggaatcccgaggtcagcaatctttggtcttctggcagcattgggatacagtgtgaagagcagca

SEQ ID NO: 207 - DNA, 113 bp, Artificial Sequence
  Description of Artificial Sequence Synthetic polynucleotide
ccctggtgtgccctctgatgtttcaacggcccttctacagcagttcggaggaatcccgaggtcagcaatcttaccctggatggcagcattgggatacagtgtgaagagcagca

SEQ ID NO: 208 - DNA, 113 bp, Artificial Sequence
  Description of Artificial Sequence Synthetic polynucleotide
ccctggtgtgccctctgatgtttttattgagcctctacagcagttcggaggaatcccgaggtcagcaatcttaaattagatggcagcattgggatacagtgtgaagagcagca

SEQ ID NO: 209 - DNA, 113 bp, Artificial Sequence
  Description of Artificial Sequence Synthetic polynucleotide
ccctggtgtgccctctgatgttttttaggggcttctacagcagttcggaggaatcccgaggtcagcaatcaaacgtatgatggcagcattgggatacagtgtgaagagcagca

SEQ ID NO: 210 - DNA, 113 bp, Artificial Sequence
  Description of Artificial Sequence Synthetic polynucleotide
ccctggtgtgccctctgatgtttttatgaggcgtctacagcagttcggaggaatcccgaggtcagcaagactaacaatgatggcagcattgggatacagtgtgaagagcagca

SEQ ID NO: 211 - DNA, 113 bp, Artificial Sequence
  Description of Artificial Sequence Synthetic polynucleotide
ccctggtgtgccctctgatgtttttattgagcctctacagcagttcggaggaatcccgaggtcagcttagcctgcaatgatggcagcattgggatacagtgtgaagagcagca

SEQ ID NO: 212 - DNA, 45 bp, Artificial Sequence
  Description of Artificial Sequence Synthetic oligonucleotide
attctacagcagtactgagcaatgccgtagtcagcaatctttgca

SEQ ID NO: 213 - RNA, 45 nt, Artificial Sequence
  Description of Artificial Sequence Synthetic oligonucleotide
guggaauaguauaacaauaugcuaaauguuguuauaguaucccac

SEQ ID NO: 214 - RNA, 10 nt, Artificial Sequence
  Description of Artificial Sequence Synthetic oligonucleotide
cuacagcauu

SEQ ID NO: 215 - RNA, 10 nt, Artificial Sequence
  Description of Artificial Sequence Synthetic oligonucleotide
uaucccgaau

SEQ ID NO: 216 - DNA, 113 bp, Artificial Sequence
  Description of Artificial Sequence Synthetic polynucleotide
ccctggtgtgccctctgatgtatgttcccccattctacagcagtacagaggactgccgaggtcaccaacgctaacaatgatggcagcattgggatacagtgtgaagagcagca

SEQ ID NO: 217 - DNA, 45 bp, Artificial Sequence
  Description of Artificial Sequence Synthetic oligonucleotide
agcagtggacaggctgtgtgtcgaagttcgccctggagaggtggg

SEQ ID NO: 218 - DNA, 46 bp, Artificial Sequence
  Description of Artificial Sequence Synthetic oligonucleotide
gagcagtggacaggctgtgtgtcgaagttcgccctggagaggtggg

SEQ ID NO: 219 - RNA, 165 nt, Artificial Sequence
  Description of Artificial Sequence Synthetic polynucleotide
uugugaaagaaggacgggucccuucucuccgucauugcaaagauugcugacuacagcauugcucaguacugcuguaauaugcuaaauguuacagcaggacugagcagugccguagugucgaaucuuugcaaugaccuucucuccgccugugacuaacugugccaa

SEQ ID NO: 220 - DNA, 165 bp, Artificial Sequence
  Description of Artificial Sequence Synthetic polynucleotide
ttgtgaaagaaggacgggtcccttctctccgtcattgcaaagattgctgactacagcattgctcagtactgctgtaatatgctaaatgttacagcaggactgagcagtgccgtagtgtcgaatctttgcaatgaccttctctccgcctgtgactaactgtgccaa

SEQ ID NO: 221 - RNA, 165 nt, Artificial Sequence
  Description of Artificial Sequence Synthetic polynucleotide
uugugaaagaaggacgggucgugguuguucgucauugcaaagauugcugacuacagcauugcucaguacugcuguaauaugcuaaauguuacagcaguacugagcagugccguagugucguuucuuugcaaugagugguuguucgccugugacuaacugugccaa

SEQ ID NO: 222 - DNA, 165 bp, Artificial Sequence
  Description of Artificial Sequence Synthetic polynucleotide
ttgtgaaagaaggacgggtcgtggttgttcgtcattgcaaagattgctgactacagcattgctcagtactgctgtaatatgctaaatgttacagcagtactgagcagtgccgtagtgtcgtttctttgcaatgagtggttgttcgcctgtgactaactgtgccaa

SEQ ID NO: 223 - RNA, 165 nt, Artificial Sequence
  Description of Artificial Sequence Synthetic polynucleotide
uugugaaagaaggacgggucccucuuguucgucauugcaaagauugcugacuacagcauugcucaguacugcuguaauaugcuaaauguuacagcaguacuggguggugccguagucagcaaucuuugcaaugaccucuuguucgccugugacuaacugugccaa

SEQ ID NO: 224 - DNA, 165 bp, Artificial Sequence
  Description of Artificial Sequence Synthetic polynucleotide
ttgtgaaagaaggacgggtccctcttgttcgtcattgcaaagattgctgactacagcattgctcagtactgctgtaatatgctaaatgttacagcagtactgggtggtgccgtagtcagcaatctttgcaatgacctcttgttcgcctgtgactaactgtgccaa

SEQ ID NO: 225 - DNA, 45 bp, Artificial Sequence
  Description of Artificial Sequence Synthetic oligonucleotide
tcattgcaaagattgctgactacagcattgctcagtactgctgta

SEQ ID NO: 226 - RNA, 165 nt, Artificial Sequence
  Description of Artificial Sequence Synthetic polynucleotide
uugugaaagaaggacgggucggcgugucuugagcaguggacaggcugugugucgaaguucgcccuggagaggugggauaugcuaaaugucccaccucuccaggacgaacucggacacacaggcuguccacugcuggcgugucuugccugugacuaacugugccaa

SEQ ID NO: 227 - DNA, 165 bp, Artificial Sequence
  Description of Artificial Sequence Synthetic polynucleotide
ttgtgaaagaaggacgggtcggcgtgtcttgagcagtggacaggctgtgtgtcgaagttcgccctggagaggtgggatatgctaaatgtcccacctctccaggacgaactcggacacacaggctgtccactgctggcgtgtcttgcctgtgactaactgtgccaa

SEQ ID NO: 228 - RNA, 165 nt, Artificial Sequence
  Description of Artificial Sequence Synthetic polynucleotide
uugugaaagaaggacgggucgguuguuguggagcaguggacaggcugugugucgaaguucgcccuggagaggugggauaugcuaaaugucccaccucuucagggcgaucuuggacacacagccuguccacugcugguuguuguggccugugacuaacugugccaa

SEQ ID NO: 229 - DNA, 165 bp, Artificial Sequence
  Description of Artificial Sequence Synthetic polynucleotide
ttgtgaaagaaggacgggtcggttgttgtggagcagtggacaggctgtgtgtcgaagttcgccctggagaggtgggatatgctaaatgtcccacctcttcagggcgatcttggacacacagcctgtccactgctggttgttgtggcctgtgactaactgtgccaa

SEQ ID NO: 230 - RNA, 163 nt, Artificial Sequence
  Description of Artificial Sequence Synthetic polynucleotide
uugugaaagaaggacgggucggucucucuggagcaguggacaggcugugugucgaaguucgcccuggagaggugggauaugcuaaaugucccaccucuccaggacgaacucggacaacagcuguccacugcuggucucucuggccugugacuaacugugccaa

SEQ ID NO: 231 - DNA, 163 bp, Artificial Sequence
  Description of Artificial Sequence Synthetic polynucleotide
ttgtgaaagaaggacgggtcggtctctctggagcagtggacaggctgtgtgtcgaagttcgccctggagaggtgggatatgctaaatgtcccacctctccaggacgaactcggacaacagctgtccactgctggtctctctggcctgtgactaactgtgccaa

SEQ ID NO: 232 - RNA, 163 nt, Artificial Sequence
  Description of Artificial Sequence Synthetic polynucleotide
uugugaaagaaggacgggucggucucucuggagcaguggacaggcugugugucgaaguucgcccuggagaggugggauaugcuaaaugucccaccucuccgggacgaacucggacaacagauguccacugcuggucucucuggccugugacuaacugugccaa

SEQ ID NO: 233 - DNA, 163 bp, Artificial Sequence
  Description of Artificial Sequence Synthetic polynucleotide
ttgtgaaagaaggacgggtcggtctctctggagcagtggacaggctgtgtgtcgaagttcgccctggagaggtgggatatgctaaatgtcccacctctccgggacgaactcggacaacagatgtccactgctggtctctctggcctgtgactaactgtgccaa

SEQ ID NO: 234 - DNA, 46 bp, Artificial Sequence
  Description of Artificial Sequence Synthetic oligonucleotide
gataaggctgtgctgaccatcgacaagaaagggactgaagctgctg

SEQ ID NO: 235 - DNA, 32 bp, Artificial Sequence
  Description of Artificial Sequence Synthetic oligonucleotide
gaccatcgacaagaaagggactgaagctgctg

SEQ ID NO: 236 - DNA, 21 bp, Artificial Sequence
  Description of Artificial Sequence Synthetic oligonucleotide
tgctgactacagcattgctca

SEQ ID NO: 237 - DNA, 21 bp, Artificial Sequence
  Description of Artificial Sequence Synthetic oligonucleotide
ctgtgtgtcgaagttcgccct

SEQ ID NO: 238 - DNA, 21 bp, Artificial Sequence
  Description of Artificial Sequence Synthetic oligonucleotide
gaccatcgacaagaaagggac

SEQ ID NO: 239 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaauuuggacauacagccuguccacugcu

SEQ ID NO: 240 - RNA, 101 nt, Artificial Sequence
  Description of Artificial Sequence Synthetic polynucleotide
agcaguggacaggcugugugucgaaguucgcccuggagaggugggauaugcuaaaugucccaccucuccgggacgaacucggacaacaguuguccacugcu

SEQ ID NO: 241 - RNA, 101 nt, Artificial Sequence
  Description of Artificial Sequence Synthetic polynucleotide
agcaguggacaggcugugugucgaaguucgcccuggagaggugggauaugcuaaaugucccaccucuccaggacgaacucggacaacagauguccacugcu

SEQ ID NO: 242 - RNA, 101 nt, Artificial Sequence
  Description of Artificial Sequence Synthetic polynucleotide
agcaguggacaggcugugugucgaaguucgcccuggagaggugggauaugcuaaaugucccaccucuccggggcgaacucggacaacaguuguccacugcu

SEQ ID NO: 243 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaauuuggucacacagccuguccacugcu

SEQ ID NO: 244 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaacuuggacacacagccuguccacugcu

SEQ ID NO: 245 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaacuuggucacacagccuguccacugcu

SEQ ID NO: 246 - RNA, 101 nt, Artificial Sequence
  Description of Artificial Sequence Synthetic polynucleotide
agcaguggacaggcugugugucgaaguucgcccuggagaggugggauaugcuaaaugucccaccucuccgggacgaacucggacaacagauguccacugcu

SEQ ID NO: 247 - RNA, 103 nt, Artificial Sequence
  Description of Artificial Sequence Synthetic polynucleotide
agcaguggacaggcugugugucgaaguucgcccuggagaggugggauaugcuaaaugucccaccucuccaggacgaacucggacacacaggcuguccacugcu

SEQ ID NO: 248 - RNA, 103 nt, Artificial Sequence
  Description of Artificial Sequence Synthetic polynucleotide
agcaguggacaggcugugugucgaaguucgcccuggagaggugggauaugcuaaaugucccaccucuucagggcgaucuuggacacacagccuguccacugcu

SEQ ID NO: 249 - RNA, 101 nt, Artificial Sequence
  Description of Artificial Sequence Synthetic polynucleotide
agcaguggacaggcugugugucgaaguucgcccuggagaggugggauaugcuaaaugucccaccucuccaggacgaacucggacaacagcuguccacugcu

SEQ ID NO: 250 - RNA, 103 nt, Artificial Sequence
  Description of Artificial Sequence Synthetic polynucleotide
agcaguggacaggcugugugucgaaguucgcccuggagaggugggauaugcuaaaugucccaccucuucagggcgaacuuggacacacagccuguccacugcu

SEQ ID NO: 251 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaacuuggacauacagccuguccacugcu

SEQ ID NO: 252 - RNA, 101 nt, Artificial Sequence
  Description of Artificial Sequence Synthetic polynucleotide
agcaguggacaggcugugugucgaaguucgcccuggagaggugggauaugcuaaaugucccaccucuccgggacgaacucggacaacagcuguccacugcu

SEQ ID NO: 253 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaauuuggucauacagccuguccacugcu

SEQ ID NO: 254 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaauuuggacauacagccuguccacugcu

SEQ ID NO: 255 - RNA, 103 nt, Artificial Sequence
  Description of Artificial Sequence Synthetic polynucleotide
agcaguggacaggcugugugucgaaguucgcccuggagaggugggauaugcuaaauguccgaccucuucagggcgaucuuggacacacagccuguccacugcu

SEQ ID NO: 256 - RNA, 103 nt, Artificial Sequence
  Description of Artificial Sequence Synthetic polynucleotide
agcaguggacaggcugugugucgaaguucgcccuggagaggugggauaugcuaaaugucccaccucuucagggcgaucuuggacacacaaccuguccacugcu

SEQ ID NO: 257 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaauuuggucauacagccuguccacugcu

SEQ ID NO: 258 - RNA, 101 nt, Artificial Sequence
  Description of Artificial Sequence Synthetic polynucleotide
agcaguggacaggcugugugucgaaguucgcccuggagaggugggauaugcuaaaugucccaccucuccaggacgaacucggacaacaguuguccacugcu

SEQ ID NO: 259 - RNA, 103 nt, Artificial Sequence
  Description of Artificial Sequence Synthetic polynucleotide
agcaguggacaggcugugugucgaaguucgcccuggagaggugggauaugcuaaauguccgaccucuccagggcgaucuuggacacacagccuguccacugcu

SEQ ID NO: 260 - RNA, 103 nt, Artificial Sequence
  Description of Artificial Sequence Synthetic polynucleotide
agcaguggacaggcugugugucgaaguucgcccuggagaggugggauaugcuaaauguccgaccucuucagggcgaucuuggacacacaaccuguccacugcu

SEQ ID NO: 261 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaacuuggacauacagccuguccacugcu

SEQ ID NO: 262 - RNA, 103 nt, Artificial Sequence
  Description of Artificial Sequence Synthetic polynucleotide
agcaguggacaggcugugugucgaaguucgcccuggagaggugggauaugcuaaauguccgaccucuucagggcgaacucggacacacagccuguccacugcu

SEQ ID NO: 263 - RNA, 103 nt, Artificial Sequence
  Description of Artificial Sequence Synthetic polynucleotide
agcaguggacaggcugugugucgaaguucgcccuggagaggugggauaugcuaaaugucccaccucuccgggacgaacucggacacacaggcuguccacugcu

SEQ ID NO: 264 - RNA, 101 nt, Artificial Sequence
  Description of Artificial Sequence Synthetic polynucleotide
agcaguggacaggcugugugucgaaguucgcccuggagaggugggauaugcuaaaugucccaccucuccgggacgaacucggacaacagguguccacugcu

SEQ ID NO: 265 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaauuuggacacacagccuguccacugcu

SEQ ID NO: 266 - RNA, 104 nt, Artificial Sequence
  Description of Artificial Sequence Synthetic polynucleotide
agcaguggacaggcugugugucgaaguucgcccuggagaggugggauaugcuaaaugucccaccgcuccagggcgaacuuaucacauacagccuguccacugcu

SEQ ID NO: 267 - RNA, 103 nt, Artificial Sequence
  Description of Artificial Sequence Synthetic polynucleotide
agcaguggacaggcugugugucgaaguucgcccuggagaggugggauaugcuaaaugucccaccucuccagggcgaacuuggucauacagccuguccacugcu

SEQ ID NO: 268 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaauuuggacacacagccuguccacugcu

SEQ ID NO: 269 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaacuuggucauacagccuguccacugcu

SEQ ID NO: 270 - RNA, 103 nt, Artificial Sequence
  Description of Artificial Sequence Synthetic polynucleotide
agcaguggacaggcugugugucgaaguucgcccuggagaggugggauaugcuaaaugucccaccgcuccagggcgaauuuggucacacagccuguccacugcu

SEQ ID NO: 271 - RNA, 103 nt, Artificial Sequence
  Description of Artificial Sequence Synthetic polynucleotide
ucauugcaaagauugcugacuacagcauugcucaguacugcuguaauaugcuaaauguuacagcaauaccgagcagugccguagucagcaaucuuugcaauga

SEQ ID NO: 272 - RNA, 103 nt, Artificial Sequence
  Description of Artificial Sequence Synthetic polynucleotide
ucauugcaaagauugcugacuacagcauugcucaguacugcuguaauaugcuaaauguuacagcaguacugagcagugccguagugucguuucuuugcaauga

SEQ ID NO: 273 - RNA, 101 nt, Artificial Sequence
  Description of Artificial Sequence Synthetic polynucleotide
ucauugcaaagauugcugacuacagcauugcucaguacugcuguaauaugcuaaauguuacagcauuacggagcagugccguaguguuuucuuugcaauga

SEQ ID NO: 274 - RNA, 101 nt, Artificial Sequence
  Description of Artificial Sequence Synthetic polynucleotide
ucauugcaaagauugcugacuacagcauugcucaguacugcuguaauaugcuaaauguuacagcauuacugagcagugccguaguguuuucuuugcaauga

SEQ ID NO: 275 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacugagcagugccguagucagcaaucuuugcaauga

SEQ ID NO: 276 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacugaguagugccguagucagcaaucuuugcaauga

SEQ ID NO: 277 - RNA, 103 nt, Artificial Sequence
  Description of Artificial Sequence Synthetic polynucleotide
ucauugcaaagauugcugacuacagcauugcucaguacugcuguaauaugcuaaauguuaaagcaguacugagcagugccguagucagcaaucuuugcaauga

SEQ ID NO: 278 - RNA, 103 nt, Artificial Sequence
  Description of Artificial Sequence Synthetic polynucleotide
ucauugcaaagauugcugacuacagcauugcucaguacugcuguaauaugcuaaauguuaaagcaguacugagcggugccguagucagcaaucuuugcaauga

SEQ ID NO: 279 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugagcagugccguagucagcaaucuuugcaauga

SEQ ID NO: 280 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugagcggugccguagucagcaaucuuugcaauga

SEQ ID NO: 281 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugggcagugccguagucagcaaucuuugcaauga

SEQ ID NO: 282 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugggcgaugccguagucagcaaucuuugcaauga

SEQ ID NO: 283 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugggcggugccguagucagcaaucuuugcaauga

SEQ ID NO: 284 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugggugaugccguagucagcaaucuuugcaauga

SEQ ID NO: 285 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacuggguggugccguagucagcaaucuuugcaauga

SEQ ID NO: 286 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacugggcagugccguagucagcaaucuuugcaauga

SEQ ID NO: 287 - RNA, 103 nt, Artificial Sequence
  Description of Artificial Sequence Synthetic polynucleotide
ucauugcaaagauugcugacuacagcauugcucaguacugcuguaauaugcuaaauguuacagcaguacugagcggugccguagucagcaaucuuugcaauga

SEQ ID NO: 288 - RNA, 103 nt, Artificial Sequence
  Description of Artificial Sequence Synthetic polynucleotide
ucauugcaaagauugcugacuacagcauugcucaguacugcuguaauaugcuaaauguuacagcaguacugaguggugccguagucagcaaucuuugcaauga

SEQ ID NO: 289 - RNA, 103 nt, Artificial Sequence
  Description of Artificial Sequence Synthetic polynucleotide
ucauugcaaagauugcugacuacagcauugcucaguacugcuguaauaugcuaaauguuacagcaguacugggcggugccguagucagcaaucuuugcaauga

SEQ ID NO: 290 - RNA, 103 nt, Artificial Sequence
  Description of Artificial Sequence Synthetic polynucleotide
ucauugcaaagauugcugacuacagcauugcucaguacugcuguaauaugcuaaauguuacagcaguacuggguggugccguagucagcaaucuuugcaauga

SEQ ID NO: 291 - RNA, 103 nt, Artificial Sequence
  Description of Artificial Sequence Synthetic polynucleotide
ucauugcaaagauugcugacuacagcauugcucaguacugcuguaauaugcuaaauguuacagcaguccugggcggugccguagucagcaaucuuugcaauga

SEQ ID NO: 292 - RNA, 103 nt, Artificial Sequence
  Description of Artificial Sequence Synthetic polynucleotide
ucauugcaaagauugcugacuacagcauugcucaguacugcuguaauaugcuaaauguuacagcaguccuggguggugccguagucagcaaucuuugcaauga

SEQ ID NO: 293 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacugagcagugccguagugucgaaucuuugcaauga

SEQ ID NO: 294 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacugaguagugccguagugucgaaucuuugcaauga

SEQ ID NO: 295 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacugggcagugccguagugucgaaucuuugcaauga

SEQ ID NO: 296 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggacuggguagugccguagugucgaaucuuugcaauga

SEQ ID NO: 297 - RNA, 103 nt, Artificial Sequence
  Description of Artificial Sequence Synthetic polynucleotide
ucauugcaaagauugcugacuacagcauugcucaguacugcuguaauaugcuaaauguuaaagcaggccugagcagugccguagugucgaaucuuugcaauga

SEQ ID NO: 298 - RNA, 103 nt, Artificial Sequence
  Description of Artificial Sequence Synthetic polynucleotide
ucauugcaaagauugcugacuacagcauugcucaguacugcuguaauaugcuaaauguuaaagcaguacugagcagugccguagugucgaaucuuugcaauga

SEQ ID NO: 299 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugagcagugccguagugucgaaucuuugcaauga

SEQ ID NO: 300 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugaguagugccguagugucgaaucuuugcaauga

SEQ ID NO: 301 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacugggcagugccguagugucgaaucuuugcaauga

SEQ ID NO: 302 - RNA, 103 nt, Artificial Sequence
  Description of Artificial Sequence Synthetic polynucleotide
ucauugcaaagauugcugacuacagcauugcucaguacugcuguaauaugcuaaauguuacagcaggacuggguagugccguagugucgaaucuuugcaauga

SEQ ID NO: 303 - RNA, 103 nt, Artificial Sequence
  Description of Artificial Sequence Synthetic polynucleotide
ucauugcaaagauugcugacuacagcauugcucaguacugcuguaauaugcuaaauguuacagcaggccugagcagugccguagugucgaaucuuugcaauga

SEQ ID NO: 304 - RNA, 103 nt, Artificial Sequence
  Description of Artificial Sequence Synthetic polynucleotide
ucauugcaaagauugcugacuacagcauugcucaguacugcuguaauaugcuaaauguuacagcaguacugagcagugccguagugucgaaucuuugcaauga

SEQ ID NO: 305 - DNA, 121 bp, Artificial Sequence
  Description of Artificial Sequence Synthetic polynucleotide
gactgacgttgcaaagattgctgactacagcattgctcagtactgctgtagaatataggctaaatcgattctaaagcaggacggggtggtgccgtagtgtcgtatctttgcacttacagtc

SEQ ID NO: 306 - DNA, 121 bp, Artificial Sequence
  Description of Artificial Sequence Synthetic polynucleotide
gactgacgttgcaaagattgctgactacagcattgctcagtactgctgtagaatataggctaaatcgattctaaagcatgactgggtggtgccgtagtcagcaatctttgcacttacagtc

SEQ ID NO: 307 - DNA, 121 bp, Artificial Sequence
  Description of Artificial Sequence Synthetic polynucleotide
gactgacgttgcaaagattgctgactacagcattgctcagtactgctgtagaatataggctaaatcgattctaaagcaggactgggtggtgccgtagtgtcgtatctttgcacttacagtc

SEQ ID NO: 308 - DNA, 121 bp, Artificial Sequence
  Description of Artificial Sequence Synthetic polynucleotide
gactgacgttgcaaagattgctgactacagcattgctcagtactgctgtagaatataggctaaatcgattctaaagcaggactgggtggtgccgtagtcagcaatctttgcacttacagtc

SEQ ID NO: 309 - DNA, 121 bp, Artificial Sequence
  Description of Artificial Sequence Synthetic polynucleotide
gactgacgttgcaaagattgctgactacagcattgctcagtactgctgtagaatataggctaaatcgattctaaagcaggaccgggtggtgccgtagtgtcgtatctttgcacttacagtc

SEQ ID NO: 310 - DNA, 121 bp, Artificial Sequence
  Description of Artificial Sequence Synthetic polynucleotide
gactgacgttgcaaagattgctgactacagcattgctcagtactgctgtagaatataggctaaatcgattctaaagcagtactgggtggtgccgtagtcagcaatctttgcacttacagtc

SEQ ID NO: 311 - DNA, 121 bp, Artificial Sequence
  Description of Artificial Sequence Synthetic polynucleotide
gactgacgttgcaaagattgctgactacagcattgctcagtactgctgtagaatataggctaaatcgattctagagcaggactgggtggtgccgtagtgtcgtatctttgcacttacagtc

SEQ ID NO: 312 - DNA, 121 bp, Artificial Sequence
  Description of Artificial Sequence Synthetic polynucleotide
gactgacgttgcaaagattgctgactacagcattgctcagtactgctgtagaatataggctaaatcgattctagagcatgactgggtggtgccgtagtcagcaatctttgcacttacagtc

SEQ ID NO: 313 - DNA, 121 bp, Artificial Sequence
  Description of Artificial Sequence Synthetic polynucleotide
gactgacgttgcaaagattgctgactacagcattgctcagtactgctgtagaatataggctaaatcgattctacagcaggactgggtggtgccgtagtgtcgtatctttgcacttacagtc

SEQ ID NO: 314 - DNA, 121 bp, Artificial Sequence
  Description of Artificial Sequence Synthetic polynucleotide
gactgacgttgcaaagattgctgactacagcattgctcagtactgctgtagaatataggctaaatcgattctagagcaggacggggtggtgccgtagtcagcaatctttgcacttacagtc

SEQ ID NO: 315 - DNA, 148 bp, Artificial Sequence
  Description of Artificial Sequence Synthetic polynucleotide
taatacgactcactatagcgttgtgaaagaaggacgggtcgtaattcctcaaagtgtattttatgttttccagtgtggtgtaaaggaattcattagccatggatgtattcatgaaaggactttcaaaggccaaggagggagttgtggc

SEQ ID NO: 316 - DNA, 175 bp, Artificial Sequence
  Description of Artificial Sequence Synthetic polynucleotide
taatacgactcactatagttggcacagttagtcacaggtcgatcggccacaactccctccttggcctttgaaagtcctttgatgcatacatccacggctatacttttcctttacaccacactggaaaacataaaatacactttgaggaattacgacccgtggaagaaacacaacg

SEQ ID NO: 317 - DNA, 105 bp, Artificial Sequence
  Description of Artificial Sequence Synthetic polynucleotide
  modified_base (1)..(1): a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 318 - DNA, 105 bp, Artificial Sequence
  Description of Artificial Sequence Synthetic polynucleotide
  modified_base (105)..(105): a, c, t, g, unknown or other
agcagccacaactccgggaacccctttgaaagtcctttcccgaatacatccacggctaaacttctcctttacaccacactgtcgtcgattaggccactcccagtn

(19) United States
(12) Patent Application Publication
(10) Pub. No.: US 2024/0102083 A1
(43) Pub. Date: Mar. 28, 2024

(54) HIGH THROUGHPUT RNA-EDITING SCREENING METHODS

(71) Applicant: Shape Therapeutics Inc., Seattle, WA (US)

(72) Inventors: Brian Booth, Seattle, WA (US); Adrian Briggs, Seattle, WA (US); Richard Sullivan, Seattle, WA (US); Yiannis Savva, Seattle, WA (US); Stephen Burleigh, Seattle, WA (US); Lina Bagepalli, Seattle, WA (US)

(21) Appl. No.: 18/255,069

(22) PCT Filed: Dec. 1, 2021
(86) PCT No.: PCT/US2021/061485

§ 371 Date: May 30, 2023

Related U.S. Application Data
(60) Provisional application No. 63/120,092, filed on Dec. 1, 2020.
(60) Provisional application No. 63/153,345, filed on Feb. 24, 2021.
(60) Provisional application No. 63/178,219, filed on Apr. 22, 2021.
(60) Provisional application No. 63/183,296, filed on May 3, 2021.
(60) Provisional application No. 63/277,663, filed on Nov. 10, 2021.

(51) Int. Cl. C12Q 1/6811; C12Q 1/6816

(52) U.S. Cl. CPC C12Q 1/6811; C12Q 1/6816

(57) ABSTRACT

Provided herein is a high throughput screening method for identifying guide RNAs (gRNAs) useful for editing a target RNA, wherein the editing is mediated by an RNA editing entity (e.g., a native human adenosine deaminase enzyme for a human subject).

### CROSS REFERENCE

This application is a U.S. national stage under 35 USC 371 of PCT Application No. PCT/US2021/061485, filed Dec. 1, 2021 which claims priority under 35 U.S.C. § 119 from Provisional Application Ser. No. 63/120,092, filed Dec. 1, 2020, Provisional Application Ser. No. 63/153,345, filed Feb. 24, 2021, Provisional Application Ser. No. 63/178,219, filed Apr. 22, 2021, Provisional Application Ser. No. 63/183,296, filed May 3, 2021, and Provisional Application Ser. No. 63/277,663, filed Nov. 10, 2021, the disclosures of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Feb. 16, 2022, is named 731601 SL.txt and is 89,912 bytes in size.

### BACKGROUND

Point mutations underlie many genetic diseases. While programmable DNA nucleases have been used to repair mutations, the use of such DNA nucleases for gene therapy poses multiple challenges. In particular, the efficiency of homologous recombination is typically low in cells and an active nuclease presents a risk of introducing permanent off-target mutations. Further, prevalent programmable nucleases typically comprise elements of non-human origin raising the potential of in vivo immunogenicity. In light of these, approaches to instead directly target RNA, and use of molecular machinery native to the host, would be highly desirable.

### SUMMARY

Disclosed herein are methods for identifying a guide RNA suitable for editing a target RNA of interest. In some embodiments, a method comprises: (a) contacting a self-annealing RNA structure with an RNA editing entity, wherein the self-annealing RNA structure comprises: (i) a target RNA, (ii) a candidate guide RNA, and (iii) a linker that covalently attaches the target RNA and the candidate guide RNA, wherein the target RNA and candidate guide RNA form a guide-target RNA scaffold that comprises one or more structural features, wherein when the candidate guide RNA hybridizes to the target RNA, a hairpin loop is formed, wherein the loop of the hairpin loop comprises at least part of the linker, and wherein the contacting occurs under conditions that allow the RNA editing entity to edit a base of a nucleotide in the target RNA in the self-annealing RNA structure; and (b) identifying an edited target RNA; wherein the edited target RNA identifies a candidate guide RNA suitable for editing the target RNA. In some embodiments, the RNA editing entity is: (a) an adenosine deaminase acting on RNA (ADAR); (b) an ADAR variant; (c) a catalytically active deaminase domain; or (d) a fusion protein comprising any one of (a)-(c). In some embodiments, the ADAR comprises human ADAR (hADAR). In some embodiments, the ADAR is ADAR1 and/or ADAR2. In some embodiments, the linker is a synthetic hairpin, a GluR2 hairpin, or a HIV-1 TAR RNA hairpin. In some embodiments, the method is a high throughput method of screening, where the method employs a plurality of self-annealing RNA structures. In some embodiments, the candidate guide RNA or the plurality of candidate guide RNAs independently comprise from 1 to about 50 structural features. In some embodiments, the one or more structural features independently comprise a mismatch, a bulge, an internal loop, a hairpin, a wobble base pair, or any combination thereof. In some embodiments, the one or more structural features comprise a bulge. In some embodiments, the bulge is a symmetrical bulge. In some embodiments, the bulge is an asymmetrical bulge. In some embodiments, the bulge comprises from about 1 to about 4 nucleotides of the candidate guide RNA and from 0 to about 4 nucleotides of the target RNA. In some embodiments, the bulge comprises from 0 to about 4 nucleotides of the candidate guide RNA and from about 1 to about 4 nucleotides of the target RNA. In some embodiments, the bulge comprises 3 nucleotides of the candidate guide RNA and 3 nucleotides of the target RNA. In some embodiments, the one or more structural features comprise an internal loop. In some embodiments, the internal loop is a symmetrical internal loop. In some embodiments, the internal loop is an asymmetrical internal loop. In some embodiments, the internal loop is formed by from about 5 to about 10 nucleotides of either the candidate guide RNA or the target RNA. In some embodiments, the one or more structural features comprise a hairpin. In some embodiments, the hairpin is a non-recruitment hairpin. In some embodiments, the hairpin comprises a loop portion of from about 3 to about 15 nucleotides in length. In some embodiments, the one or more structural features comprise a mismatch. In some embodiments, the mismatch comprises a base in the candidate guide RNA opposite to and unpaired with a base in the target RNA. In some embodiments, the mismatch comprises an A/A mismatch, an A/G mismatch, an A/C mismatch, a G/G mismatch, a C/C mismatch, or a C/U mismatch. In some embodiments, the mismatch comprises an A/C mismatch and wherein the A is in the target RNA molecule and the C is in the candidate guide RNA. In some embodiments, the A in the A/C mismatch is the base of the nucleotide in the target RNA molecule chemically modified by the RNA editing entity. In some embodiments, the one or more structural features comprise a wobble base pair. In some embodiments, the wobble base pair comprises a guanine paired with a uracil. In some embodiments, each of the one or more structural features are present in a micro-footprint of each candidate guide RNA. In some embodiments, at least one of the one or more structural features are present in a micro-footprint of each candidate guide RNA. In some embodiments, at least one of the one or more structural features is present in a macro-footprint of each candidate guide RNA. In some embodiments, the macro-footprint comprises at least two structural features, wherein the at least two structural features comprise a first barbell and a second barbell, wherein the first barbell and the second barbell are adjacent to opposing ends of the micro-footprint. In some embodiments, each of the one or more structural features are present in a micro-footprint and a macro-footprint of each candidate guide RNA. In some embodiments, the macro-footprint comprises at least two structural features, wherein the at least two structural features comprise a first barbell and a second barbell, wherein the first barbell and the second barbell are adjacent to opposing ends of the micro-footprint. In some embodiments, at least one candidate guide RNA, upon hybridization to the target RNA, comprises a first 6/6 symmetrical internal loop and a second 6/6 symmetrical internal loop adjacent to one or more structural features selected from a bulge, internal loop, mismatch, and wobble base pair, wherein the first 6/6 symmetrical internal loop and the second 6/6 symmetrical internal loop each comprise 6 nucleotides of the candidate guide RNA and 6 nucleotides of the target RNA. In some embodiments, at least one candidate guide RNA further comprises at least one recruitment hairpin. In some embodiments, the recruitment hairpin is a GluR2 hairpin. In some embodiments, the target RNA of each of self-annealing RNA structures in the plurality are the same. In some embodiments, the plurality of self-annealing RNA structures comprises at least about 10 to 1×108 different self-annealing RNA structures with comprising structural features. In some embodiments, the identifying comprises sequencing amplicons derived from the plurality of self-annealing RNA structures and the self-annealing RNA structures that comprise an edited target RNA are identified based on the sequence of the amplicons. In some embodiments, the sequencing is next generation sequencing (NGS). In some embodiments, each of the self-annealing RNA structures further comprise one or more universal primer binding sites and the amplicons are derived by amplification of the self-annealing RNA structures using one or more universal primers. In some embodiments, each of the self-annealing RNA structures further comprises a barcode, and wherein the barcode is unique for each of the self-annealing RNA structures of the plurality. In some embodiments, the self-annealing RNA structure comprising the candidate guide RNA and the target RNA is as shown in FIG. 34. In some embodiments, the candidate guide RNA is at least 100 nucleotides. In some embodiments, the method further comprises generating the self-annealing RNA structure prior to the contacting of (a). In some embodiments, the self-annealing RNA structure is generated from a precursor RNA sequence that comprises: a promoter, the candidate guide RNA, a universal partial NGS adapter sequence, a barcode unique to the candidate guide RNA, a sequence comprising more than one deoxy uracil. In some embodiments, the sequence comprising more than one deoxy uracil is a sequence comprising a USER (Uracil-specific excision reagent) enzyme cleavage site. In some embodiments, the generating comprises: (a) amplifying the RNA sequence using a forward primer with complementarity to the guide RNA and a reverse primer having complementarity to the barcode unique to the candidate guide RNA and the sequence comprising more than one deoxy uracil; (b) amplifying a target sequence also comprising the sequence comprising more than one deoxy uracil with a forward primer having complementarity to the sequence comprising more than one deoxy uracil and a reverse primer having complementarity to the target sequence; (c) treating the amplified transcripts of (b) with a uracil-DNA glycosylase enzyme; and (d) contacting the amplified transcripts with each other and ligating the amplified transcripts, thereby generating the self-annealing RNA structure. In some embodiments, the uracil-DNA glycosylase enzyme is a USER (Uracil-specific excision reagent) enzyme. In some embodiments, the self-annealing RNA structure is according to the formula, from 5′- to 3′: promoter-candidate guide RNA-universal partial NGS adapter sequence-first barcode-at least a portion of a USER enzyme cleavage site-target RNA. In some embodiments, the promoter comprises a T7 promoter. In some embodiments, the self-annealing RNA structures is according to the formula, from 5′- to 3′-: UPBS1-BC1-target RNA—loop—candidate guide RNA—BC2-UPBS2, wherein UPB S1 and UPBS2 are a first universal primer binding site and a second universal primer binding site, respectively, and wherein BC1 is an optional first barcode and BC2 is an optional second bar code. In some embodiments, the self-annealing RNA structure comprises a first universal primer binding site, a second universal primer binding site, a first barcode, and a second barcode. In some embodiments, the target RNA is encoded by one of the following genes or a fragment thereof: APP, ABCA4, SERPINA1, HEXA, LRRK2, CFTR, SNCA, MAPT, DUX4, PMP22, DMPK, SOD1, GRN, LIPA, or DMD. In some embodiments, the self-annealing RNA structure is contacted with the RNA editing entity in the presence of one or more ADAR inhibitors and/or trans-regulators. In some embodiments, the self-annealing RNA structure has a length of from about 50 nucleotides to about 500 nucleotides. In some embodiments, the self-annealing RNA structure has a length of about 100. In some embodiments, the self-annealing RNA structure has a length of about 230.

Also disclosed herein are methods for identifying a guide RNA suitable for editing a target RNA of interest. In some embodiments, a method comprises (a) contacting an engineered RNA structure with an RNA editing entity, wherein the engineered RNA structure comprises: (i) a target RNA, (ii) a candidate guide RNA, and (iii) a linker that attaches the target RNA and the candidate guide RNA, wherein the target RNA and candidate guide RNA form a guide-target RNA scaffold that comprises one or more structural features, wherein the linker comprises a first barcode attached to the target RNA and a second barcode attached to the candidate guide RNA, wherein the first barcode and the second barcode are the same, and wherein the linker does not covalently attach the candidate guide RNA to the target RNA, and wherein the contacting occurs under conditions that allows the RNA editing entity to edit a base of a nucleotide in the target RNA in the plurality of the engineered RNA structures; and (b) identifying an edited target RNA; wherein the edited target RNA identifies a candidate guide RNA suitable for editing the target RNA. In some embodiments, the method is a high throughput method of screening, and wherein the method employs a plurality of self-annealing RNA structures. In some embodiments, the candidate guide RNA is at least 100 nucleotides. In some embodiments, the method further comprises generating the engineered RNA structure prior to the contacting of (a). In some embodiments, the generating comprises: (a) contacting a precursor candidate guide RNA comprising, from 5′- to 3′-: first barcode-candidate guide RNA-Universal reverse primer site; with a precursor target RNA that does not comprise a barcode, thereby forming a duplex with a 5′ overhang comprising the first barcode; (b) contacting the duplex with a DNA polymerase enzyme, thereby imprinting the second barcode and Universal reverse primer site onto the target RNA via the overhang and forming a DNA-RNA hybrid; and (c) contacting the DNA-RNA hybrid with a forward primer with complementarity to the target RNA, a reverse primer with complementarity to the universal reverse primer binding site, and a reverse transcriptase enzyme; thereby generating the RNA structure. In some embodiments, the DNA polymerase is Bst3.0. In some embodiments, the duplex with the 5′ overhang is according to the formula: strand 1: from 5′- to 3′-: UPBS1-BC1-candidate guide RNA; strand 2: from 5′- to 3′-: target RNA, wherein UPBS1 is the first universal primer binding site, and wherein BC1 is the first barcode. In some embodiments, the RNA structure after the generating is according to the formula, from 5′- to 3′-: UPBS1-BC1-candidate guide RNA, and wherein each target RNA is according to the formula, from 5′- to 3′-: target RNA-BC2-UPBS2, wherein UPBS1 and UPBS2 are a first universal primer binding site and a second universal primer binding site, respectively, and wherein BC1 is the first barcode and BC2 is the second bar code.

Also disclosed herein is a method for producing a vector encoding a guide RNA comprising: (a) contacting a plurality of self-annealing RNA structures with an RNA editing entity, wherein a self-annealing RNA structure of the plurality comprises: (i) a target RNA, (ii) a candidate guide RNA, and (iii) a linker that covalently attaches the target RNA and the candidate guide RNA, wherein the target RNA and candidate guide RNA form a guide-target RNA scaffold that comprises one or more structural features, wherein the linker forms a hairpin loop, and wherein the contacting occurs under conditions that allows the RNA editing entity to edit a target RNA in the plurality of self-annealing RNA structures; (b) identifying one or more self-annealing RNA structures in the plurality of self-annealing RNA structures that comprise an edited target RNA; wherein the one or more identified self-annealing RNA structures that comprise an edited target RNA each comprise a candidate guide RNA suitable for editing the target RNA; and (c) formulating the candidate guide RNA in a vector.

Also disclosed herein are engineered RNA structures. In some embodiments, an engineered RNA structure comprises: (i) a target RNA, (ii) a candidate guide RNA that facilitates or is configured to facilitate editing of a base of a nucleotide of the target RNA via an RNA editing entity, and (iii) a linker that attaches the target RNA and the candidate guide RNA, wherein the target RNA and candidate guide RNA form a guide-target RNA scaffold that comprises one or more structural features, and wherein the one or more structural features are substantially formed upon hybridization of the candidate guide RNA to the target RNA. In some embodiments, the engineered RNA structure is a self-annealing RNA structure, and wherein the linker covalently attaches the target RNA to the candidate guide RNA. In some embodiments, when the candidate guide RNA hybridizes to the target RNA, a hairpin loop is formed, where the loop of the hairpin loop comprises at least part of the linker. In some embodiments, the engineered RNA structure is according to the formula, from 5′- to 3′: promoter-candidate guide RNA-universal partial NGS adapter sequence-first barcode-at least a portion of a USER enzyme cleavage site-target RNA. In some embodiments, the linker comprises a first barcode attached to the target RNA and a second barcode attached to the candidate guide RNA, wherein the first barcode has complementarity with the second barcode. In some embodiments, the linker does not covalently attach the candidate guide RNA to the target RNA. In some embodiments, the linker comprises one or more universal primer binding sites. In some embodiments, the candidate guide RNA is according to the formula, from 5′- to 3′-: UPBS1-BC1-candidate guide RNA, and wherein each target RNA is according to the formula, from 5′- to 3′-: target RNA-BC2-UPBS2, wherein UPBS1 and UPBS2 are a first universal primer binding site and a second universal primer binding site, respectively, and wherein BC1 is the first barcode and BC2 is the second bar code. In some embodiments, the candidate guide RNA is generated from a duplex with the 5′ overhang according to the formula: strand 1: from 5′- to 3′-: UPBS1-BC1-candidate guide RNA; strand 2: from 5′- to 3′-: target RNA, wherein UPBS1 is the first universal primer binding site, and wherein BC1 is the first barcode. In some embodiments, the one or more structural features of the engineered RNA structure comprises a mismatch, a bulge, an internal loop, a hairpin, a mismatch, a wobble base pair, or any combination thereof. In some embodiments, the one or more structural features comprise a bulge. In some embodiments, the bulge is a symmetrical bulge. In some embodiments, the bulge is an asymmetrical bulge. In some embodiments, the bulge comprises from about 1 to about 4 nucleotides of the candidate guide RNA and from about 0 to about 4 nucleotides of the target RNA. In some embodiments, the bulge comprises from about 0 to about 4 nucleotides of the candidate guide RNA and from about 1 to about 4 nucleotides of the target RNA. In some embodiments, the bulge comprises 3 nucleotides of the candidate guide RNA and 3 nucleotides of the target RNA. In some embodiments, the one or more structural features comprise an internal loop. In some embodiments, the internal loop is a symmetrical internal loop. In some embodiments, the internal loop is an asymmetrical internal loop. In some embodiments, the internal loop is formed by from about 5 to about 10 nucleotides of either the candidate guide RNA or the target RNA. In some embodiments, the one or more structural features comprise a hairpin. In some embodiments, the hairpin is a non-recruitment hairpin. In some embodiments, the hairpin comprises a loop portion of from about 3 to about 15 nucleotides in length. In some embodiments, the one or more structural features comprise a mismatch. In some embodiments, the mismatch comprises a base in the candidate guide RNA opposite to and unpaired with a base in the target RNA. In some embodiments, the mismatch comprises an A/A mismatch, an A/G mismatch, an A/C mismatch, a G/G mismatch, a C/C mismatch, or a C/U mismatch. In some embodiments, the mismatch comprises an A/C mismatch and wherein the A is in the target RNA molecule and the C is in the candidate guide RNA. In some embodiments, the A in the A/C mismatch is the base of the nucleotide in the target RNA that is edited by the RNA editing entity. In some embodiments, the one or more structural features comprise a wobble base pair. In some embodiments, the wobble base pair comprises a guanine paired with a uracil. In some embodiments, each of the one or more structural features are present in a micro-footprint of the candidate guide RNA. In some embodiments, at least one of the one or more structural features are present in a micro-footprint of the candidate guide RNA. In some embodiments, at least one of the one or more structural features is present in a macro-footprint of the candidate guide RNA. In some embodiments, the macro-footprint comprises at least two structural features, wherein the at least two structural features comprise a first barbell and a second barbell, wherein the first barbell and the second barbell flank opposing ends of the micro-footprint. In some embodiments, each of the one or more structural features are present in a micro-footprint and a macro-footprint of the candidate guide RNA. In some embodiments, the macro-footprint comprises at least two structural features, wherein the at least two structural features comprise a first barbell and a second barbell, wherein the first barbell and the second barbell flank opposing ends of the micro-footprint. In some embodiments, the candidate guide RNA, upon hybridization to the target RNA, comprises a first 6/6 symmetrical internal loop and a second 6/6 symmetrical internal loop flanking one or more structural features selected from a bulge, internal loop, mismatch, and wobble base pair, wherein the first 6/6 symmetrical internal loop and the second 6/6 symmetrical internal loop each comprise 6 nucleotides of the candidate guide RNA and 6 nucleotides of the target RNA. In some embodiments, at least one candidate guide RNA further comprises at least one recruitment hairpin. In some embodiments, the recruitment hairpin is a GluR2 hairpin. In some embodiments, the RNA editing entity is; (a) an ADAR; (b) a variant ADAR; (c) a catalytically active deaminase domain; or (d) a fusion protein comprising any one of (a)-(c). In some embodiments, the engineered RNA structure has a length of from about 50 nucleotides to about 500 nucleotides. In some embodiments, the engineered RNA structure has a length of about 100 nucleotides. In some embodiments, the candidate guide RNA has a length of about 45 nucleotides. In some embodiments, the engineered RNA structure has a length of about 230 nucleotides. In some embodiments, the candidate guide RNA has a length of about 100 nucleotides.

Also disclosed herein is a library of engineered RNA structures comprising a plurality of engineered RNA structures as disclosed herein, where the plurality of engineered RNA structures comprises from 10 to 1×10⁸ different engineered RNA structures comprising different structural features.

Also disclosed herein are methods of screening a macro-footprint sequence in a candidate guide RNA having a micro-footprint sequence that configures the candidate guide RNA to facilitate an edit of a base in a target RNA via an RNA editing entity, wherein: (a) the candidate guide RNA hybridizes to a sequence of a target RNA; and (b) upon hybridization, the candidate guide RNA and the sequence of the target RNA form a guide-target RNA scaffold, wherein the guide-target RNA scaffold comprises: a micro-footprint that comprises at least one structural feature selected from the group consisting of: a bulge, an internal loop, a mismatch, a hairpin, and any combination thereof, wherein, upon contacting the guide-target RNA scaffold with an RNA editing entity, the RNA editing entity edits an on-target adenosine in the target RNA within the guide-target RNA scaffold. In some embodiments, the method comprises: inserting a first sequence and a second sequence into the candidate guide RNA that, when the candidate guide RNA is hybridized to the target RNA, form a first internal loop and a second internal loop, respectively, on opposing ends of the micro-footprint; wherein the first internal loop and the second internal loop facilitate an increase in the amount of the editing of the on-target adenosine in the target RNA relative to an otherwise comparable candidate guide RNA lacking the first internal loop and the second internal loop, thereby improving the editing efficiency of the candidate guide RNA. In some embodiments, the method further comprises screening a position of the first internal loop, relative to the on-target adenosine of the micro-footprint. In some embodiments, the first internal loop is positioned about 2 bases to about 20 bases upstream of the on-target adenosine. In some embodiments, the method further comprises screening a position of the second internal loop, relative to the on-target adenosine of the micro-footprint. In some embodiments, the second internal loop is positioned about 12 bases to about 40 bases downstream of the on-target adenosine. In some embodiments, the method further comprises optimizing the number of bases of the candidate guide RNA and the target RNA that comprise the first internal loop and the second internal loop. In some embodiments, the first internal loop and the second internal loop independently comprises about 5 to about 10 bases of either the candidate guide RNA or the target RNA.

### BRIEF DESCRIPTION OF THE DRAWINGS

FIG. 1 depicts an exemplary overview of an embodiment of the subject methods provided herein.

FIG. 2 depicts a schematic of an exemplary embodiment of the subject methods provided herein. In a first step, an initial seed guide RNA is designed to a target RNA based on natural RNA editing entity (e.g., ADAR) substrates. Next, a library of self-annealing RNA structures (also referred to as “gRNA:target RNA library”) is created. Each of the self-annealing RNA structures in the library includes a variable guide RNAs that is covalently attached to an invariant target RNA. The variable guide RNA hybridizes to the target RNA to form an editable guide-target RNA scaffold that includes one or more structural features. The diversity of the guide RNAs in the library is generated, for example, by modifying the initial seed guide RNA based natural and/or random solutions. The library is contacted with an RNA editing entity (e.g., ADAR) under conditions that allow the editing of the self-anneal RNA structures by the RNA editing entity and the library is sequenced to identify self-annealing RNA structures that include an edited target RNA.

FIG. 3 depicts the summary of studies to identify guide RNAs for editing three target genes of interest using the subject methods provided herein: ABCA4, LRRK2, and SERPINA1.

FIG. 4 provides the structure of an exemplary self-annealing RNA structure used with the subject high throughput screening methods disclosed herein.

FIG. 5A and FIG. 5B show next generation sequencing (NGS) readouts of the high throughput screens for guides against 3 targets (ABCA4, LRRK2, and SERPINA1) summarized in FIG. 3. The x-axis indicates the target sequence with each position denoted to the target A edit site (denoted as 0). The y-axis represents the individual guides that were tested. Results are shown for in vitro ADAR2 (FIG. 5A) and ADAR1 and ADAR2 (FIG. 5B) mediated RNA editing of the target A in the target sequence using a library of self-annealing RNA structures that have the structure depicted in FIG. 4.

FIG. 6 depicts metrics for selecting candidate guide RNAs that are capable of editing a target RNA of interest. Selection metrics include, for example, 1) target RNA editing rate kinetics; 2) on-target vs. off-target editing specificity; and 3) ADAR1 vs, ADAR2 agreement (a comparison of the ADAR1 vs. ADAR2 editing profile of a candidate gRNA).

FIG. 7 provides a summary of a study to assess the editing rate kinetics of a self-annealing RNA structure library for editing LRRK2 target RNA, as well as exemplary method for determining such kinetics. As shown in FIG. 7, the library includes a diverse population of gRNAs that are capable of mediating on-target LRRK2 editing at different rates.

FIG. 8 provides an illustration of the ADAR1 vs. ADAR2 agreement selection metric for characterizing candidate gRNAs according to the subject methods provided herein. In this figure, a difference is observed between ADAR1 vs. ADAR2 mediated editing of ABCA4 target gene by this particular gRNA. This gRNA shows strong +1 off target editing in the presence of ADAR2 (bottom), but not with ADAR1. The off-target effects of ADAR2 mediated editing, however, can be less relevant in this example if the cells wherein ABCA4 editing is desired (e.g., retinal cells) do not express ADAR2. FIG. 8 discloses SEQ ID NOS 217-218, respectively, in order of appearance.

FIG. 9 provides a summary of a kinetics study to identify gRNAs for LRRK2 target RNA using a subject self-annealing RNA structure library and high throughput screen as described herein. As shown in FIG. 9, the screen identified gRNAs capable of on-target LRRK2 editing at a diverse range of kinetics, including high ranked design that exhibited a 30-fold increase in Kobs as compared to seed gRNAs.

FIG. 10 provides a summary of the gRNAs identified in the LRRK2 screen described in FIG. 3 and FIG. 9 and the examples provided herein characterized by three metrics: 1) target RNA editing rate kinetics; 2) on-target vs. off-target editing specificity; and 3) ADAR1 vs, ADAR2 agreement.

FIG. 11A-FIG. 11D provide a summary of characterization studies for a self-annealing RNA structure identified from the LRRK2 screen described in FIG. 3 and FIG. 9 and the examples. FIG. 11A shows the self-annealing RNA structure used in the studies. The self-annealing RNA structure includes the candidate guide RNA and target LRRK2 RNA. FIG. 11A further depicts a study to assess the editing specificity of LRRK2 RNA by the candidate guide RNA in the presence of either ADAR1 or ADAR2 after 100 minutes. FIG. 11B depicts a kinetics study to assess ADAR1/ADAR2 editing of LRRK2 RNA at various time points using the self-annealing RNA structure depicted in FIG. 11A. FIG. 11C and FIG. 11D depict a study of ADAR2 editing specificity of LRRK2 RNA at various time points using the self-annealing RNA structure depicted in FIG. 11A. FIG. 11A discloses SEQ ID NOS 219-220, and 220, FIG. 11C discloses SEQ ID NOS 220 and 220, and FIG. 11D discloses SEQ ID NOS 220 and 220, all respectively, in order of appearance.

FIG. 12A-FIG. 12D provide a summary of characterization studies for a self-annealing RNA structure identified from the LRRK2 screen described in FIG. 3 and FIG. 9 and the examples. FIG. 12A shows the self-annealing RNA structure used in the studies. The self-annealing RNA structure includes the candidate guide RNA and LRRK2 RNA. FIG. 12A further depicts a study to assess the editing specificity of LRRK2 RNA by the candidate guide RNA in the presence of either ADAR1 or ADAR2 after 100 minutes. FIG. 11B depicts a kinetics study to assess ADAR1/ADAR2 editing of LRRK2 RNA at various time points using the self-annealing RNA structure depicted in FIG. 12A. FIG. 12C and FIG. 12D depict a study of ADAR2 editing specificity of LRRK2 RNA at various time points using the self-annealing RNA structure depicted in FIG. 12A. FIG. 12A discloses SEQ ID NOS 221-222, and 222, FIG. 12C discloses SEQ ID NOS 222 and 222, and FIG. 12D discloses SEQ ID NOS 222 and 222, all respectively, in order of appearance.

FIG. 13A-FIG. 13D provide a summary of characterization studies for a self-annealing RNA structure identified from the LRRK2 screen described in FIG. 3 and FIG. 9 and the examples. FIG. 13A shows the self-annealing RNA structure used in the studies. The self-annealing RNA structure includes the candidate guide RNA and LRRK2 RNA. FIG. 13A further depicts a study to assess the editing specificity of LRRK2 RNA by the candidate guide RNA in the presence of either ADAR1 or ADAR2 after 100 minutes. FIG. 13B depicts a kinetics study to assess ADAR1/ADAR2 editing of LRRK2 RNA at various time points using the self-annealing RNA structure depicted in FIG. 13A. FIG. 13C and FIG. 13D depict a study of ADAR2 editing specificity of LRRK2 RNA at various time points using the self-annealing RNA structure depicted in FIG. 13A. FIG. 13A discloses SEQ ID NOS 223-224, and 224, FIG. 13C discloses SEQ ID NOS 224 and 224, and FIG. 13D discloses SEQ ID NOS 224 and 224, all respectively, in order of appearance.

FIGS. 14A-C depict a summary of the in vitro study using the subject methods provided herein to screen for guide RNAs (gRNAs) for ADAR mediated editing of the LRRK2*G2019S mutation (FIGS. 3 and 9 and the examples). A library of guide-target RNA scaffolds that included 2,536 different gRNAs was screened. The subject screening methods identified gRNAs that exhibited superior on targeting editing of the LRRK2*G2019S mutation as compared to previous gRNAs (FIG. 14A and FIG. 14C). FIG. 14B is an NGS readout that shows a profile of the ADAR2 mediated edits of the LRRK2*G2019S target by the guide-target RNA scaffold library. The x-axis indicates the LRRK2 target sequence with each position denoted to the target A edit site (denoted as 0). The y-axis represents the individual guides that were tested. FIG. 14A discloses SEQ ID NOS 225 and 225, respectively, in order of appearance and FIG. 14C discloses SEQ ID NO: 225.

FIG. 15 provides a summary of the gRNAs identified in the ABCA4 screen described in FIG. 3 and the examples provided herein characterized by three metrics: 1) target RNA editing rate kinetics; 2) on-target vs. off-target editing specificity; and 3) ADAR1 vs, ADAR2 agreement.

FIG. 16A and FIG. 16B provide a summary of characterization studies for a self-annealing RNA structure identified from the ABCA4 screen described in FIG. 3 and the examples. FIG. 16A shows that self-annealing RNA structure used in the studies. The self-annealing RNA structure includes the candidate guide RNA and ABCA4 RNA. FIG. 16A further depicts a study to assess the editing specificity of ABCA4 RNA by the candidate guide RNA in the presence of either ADAR1 or ADAR2 after 100 minutes. FIG. 16B depicts a kinetics study to assess ADAR1/ADAR2 editing of ABCA4 RNA at various time points using the self-annealing RNA structure depicted in FIG. 16A. FIG. 16A discloses SEQ ID NOS 226-227 and 227, respectively, in order of appearance

FIG. 17A and FIG. 17B provide a summary of characterization studies for a self-annealing RNA structure identified from the ABCA4 screen described in FIG. 3 and the examples. FIG. 17A shows the self-annealing RNA structure used in the studies. The self-annealing RNA structure includes the candidate guide RNA and ABCA4 RNA. FIG. 17A further depicts a study to assess the editing specificity of ABCA4 RNA by the candidate guide RNA in the presence of either ADAR1 or ADAR2 after 100 minutes. FIG. 17B depicts a kinetics study to assess ADAR1/ADAR2 editing of target RNA X at various time points using the self-annealing RNA structure depicted in FIG. 17A. FIG. 17A discloses SEQ ID NOS 228-229 and 229, respectively, in order of appearance

FIG. 18A and FIG. 18B provide a summary of characterization studies for a self-annealing RNA structure identified from the ABCA4 screen described in FIG. 3 and the examples. FIG. 18A shows the self-annealing RNA structure used in the studies. The self-annealing RNA structure includes the candidate guide RNA and ABCA4 RNA. FIG. 18A further depicts a study to assess the editing specificity of ABCA4 RNA by the candidate guide RNA in the presence of either ADAR1 or ADAR2 after 100 minutes. FIG. 18B depicts a kinetics study to assess ADAR1/ADAR2 editing of ABCA4 RNA at various time points using the self-annealing RNA structure depicted in FIG. 18A. FIG. 18A discloses SEQ ID NOS 230-231 and 231, respectively, in order of appearance

FIG. 19A and FIG. 19B provide a summary of characterization studies for a self-annealing RNA structure identified from the ABCA4 screen described in FIG. 3 and the examples. FIG. 19A shows the self-annealing RNA structure used in the studies. The self-annealing RNA structure includes the candidate guide RNA and ABCA4 RNA. FIG. 19A further depicts a study to assess the editing specificity of ABCA4 RNA by the candidate guide RNA in the presence of either ADAR1 or ADAR2 after 100 minutes. FIG. 19B depicts a kinetics study to assess ADAR1/ADAR2 editing of ABCA4 RNA at various time points using the self-annealing RNA structure depicted in FIG. 19A. FIG. 19A discloses SEQ ID NOS 232-233 and 233, respectively, in order of appearance

FIG. 20A-FIG. 20C depict a summary of the in vitro study using the subject methods provided herein to screen for guide RNAs (gRNAs) for ADAR mediated editing of the ABCA4*G1961E mutation (FIG. 3 and the examples). A library of guide-target RNA scaffolds that included 2,688 different gRNAs was screened. The subject screening methods identified gRNAs that exhibited superior on-target editing of the ABCA4*G1961E mutation as compared to previous gRNAs (FIG. 20A and FIG. 20C). FIG. 20B is an NGS readout that shows a profile of the ADAR1 mediated edits of the ABCA4*G1961E target by the guide-target RNA scaffold library. The x-axis indicates the LRRK2 target sequence with each position denoted to the target A edit site (denoted as 0). The y-axis represents the individual guides that were tested.

FIG. 21 provides a summary of kinetics studies for the self-annealing RNA structure identified from the ABCA4 screen described in FIG. 3. As shown on the left, a variety of different self-annealing RNA structure that mediated on-target ADAR2 editing of the ABCA4 target RNA were identified. Further, the lead self-annealing RNA structures identified in the screen exhibited enhanced kinetics for editing of ABCA4 as compared to previous gRNA designs (middle and right). This demonstrates for the first time that endogenous ADAR can be made to edit a novel “5′G” site with the right guide sequence. FIG. 21 discloses SEQ ID NOS 217, 217, 217, and 217, respectively, in order of appearance

FIG. 22 depicts the summary of a screen for guide RNAs (gRNAs) for ADAR2 mediated editing of SERPINA1 using the methods and self-annealing RNA structures described herein. The screen was able to identify candidate RNAs that exhibit high on-target ADAR2 mediated editing for SERPINA1. FIG. 22 discloses SEQ ID NOS 234-235, respectively, in order of appearance

FIG. 23 shows a schematic of the high throughput screening (HTS) platform disclosed herein for discovery of guide RNAs (gRNA). The HTS platform scans a diverse secondary structure landscape to assess gRNA efficiency and selectivity. Disclosed herein is an iterative design process along with machine learning, which can further refine gRNA design to identify an exemplary superior gRNA for a given disease target.

FIG. 24 shows results from a high throughput screen designed to screen a library of over 100,000 guide designs targeting the LRRK2 G2019S mutation. The fraction edited at the −2 position (x-axis) and target position (y-axis) are depicted in the graph at left and the graph at center. The box in the top left section of each graph details the designs with >80% on target editing and <20% off-target editing at the −2 position. The Venn diagram on the right shows the overlap between the ADAR1 and ADAR2 designs that have >80% on target editing and <20% off target editing at the −2 position.

FIG. 25A, FIG. 25B, and FIG. 25C show results from the methods of high throughput screening disclosed herein to identify novel guides. FIG. 25A shows results from high throughput screening for gRNAs targeting LRRK2 G2019S. FIG. 25B shows results from high throughput screening for gRNAs targeting ABCA4 G1961E. FIG. 25C shows results from high throughput screening for gRNAs targeting SERPINA1 E342K. Data in FIG. 25A-FIG. 25C show the 30-minute time point with human ADAR2. Heat maps display the editing profile of the entire library (y-axis) at each target position (x-axis) with yellow (lighter shade) representing 100% editing and purple (darker shade) representing 0% editing. The bar graphs depict the performance of the standard A-C mismatch gRNA in comparison with a representative lead design from the high throughput screen. FIG. 25A discloses SEQ ID NOS 236, and 236, FIG. 25B discloses SEQ ID NOS 237 and 237, and FIG. 25C discloses SEQ ID NOS 238 and 238, all respectively, in order of appearance

FIG. 26 shows the sequence and structure of the following ABCA4 guides bound to target: guide01_CAPS1_128 gID_00001_v0114; guide06_Shaker5G_256_gID_01981_v0156; guide06_Shaker5G_256_gID_01981_v0025; guide06_Shaker5G_256_gID_01981_v0220; guide01_CAPS1_128_gID_00001_v0115; guide01_CAPS1_128_gID_00001_v0081; guide01_CAPS1_128_gID_00001_v0019; and guide06_Shaker5G_256_gID_01981_v0153. FIG. 26 discloses SEQ ID NOS 239-246 on the left and SEQ ID NOS 3-10 on the right, all respectively, in order of appearance.

FIG. 27 shows the sequence and structure of the following ABCA4 guides bound to target: guide05_Shaker5G_256_gID_01585_v0027; guide08_AJUBA_512_gID_02773_v0446; guide06_Shaker5G_256_gID_01981_v0025; guide08_AJUBA_512_gID_02773_v0414; guide01_CAPS1_128_gID_00001_v0018; guide06_Shaker5G_256_gID_01981_v0154; guide01_CAPS1_128_gID_00001_v0052; and guide01_CAPS1_128_gID_00001_v0050. FIG. 27 discloses SEQ ID NOS 247-254 on the left and SEQ ID NOS 11-18 on the right, all respectively, in order of appearance.

FIG. 28 shows the sequence and structure of the following ABCA4 guides bound to target: guide08_AJUBA_512_gID_02773_v0190; guide08_AJUBA_512_gID_02773_v0445; guide01_CAPS1_128_gID_00001_v0116; guide06_Shaker5G_256_gID_01981_v0028; guide08_AJUBA_512_gID_02773_v0062; guide08_AJUBA_512_gID_02773_v0189; guide01_CAPS1_128_gID_00001_v0082; and guide08_AJUBA_512_gID_02773_v0142.

FIG. 28 discloses SEQ ID NOS 255-262 on the left and SEQ ID NOS 19-26 on the right, all respectively, in order of appearance.

FIG. 29 shows the sequence and structure of the following ABCA4 guides bound to target: guide05_Shaker5G_256_gID_01585_v0155; guide06_Shaker5G_256_gID_01981_v0155; guide01_CAPS1_128_gID_00001_v0113; guide01_CAPS1_128_gID_00001_v0030; guide01_CAPS1_128_gID_00001_v0084; guide01_CAPS1_128_gID_00001_v0049; guide01_CAPS1_128_gID_00001_v0020; and guide01_CAPS1_128_gID_00001_v0051. FIG. 29 discloses SEQ ID NOS 263-270 on the left and SEQ ID NOS 27-34 on the right, all respectively, in order of appearance.

FIG. 30 shows a schematic of the structural features formed in the guide-target RNA scaffold of various candidate engineered guide RNAs of the present disclosure targeting LRRK2. FIG. 30 discloses SEQ ID NOS 271-281, respectively, in order of appearance.

FIG. 31 shows a schematic of the structural features formed in the guide-target RNA scaffold of various candidate engineered guide RNAs of the present disclosure targeting LRRK2. FIG. 31 discloses SEQ ID NOS 282-292, respectively, in order of appearance.

FIG. 32 shows a schematic of the structural features formed in the guide-target RNA scaffold of various candidate engineered guide RNAs of the present disclosure targeting LRRK2. FIG. 32 discloses SEQ ID NOS 293-304, respectively, in order of appearance.

FIG. 33 shows heat maps and structures for exemplary candidate engineered guide RNA sequences targeting a LRRK2 mRNA. The heat map provides visualization of the editing profile at the 10-minute time point. 5 candidate engineered guide RNAs for on-target editing (with no-2 filter) are in the left graph and 5 engineered guide RNAs for on-target editing with minimal-2 editing are depicted on the right graph. The corresponding predicted secondary structures are below the heat maps. FIG. 33 discloses SEQ ID NOS 305-314, respectively, in order of appearance.

FIG. 34 shows a summary of how a library for screening longer self-annealing RNA structures was generated.

FIG. 35 shows a comparison of cell-free RNA editing using the high throughput described here versus in-cell RNA editing facilitated via the same candidate engineered guide RNA sequence at various timepoints.

FIG. 36 shows heatmaps of all self-annealing RNA structures tested for the 4 micro-footprints described above formed within varying placement of a barbell macro-footprint. The y-axis shows all candidate engineered guide RNAs tested and the x-axis shows the target sequence positions, with position 0 representing the target adenosine to be edited.

FIG. 37 shows a schematic of a library design for screening of longer candidate engineered guide RNAs (e.g., 100mers) in-trans, which includes an RNA having the target sequence and a heterologous RNA having the candidate engineered guide RNA and where the RNA having the target sequence and the heterologous RNA having the candidate engineered guide RNA are not linked together or, in other words, are not a single contiguous sequence. FIG. 37 discloses SEQ ID NOS 315-316, respectively, in order of appearance.

FIG. 38 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). FIG. 38 discloses SEQ ID NOS 317-318, respectively, in order of appearance.

### DETAILED DESCRIPTION

Traditionally, gRNAs were designed to be complementary to the RNA of interest with an A-C mismatch at the target adenosine, a known preference of ADAR. However, the therapeutic potential of this approach is limited by the promiscuous nature of ADAR for guide-target RNA scaffolds resulting in bystander editing of adjacent adenosines.

Disclosed herein is a high throughput screening (HTS) platform to scan the secondary structures of gRNAs and identify those that promote specific and efficient ADAR editing. Using this approach, features are identified for direct incorporation into gRNAs and for machine learning to further refine gRNA design and develop more complex heuristics.

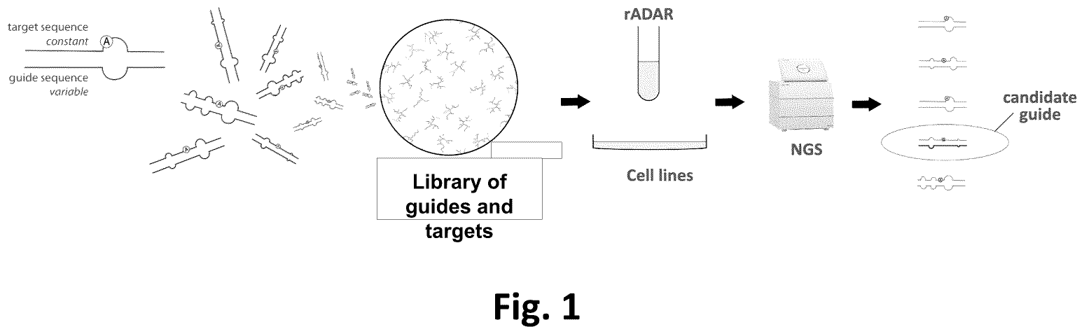

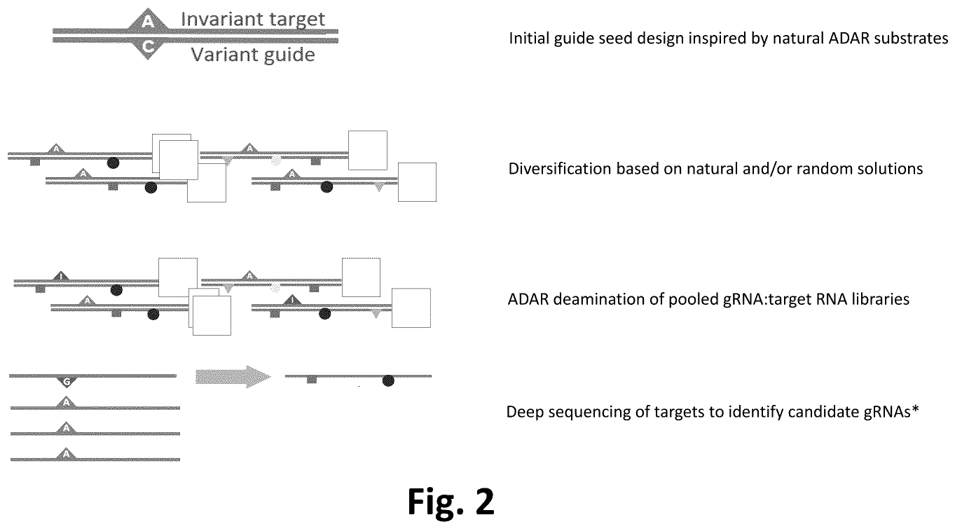

Provided herein a screening method for identifying gRNAs useful for editing of a target gene (see, e.g., FIG. 1) by a targeting entity. In some embodiments, the targeting entity is an ADAR, such as ADAR1 and ADAR2. ADAR1 and ADAR2 chemically modify double stranded RNA by deamination of adenosine to produce inosine, which is interpreted by cellular machinery as guanosine. On a select number of evolutionarily conserved transcripts, the editing is highly specific and results in non-synonymous mutations at the RNA level that create proteins with altered functionality. These substrates often have complex secondary structural features that include mismatches, bulges, and internal loops that are formed by imperfect cis-hybridization of exonic and intronic sequence. Evidence supports that these secondary structures contribute to the specificity of ADAR for the target adenosine. Without being bound by any particular theory of operation it is believed that incorporation of these secondary structural features into the trans interaction of a guide RNA and target pre-mRNA can promote the targeted recoding of the transcript to restore, modulate, or abolish protein function.

The method disclosed herein uses a library of self-annealing RNA structures that each include an candidate guide RNA and a target RNA (See FIG. 1). Hybridization of the candidate guide RNA and target RNA form a double stranded RNA substrate that includes one or more secondary structural features (e.g., a bulge, an internal loop or a hairpin). In exemplary embodiments, the library of self-annealing RNA structures includes a plurality of different candidate gRNAs that each hybridize to a constant target RNA, thereby forming a plurality of dsRNAs with different structural features. The library of self-annealing RNA structures is contacted with an RNA editing entity (e.g., a recombinant ADAR1 and/or ADAR2) under conditions that allow for the editing of one or more guide-target RNA scaffolds in the library and the library is subsequently assessed for edited targeted RNA, for example, using next generation sequencing (NGS). The subject methods disclosed herein advantageously allow for the high throughput screening of a large number of candidate gRNAs to identify gRNAs sequences and structures that contribute to editing of a target RNA of interest by a particular RNA editing entity (e.g., ADAR). Detailed knowledge of editing selectivity and efficiency for such a large and diverse set of dsRNA designs can be used to select features for incorporation into guide RNA molecules for trans-mediated editing of particular target RNAs of interest, e.g., mutant alleles associated with a particular disease or disorder.

A. Self-Annealing RNA Structure Library

Provided herein are self-annealing RNA structures and libraries that include a plurality of such self-annealing RNA structures. Such libraries are useful in the high throughput screening methods described herein for identifying guide RNAs suitable for editing a target RNA.

In some embodiments, the self-annealing RNA structures each include: 1) a target RNA of interest; 2) a candidate guide RNA; and 3) a linker that covalently attaches the target RNA and the candidate guide RNA. The candidate guide RNA and target RNA of the self-annealing RNA structures hybridize to form a guide-target RNA scaffolds that include one or more structural features. Examples of structural features include a mismatch, a bulge (symmetrical bulge or asymmetrical bulge), an internal loop (symmetrical internal loop or asymmetrical internal loop), a hairpin (a recruitment hairpin or a non-recruitment hairpin), or a wobble base pair.

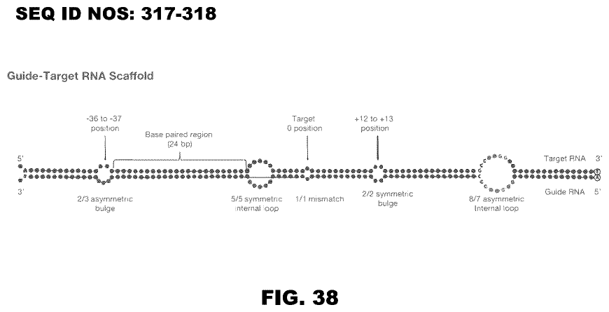

FIG. 38 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side).

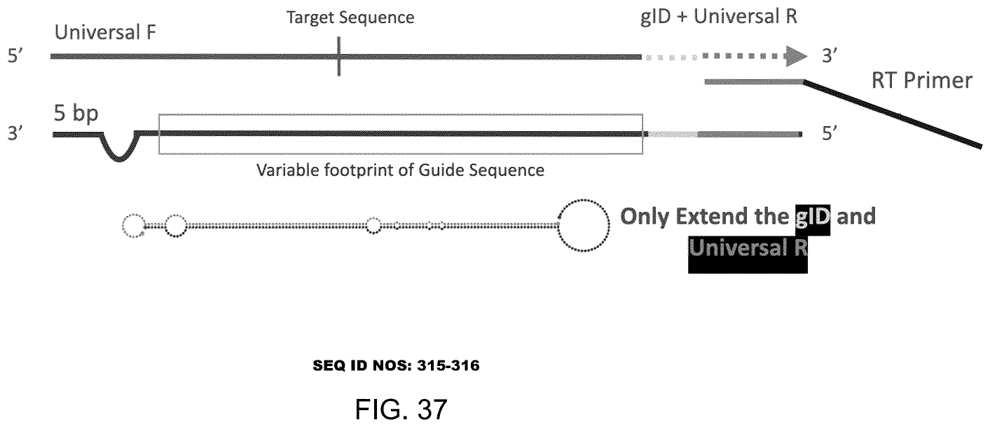

In some embodiments, the self-annealing RNA structures each include: 1) a first RNA having a target RNA sequence of interest and 2) a heterologous RNA having a candidate guide RNA. In this embodiment, the first RNA and the heterologous RNA are not covalently attached via a linker, but the candidate guide RNA and target RNA still hybridize to form a guide-target RNA scaffold that includes one or more structural features. Examples of structural features include a mismatch, a bulge (symmetrical bulge or asymmetrical bulge), an internal loop (symmetrical internal loop or asymmetrical internal loop), a hairpin (a recruitment hairpin or a non-recruitment hairpin), or a wobble base pair. Assays using this format of the self-annealing RNA structures can be referred to as a cell-free, in-trans high throughput screen. FIG. 37 shows a schematic of a library design for screening of longer candidate engineered guide RNAs (e.g., 100mers) in-trans, which includes an RNA having the target sequence and a heterologous RNA having the candidate engineered guide RNA and where the RNA having the target sequence and the heterologous RNA having the candidate engineered guide RNA are not linked together or, in other words, are not a single contiguous sequence. In such assays and compositions used in said assays, the components of the self-annealing RNA structures (the first RNA having the target RNA sequence and the heterologous RNA having the candidate guide RNA) can have additional identifiers. As shown in FIG. 37, a universal R and gID sequence can be mapped onto a target sequence using a polymerase enzyme (e.g. Bst3.0) after annealing of the target RNA sequence to the heterologous RNA having the universal R and gID sequences. An RT primer that recognizes the universal R can then be used along with an F primer to amplify the target sequence after editing, with the specific gID sequence being tied to each candidate guide RNA. The RT primer can contain 7 N nucleotides, followed by a partial sequence that can be recognized in a next generation sequencing reaction (e.g. Illumina sequencing). Further, the candidate guide sequence can be designed with base complementarity near the 3′ end with mismatches and loops when bound to the target sequence, as depicted in FIG. 37. In such an embodiment, the 5′ end of the target and the 3′ end of the guide can anneal completely, and there may be no extension on this end during the subsequent extension reaction. The extension may occur only on the 3′ end of the target region. Including loops near the 3′ end of the guide, which anneals to the target, prevents the F primer from amplifying/binding to any guide sequence making it specific for target amplification post editing.

The self-annealing RNA structures provided herein can have from 1 to 50 structural features. In some embodiments, the guide-target RNA scaffolds of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 structural features.

The self-annealing RNA structure libraries can be synthesized as DNA oligo pools containing discrete designs or as a single DNA oligos containing degenerate bases at positions of interest (e.g., variant gRNA positions). In some embodiments, the guide RNAs are designed based on an RNA editing entity (e.g., ADAR) substrate mimics and/or sequence randomization to intelligently cover as much sequence and structural space as possible. The total theoretical diversity in the guide-target RNA scaffold libraries disclosed herein can range from thousands to millions of designs. Amplification and incorporation of a T7 promoter sequence is performed to create a template for reverse transcription.

In some embodiments, the library of self-annealing RNA structures used in the methods disclosed herein include guide-target RNA scaffolds with a plurality of different gRNAs that each hybridize with a target RNA having the same sequence, to form a plurality of guide-target RNA scaffolds each having different structural features. The diversity of the structural features exhibited by a self-annealing RNA structure library is designed to capture a wide range of secondary structure diversity that is found in natural ADAR substrates.

In some embodiments, the self-annealing RNA structure library includes at least about 2, 10, 20, 30, 40, 50, 100, 200, 300, 400, 500, 600, 700, 800, 900 or 1,000 different gRNAs that hybridize to a common target RNA sequence to form a plurality of guide-target RNA scaffolds each having different structural features. In exemplary embodiments, the self-annealing RNA structure library used in the methods disclosed herein include guide-target RNA scaffolds that have at least about 1×10³, 0.5×10⁴, 1×10⁴, 0.5×10⁵, 1×10⁵, 0.5×10⁶, 1×10⁶, 0.5×10⁷, or 1×10⁷ different gRNAs that form a plurality of guide-target RNA scaffolds each having different structural features. In some embodiments, the self-annealing RNA structure library used in the methods disclosed herein include guide-target RNA scaffolds that have from 1×10³ to 2.5×10³, from 2.5×10³ to 5.0×10³, from 5.0×10³ to 1.0×10⁴, from 1.0×10⁴ to 5.0×10⁴, from 5.0×10⁴ to 1.0×10⁵, from 1.0×10⁵ to 5.0×10⁵, or from 5.0×10⁵ to 1.0×10⁶ different gRNAs that form a plurality of guide-target RNA scaffolds each having different structural features. In some embodiments, the self-annealing RNA structure library includes about 100,000 guide RNAs.

An engineered RNA structure (for example, a self-annealing RNA structure) can have a length of from about 10 nucleotides to about 500 nucleotides. For example, an engineered RNA structure can have a length of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides. In some embodiments, an engineered RNA structure can have a length of from about 10 nucleotides to about 500 nucleotides, from about 15 nucleotides to about 500 nucleotides, from about 20 nucleotides to about 500 nucleotides, from about 25 nucleotides to about 500 nucleotides, from about 30 nucleotides to about 500 nucleotides, from about 35 nucleotides to about 500 nucleotides, from about 40 nucleotides to about 500 nucleotides, from about 45 nucleotides to about 500 nucleotides, from about 50 nucleotides to about 500 nucleotides, from about 55 nucleotides to about 500 nucleotides, from about 60 nucleotides to about 500 nucleotides, from about 65 nucleotides to about 500 nucleotides, from about 70 nucleotides to about 500 nucleotides, from about 75 nucleotides to about 500 nucleotides, from about 80 nucleotides to about 500 nucleotides, from about 85 nucleotides to about 500 nucleotides, from about 90 nucleotides to about 500 nucleotides, from about 95 nucleotides to about 500 nucleotides, from about 100 nucleotides to about 500 nucleotides, from about 105 nucleotides to about 500 nucleotides, from about 110 nucleotides to about 500 nucleotides, from about 115 nucleotides to about 500 nucleotides, from about 120 nucleotides to about 500 nucleotides, from about 125 nucleotides to about 500 nucleotides, from about 130 nucleotides to about 500 nucleotides, from about 135 nucleotides to about 500 nucleotides, from about 140 nucleotides to about 500 nucleotides, from about 145 nucleotides to about 500 nucleotides, from about 150 nucleotides to about 500 nucleotides, from about 155 nucleotides to about 500 nucleotides, from about 160 nucleotides to about 500 nucleotides, from about 165 nucleotides to about 500 nucleotides, from about 170 nucleotides to about 500 nucleotides, from about 175 nucleotides to about 500 nucleotides, from about 180 nucleotides to about 500 nucleotides, from about 185 nucleotides to about 500 nucleotides, from about 190 nucleotides to about 500 nucleotides, from about 195 nucleotides to about 500 nucleotides, or from about 200 nucleotides to about 500 nucleotides.

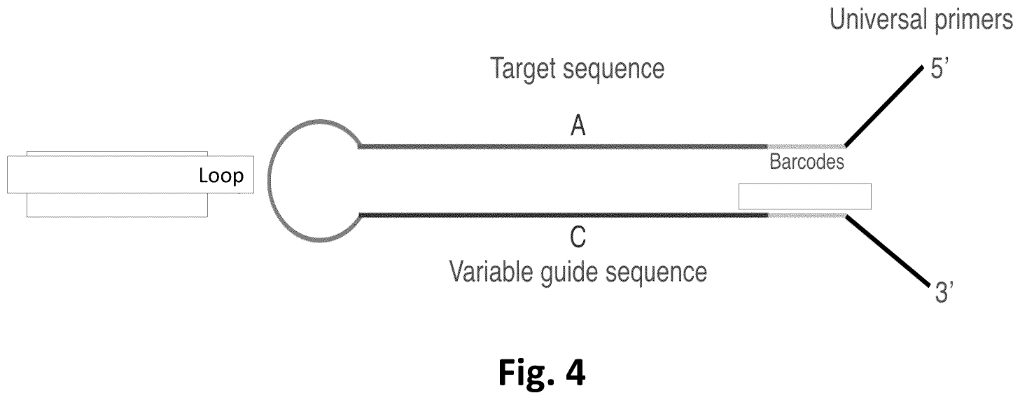

In some embodiments, the self-annealing RNA structure includes a hairpin linker that covalently attaches a target RNA and a gRNA. See, e.g., FIG. 4. Upon hybridization of the gRNA to the target RNA, the hairpin linker forms a hairpin loop. These structures mimic the common hairpin loop structure of natural substrates that is often formed between exonic and intronic sequences. Suitable hairpin linkers include those that form GluR and TAR hairpins, or other hairpins that are substrates for ADAR, APOBEC, or other RNA editing enzymes, including those disclosed herein.

In some embodiments, the 5′ and 3′ ends of the dsRNA contain unique guide ID sequences and universal primer binding sites that allow for analysis of the library for substrates edited by a RNA editing entity (e.g., ADAR). Such universal primer binding sites and barcodes allow, for example, amplification and identification of edited guide-target RNA scaffolds using high throughput multiplex sequencing methods (e.g., next generation sequencing).

The self-annealing RNA structures provided herein can further include primer binding sites to facilitate application of the self-annealing RNA structure. In some embodiments, the self-annealing RNA structure further includes one or more nucleic acid barcodes that allow for the identification of the self-annealing RNA structure. The structure of an exemplary self-annealing RNA structure of the self-annealing RNA structure libraries disclosed herein is depicted in FIG. 4. As shown in FIG. 4, the self-annealing RNA structure includes two universal primer binding sites and two optional barcode sequences. In some embodiments, the barcode sequences are unique for each of the self-annealing RNA structures in a library and allow for multiplex sequencing of amplicons derived from each of the self-annealing RNA structure using a high throughput sequencing method (e.g., NGS sequencing). In some embodiments, each of the self-annealing RNA structure s are according to the formula, from 5′-to-3′: UPBS1-BC1-target RNA-linker-guide RNA-BC2-UPB S2, wherein UPB S1 and UPBS2 are a first universal primer binding site and second universal primer binding site respectively, BC1 and BC2 are a first optional barcode and second optional code respectively, and the linker can be any suitable linker. In some embodiments, the linker forms a hairpin loop. In some embodiments, the self-annealing RNA structure includes one barcode. In certain embodiments, the self-annealing RNA structure includes two barcodes. In yet other embodiments, the self-annealing RNA structure does not include barcodes.

1. Structural Features

Engineered Candidate Guide RNAs with a Micro-Footprint Sequence Having Latent Structure

Each of the self-annealing RNA structures in a library include one or more structural features formed by the hybridization of the target RNA and a candidate guide RNA. Such structures, prior to forming, can be included in a guide RNA as a latent structure. Such guides containing latent structures are also referred to as “latent guide RNAs.” A micro-footprint sequence of a candidate guide RNA comprising latent structures (e.g., a “latent structure guide RNA”) can comprise a portion of sequence that, upon hybridization to a target RNA, forms at least a portion of a structural feature, other than a single A/C mismatch feature at the target adenosine to be edited. Such micro-footprint sequences of candidate guide RNAs can be elucidated using the high-throughput methods described herein. Accordingly, in some embodiments, disclosed herein are methods of generating a micro-footprint sequence having one or more structural features described herein, where the structural features of the micro-footprint sequence configure the candidate guide RNA to facilitate editing of a target RNA via an RNA editing entity. “Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked. Exemplary structural features include, but are not limited to mismatches, bulges, internal loops, hairpins, and wobble base pairs, as detailed below.

In some embodiments, the library of guide-target RNA scaffolds includes one or more guide-target RNA scaffolds with a mismatch. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. A mismatch can include an A/A mismatch, an A/G mismatch, an A/C mismatch, a G/G mismatch, a C/C mismatch, or a C/U mismatch. In some embodiments, a mismatch is an A/C mismatch. An A/C mismatch can comprise a C in a candidate engineered guide RNA of the present disclosure opposite an A in a target RNA. An A/C mismatch can comprise an A in a candidate engineered guide RNA of the present disclosure opposite a C in a target RNA. A G/G mismatch can comprise a G in a candidate engineered guide RNA of the present disclosure opposite a G in a target RNA. In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA

In some embodiments, the self-annealing RNA structure library include one or more self-annealing RNA structures with a bulge. As disclosed herein, a bulge refers to the structure substantially formed only upon formation of the guide-target RNA scaffold, where contiguous nucleotides in either the candidate engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the candidate engineered guide RNA and a single participating nucleotide of the target RNA do not base pair—a single participating nucleotide of the candidate engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA. In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

Exemplary bulges that can be included in the self-annealing RNA structure library disclosed herein include symmetrical and asymmetrical bulges. A symmetrical bulge is formed when the same number of nucleotides is present on each side of the bulge. For example, a symmetrical bulge in a guide-target RNA scaffold of the present disclosure can have the same number of nucleotides on the candidate engineered guide RNA side and the target RNA side of the guide-target RNA scaffold. A symmetrical bulge of the present disclosure can be formed by 2 nucleotides on the candidate engineered guide RNA side of the guide-target RNA scaffold target and 2 nucleotides on the target RNA side of the guide-target RNA scaffold. A symmetrical bulge of the present disclosure can be formed by 3 nucleotides on the candidate engineered guide RNA side of the guide-target RNA scaffold target and 3 nucleotides on the target RNA side of the guide-target RNA scaffold. A symmetrical bulge of the present disclosure can be formed by 4 nucleotides on the candidate engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

An asymmetrical bulge is formed when a different number of nucleotides is present on each side of the bulge. For example, an asymmetrical bulge in a guide-target RNA scaffold of the present disclosure can have different numbers of nucleotides on the candidate engineered guide RNA side and the target RNA side of the guide-target RNA scaffold. An asymmetrical bulge of the present disclosure can be formed by 0 nucleotides on the candidate engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the candidate engineered guide RNA side of the guide-target RNA scaffold. An asymmetrical bulge of the present disclosure can be formed by 0 nucleotides on the candidate engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the candidate engineered guide RNA side of the guide-target RNA scaffold. An asymmetrical bulge of the present disclosure can be formed by 0 nucleotides on the candidate engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the candidate engineered guide RNA side of the guide-target RNA scaffold. An asymmetrical bulge of the present disclosure can be formed by 0 nucleotides on the candidate engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the candidate engineered guide RNA side of the guide-target RNA scaffold. An asymmetrical bulge of the present disclosure can be formed by 1 nucleotide on the candidate engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the candidate engineered guide RNA side of the guide-target RNA scaffold. An asymmetrical bulge of the present disclosure can be formed by 1 nucleotide on the candidate engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the candidate engineered guide RNA side of the guide-target RNA scaffold. An asymmetrical bulge of the present disclosure can be formed by 1 nucleotide on the candidate engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the candidate engineered guide RNA side of the guide-target RNA scaffold. An asymmetrical bulge of the present disclosure can be formed by 2 nucleotides on the candidate engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the candidate engineered guide RNA side of the guide-target RNA scaffold. An asymmetrical bulge of the present disclosure can be formed by 2 nucleotides on the candidate engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the candidate engineered guide RNA side of the guide-target RNA scaffold. An asymmetrical bulge of the present disclosure can be formed by 3 nucleotides on the candidate engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the candidate engineered guide RNA side of the guide-target RNA scaffold. Thus, an asymmetrical bulge can be a structural feature formed from latent structure provided by an engineered latent guide RNA.

In some embodiments, the library of self-annealing RNA structures include one or more self-annealing RNA structures with an internal loop. As disclosed herein, an internal loop refers to the structure substantially formed only upon formation of the guide-target RNA scaffold, where nucleotides in either the candidate engineered guide RNA or the target RNA are not complementary to their positional counterparts on the opposite strand and where one side of the internal loop, either on the target RNA side or the candidate engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the candidate engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

In an aspect, a guide-target RNA scaffold is formed upon hybridization of a candidate engineered guide RNA of the present disclosure to a target RNA. An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the candidate engineered guide RNA side and the target RNA side of the guide-target RNA scaffold. A symmetrical internal loop of the present disclosure can be formed by 5 nucleotides on the candidate engineered guide RNA side of the guide-target RNA scaffold target and 5 nucleotides on the target RNA side of the guide-target RNA scaffold. A symmetrical internal loop of the present disclosure can be formed by 6 nucleotides on the candidate engineered guide RNA side of the guide-target RNA scaffold target and 6 nucleotides on the target RNA side of the guide-target RNA scaffold. A symmetrical internal loop of the present disclosure can be formed by 7 nucleotides on the candidate engineered guide RNA side of the guide-target RNA scaffold target and 7 nucleotides on the target RNA side of the guide-target RNA scaffold. A symmetrical internal loop of the present disclosure can be formed by 8 nucleotides on the candidate engineered guide RNA side of the guide-target RNA scaffold target and 8 nucleotides on the target RNA side of the guide-target RNA scaffold. A symmetrical internal loop of the present disclosure can be formed by 9 nucleotides on the candidate engineered guide RNA side of the guide-target RNA scaffold target and 9 nucleotides on the target RNA side of the guide-target RNA scaffold. A symmetrical internal loop of the present disclosure can be formed by 10 nucleotides on the candidate engineered guide RNA side of the guide-target RNA scaffold target and 10 nucleotides on the target RNA side of the guide-target RNA scaffold. A symmetrical internal loop of the present disclosure can be formed by 15 nucleotides on the candidate engineered guide RNA side of the guide-target RNA scaffold target and 15 nucleotides on the target RNA side of the guide-target RNA scaffold. A symmetrical internal loop of the present disclosure can be formed by 20 nucleotides on the candidate engineered guide RNA side of the guide-target RNA scaffold target and 20 nucleotides on the target RNA side of the guide-target RNA scaffold. A symmetrical internal loop of the present disclosure can be formed by 30 nucleotides on the candidate engineered guide RNA side of the guide-target RNA scaffold target and 30 nucleotides on the target RNA side of the guide-target RNA scaffold. A symmetrical internal loop of the present disclosure can be formed by 40 nucleotides on the candidate engineered guide RNA side of the guide-target RNA scaffold target and 40 nucleotides on the target RNA side of the guide-target RNA scaffold. A symmetrical internal loop of the present disclosure can be formed by 50 nucleotides on the candidate engineered guide RNA side of the guide-target RNA scaffold target and 50 nucleotides on the target RNA side of the guide-target RNA scaffold. A symmetrical internal loop of the present disclosure can be formed by 60 nucleotides on the candidate engineered guide RNA side of the guide-target RNA scaffold target and 60 nucleotides on the target RNA side of the guide-target RNA scaffold. A symmetrical internal loop of the present disclosure can be formed by 70 nucleotides on the candidate engineered guide RNA side of the guide-target RNA scaffold target and 70 nucleotides on the target RNA side of the guide-target RNA scaffold. A symmetrical internal loop of the present disclosure can be formed by 80 nucleotides on the candidate engineered guide RNA side of the guide-target RNA scaffold target and 80 nucleotides on the target RNA side of the guide-target RNA scaffold. A symmetrical internal loop of the present disclosure can be formed by 90 nucleotides on the candidate engineered guide RNA side of the guide-target RNA scaffold target and 90 nucleotides on the target RNA side of the guide-target RNA scaffold. A symmetrical internal loop of the present disclosure can be formed by 100 nucleotides on the candidate engineered guide RNA side of the guide-target RNA scaffold target and 100 nucleotides on the target RNA side of the guide-target RNA scaffold. A symmetrical internal loop of the present disclosure can be formed by 110 nucleotides on the candidate engineered guide RNA side of the guide-target RNA scaffold target and 110 nucleotides on the target RNA side of the guide-target RNA scaffold. A symmetrical internal loop of the present disclosure can be formed by 120 nucleotides on the candidate engineered guide RNA side of the guide-target RNA scaffold target and 120 nucleotides on the target RNA side of the guide-target RNA scaffold. A symmetrical internal loop of the present disclosure can be formed by 130 nucleotides on the candidate engineered guide RNA side of the guide-target RNA scaffold target and 130 nucleotides on the target RNA side of the guide-target RNA scaffold. A symmetrical internal loop of the present disclosure can be formed by 140 nucleotides on the candidate engineered guide RNA side of the guide-target RNA scaffold target and 140 nucleotides on the target RNA side of the guide-target RNA scaffold. A symmetrical internal loop of the present disclosure can be formed by 150 nucleotides on the candidate engineered guide RNA side of the guide-target RNA scaffold target and 150 nucleotides on the target RNA side of the guide-target RNA scaffold. A symmetrical internal loop of the present disclosure can be formed by 200 nucleotides on the candidate engineered guide RNA side of the guide-target RNA scaffold target and 200 nucleotides on the target RNA side of the guide-target RNA scaffold. A symmetrical internal loop of the present disclosure can be formed by 250 nucleotides on the candidate engineered guide RNA side of the guide-target RNA scaffold target and 250 nucleotides on the target RNA side of the guide-target RNA scaffold. A symmetrical internal loop of the present disclosure can be formed by 300 nucleotides on the candidate engineered guide RNA side of the guide-target RNA scaffold target and 300 nucleotides on the target RNA side of the guide-target RNA scaffold. A symmetrical internal loop of the present disclosure can be formed by 350 nucleotides on the candidate engineered guide RNA side of the guide-target RNA scaffold target and 350 nucleotides on the target RNA side of the guide-target RNA scaffold. A symmetrical internal loop of the present disclosure can be formed by 400 nucleotides on the candidate engineered guide RNA side of the guide-target RNA scaffold target and 400 nucleotides on the target RNA side of the guide-target RNA scaffold. A symmetrical internal loop of the present disclosure can be formed by 450 nucleotides on the candidate engineered guide RNA side of the guide-target RNA scaffold target and 450 nucleotides on the target RNA side of the guide-target RNA scaffold. A symmetrical internal loop of the present disclosure can be formed by 500 nucleotides on the candidate engineered guide RNA side of the guide-target RNA scaffold target and 500 nucleotides on the target RNA side of the guide-target RNA scaffold. A symmetrical internal loop of the present disclosure can be formed by 600 nucleotides on the candidate engineered guide RNA side of the guide-target RNA scaffold target and 600 nucleotides on the target RNA side of the guide-target RNA scaffold. A symmetrical internal loop of the present disclosure can be formed by 700 nucleotides on the candidate engineered guide RNA side of the guide-target RNA scaffold target and 700 nucleotides on the target RNA side of the guide-target RNA scaffold. A symmetrical internal loop of the present disclosure can be formed by 800 nucleotides on the candidate engineered guide RNA side of the guide-target RNA scaffold target and 800 nucleotides on the target RNA side of the guide-target RNA scaffold. A symmetrical internal loop of the present disclosure can be formed by 900 nucleotides on the candidate engineered guide RNA side of the guide-target RNA scaffold target and 900 nucleotides on the target RNA side of the guide-target RNA scaffold. A symmetrical internal loop of the present disclosure can be formed by 1000 nucleotides on the candidate engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

In an aspect, a guide-target RNA scaffold is formed upon hybridization of a candidate engineered guide RNA of the present disclosure to a target RNA. An internal loop can be a symmetrical internal loop or an asymmetrical internal loop. An asymmetrical internal loop is formed when a different number of nucleotides is present on each side of the internal loop. For example, an asymmetrical internal loop in a guide-target RNA scaffold of the present disclosure can have different numbers of nucleotides on the candidate engineered guide RNA side and the target RNA side of the guide-target RNA scaffold.

An asymmetrical internal loop of the present disclosure can be formed by from 5 to 150 nucleotides on the candidate engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the candidate engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the candidate engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the candidate engineered guide RNA side of the guide-target RNA scaffold. An asymmetrical internal loop of the present disclosure can be formed by 5 nucleotides on the candidate engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the candidate engineered guide RNA side of the guide-target RNA scaffold. An asymmetrical internal loop of the present disclosure can be formed by 5 nucleotides on the candidate engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the candidate engineered guide RNA side of the guide-target RNA scaffold. An asymmetrical internal loop of the present disclosure can be formed by 5 nucleotides on the candidate engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the candidate engineered guide RNA side of the guide-target RNA scaffold. An asymmetrical internal loop of the present disclosure can be formed by 5 nucleotides on the candidate engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the candidate engineered guide RNA side of the guide-target RNA scaffold. An asymmetrical internal loop of the present disclosure can be formed by 6 nucleotides on the candidate engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the candidate engineered guide RNA side of the guide-target RNA scaffold. An asymmetrical internal loop of the present disclosure can be formed by 6 nucleotides on the candidate engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the candidate engineered guide RNA side of the guide-target RNA scaffold. An asymmetrical internal loop of the present disclosure can be formed by 6 nucleotides on the candidate engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the candidate engineered guide RNA side of the guide-target RNA scaffold. An asymmetrical internal loop of the present disclosure can be formed by 6 nucleotides on the candidate engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the candidate engineered guide RNA side of the guide-target RNA scaffold. An asymmetrical internal loop of the present disclosure can be formed by 7 nucleotides on the candidate engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the candidate engineered guide RNA side of the guide-target RNA scaffold. An asymmetrical internal loop of the present disclosure can be formed by 7 nucleotides on the candidate engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the candidate engineered guide RNA side of the guide-target RNA scaffold. An asymmetrical internal loop of the present disclosure can be formed by 7 nucleotides on the candidate engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the candidate engineered guide RNA side of the guide-target RNA scaffold. An asymmetrical internal loop of the present disclosure can be formed by 8 nucleotides on the candidate engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the candidate engineered guide RNA side of the guide-target RNA scaffold. An asymmetrical internal loop of the present disclosure can be formed by 8 nucleotides on the candidate engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the candidate engineered guide RNA side of the guide-target RNA scaffold. An asymmetrical internal loop of the present disclosure can be formed by 9 nucleotides on the candidate engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the candidate engineered guide RNA side of the guide-target RNA scaffold. An asymmetrical internal loop of the present disclosure can be formed by 5 nucleotides on the target RNA side of the guide-target RNA scaffold and 50 nucleotides on the candidate engineered guide RNA side of the guide-target RNA scaffold. An asymmetrical internal loop of the present disclosure can be formed by 5 nucleotides on the target RNA side of the guide-target RNA scaffold and 100 nucleotides on the candidate engineered guide RNA side of the guide-target RNA scaffold. An asymmetrical internal loop of the present disclosure can be formed by 5 nucleotides on the target RNA side of the guide-target RNA scaffold and 150 nucleotides on the candidate engineered guide RNA side of the guide-target RNA scaffold. An asymmetrical internal loop of the present disclosure can be formed by 5 nucleotides on the target RNA side of the guide-target RNA scaffold and 200 nucleotides on the candidate engineered guide RNA side of the guide-target RNA scaffold. An asymmetrical internal loop of the present disclosure can be formed by 5 nucleotides on the target RNA side of the guide-target RNA scaffold and 300 nucleotides on the candidate engineered guide RNA side of the guide-target RNA scaffold. An asymmetrical internal loop of the present disclosure can be formed by 5 nucleotides on the target RNA side of the guide-target RNA scaffold and 400 nucleotides on the candidate engineered guide RNA side of the guide-target RNA scaffold. An asymmetrical internal loop of the present disclosure can be formed by 5 nucleotides on the target RNA side of the guide-target RNA scaffold and 500 nucleotides on the candidate engineered guide RNA side of the guide-target RNA scaffold. An asymmetrical internal loop of the present disclosure can be formed by 5 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 5 nucleotides on the candidate engineered guide RNA side of the guide-target RNA scaffold. An asymmetrical internal loop of the present disclosure can be formed by 500 nucleotides on the target RNA side of the guide-target RNA scaffold and 5 nucleotides on the candidate engineered guide RNA side of the guide-target RNA scaffold. An asymmetrical internal loop of the present disclosure can be formed by 400 nucleotides on the target RNA side of the guide-target RNA scaffold and 5 nucleotides on the candidate engineered guide RNA side of the guide-target RNA scaffold. An asymmetrical internal loop of the present disclosure can be formed by 300 nucleotides on the target RNA side of the guide-target RNA scaffold and 5 nucleotides on the candidate engineered guide RNA side of the guide-target RNA scaffold. An asymmetrical internal loop of the present disclosure can be formed by 200 nucleotides on the target RNA side of the guide-target RNA scaffold and 5 nucleotides on the candidate engineered guide RNA side of the guide-target RNA scaffold. An asymmetrical internal loop of the present disclosure can be formed by 150 nucleotides on the target RNA side of the guide-target RNA scaffold and 5 nucleotides on the candidate engineered guide RNA side of the guide-target RNA scaffold. An asymmetrical internal loop of the present disclosure can be formed by 100 nucleotides on the target RNA side of the guide-target RNA scaffold and 5 nucleotides on the candidate engineered guide RNA side of the guide-target RNA scaffold. An asymmetrical internal loop of the present disclosure can be formed by 50 nucleotides on the target RNA side of the guide-target RNA scaffold and 5 nucleotides on the candidate engineered guide RNA side of the guide-target RNA scaffold. An asymmetrical internal loop of the present disclosure can be formed by 50 nucleotides on the target RNA side of the guide-target RNA scaffold and 100 nucleotides on the candidate engineered guide RNA side of the guide-target RNA scaffold. An asymmetrical internal loop of the present disclosure can be formed by 50 nucleotides on the target RNA side of the guide-target RNA scaffold and 150 nucleotides on the candidate engineered guide RNA side of the guide-target RNA scaffold. An asymmetrical internal loop of the present disclosure can be formed by 50 nucleotides on the target RNA side of the guide-target RNA scaffold and 200 nucleotides on the candidate engineered guide RNA side of the guide-target RNA scaffold. An asymmetrical internal loop of the present disclosure can be formed by 50 nucleotides on the target RNA side of the guide-target RNA scaffold and 300 nucleotides on the candidate engineered guide RNA side of the guide-target RNA scaffold. An asymmetrical internal loop of the present disclosure can be formed by 50 nucleotides on the target RNA side of the guide-target RNA scaffold and 400 nucleotides on the candidate engineered guide RNA side of the guide-target RNA scaffold. An asymmetrical internal loop of the present disclosure can be formed by 50 nucleotides on the target RNA side of the guide-target RNA scaffold and 500 nucleotides on the candidate engineered guide RNA side of the guide-target RNA scaffold. An asymmetrical internal loop of the present disclosure can be formed by 50 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 50 nucleotides on the candidate engineered guide RNA side of the guide-target RNA scaffold. An asymmetrical internal loop of the present disclosure can be formed by 500 nucleotides on the target RNA side of the guide-target RNA scaffold and 50 nucleotides on the candidate engineered guide RNA side of the guide-target RNA scaffold. An asymmetrical internal loop of the present disclosure can be formed by 400 nucleotides on the target RNA side of the guide-target RNA scaffold and 50 nucleotides on the candidate engineered guide RNA side of the guide-target RNA scaffold. An asymmetrical internal loop of the present disclosure can be formed by 300 nucleotides on the target RNA side of the guide-target RNA scaffold and 50 nucleotides on the candidate engineered guide RNA side of the guide-target RNA scaffold. An asymmetrical internal loop of the present disclosure can be formed by 200 nucleotides on the target RNA side of the guide-target RNA scaffold and 50 nucleotides on the candidate engineered guide RNA side of the guide-target RNA scaffold. An asymmetrical internal loop of the present disclosure can be formed by 150 nucleotides on the target RNA side of the guide-target RNA scaffold and 50 nucleotides on the candidate engineered guide RNA side of the guide-target RNA scaffold. An asymmetrical internal loop of the present disclosure can be formed by 100 nucleotides on the target RNA side of the guide-target RNA scaffold and 50 nucleotides on the candidate engineered guide RNA side of the guide-target RNA scaffold. An asymmetrical internal loop of the present disclosure can be formed by 100 nucleotides on the target RNA side of the guide-target RNA scaffold and 150 nucleotides on the candidate engineered guide RNA side of the guide-target RNA scaffold. An asymmetrical internal loop of the present disclosure can be formed by 100 nucleotides on the target RNA side of the guide-target RNA scaffold and 200 nucleotides on the candidate engineered guide RNA side of the guide-target RNA scaffold. An asymmetrical internal loop of the present disclosure can be formed by 100 nucleotides on the target RNA side of the guide-target RNA scaffold and 300 nucleotides on the candidate engineered guide RNA side of the guide-target RNA scaffold. An asymmetrical internal loop of the present disclosure can be formed by 100 nucleotides on the target RNA side of the guide-target RNA scaffold and 400 nucleotides on the candidate engineered guide RNA side of the guide-target RNA scaffold. An asymmetrical internal loop of the present disclosure can be formed by 100 nucleotides on the target RNA side of the guide-target RNA scaffold and 500 nucleotides on the candidate engineered guide RNA side of the guide-target RNA scaffold. An asymmetrical internal loop of the present disclosure can be formed by 100 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 100 nucleotides on the candidate engineered guide RNA side of the guide-target RNA scaffold. An asymmetrical internal loop of the present disclosure can be formed by 500 nucleotides on the target RNA side of the guide-target RNA scaffold and 100 nucleotides on the candidate engineered guide RNA side of the guide-target RNA scaffold. An asymmetrical internal loop of the present disclosure can be formed by 400 nucleotides on the target RNA side of the guide-target RNA scaffold and 100 nucleotides on the candidate engineered guide RNA side of the guide-target RNA scaffold. An asymmetrical internal loop of the present disclosure can be formed by 300 nucleotides on the target RNA side of the guide-target RNA scaffold and 100 nucleotides on the candidate engineered guide RNA side of the guide-target RNA scaffold. An asymmetrical internal loop of the present disclosure can be formed by 200 nucleotides on the target RNA side of the guide-target RNA scaffold and 100 nucleotides on the candidate engineered guide RNA side of the guide-target RNA scaffold. An asymmetrical internal loop of the present disclosure can be formed by 150 nucleotides on the target RNA side of the guide-target RNA scaffold and 100 nucleotides on the candidate engineered guide RNA side of the guide-target RNA scaffold. An asymmetrical internal loop of the present disclosure can be formed by 150 nucleotides on the target RNA side of the guide-target RNA scaffold and 200 nucleotides on the candidate engineered guide RNA side of the guide-target RNA scaffold. An asymmetrical internal loop of the present disclosure can be formed by 150 nucleotides on the target RNA side of the guide-target RNA scaffold and 300 nucleotides on the candidate engineered guide RNA side of the guide-target RNA scaffold. An asymmetrical internal loop of the present disclosure can be formed by 150 nucleotides on the target RNA side of the guide-target RNA scaffold and 400 nucleotides on the candidate engineered guide RNA side of the guide-target RNA scaffold. An asymmetrical internal loop of the present disclosure can be formed by 150 nucleotides on the target RNA side of the guide-target RNA scaffold and 500 nucleotides on the candidate engineered guide RNA side of the guide-target RNA scaffold. An asymmetrical internal loop of the present disclosure can be formed by 150 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 150 nucleotides on the candidate engineered guide RNA side of the guide-target RNA scaffold. An asymmetrical internal loop of the present disclosure can be formed by 500 nucleotides on the target RNA side of the guide-target RNA scaffold and 5 nucleotides on the candidate engineered guide RNA side of the guide-target RNA scaffold. An asymmetrical internal loop of the present disclosure can be formed by 400 nucleotides on the target RNA side of the guide-target RNA scaffold and 150 nucleotides on the candidate engineered guide RNA side of the guide-target RNA scaffold. An asymmetrical internal loop of the present disclosure can be formed by 300 nucleotides on the target RNA side of the guide-target RNA scaffold and 150 nucleotides on the candidate engineered guide RNA side of the guide-target RNA scaffold. An asymmetrical internal loop of the present disclosure can be formed by 200 nucleotides on the target RNA side of the guide-target RNA scaffold and 300 nucleotides on the candidate engineered guide RNA side of the guide-target RNA scaffold. An asymmetrical internal loop of the present disclosure can be formed by 200 nucleotides on the target RNA side of the guide-target RNA scaffold and 400 nucleotides on the candidate engineered guide RNA side of the guide-target RNA scaffold. An asymmetrical internal loop of the present disclosure can be formed by 200 nucleotides on the target RNA side of the guide-target RNA scaffold and 500 nucleotides on the candidate engineered guide RNA side of the guide-target RNA scaffold. An asymmetrical internal loop of the present disclosure can be formed by 200 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 200 nucleotides on the candidate engineered guide RNA side of the guide-target RNA scaffold. An asymmetrical internal loop of the present disclosure can be formed by 500 nucleotides on the target RNA side of the guide-target RNA scaffold and 200 nucleotides on the candidate engineered guide RNA side of the guide-target RNA scaffold. An asymmetrical internal loop of the present disclosure can be formed by 400 nucleotides on the target RNA side of the guide-target RNA scaffold and 200 nucleotides on the candidate engineered guide RNA side of the guide-target RNA scaffold. An asymmetrical internal loop of the present disclosure can be formed by 300 nucleotides on the target RNA side of the guide-target RNA scaffold and 200 nucleotides on the candidate engineered guide RNA side of the guide-target RNA scaffold. An asymmetrical internal loop of the present disclosure can be formed by 300 nucleotides on the target RNA side of the guide-target RNA scaffold and 400 nucleotides on the candidate engineered guide RNA side of the guide-target RNA scaffold. An asymmetrical internal loop of the present disclosure can be formed by 300 nucleotides on the target RNA side of the guide-target RNA scaffold and 500 nucleotides on the candidate engineered guide RNA side of the guide-target RNA scaffold. An asymmetrical internal loop of the present disclosure can be formed by 300 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 300 nucleotides on the candidate engineered guide RNA side of the guide-target RNA scaffold. An asymmetrical internal loop of the present disclosure can be formed by 500 nucleotides on the target RNA side of the guide-target RNA scaffold and 300 nucleotides on the candidate engineered guide RNA side of the guide-target RNA scaffold. An asymmetrical internal loop of the present disclosure can be formed by 400 nucleotides on the target RNA side of the guide-target RNA scaffold and 300 nucleotides on the candidate engineered guide RNA side of the guide-target RNA scaffold. An asymmetrical internal loop of the present disclosure can be formed by 400 nucleotides on the target RNA side of the guide-target RNA scaffold and 500 nucleotides on the candidate engineered guide RNA side of the guide-target RNA scaffold. An asymmetrical internal loop of the present disclosure can be formed by 400 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 400 nucleotides on the candidate engineered guide RNA side of the guide-target RNA scaffold. An asymmetrical internal loop of the present disclosure can be formed by 500 nucleotides on the target RNA side of the guide-target RNA scaffold and 400 nucleotides on the candidate engineered guide RNA side of the guide-target RNA scaffold. An asymmetrical internal loop of the present disclosure can be formed by 500 nucleotides on the target RNA side of the guide-target RNA scaffold and 1000 nucleotides on the candidate engineered guide RNA side of the guide-target RNA scaffold. An asymmetrical internal loop of the present disclosure can be formed by 1000 nucleotides on the target RNA side of the guide-target RNA scaffold and 500 nucleotides on the candidate engineered guide RNA side of the guide-target RNA scaffold. Thus, an asymmetrical internal loop can be a structural feature formed from latent structure provided by an engineered latent guide RNA.

Structural features that comprise an internal loop can be of any size greater than 5 bases. In some cases, an internal loop comprises at least: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 bases. In some cases, an internal loop comprise at least about 5-10, 5-15, 10-20, 15-25, 20-30, 5-30, 5-40, 5-50, 5-60, 5-70, 5-80, 5-90, 5-100, 5-110, 5-120, 5-130, 5-140, 5-150, 5-200, 5-250, 5-300, 5-350, 5-400, 5-450, 5-500, 5-600, 5-700, 5-800, 5-900, 5-1000, 20-50, 20-60, 20-70, 20-80, 20-90, 20-100, 20-110, 20-120, 20-130, 20-140, 20-150, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 bases in total.

In some embodiments, the library of guide-target RNA scaffolds can include one or more guide-target RNA scaffolds with a hairpin formed by the hybridization of a gRNA and a target RNA. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the candidate engineered guide RNAs disclosed herein. The candidate engineered guide RNAs disclosed herein can have from 1 to 10 hairpins. In some embodiments, the candidate engineered guide RNAs disclosed herein have 1 hairpin. In some embodiments, the candidate engineered guide RNAs disclosed herein have 2 hairpins. As disclosed herein, a hairpin can include a recruitment hairpin or a non-recruitment hairpin. A hairpin can be located anywhere within the candidate engineered guide RNAs of the present disclosure. In some embodiments, one or more hairpins is proximal to or present at the 3′ end of a candidate engineered guide RNA of the present disclosure, proximal to or at the 5′ end of a candidate engineered guide RNA of the present disclosure, proximal to or within the targeting domain of the candidate engineered guide RNAs of the present disclosure, or any combination thereof.

Hairpins formed by the hybridization of a gRNA and a target RNA include for example, recruitment hairpins and non-recruitment hairpins. A recruitment hairpin, as disclosed herein, can recruit at least in part an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that can be present in constructs comprising a candidate engineered guide RNA, not a structural feature formed by latent structure provided in an engineered latent guide RNA. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the candidate engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by hybridization of the guide RNA to the target RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

In some embodiments, the library of guide-target RNA scaffolds can include one or more guide-target RNA scaffolds with a wobble base pair formed by the hybridization of a gRNA and a target RNA. A wobble base pair refers to two bases that weakly pair. For example, a wobble base pair of the present disclosure can refer to a G paired with a U.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

Barbell Macro-Footprints

In some embodiments, a candidate guide RNA can comprise a macro-footprint sequence such as a barbell macro-footprint. As disclosed herein, a barbell macro-footprint sequence of a candidate guide RNA, upon hybridization to a target RNA, produces a pair of internal loop structural features that improve one or more aspects of editing, as compared to an otherwise comparable guide RNA lacking the pair of internal loop structural features. In some instances, inclusion of a barbell macro-footprint sequence improves an amount of editing of an adenosine of interest (e.g., an on-target adenosine), relative to an amount of editing of on-target adenosine in a comparable guide RNA lacking the barbell macro-footprint sequence. In some instances, inclusion of a barbell macro-footprint sequence decreases an amount of editing of adenosines other than the adenosine of interest (e.g., decreases off-target adenosine), relative to an amount of off-target adenosine in a comparable guide RNA lacking the barbell macro-footprint sequence.

A macro-footprint sequence can be positioned such that it flanks, is proximal to, or is adjacent to, a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

In some embodiments, a macro-footprint sequence can comprise a barbell macro-footprint sequence comprising latent structures that, when manifested, produce a first internal loop and a second internal loop. The positioning of the first and second internal loop with respect to structural features of the micro-footprint (such as the A/C mismatch), as well as the number of nucleotides present in each internal loop, can be selected using the high-throughput methods described herein. Accordingly, disclosed herein are methods of screening a macro-footprint sequence in a candidate guide RNA having a micro-footprint sequence, where the position of the macro-footprint sequence relative to the micro-footprint sequence is optimized to improve one or more facets of editing, relative to an otherwise comparable guide RNA lacking the macro-footprint sequence.

In some examples, a first internal loop is positioned “near the 5′ end of the guide-target RNA scaffold” and a second internal loop is positioned near the 3′ end of the guide-target RNA scaffold. The length of the dsRNA comprises a 5′ end and a 3′ end, where up to half of the length of the guide-target RNA scaffold at the 5′ end can be considered to be “near the 5′ end” while up to half of the length of the guide-target RNA scaffold at the 3′ end can be considered “near the 3′ end.” Non-limiting examples of the 5′ end can include about 50% or less of the total length of the dsRNA at the 5′ end, about 45%, about 40%, about 35%, about 30%, about 25%, about 20%, about 15%, about 10%, or about 5%. Non-limiting examples of the 3′ end can include about 50% or less of the total length of the dsRNA at the 3′ end about 45%, about 40%, about 35%, about 30%, about 25%, about 20%, about 15%, about 10%, or about 5%.

The candidate guide RNAs of the disclosure comprising a barbell macro-footprint sequence (that manifests as a first internal loop and a second internal loop) can improve RNA editing efficiency of a guide RNA for facilitating editing a target RNA, increase the amount or percentage of RNA editing generally, as well as for on-target nucleotide editing, such as on-target adenosine. In some embodiments, the candidate guide RNAs of the disclosure comprising a first internal loop and a second internal loop can also facilitate a decrease in the amount of or reduce off-target nucleotide editing, such as off-target adenosine or unintended adenosine editing. The decrease or reduction in some examples can be of the number of off-target edits or the percentage of off-target edits.

Each of the first and second internal loops of the barbell macro-footprint can independently be symmetrical or asymmetrical, where symmetry is determined by the number of bases or nucleotides of the candidate guide RNA and the number of bases or nucleotides of the target RNA, that together form each of the first and second internal loops.

As described herein, a double stranded RNA (dsRNA) substrate (a guide-target RNA scaffold) is formed upon hybridization of a candidate guide RNA of the present disclosure to a target RNA. An internal loop can be a symmetrical internal loop or an asymmetrical internal loop. A “symmetrical internal loop” is formed when the same number of nucleotides is present on each side of the internal loop. For example, a symmetrical internal loop in a guide-target RNA scaffold of the present disclosure can have the same number of nucleotides on the candidate guide RNA side and the target RNA side of the guide-target RNA scaffold. A symmetrical internal loop of the present disclosure can be formed by 5 nucleotides on the candidate guide RNA side of the guide-target RNA scaffold target and 5 nucleotides on the target RNA side of the guide-target RNA scaffold. A symmetrical internal loop of the present disclosure can be formed by 6 nucleotides on the candidate guide RNA side of the guide-target RNA scaffold target and 6 nucleotides on the target RNA side of the guide-target RNA scaffold. A symmetrical internal loop of the present disclosure can be formed by 7 nucleotides on the candidate guide RNA side of the guide-target RNA scaffold target and 7 nucleotides on the target RNA side of the guide-target RNA scaffold. A symmetrical internal loop of the present disclosure can be formed by 8 nucleotides on the candidate guide RNA side of the guide-target RNA scaffold target and 8 nucleotides on the target RNA side of the guide-target RNA scaffold. A symmetrical internal loop of the present disclosure can be formed by 9 nucleotides on the candidate guide RNA side of the guide-target RNA scaffold target and 9 nucleotides on the target RNA side of the guide-target RNA scaffold. A symmetrical internal loop of the present disclosure can be formed by 10 nucleotides on the candidate guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

As described herein, a double stranded RNA (dsRNA) substrate (e.g., a guide-target RNA scaffold) is formed upon hybridization of a candidate guide RNA of the present disclosure to a target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the candidate guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the candidate guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the candidate guide RNA side of the guide-target RNA scaffold. An asymmetrical internal loop of the present disclosure can be formed by 5 nucleotides on the candidate guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the candidate guide RNA side of the guide-target RNA scaffold. An asymmetrical internal loop of the present disclosure can be formed by 5 nucleotides on the candidate guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the candidate guide RNA side of the guide-target RNA scaffold. An asymmetrical internal loop of the present disclosure can be formed by 5 nucleotides on the candidate guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the candidate guide RNA side of the guide-target RNA scaffold. An asymmetrical internal loop of the present disclosure can be formed by 5 nucleotides on the candidate guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the candidate guide RNA side of the guide-target RNA scaffold. An asymmetrical internal loop of the present disclosure can be formed by 6 nucleotides on the candidate guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the candidate guide RNA side of the guide-target RNA scaffold. An asymmetrical internal loop of the present disclosure can be formed by 6 nucleotides on the candidate guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the candidate guide RNA side of the guide-target RNA scaffold. An asymmetrical internal loop of the present disclosure can be formed by 6 nucleotides on the candidate guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the candidate guide RNA side of the guide-target RNA scaffold. An asymmetrical internal loop of the present disclosure can be formed by 6 nucleotides on the candidate guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the candidate guide RNA side of the guide-target RNA scaffold. An asymmetrical internal loop of the present disclosure can be formed by 7 nucleotides on the candidate guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the candidate guide RNA side of the guide-target RNA scaffold. An asymmetrical internal loop of the present disclosure can be formed by 7 nucleotides on the candidate guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the candidate guide RNA side of the guide-target RNA scaffold. An asymmetrical internal loop of the present disclosure can be formed by 7 nucleotides on the candidate guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the candidate guide RNA side of the guide-target RNA scaffold. An asymmetrical internal loop of the present disclosure can be formed by 8 nucleotides on the candidate guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the candidate guide RNA side of the guide-target RNA scaffold. An asymmetrical internal loop of the present disclosure can be formed by 8 nucleotides on the candidate guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the candidate guide RNA side of the guide-target RNA scaffold. An asymmetrical internal loop of the present disclosure can be formed by 9 nucleotides on the candidate guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the candidate guide RNA side of the guide-target RNA scaffold. An asymmetrical internal loop of the present disclosure can be formed by 5 nucleotides on the target RNA side of the guide-target RNA scaffold and 50 nucleotides on the engineered polynucleotide side of the guide-target RNA scaffold. An asymmetrical internal loop of the present disclosure can be formed by 5 nucleotides on the target RNA side of the guide-target RNA scaffold and 100 nucleotides on the engineered polynucleotide side of the guide-target RNA scaffold. An asymmetrical internal loop of the present disclosure can be formed by 5 nucleotides on the target RNA side of the guide-target RNA scaffold and 150 nucleotides on the engineered polynucleotide side of the guide-target RNA scaffold. An asymmetrical internal loop of the present disclosure can be formed by 5 nucleotides on the target RNA side of the guide-target RNA scaffold and 200 nucleotides on the engineered polynucleotide side of the guide-target RNA scaffold. An asymmetrical internal loop of the present disclosure can be formed by 5 nucleotides on the target RNA side of the guide-target RNA scaffold and 300 nucleotides on the engineered polynucleotide side of the guide-target RNA scaffold. An asymmetrical internal loop of the present disclosure can be formed by 5 nucleotides on the target RNA side of the guide-target RNA scaffold and 400 nucleotides on the engineered polynucleotide side of the guide-target RNA scaffold. An asymmetrical internal loop of the present disclosure can be formed by 5 nucleotides on the target RNA side of the guide-target RNA scaffold and 500 nucleotides on the engineered polynucleotide side of the guide-target RNA scaffold. An asymmetrical internal loop of the present disclosure can be formed by 5 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 5 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 5 nucleotides on the engineered polynucleotide side of the guide-target RNA scaffold. An asymmetrical internal loop of the present disclosure can be formed by 300 nucleotides on the target RNA side of the guide-target RNA scaffold and 5 nucleotides on the engineered polynucleotide side of the guide-target RNA scaffold. An asymmetrical internal loop of the present disclosure can be formed by 200 nucleotides on the target RNA side of the guide-target RNA scaffold and 5 nucleotides on the engineered polynucleotide side of the guide-target RNA scaffold. An asymmetrical internal loop of the present disclosure can be formed by 150 nucleotides on the target RNA side of the guide-target RNA scaffold and 5 nucleotides on the engineered polynucleotide side of the guide-target RNA scaffold. An asymmetrical internal loop of the present disclosure can be formed by 100 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 100 nucleotides on the engineered polynucleotide disclosure can be formed by 50 nucleotides on the target RNA side of the guide-target RNA scaffold and 150 nucleotides on the engineered polynucleotide side of the guide-target RNA scaffold. An asymmetrical internal loop of the present disclosure can be formed by 50 nucleotides on the target RNA side of the guide-target RNA scaffold and 200 nucleotides on the engineered polynucleotide side of the guide-target RNA scaffold. An asymmetrical internal loop of the present disclosure can be formed by 50 nucleotides on the target RNA side of the guide-target RNA scaffold and 300 nucleotides on the engineered polynucleotide side of the guide-target RNA scaffold. An asymmetrical internal loop of the present disclosure can be formed by 50 nucleotides on the target RNA side of the guide-target RNA scaffold and 400 nucleotides on the engineered polynucleotide side of the guide-target RNA scaffold. An asymmetrical internal loop of the present disclosure can be formed by 50 nucleotides on the target RNA side of the guide-target RNA scaffold and 500 nucleotides on the engineered polynucleotide side of the guide-target RNA scaffold. An asymmetrical internal loop of the present disclosure can be formed by 50 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 50 nucleotides on the engineered polynucleotide side of the guide-target RNA scaffold. An asymmetrical internal loop of the present disclosure can be formed by 500 nucleotides on the target RNA side of the guide-target RNA scaffold and 50 nucleotides on the engineered polynucleotide side of the guide-target RNA scaffold. An asymmetrical internal loop of the present disclosure can be formed by 400 nucleotides on the target RNA side of the guide-target RNA scaffold and 50 nucleotides on the engineered polynucleotide side of the guide-target RNA scaffold. An asymmetrical internal loop of the present disclosure can be formed by 300 nucleotides on the target RNA side of the guide-target RNA scaffold and 50 nucleotides on the engineered polynucleotide side of the guide-target RNA scaffold. An asymmetrical internal loop of the present disclosure can be formed by 200 nucleotides on the target RNA side of the guide-target RNA scaffold and 50 nucleotides on the engineered polynucleotide side of the guide-target RNA scaffold. An asymmetrical internal loop of the present disclosure can be formed by 150 nucleotides on the target RNA side of the guide-target RNA scaffold and 50 nucleotides on the engineered polynucleotide disclosure can be formed by 100 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 150 nucleotides on the engineered polynucleotide side of the guide-target RNA scaffold. An asymmetrical internal loop of the present disclosure can be formed by 100 nucleotides on the target RNA side of the guide-target RNA scaffold and 200 nucleotides on the engineered polynucleotide side of the guide-target RNA scaffold. An asymmetrical internal loop of the present disclosure can be formed by 100 nucleotides on the target RNA side of the guide-target RNA scaffold and 300 nucleotides on the engineered polynucleotide side of the guide-target RNA scaffold. An asymmetrical internal loop of the present disclosure can be formed by 100 nucleotides on the target RNA side of the guide-target RNA scaffold and 400 nucleotides on the engineered polynucleotide side of the guide-target RNA scaffold. An asymmetrical internal loop of the present disclosure can be formed by 100 nucleotides on the target RNA side of the guide-target RNA scaffold and 500 nucleotides on the engineered polynucleotide side of the guide-target RNA scaffold. An asymmetrical internal loop of the present disclosure can be formed by 100 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 100 nucleotides on the engineered polynucleotide side of the guide-target RNA scaffold. An asymmetrical internal loop of the present disclosure can be formed by 500 nucleotides on the target RNA side of the guide-target RNA scaffold and 100 nucleotides on the engineered polynucleotide side of the guide-target RNA scaffold. An asymmetrical internal loop of the present disclosure can be formed by 400 nucleotides on the target RNA side of the guide-target RNA scaffold and 100 nucleotides on the engineered polynucleotide side of the guide-target RNA scaffold. An asymmetrical internal loop of the present disclosure can be formed by 300 nucleotides on the target RNA side of the guide-target RNA scaffold and 100 nucleotides on the engineered polynucleotide side of the guide-target RNA scaffold. An asymmetrical internal loop of the present disclosure can be formed by 200 nucleotides on the target RNA side of the guide-target RNA scaffold and 100 nucleotides on the engineered polynucleotide disclosure can be formed by 150 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 200 nucleotides on the engineered polynucleotide side of the guide-target RNA scaffold. An asymmetrical internal loop of the present disclosure can be formed by 150 nucleotides on the target RNA side of the guide-target RNA scaffold and 300 nucleotides on the engineered polynucleotide side of the guide-target RNA scaffold. An asymmetrical internal loop of the present disclosure can be formed by 150 nucleotides on the target RNA side of the guide-target RNA scaffold and 400 nucleotides on the engineered polynucleotide side of the guide-target RNA scaffold. An asymmetrical internal loop of the present disclosure can be formed by 150 nucleotides on the target RNA side of the guide-target RNA scaffold and 500 nucleotides on the engineered polynucleotide side of the guide-target RNA scaffold. An asymmetrical internal loop of the present disclosure can be formed by 150 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 150 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 150 nucleotides on the engineered polynucleotide side of the guide-target RNA scaffold. An asymmetrical internal loop of the present disclosure can be formed by 300 nucleotides on the target RNA side of the guide-target RNA scaffold and 150 nucleotides on the engineered polynucleotide side of the guide-target RNA scaffold. An asymmetrical internal loop of the present disclosure can be formed by 200 nucleotides on the target RNA side of the guide-target RNA scaffold and 300 nucleotides on the engineered polynucleotide side of the guide-target RNA scaffold. An asymmetrical internal loop of the present disclosure can be formed by 200 nucleotides on the target RNA side of the guide-target RNA scaffold and 400 nucleotides on the engineered polynucleotide side of the guide-target RNA scaffold. An asymmetrical internal loop of the present disclosure can be formed by 200 nucleotides on the target RNA side of the guide-target RNA scaffold and 500 nucleotides on the engineered polynucleotide side of the guide-target RNA scaffold. An asymmetrical internal loop of the present disclosure can be formed by 200 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 200 nucleotides on the engineered polynucleotide side of the guide-target RNA scaffold. An asymmetrical internal loop of the present disclosure can be formed by 500 nucleotides on the target RNA side of the guide-target RNA scaffold and 200 nucleotides on the engineered polynucleotide side of the guide-target RNA scaffold. An asymmetrical internal loop of the present disclosure can be formed by 400 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 400 nucleotides on the engineered polynucleotide side of the guide-target RNA scaffold. An asymmetrical internal loop of the present disclosure can be formed by 300 nucleotides on the target RNA side of the guide-target RNA scaffold and 500 nucleotides on the engineered polynucleotide side of the guide-target RNA scaffold. An asymmetrical internal loop of the present disclosure can be formed by 300 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 300 nucleotides on the engineered polynucleotide side of the guide-target RNA scaffold. An asymmetrical internal loop of the present disclosure can be formed by 500 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 500 nucleotides on the engineered polynucleotide side of the guide-target RNA scaffold. An asymmetrical internal loop of the present disclosure can be formed by 400 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 500 nucleotides on the engineered polynucleotide side of the guide-target RNA scaffold. Thus, an asymmetrical internal loop can be a structural feature formed from latent structure provided by an engineered latent guide RNA.

In some embodiments, a first internal loop or a second internal loop can independently comprise a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the candidate guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, a candidate guide RNA comprising a barbell macro-footprint (e.g., a latent structure that manifests as a first internal loop and a second internal loop) comprises a cytosine in a micro-footprint sequence in between the macro-footprint sequence that, when the candidate guide RNA is hybridized to the target RNA, is present in the guide-target RNA scaffold opposite an adenosine that is edited by the RNA editing entity (e.g., an on-target adenosine). In such embodiments, the cytosine of the micro-footprint is comprised in an A/C mismatch with the on-target adenosine of the target RNA in the guide-target RNA scaffold.

A first internal loop and a second internal loop of the barbell macro-footprint can be positioned a certain distance from the A/C mismatch, with respect to the base of the first internal loop and the base of the second internal loop that is the most proximal to the A/C mismatch. In some embodiments, the first internal loop and the second internal loop can be positioned the same number of bases from the A/C mismatch, with respect to the base of the first internal loop and the base of the second internal loop that is the most proximal to the A/C mismatch. In some embodiments, the first internal loop and the second internal loop can be positioned a different number of bases from the A/C mismatch, with respect to the base of the first internal loop and the base of the second internal loop that is the most proximal to the A/C mismatch.

In some embodiments, the first internal loop of the barbell or the second internal loop of the barbell can be positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell can be positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, the first internal loop can be positioned from about 5 bases away from the A/C mismatch to about 15 bases away from the A/C mismatch (e.g., 6-14, 7-13, 8-12, 9-11) with respect to the base of the first internal loop that is most proximal to the A/C mismatch. In some examples, the first internal loop can be positioned from about 9 bases away from the A/C mismatch to about 15 bases away from the A/C mismatch (e.g., 10-14, 11-13) with respect to the base of the first internal loop that is the most proximal to the A/C mismatch.

In some embodiments, the second internal loop can be positioned from about 12 bases away from the A/C mismatch to about 40 bases away from the A/C mismatch (e.g., 13-39, 14-38, 15-37, 16-36, 17-35, 18-34, 19-33, 20-32, 21-31, 22-30, 23-29, 24-28, 25-27) with respect to the base of the second internal loop that is the most proximal to the A/C mismatch. In some embodiments, the second internal loop can be positioned from about 20 bases away from the A/C mismatch to about 33 bases away from the A/C mismatch with respect to the base of the second internal loop that is most proximal to the A/C mismatch.

In some examples, a candidate engineered guide RNA can comprise an RNA editing entity recruiting domain (such as a recruitment hairpin) formed and present in the absence of binding to a target RNA. An RNA editing entity can be recruited by an RNA editing entity recruiting domain on a candidate engineered guide RNA. In some examples, a candidate engineered guide RNA comprising an RNA editing entity recruiting domain can be configured to facilitate editing of a base of a nucleotide of a polynucleotide of a region of a subject target RNA, modulation expression of a polypeptide encoded by the subject target RNA, or both. In some cases, a candidate engineered guide RNA can be configured to facilitate an editing of a base of a nucleotide or polynucleotide of a region of an RNA by a subject RNA editing entity. In order to facilitate editing, a candidate engineered guide RNA of the disclosure can recruit an RNA editing entity.

Various RNA editing entity recruiting domains can be utilized. In some examples, a recruiting domain comprises: Glutamate ionotropic receptor AMPA type subunit 2 (GluR2), APOBEC, MS2-bacteriophage-coat-protein-recruiting domain, Alu, a TALEN recruiting domain, a Zn-finger polypeptide recruiting domain, a mega-TAL recruiting domain, or a Cas13 recruiting domain, combinations thereof, or modified versions thereof. In some examples, more than one recruiting domain can be included in a candidate engineered guide of the disclosure. In examples where a recruiting sequence is present, the recruiting sequence can be utilized to position the RNA editing entity to effectively react with a subject target RNA after the targeting sequence, for example an antisense sequence, hybridizes to a target RNA. In some cases, a recruiting sequence can allow for transient binding of the RNA editing entity to the candidate engineered guide RNA. In some examples, the recruiting sequence allows for permanent binding of the RNA editing entity to the candidate engineered guide. A recruiting sequence can be of any length. In some cases, a recruiting sequence can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting sequence can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting sequence can be about 45 nucleotides in length. In some cases, at least a portion of a recruiting sequence comprises at least 1 to about 75 nucleotides. In some cases, at least a portion of a recruiting sequence comprises about 45 nucleotides to about 60 nucleotides. In some aspects, an RNA editing entity recruiting domain can form a recruitment hairpin, as disclosed herein. A recruitment hairpin can recruit an RNA editing entity, such as ADAR. In some embodiments, a recruitment hairpin comprises a GluR2 domain. In some embodiments, a recruitment hairpin comprises an Alu domain.

In an embodiment, an RNA editing entity recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 213). In some cases, a recruiting domain can comprise at least about 80% sequence homology to at least about 10, 15, 20, 25, or 30 nucleotides of SEQ ID NO: 3. In some examples, a recruiting domain can comprise at least about 90%, 95%, 96%, 97%, 98%, or 99% sequence homology and/or length to GUGGAAUAGUAUAACAAUAUGCUAAAUGUUGUUAUAGUAUCCCAC (SEQ ID NO: 213).

Additional RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In some examples, a recruiting domain can be from an MS2-bacteriophage-coat-protein-recruiting domain. In another embodiment, a recruiting domain can be from an Alu domain. In some examples, a recruiting domain can comprise at least about: 70%, 80%, 85%, 90%, or 95% sequence homology and/or length to at least about: 15, 20, 25, 30, or 35 nucleotides of an APOBEC, MS2-bacteriophage-coat-protein-recruiting domain, or Alu domain.

In some embodiments, a candidate guide RNA can comprise at least one internal recruitment hairpin embedded within structural features present in a micro-footprint. For example, a recruitment hairpin can be embedded within an internal loop described herein to form a junctioned candidate guide RNA. In some instances, a junctioned candidate guide RNA can comprise a recruitment hairpin (for example, a GluR2 hairpin) embedded on an internal loop substantially centered in a micro-footprint, thus forming a junctioned candidate guide RNA with multiple hairpin protrusions. Each hairpin protrusion can be separated by varying number of nucleotides along the internal loop. For example, each hairpin protrusion can be independently separated by about 1, 2, 3, 4, 5, 6, 7, 8, 9, 10, 11, 12, 13, 14, 15, 16, 17, 18, 19, or more than 20 nucleotides.

In some embodiments, a recruitment hairpin such as a GluR2 hairpin can be embedded in an internal loop substantially centered in a micro-footprint. Such a configuration produces a 3-way junction, where the resulting junctioned candidate guide RNA has 3 hairpin protrusions. In some embodiments, multiple recruitment hairpins such as a GluR2 hairpin can be embedded in an internal loop substantially centered in a micro-footprint. For instance, where two GluR2 hairpins are embedded producing a 4-way junction, the resulting junctioned candidate guide RNA can have 4 hairpin protrusions. Additional hairpins, whether recruitment hairpins or otherwise, can be embedded to produce such multiply functioned candidate guide RNAs with improved editing efficiencies.

2. Target RNAs

The subject methods provided herein can be used for the screening of gRNAs for the editing of any suitable target RNA, including for example, mutant alleles that are associated with a particular disorder. In some examples, the target RNA is an mRNA molecule. In some examples, the mRNA molecule comprises a premature stop codon. In some examples, the mRNA comprises 1, 2, 3, 4 or 5 premature stop codons. In some examples, the stop codon is an amber stop codon (UAG), an ochre stop codon (UAA), or an opal stop codon (UGA), or a combination thereof. In some examples, the premature stop codon is created by a point mutation. In some examples, the premature stop codon causes translation termination of an expression product expressed by the mRNA molecule. In some examples, the premature stop codon is produced by a point mutation on an mRNA molecule in combination with two additional nucleotides. In some examples, the two additional nucleotides are (i) a U and (ii) an A or a G, on a 5′ and a 3′ end of the point mutation.

In some examples, the target RNA is a pre-mRNA molecule. In some examples, the pre-mRNA molecule comprises a splice site mutation. In some examples, the splice site mutation facilitates unintended splicing of a pre-mRNA molecule. In some examples, the splice site mutation results in mistranslation and/or truncation of a protein encoded by the pre-mRNA molecule.

In some examples, the target RNA molecule is a pre-mRNA or mRNA molecule encoded by a APP, ABCA4, SERPINA1, HEXA, LRRK2, CFTR, SNCA, MAPT, DUX4, PMP22, DMPK, SOD1, GRN, LIPA, DMD gene, a fragment of any of these, or any combination thereof. In some examples, the target RNA molecule is encoded by APP, ABCA4, SERPINA1, HEXA, LRRK2, CFTR, SNCA, MAPT, DUX4, PMP22, DMPK, SOD1, GRN, LIPA, DMD, a fragment any of these, or any combination thereof. In some examples, the target RNA molecule encodes a ABCA4, APP, SERPINA1, HEXA, LRRK2, SNCA, CFTR, APP, GBA, PINK1, DUX4, PMP22, DMPK, SOD1, progranulin, Tau, or LIPA protein, a fragment of any of these, or a combination thereof. In some examples, the target RNA molecule encodes ABCA4, AAT, SERPINA1, SERPINA1 E342K, HEXA, LRRK2, SNCA, APP, DUX4, PMP22, DMPK, SOD1, progranulin, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, the DNA encoding the RNA molecule comprises a mutation relative to an otherwise identical reference DNA molecule. In some examples, the RNA molecule comprises a mutation relative to an otherwise identical reference RNA molecule. In some examples, the protein encoded for by the target RNA molecule comprises a mutation relative to an otherwise identical reference protein.

In some embodiments, the target RNA molecule contains a mutation implicated in a disease pathway. For example, the target RNA molecule can be LRRK2 and the mutation can be the LRRK2 G2019S mutation.

In some examples, the HTS methods disclosed herein screen for guide RNAs against a target RNA molecule from a gene implicated in a disease, for example, any neurological disease (e.g., Parkinson's disease, Alzheimer's disease, Tauopathies, FTD), Stargardt disease, Duchenne's muscular dystrophy, cystic fibrosis, or alpha-1 antitrypsin deficiency.

B. RNA-Editing Entities

In some embodiments, the self-annealing RNA structure library are contacted with an RNA editing entity to edit under conditions that allow the RNA editing entity to edit one or more of the target RNAs in the library. In exemplary embodiments, the RNA editing entity is native to the subject from which the target RNA is derived. In some embodiments, the RNA editing entity is a human RNA editing entity and the target gene is a derived from an allele of a human gene of interest (e.g., a mutant allele of a human gene of interest).

In some examples, an RNA editing entity comprises an adenosine deaminase acting on RNA (ADAR). ADARs catalyze adenosine to inosine (A to I) editing in RNA. Inosine is a deaminated form of adenosine and is biochemically recognized as guanine. In some examples, an ADAR comprises any one of: ADAR1, ADAR1p110, ADAR1p150, ADAR2, ADAR3, APOBEC protein, or any combination thereof. In some examples, the ADAR RNA editing entity is ADAR1. In some examples, additionally, or alternatively, the ADAR RNA editing entity is ADAR2. In some examples, additionally, or alternatively, the ADAR RNA editing entity is ADAR3. In an aspect, an RNA editing entity can be a non-ADAR. In some examples, the RNA editing entity is an APOBEC protein. In some examples, the RNA editing entity is APOBEC1, APOBEC2, APOBEC3A, APOBEC3B, APOBEC3C, APOBEC3E, APOBEC3F, APOBEC3G, APOBEC3H, APOBEC4, or any combination thereof. In some examples, the ADAR or APOBEC is mammalian. In some examples, the ADAR or APOBEC protein is human. In some examples, the ADAR or APOBEC protein is recombinant (e.g., an exogenously delivered recombinant ADAR or APOBEC protein), modified (e.g., an exogenously delivered modified ADAR or APOBEC protein), endogenous, or any combination thereof. In some examples, the RNA editing entity can be a fusion protein. In some examples, the RNA editing entity can be a functional portion of an RNA editing entity, such as any of the RNA editing proteins provided herein. In some instances, an RNA editing entity can comprise at least about 70% sequence homology and/or length to APOBEC1, APOBEC2, ADAR1, ADAR1p110, ADAR1p150, ADAR2, ADAR3, or any combination thereof.

The cells that include the self-annealing RNA structure library are exposed to the RNA editing entity (e.g., ADAR1 and/or ADAR2) over a time course reaction. In some embodiments, the cells are contacted with ADAR1 or ADAR2. In certain embodiments, the cells are contacted with a combination of ADAR1 and ADAR2. In exemplary embodiments, the cells are contacted with ADAR1 and/or ADAR2 in the presence of one or more ADAR inhibitors or trans-regulators. In some embodiments, the self-annealing RNA structure library is exposed to the RNA editing entity for at least about 5, 10, 15, 20, 25, 30, 35, 40, 45, 50, 55, or 60 second seconds. In some embodiments, the self-annealing RNA structure library is exposed to the RNA editing entity for about 5-10, 10-15, 15-20, 20-25, 25-30, 35-40, 45-50, 50-55, 55-60, 1-30, 20-40, 30-50, or 40-60 seconds. In some embodiments, the self-annealing RNA structure library is exposed to the RNA editing entity for at least about 1, 2, 3, 4, 5, 6, 7, 8, 9, 10, 20, 25, 30, 35, 40, 45, 50, 55, 60, 65, 70, 75, 80, 85, 90, 95 or 100 minutes. In certain embodiments, the self-annealing RNA structure library is exposed to the RNA editing entity for about 1-5, 5-10, 10-15, 15-20, 25-30, 35-40, 45-50, 55-60, 60-65, 65-70, 70-75, 75-80, 85-90, 95-100, 1-20, 20-40, 40-60, 60-80, or 70-90 minutes. In certain embodiments, the self-annealing RNA structure library is exposed to the RNA editing entity for at least about 100, 150, 200, 250, 300, 350, 400, 450, 450, 500, 550, 600, 650, 700, 750, 800, 850, 900, 950, or 1,000 minutes. In exemplary embodiments, the self-annealing RNA structure library is exposed to the RNA editing entity for at least about 100-150, 150-200, 200-250, 250-300, 300-350, 350-400, 400-450, 450-500, 500-550, 550-600, 600-650, 650-700, 700-750, 750-800, 800-850, 850-900, 950-1,000, 100-300, 200-400, 300-500, 400-600, 500-700, 600-800, or 700-900 minutes.

The methods provided herein can be performed in cell culture systems and cell-free systems. In some embodiments where the method is performed in a cell culture system that includes a plurality of cells, each of the cells includes one or more of the guide-target RNA scaffolds of the library and the cells are contacted with an expression vector that is capable of expressing an RNA editing entity within the cell.

C. Identification of Edited Self-Annealing RNA Structure

After exposing the self-annealing RNA structure library to the RNA editing entity, the self-annealing RNA structure substrate library is isolated and RT-PCR is carried out to produce amplicons of the guide-target RNA scaffolds. In some embodiments, each of the self-annealing RNA structures include one or more universal primer binding sites that facilitate the production of amplicons (see FIG. 4).

The amplicons are then analyzed for editing events mediated by the RNA editing entity for example, by high throughput sequencing methods such as next generation sequencing (NGS). In some embodiments each of the self-annealing RNA structures in the library include one or more unique barcodes that facilitate multiplex sequencing and identification of edited self-annealing RNA structures based on analysis of the amplicons. In some embodiments wherein the RNA editing entity is an ADAR, the substrates are analyzed for an adenosine deamination event at a site of interest in the target gene.

In embodiments wherein the target RNA and gRNA are covalently attached by a hairpin linker, the physical linkage of target and guide strand in a hairpin structure and NGS allow for editing selectivity and kinetics to be quantified for each guide design. Detailed knowledge of editing selectivity and efficiency for such a large and diverse set of designs can be used to select features for incorporation into guide RNA molecules for trans-mediated RNA editing, and can be used for machine learning to inform future designs.

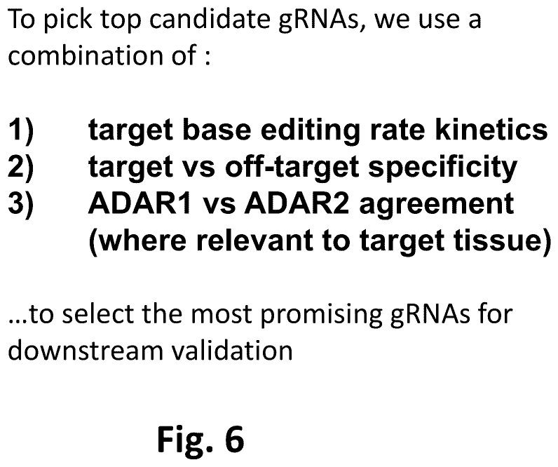

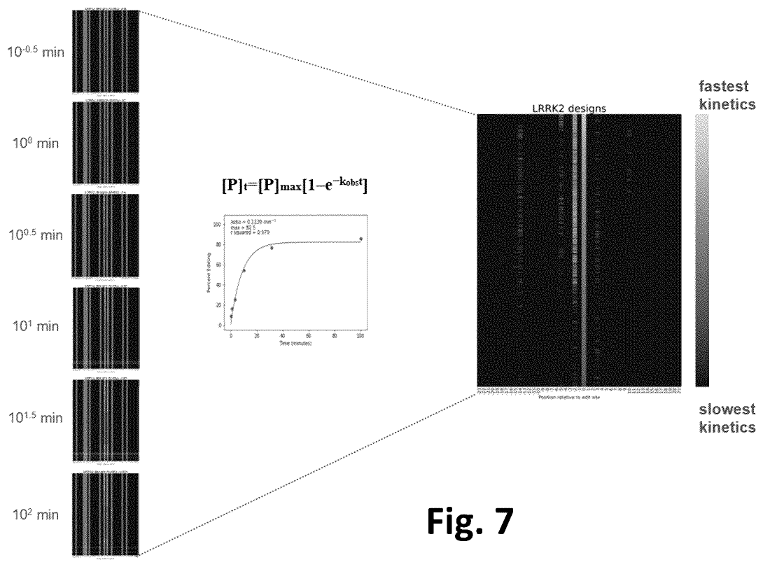

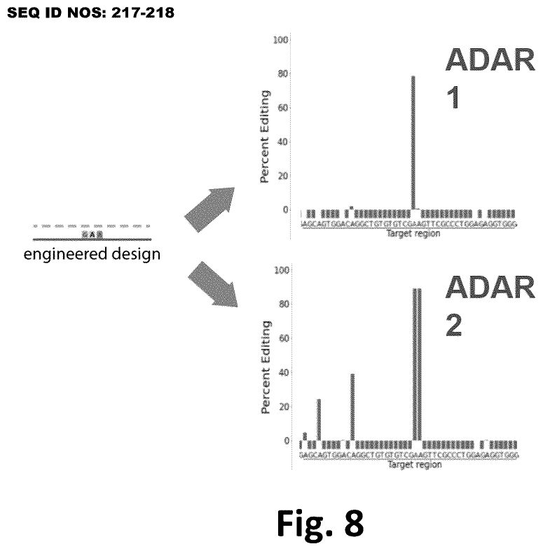

In some embodiments, the self-annealing RNA structures are assessed for the ability to mediate target specific editing by an RNA editing entity (e.g., an ADAR) based on one or more metrics (FIG. 6). In some embodiments, amplicons of self-annealing RNA structures are assessed for on-target editing events as compared to off-target editing events. In some embodiments, a candidate guide RNA is considered suitable for editing a particular target RNA if at least about 50%, 55%, 60%, 65%, 70%, 75%, 80%, 85%, 90%, 91%, 92%, 93%, 94%, 95%, 96%, 97%, 98% or 99% of the edits mediated by the candidate guide RNA are on-target specific edits. In some embodiments, the editing of a target RNA mediated by a candidate guide RNA is assessed over a period of time. In exemplary embodiments, ADAR (e.g., ADAR1 and ADAR 2) editing of a target RNA mediated by a candidate guide RNA is assessed over a period of time (FIG. 7). In some embodiments, such kinetic studies are performed over a period of about 1, 2, 3, 4, 5, 10, 15, 20, 25, 30, 35, 40, 45, 50, 55, or 60 seconds. In some embodiments, such kinetic studies are performed over a period of about 1, 2, 3, 4, 5, 10, 15, 20, 25, 30, 35, 40, 45, 50, 55, 60, 65, 70, 75, 80, 85, 90, 95, 100, 150, 200, 250 or 500 minutes. In some embodiments, the self-annealing RNA structures are assessed for the ability to mediate on-target editing using two or more different RNA editing entities (e.g., ADAR1 and ADAR2). In certain embodiments, a candidate guide RNA is considered suitable for editing a particular target RNA if it is capable of mediating both ADAR1 and ADAR2 on-target editing of the target RNA. In some embodiments, a candidate guide RNA is considered suitable for editing a particular target RNA if at least about 50%, 55%, 60%, 65%, 70%, 75%, 80%, 85%, 90%, 91%, 92%, 93%, 94%, 95%, 96%, 97%, 98% or 99% of the ADAR1 and ADAR2 edits mediated by the candidate guide are on-target specific edits. In exemplary embodiments, the RNA editing entity is assessed for ADAR1 and/or ADAR2 on-target editing in the presence of one or more ADAR inhibitors and/or trans-regulators. In certain embodiments, candidate self-annealing RNA structures are assessed for stability. In exemplary embodiments, stability is assessed based on the rate of degradation of the self-annealing RNA structures under a particular condition using any suitable method.

Such methods can be used to identify an optimal set of structural features that form upon hybridization of a candidate guide RNA to a target RNA that enable the candidate guide RNA to facilitate editing of the target RNA. As described herein, the structural features can be present in a micro-footprint sequence of the candidate guide RNA, the macro-footprint sequence of the candidate guide RNA, or both. In some instances, an optimized micro-footprint sequence can be determined using methods described herein, thus producing a candidate guide RNA with a unique micro-footprint sequence that can vary on a target-by-target basis. In some instances, a guide RNA having an optimized micro-footprint sequence can be further optimized to improve one or more facets of editing by adding a macro-footprint sequence as described herein. For example, a barbell macro-footprint sequence having a first internal loop and a second internal loop can be incorporated, such that the first internal loop and the second internal loop flank the optimized micro-footprint sequence. Further optimization of the barbell macro-footprint can include determining an optimal size for each of the first internal loop and the second internal loop, as well as the relative position of the first internal loop and the second internal loop relative to a structural feature of the optimized micro-footprint (such as an A/C mismatch). In some instances, an optimized micro-footprint and an optimized macro-footprint can be simultaneously developed using the high-throughput screening methods described herein. In some instances, an optimized micro-footprint can be developed prior to screening of the barbell macro-footprint using the high-throughput screening methods described herein. In some instances, a barbell macro-footprint can be optimized for a candidate guide RNA having an optimized micro-footprint against a first target, and subsequently the optimized barbell macro-footprint can be incorporated into a candidate guide RNA against a second or subsequent targets without the need for further optimization.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

A “barbell” as described herein refers to a pair of internal loop latent structures that manifest upon hybridization of the guide RNA to the target RNA.

As disclosed herein, a “bulge” refers to the structure substantially formed only upon formation of the guide-target RNA scaffold, where contiguous nucleotides in either the candidate engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the candidate engineered guide RNA and a single participating nucleotide of the target RNA do not base pair—a single participating nucleotide of the candidate engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element may be present or not (for example, detection). These terms can include quantitative, qualitative or quantitative and qualitative determinations. Assessing can be relative or absolute. “Detecting the presence of” can include determining the amount of something present in addition to determining whether it may be present or absent depending on the context.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

An “engineered latent guide RNA” refers to a candidate engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by a candidate engineered guide RNA refers to the ability of the candidate engineered guide RNA when associated with an RNA editing entity and a target RNA to provide a targeted edit of the target RNA by the RNA edited entity. In some instances, the candidate engineered guide RNA can directly recruit or position/orient the RNA editing entity to the proper location for editing of the target RNA. In other instances, the candidate engineered guide RNA when hybridized to the target RNA forms a guide-target RNA scaffold with one or more structural features as described herein, where the guide-target RNA scaffold with one or more structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

As disclosed herein, an “internal loop” refers to the structure substantially formed only upon formation of the guide-target RNA scaffold, where nucleotides in either the candidate engineered guide RNA or the target RNA are not complementary to their positional counterparts on the opposite strand and where one side of the internal loop, either on the target RNA side or the candidate engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

As disclosed herein, a “macro-footprint” sequence can be positioned such that it flanks, is proximal to, or is adjacent to a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint. In some embodiments, a macro-footprint sequence can comprise a barbell macro-footprint sequence comprising latent structures that, when manifested, produce a first internal loop and a second internal loop.

As described herein, a “micro-footprint” sequence refers to a sequence with latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” can refer to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject may be not necessarily diagnosed or suspected of being at high risk for the disease

The term “in vivo” refers to an event that takes place in a subject's body.

The term “ex vivo” refers to an event that takes place outside of a subject's body. An ex vivo assay may be not performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g. physiological pH, physiological temperature, physiological salt concentration, etc.).

### Numbered Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A high throughput screening method for identifying a guide RNA suitable for editing a target RNA of interest comprising: (a) contacting a plurality of self-annealing RNA structures with an RNA editing entity, wherein a self-annealing RNA structure of the plurality comprises: (i) a target RNA, (ii) a candidate guide RNA, and (iii) a linker that covalently attaches the target RNA and the candidate guide RNA, wherein the target RNA and candidate guide RNA form a guide-target RNA scaffold that comprises one or more structural features, wherein when the candidate guide RNA hybridizes to the target RNA, a hairpin loop is formed, where the loop of the hairpin loop comprises at least part of the linker, and wherein the contacting occurs under conditions that allows the RNA editing entity to edit a base of a nucleotide in the target RNA in the plurality of self-annealing RNA structures; and (b) identifying one or more self-annealing RNA structures in the plurality of self-annealing RNA structures that comprise an edited target RNA; wherein the one or more identified self-annealing RNA structures that comprise an edited target RNA each comprise a candidate guide RNA suitable for editing the target RNA. Embodiment 2. A high throughput screening method for identifying a guide RNA suitable for editing a target RNA of interest comprising: (a) contacting a plurality of engineered RNA structures with an RNA editing entity, wherein the engineered RNA structure of the plurality comprises: (i) a target RNA, (ii) a candidate guide RNA, and (iii) a linker that attaches the target RNA and the candidate guide RNA, wherein the target RNA and candidate guide RNA form a guide-target RNA scaffold that comprises one or more structural features, wherein the linker comprises a first barcode attached to the target RNA and a second barcode attached to the candidate guide RNA, wherein the first barcode has complementarity with the second barcode, and wherein the linker does not covalently attach the candidate guide RNA to the target RNA, and wherein the contacting occurs under conditions that allows the RNA editing entity to edit a base of a nucleotide in the target RNA in the plurality of the engineered RNA structures; and (b) identifying one or more engineered RNA structures in the plurality of engineered RNA structures that comprise an edited target RNA; wherein the one or more identified engineered RNA structures that comprise an edited target RNA each comprise a candidate guide RNA suitable for editing the target RNA. Embodiment 3. The method of Embodiment 1, wherein the RNA editing entity is: (a) an adenosine deaminase acting on RNA (ADAR); (b) an ADAR variant; (c) a catalytically active deaminase domain; or (d) a fusion protein comprising any one of (a)-(c). Embodiment 4. The method of Embodiment 3, wherein the ADAR comprises human ADAR (hADAR). Embodiment 5. The method of Embodiment 3, wherein the ADAR is ADAR1 and/or ADAR2. Embodiment 6. The method of any one of Embodiment 1-Embodiment 5, wherein the candidate guide RNA each comprise 1 to 50 structural features. Embodiment 7. The method of any one of Embodiment 1-Embodiment 6, wherein the one or more structural features of each candidate guide RNA comprises a mismatch, a bulge, an internal loop, a hairpin, a wobble base pair, or any combination thereof. Embodiment 8. The method of Embodiment 7, wherein the one or more structural features comprise a bulge. Embodiment 9. The method of Embodiment 8, wherein the bulge is a symmetrical bulge. Embodiment 10. The method of Embodiment 8, wherein the bulge is an asymmetrical bulge. Embodiment 11. The method of Embodiment 8, wherein the bulge comprises about 1 to about 4 nucleotides of the candidate guide RNA and about 0 to about 4 nucleotides of the target RNA. Embodiment 12. The method of Embodiment 8, wherein the bulge comprises about 0 to about 4 nucleotides of the candidate guide RNA and about 1 to about 4 nucleotides of the target RNA. Embodiment 13. The method of Embodiment 8, wherein the bulge comprises 3 nucleotides of the candidate guide RNA and 3 nucleotides of the target RNA. Embodiment 14. The method of Embodiment 7, wherein the one or more structural features comprise an internal loop. Embodiment 15. The method of Embodiment 14, wherein the internal loop is a symmetrical internal loop. Embodiment 16. The method of Embodiment 14, wherein the internal loop is an asymmetrical internal loop. Embodiment 17. The method of Embodiment 14, wherein the internal loop is formed by about 5 to about 10 nucleotides of either the candidate guide RNA or the target RNA. Embodiment 18. The method of any one of Embodiment 1-Embodiment 7, wherein the one or more structural features comprise a hairpin. Embodiment 19. The method of Embodiment 18, wherein the hairpin is a non-recruitment hairpin. Embodiment 20. The method of Embodiment 18, wherein the hairpin comprises a loop portion of about 3 to about 15 nucleotides in length. Embodiment 21. The method of Embodiment 7, wherein the one or more structural features comprise a mismatch. Embodiment 22. The method of Embodiment 21, wherein the mismatch comprises a base in the candidate guide RNA opposite to and unpaired with a base in the target RNA. Embodiment 23. The method of Embodiment 21, wherein the mismatch comprises an A/A mismatch, an A/G mismatch, an A/C mismatch, a G/G mismatch, a C/C mismatch, or a C/U mismatch. Embodiment 24. The method of Embodiment 21, wherein the mismatch comprises an A/C mismatch and wherein the A is in the target RNA molecule and the C is in the candidate guide RNA. Embodiment 25. The method of Embodiment 24, wherein the A in the A/C mismatch is the base of the nucleotide in the target RNA molecule chemically modified by the RNA editing entity. Embodiment 26. The method of Embodiment 7, wherein the one or more structural features comprise a wobble base pair. Embodiment 27. The method of Embodiment 26, wherein the wobble base pair comprises a guanine paired with a uracil. Embodiment 28. The method of any one of Embodiment 1-Embodiment 27, wherein each of the one or more structural features are present in a micro-footprint of each candidate guide RNA. Embodiment 29. The method of any one of Embodiment 1-Embodiment 27, wherein at least one of the one or more structural features are present in a micro-footprint of each candidate guide RNA. Embodiment 30. The method of Embodiment 29, wherein at least one of the one or more structural features is present in a macro-footprint of each candidate guide RNA. Embodiment 31. The method of Embodiment 30, wherein the macro-footprint comprises at least two structural features, wherein the at least two structural features comprise a first barbell and a second barbell, wherein the first barbell and the second barbell flank opposing ends of the micro-footprint. Embodiment 32. The method of any one of Embodiment 1-Embodiment 27, wherein each of the one or more structural features are present in a micro-footprint and a macro-footprint of each candidate guide RNA. Embodiment 33. The method of Embodiment 32, wherein the macro-footprint comprises at least two structural features, wherein the at least two structural features comprise a first barbell and a second barbell, wherein the first barbell and the second barbell flank opposing ends of the micro-footprint. Embodiment 34. The method of any one of Embodiment 1-Embodiment 27, wherein at least one candidate guide RNA, upon hybridization to the target RNA, comprises a first 6/6 symmetrical internal loop and a second 6/6 symmetrical internal loop flanking one or more structural features selected from a bulge, internal loop, mismatch, and wobble base pair, wherein the first 6/6 symmetrical internal loop and the second 6/6 symmetrical internal loop each comprise 6 nucleotides of the candidate guide RNA and 6 nucleotides of the target RNA. Embodiment 35. The method of any one of Embodiment 1-Embodiment 34, wherein at least one candidate guide RNA further comprises at least one recruitment hairpin. Embodiment 36. The method of Embodiment 35, wherein the recruitment hairpin is a GluR2 hairpin. Embodiment 37. The method of any one of Embodiment 3-Embodiment 37, wherein the target RNA of each of self-annealing RNA structures in the plurality are the same. Embodiment 38. The method of any one of Embodiment 3-Embodiment 37, where the plurality of self-annealing RNA structures comprises at least about 10 to 1×108 different self-annealing RNA structures with comprising structural features. Embodiment 39. The method of any one of Embodiment 3-Embodiment 38, wherein the identifying comprises sequencing amplicons derived from the plurality of self-annealing RNA structures and the self-annealing RNA structures that comprise an edited target RNA are identified based on the sequence of the amplicons. Embodiment 40. The method of Embodiment 39, wherein the sequencing is next generation sequencing (NGS). Embodiment 41. The method of Embodiment 39 or Embodiment 40, wherein each of the self-annealing RNA structures further comprise one or more universal primer binding sites and the amplicons are derived by amplification of the self-annealing RNA structures using one or more universal primers. Embodiment 42. The method of any one of Embodiment 39-Embodiment 41, wherein each of the self-annealing RNA structures further comprises a barcode, and wherein the barcode is unique for each of the self-annealing RNA structures of the plurality. Embodiment 43. The method of any one of Embodiment 3-Embodiment 40, wherein each of the self-annealing RNA structures is according to the formula, from 5′- to 3′-: UPBS1-BC1-target RNA—loop—candidate guide RNA—BC2-UPBS2, wherein UPB S1 and UPB S2 are a first universal primer binding site and a second universal primer binding site, respectively, and wherein BC1 is an optional first barcode and BC2 is an optional second bar code. Embodiment 44. The method of any one of Embodiment 3-Embodiment 40, wherein each of the self-annealing RNA structures comprise a first universal primer binding site, a second universal primer binding site, a first barcode, and a second bar code. Embodiment 45. The method of any one of Embodiment 3-Embodiment 40, wherein each candidate guide RNA is according to the formula, from 5′- to 3′-: UPBS1-BC1-candidate guide RNA, and wherein each target RNA is according to the formula, from 5′- to 3′-: target RNA-BC2-UPBS2, wherein UPBS1 and UPBS2 are a first universal primer binding site and a second universal primer binding site, respectively, and wherein BC1 is an optional first barcode and BC2 is an optional second bar code. Embodiment 46. The method of any one of Embodiment 1-Embodiment 45, wherein the target RNA is encoded by one of the following genes or a fragment thereof: APP, ABCA4, SERPINA1, HEXA, LRRK2, CFTR, SNCA, MAPT, DUX4, PMP22, DMPK, SOD1, GRN, LIPA, or DMD. Embodiment 47. The method of any one of Embodiment 1-Embodiment 46, wherein the plurality of self-annealing RNA structures is contacted with the RNA editing entity in the presence of one or more ADAR inhibitors and/or trans-regulators. Embodiment 48. The method of any one of Embodiment 1-Embodiment 47, wherein each self-annealing RNA structure of the plurality of self-annealing RNA structures has a length of from about 50 nucleotides to about 500 nucleotides. Embodiment 49. The method of Embodiment 48, wherein each self-annealing RNA structure of the plurality of self-annealing RNA structures has a length of about 100. Embodiment 50. The method of Embodiment 48, wherein each self-annealing RNA structure of the plurality of self-annealing RNA structures has a length of about 230. Embodiment 51. A high throughput screening method for identifying a guide RNA suitable for editing a target RNA of interest comprising: (a) providing a plurality of self-annealing RNA structures, wherein each self-annealing RNA structure of the plurality comprises: (i) a target RNA, (ii) a candidate guide RNA, and (iii) a linker that covalently attaches the target RNA and the candidate guide RNA, wherein the target RNA and candidate guide RNA form a guide-target RNA scaffold that comprises one or more structural features, wherein the linker forms a hairpin loop, and wherein the plurality of self-annealing RNA structures comprises a diversity of structural features; (b) contacting the plurality of self-annealing RNA structures with an RNA editing entity under conditions wherein the RNA editing entity is capable of editing a target RNA in the plurality of self-annealing RNA structures; and (c) identify self-annealing RNA structures in the plurality of self-annealing RNA structures that comprise an edited target RNA; wherein the identified self-annealing RNA structures that comprise an edited target RNA each comprise a candidate guide RNA suitable for editing the target RNA. Embodiment 52. The method of Embodiment 51, wherein the RNA editing entity is: (a) an adenosine deaminase acting on RNA (ADAR); (b) an ADAR variant; (c) a catalytically active deaminase domain; or (d) a fusion protein comprising any one of (a)-(c). Embodiment 53. The method of Embodiment 52, wherein the ADAR comprises human ADAR (hADAR). Embodiment 54. The method of Embodiment 52, wherein the ADAR is ADAR1 and/or ADAR2. Embodiment 55. The method of any one of Embodiment 51-Embodiment 54, wherein the self-annealing RNA structures of the plurality of self-annealing RNA structures each comprise 1 to 50 structural features. Embodiment 56. The method any one of Embodiment 51-Embodiment 55, wherein the one or more structural features of at least one of the self-annealing RNA structures comprises a mismatch, a bulge, an internal loop, a hairpin, a mismatch, a wobble base pair, or any combination thereof. Embodiment 57. The method of Embodiment 56, wherein the one or more structural features comprise a bulge. Embodiment 58. The method of Embodiment 57, wherein the bulge is a symmetrical bulge. Embodiment 59. The method of Embodiment 57, wherein the bulge is an asymmetrical bulge. Embodiment 60. The method of Embodiment 57, wherein the bulge comprises about 1 to about 4 nucleotides of the candidate guide RNA and about 0 to about 4 nucleotides of the target RNA. Embodiment 61. The method of Embodiment 57, wherein the bulge comprises about 0 to about 4 nucleotides of the candidate guide RNA and about 1 to about 4 nucleotides of the target RNA. Embodiment 62. The method of Embodiment 57, wherein the bulge comprises 3 nucleotides of the candidate guide RNA and 3 nucleotides of the target RNA. Embodiment 63. The method of Embodiment 56, wherein the one or more structural features comprise an internal loop. Embodiment 64. The method of Embodiment 63, wherein the internal loop is a symmetrical internal loop. Embodiment 65. The method of Embodiment 63, wherein the internal loop is an asymmetrical internal loop. Embodiment 66. The method of Embodiment 63, wherein the internal loop is formed by about 5 to about 10 nucleotides of either the candidate guide RNA or the target RNA. Embodiment 67. The method of any one of Embodiment 51-Embodiment 56, wherein the one or more structural features comprise a hairpin. Embodiment 68. The method of Embodiment 67, wherein the hairpin is a non-recruitment hairpin. Embodiment 69. The method of Embodiment 67, wherein the hairpin comprises a loop portion of about 3 to about 15 nucleotides in length. Embodiment 70. The method of Embodiment 56, wherein the one or more structural features comprise a mismatch. Embodiment 71. The method of Embodiment 70, wherein the mismatch comprises a base in the candidate guide RNA opposite to and unpaired with a base in the target RNA. Embodiment 72. The method of Embodiment 70, wherein the mismatch comprises an A/A mismatch, an A/G mismatch, an A/C mismatch, a G/G mismatch, a C/C mismatch, or a C/U mismatch. Embodiment 73. The method of Embodiment 70, wherein the mismatch comprises an A/C mismatch and wherein the A is in the target RNA molecule and the C is in the candidate guide RNA. Embodiment 74. The method of Embodiment 73, wherein the A in the A/C mismatch is the base of the nucleotide in the target RNA molecule chemically modified by the RNA editing entity. Embodiment 75. The method of Embodiment 56, wherein the one or more structural features comprise a wobble base pair. Embodiment 76. The method of Embodiment 75, wherein the wobble base pair comprises a guanine paired with a uracil. Embodiment 77. The method of any one of Embodiment 51-Embodiment 76, wherein each of the one or more structural features are present in a micro-footprint of each candidate guide RNA. Embodiment 78. The method of any one of Embodiment 51-Embodiment 76, wherein at least one of the one or more structural features are present in a micro-footprint of each candidate guide RNA. Embodiment 79. The method of Embodiment 78, wherein at least one of the one or more structural features is present in a macro-footprint of each candidate guide RNA. Embodiment 80. The method of Embodiment 79, wherein the macro-footprint comprises at least two structural features, wherein the at least two structural features comprise a first barbell and a second barbell, wherein the first barbell and the second barbell flank opposing ends of the micro-footprint. Embodiment 81. The method of any one of Embodiment 51-Embodiment 76, wherein each of the one or more structural features are present in a micro-footprint and a macro-footprint of each candidate guide RNA. Embodiment 82. The method of Embodiment 81, wherein the macro-footprint comprises at least two structural features, wherein the at least two structural features comprise a first barbell and a second barbell, wherein the first barbell and the second barbell flank opposing ends of the micro-footprint. Embodiment 83. The method of any one of Embodiment 51-Embodiment 76, wherein at least one candidate guide RNA, upon hybridization to the target RNA, comprises a first 6/6 symmetrical internal loop and a second 6/6 symmetrical internal loop flanking one or more structural features selected from a bulge, internal loop, mismatch, and wobble base pair, wherein the first 6/6 symmetrical internal loop and the second 6/6 symmetrical internal loop each comprise 6 nucleotides of the candidate guide RNA and 6 nucleotides of the target RNA. Embodiment 84. The method of any one of Embodiment 51-Embodiment 83, wherein at least one candidate guide RNA further comprises at least one recruitment hairpin. Embodiment 85. The method of Embodiment 84, wherein the recruitment hairpin is a GluR2 hairpin. Embodiment 86. The method of any one of Embodiment 51-Embodiment 85, wherein the target RNA of each of the self-annealing RNA structures in the plurality are the same. Embodiment 87. The method of any one of Embodiment 51-Embodiment 86, where the plurality of self-annealing RNA structures comprises at least about 10 to 1×108 different self-annealing RNA structures comprising different structural features. Embodiment 88. The method of any one of Embodiment 51-Embodiment 87, wherein the identifying comprises sequencing amplicons derived from the plurality of self-annealing RNA structures and the self-annealing RNA structures that comprise an edited targeted RNA are identified based on the sequence of the amplicons. Embodiment 89. The method of Embodiment 88, wherein the sequencing is next generation sequencing (NGS). Embodiment 90. The method of Embodiment 88 or Embodiment 89, wherein each of the self-annealing RNA structures further comprise one or more universal primer binding sites and the amplicons are derived by amplification of the self-annealing RNA structures using one or more universal primers. Embodiment 91. The method of any one of Embodiment 88-Embodiment 90, wherein each of the self-annealing RNA structures further comprises a barcode, and wherein the barcode is unique for each of the self-annealing RNA structures of the plurality. Embodiment 92. The method of any one of Embodiment 51-Embodiment 89, wherein each of the self-annealing RNA structures is according to the formula, from 5′- to 3′-: UPBS 1-BC1-target RNA—loop—candidate guide RNA—BC2-UPBS 2, wherein UPBS1 and UPBS2 are a first universal primer binding site and a second universal primer binding site, respectively, and wherein BC1 is an optional first barcode and BC2 is an optional second bar code. Embodiment 93. The method of any one of Embodiment 51-Embodiment 92, wherein the target RNA is encoded by one of the following genes or a fragment thereof: APP, ABCA4, SERPINA1, HEXA, LRRK2, CFTR, SNCA, MAPT, DUX4, PMP22, DMPK, SOD1, GRN, LIPA, or DMD. Embodiment 94. The method of any one of Embodiment 51-Embodiment 93, wherein the plurality of self-annealing RNA structures is contacted with the RNA editing entity in the presence of one or more ADAR inhibitors and/or trans-regulators. Embodiment 95. The method of any one of Embodiment 51-Embodiment 94, wherein each self-annealing RNA structure in the plurality of self-annealing RNA structures has a length of from about 50 nucleotides to about 500 nucleotides. Embodiment 96. The method of Embodiment 95, wherein each self-annealing RNA structure in the plurality of self-annealing RNA structures has a length of about 100. Embodiment 97. The method of Embodiment 95, wherein each self-annealing RNA structure in the plurality of self-annealing RNA structures has a length of about 230. Embodiment 98. A method for analyzing nucleic acids comprising: (a) contacting a plurality of self-annealing RNA structures with an RNA editing entity, wherein each self-annealing RNA structures of the plurality comprises: (i) a target RNA, (ii) a candidate guide RNA, and (iii) a linker that covalently attaches the target RNA and the candidate guide RNA, wherein the target RNA and candidate guide RNA form a guide-target RNA scaffold that comprises one or more structural features, wherein the linker forms hairpin loop, and wherein the contacting occurs under conditions that allows the RNA editing entity to edit a target RNA in the plurality of self-annealing RNA structures; and (b) identifying an edited target RNA in the plurality of self-annealing RNA structures. Embodiment 99. The method of Embodiment 98, wherein the RNA editing entity is: (a) an adenosine deaminase acting on RNA (ADAR); (b) an ADAR variant; (c) a catalytically active deaminase domain; or (d) a fusion protein comprising any one of (a)-(c). Embodiment 100. The method of Embodiment 99, wherein the ADAR comprises human ADAR (hADAR). Embodiment 101. The method of Embodiment 99, wherein the ADAR is ADAR1 and/or ADAR2. Embodiment 102. The method of any one of Embodiment 98-Embodiment 101, wherein the self-annealing RNA structures of the plurality of self-annealing RNA structures each comprise 1 to 50 structural features. Embodiment 103. The method of any one of Embodiment 98-Embodiment 102, wherein the one or more structural features of at least one of the self-annealing RNA structures comprises a mismatch, a bulge, an internal loop, a hairpin, a mismatch, a wobble base pair, or any combination thereof. Embodiment 104. The method of Embodiment 103, wherein the one or more structural features comprise a bulge. Embodiment 105. The method of Embodiment 104, wherein the bulge is a symmetrical bulge. Embodiment 106. The method of Embodiment 104, wherein the bulge is an asymmetrical bulge. Embodiment 107. The method of Embodiment 104, wherein the bulge comprises about 1 to about 4 nucleotides of the candidate guide RNA and about 0 to about 4 nucleotides of the target RNA. Embodiment 108. The method of Embodiment 104, wherein the bulge comprises about 0 to about 4 nucleotides of the candidate guide RNA and about 1 to about 4 nucleotides of the target RNA. Embodiment 109. The method of Embodiment 104, wherein the bulge comprises 3 nucleotides of the candidate guide RNA and 3 nucleotides of the target RNA. Embodiment 110. The method of Embodiment 103, wherein the one or more structural features comprise an internal loop. Embodiment 111. The method of Embodiment 110, wherein the internal loop is a symmetrical internal loop. Embodiment 112. The method of Embodiment 110, wherein the internal loop is an asymmetrical internal loop. Embodiment 113. The method of Embodiment 110, wherein the internal loop is formed by about 5 to about 10 nucleotides of either the candidate guide RNA or the target RNA. Embodiment 114. The method of any one of Embodiment 98-Embodiment 103, wherein the one or more structural features comprise a hairpin. Embodiment 115. The method of Embodiment 114, wherein the hairpin is a non-recruitment hairpin. Embodiment 116. The method of Embodiment 114, wherein the hairpin comprises a loop portion of about 3 to about 15 nucleotides in length. Embodiment 117. The method of Embodiment 103, wherein the one or more structural features comprise a mismatch. Embodiment 118. The method of Embodiment 117, wherein the mismatch comprises a base in the candidate guide RNA opposite to and unpaired with a base in the target RNA. Embodiment 119. The method of Embodiment 117, wherein the mismatch comprises an A/A mismatch, an A/G mismatch, an A/C mismatch, a G/G mismatch, a C/C mismatch, or a C/U mismatch. Embodiment 120. The method of Embodiment 117, wherein the mismatch comprises an A/C mismatch and wherein the A is in the target RNA molecule and the C is in the candidate guide RNA. Embodiment 121. The method of Embodiment 120, wherein the A in the A/C mismatch is the base of the nucleotide in the target RNA molecule chemically modified by the RNA editing entity. Embodiment 122. The method of Embodiment 103, wherein the one or more structural features comprise a wobble base pair. Embodiment 123. The method of Embodiment 122, wherein the wobble base pair comprises a guanine paired with a uracil. Embodiment 124. The method of any one of Embodiment 98-Embodiment 123, wherein each of the one or more structural features are present in a micro-footprint of each candidate guide RNA. Embodiment 125. The method of any one of Embodiment 98-Embodiment 123, wherein at least one of the one or more structural features are present in a micro-footprint of each candidate guide RNA. Embodiment 126. The method of Embodiment 125, wherein at least one of the one or more structural features is present in a macro-footprint of each candidate guide RNA. Embodiment 127. The method of Embodiment 126, wherein the macro-footprint comprises at least two structural features, wherein the at least two structural features comprise a first barbell and a second barbell, wherein the first barbell and the second barbell flank opposing ends of the micro-footprint. Embodiment 128. The method of any one of Embodiment 98-Embodiment 123, wherein each of the one or more structural features are present in a micro-footprint and a macro-footprint of each candidate guide RNA. Embodiment 129. The method of Embodiment 128, wherein the macro-footprint comprises at least two structural features, wherein the at least two structural features comprise a first barbell and a second barbell, wherein the first barbell and the second barbell flank opposing ends of the micro-footprint. Embodiment 130. The method of any one of Embodiment 98-Embodiment 123, wherein at least one candidate guide RNA, upon hybridization to the target RNA, comprises a first 6/6 symmetrical internal loop and a second 6/6 symmetrical internal loop flanking one or more structural features selected from a bulge, internal loop, mismatch, and wobble base pair, wherein the first 6/6 symmetrical internal loop and the second 6/6 symmetrical internal loop each comprise 6 nucleotides of the candidate guide RNA and 6 nucleotides of the target RNA. Embodiment 131. The method of any one of Embodiment 98-Embodiment 130, wherein at least one candidate guide RNA further comprises at least one recruitment hairpin. Embodiment 132. The method of Embodiment 131, wherein the recruitment hairpin is a GluR2 hairpin. Embodiment 133. The method of any one of Embodiment 98-Embodiment 132, wherein the target RNA of each of the self-annealing RNA structures in the plurality are the same. Embodiment 134. The method of any one of Embodiment 98-Embodiment 133, where the plurality of self-annealing RNA structures comprises at least about 10 to 1×108 different self-annealing RNA structures comprising different structural features. Embodiment 135. The method of any one of Embodiment 98-Embodiment 134, wherein the identifying comprises sequencing amplicons derived from the plurality of self-annealing RNA structures and the self-annealing RNA structures that comprise an edited guide-target RNA scaffold are identified based on the sequence of the amplicons. Embodiment 136. The method of Embodiment 135, wherein the sequencing is next generation sequencing (NGS). Embodiment 137. The method of Embodiment 135 or Embodiment 136, wherein each of the self-annealing RNA structures further comprise one or more universal primer binding sites and the amplicons are derived by amplification of the self-annealing RNA structures using one or more universal primers. Embodiment 138. The method of any one of Embodiment 135-Embodiment 137, wherein each of the self-annealing RNA structures further comprises a barcode, and wherein the barcode is unique for each of the self-annealing RNA structures of the plurality. Embodiment 139. The method of any one of Embodiment 98-Embodiment 136, wherein each of the self-annealing RNA structures is according to the formula, from 5′- to 3′-: UPBS 1-BC1-target RNA—loop—candidate guide RNA—BC2-UPBS 2, wherein UPBS1 and UPB S2 are a first universal primer binding site and a second universal primer binding site, respectively, and wherein BC1 is an optional first barcode and BC2 is an optional second bar code. Embodiment 140. The method of any one of Embodiment 98-Embodiment 139, wherein the target RNA is encoded by one of the following genes or a fragment thereof: APP, ABCA4, SERPINA1, HEXA, LRRK2, CFTR, SNCA, MAPT, DUX4, PMP22, DMPK, SOD1, GRN, LIPA, or DMD. Embodiment 141. The method of any one of Embodiment 99-Embodiment 101, wherein the plurality of self-annealing RNA structures is contacted with the RNA editing entity in the presence of one or more ADAR inhibitors and/or trans-regulators. Embodiment 142. The method of any one of Embodiment 98-Embodiment 141, wherein each self-annealing RNA structure in the plurality of self-annealing RNA structures has a length of from about 50 nucleotides to about 500 nucleotides. Embodiment 143. The method of Embodiment 142, wherein each self-annealing RNA structure in the plurality of self-annealing RNA structures has a length of about 100. Embodiment 144. The method of Embodiment 142, wherein each self-annealing RNA structure in the plurality of self-annealing RNA structures has a length of about 230. Embodiment 145. An engineered RNA structure comprising: (a) a target RNA, (b) a candidate guide RNA that facilitates editing of a base of a nucleotide of the target RNA via an RNA editing entity, and (c) a linker that attaches the target RNA and the candidate guide RNA, wherein the target RNA and candidate guide RNA form a guide-target RNA scaffold that comprises one or more structural features, and wherein the one or more structural features are substantially formed upon hybridization of the candidate guide RNA to the target RNA. Embodiment 146. The engineered RNA structure of Embodiment 145, wherein the engineered RNA structure is a self-annealing RNA structure, and wherein the linker covalently attaches the target RNA to the candidate guide RNA. Embodiment 147. The engineered RNA structure of Embodiment 146, wherein when the candidate guide RNA hybridizes to the target RNA, a hairpin loop is formed, where the loop of the hairpin loop comprises at least part of the linker. Embodiment 148. The engineered RNA structure of any one of Embodiment 145-Embodiment 147, wherein the linker comprises RNA. Embodiment 149. The engineered RNA structure of any one of Embodiment 145-Embodiment 148, wherein the target RNA further comprises a first barcode and the candidate guide RNA further comprises a second barcode. Embodiment 150. The engineered RNA structure of Embodiment 149, wherein the first barcode and second barcode are complementary. Embodiment 151. The engineered RNA structure of Embodiment 150, wherein the first barcode and the second barcode link the candidate guide RNA to the target RNA when the first barcode and the second barcode hybridize to each other. Embodiment 152. The engineered RNA structure of any one of Embodiment 149-Embodiment 151, wherein the target RNA further comprises a first universal primer binding site and the candidate guide RNA further comprises a second universal primer binding site. Embodiment 153. The engineered RNA structure of Embodiment 152, wherein the engineered RNA structure is according to the formula, from 5′- to 3′-: UPBS1-BC1-target RNA—loop—candidate guide RNA—BC2-UPB S2, wherein UPBS1 and UPB S2 are the first universal primer binding site and the second universal primer binding site, respectively, and wherein BC1 is the first barcode and BC2 is the second bar code. Embodiment 154. The engineered RNA structure of Embodiment 152, wherein the engineered RNA structure comprises a first universal primer binding site, a second universal primer binding site, a first barcode, and a second bar code. Embodiment 155. The engineered RNA structure of Embodiment 145, wherein the linker comprises a first barcode attached to the target RNA and a second barcode attached to the candidate guide RNA, wherein the first barcode has complementarity with the second barcode. Embodiment 156. The engineered RNA structure of Embodiment 155, wherein the linker does not covalently attach the candidate guide RNA to the target RNA. Embodiment 157. The engineered RNA structure of Embodiment 155 or Embodiment 156, wherein the linker comprises one or more universal primer binding sites. Embodiment 158. The engineered RNA structure of any one of Embodiment 155-Embodiment 157, wherein each candidate guide RNA is according to the formula, from 5′- to 3′-: UPBS1-BC1-candidate guide RNA, and wherein each target RNA is according to the formula, from 5′- to 3′-: target RNA-BC2-UPB S2, wherein UPBS1 and UPB S2 are a first universal primer binding site and a second universal primer binding site, respectively, and wherein BC1 is an optional first barcode and BC2 is an optional second bar code. Embodiment 159. The engineered RNA structure of any one of Embodiment 145-Embodiment 158, wherein the one or more structural features of the engineered RNA structure comprises a mismatch, a bulge, an internal loop, a hairpin, a mismatch, a wobble base pair, or any combination thereof. Embodiment 160. The engineered RNA structure of Embodiment 159, wherein the one or more structural features comprise a bulge. Embodiment 161. The engineered RNA structure of Embodiment 160, wherein the bulge is a symmetrical bulge. Embodiment 162. The engineered RNA structure of Embodiment 160, wherein the bulge is an asymmetrical bulge. Embodiment 163. The engineered RNA structure of Embodiment 160, wherein the bulge comprises from about 1 to about 4 nucleotides of the candidate guide RNA and from about 0 to about 4 nucleotides of the target RNA. Embodiment 164. The engineered RNA structure of Embodiment 160, wherein the bulge comprises from about 0 to about 4 nucleotides of the candidate guide RNA and from about 1 to about 4 nucleotides of the target RNA. Embodiment 165. The engineered RNA structure of Embodiment 160, wherein the bulge comprises 3 nucleotides of the candidate guide RNA and 3 nucleotides of the target RNA. Embodiment 166. The engineered RNA structure of Embodiment 159, wherein the one or more structural features comprise an internal loop. Embodiment 167. The engineered RNA structure of Embodiment 166, wherein the internal loop is a symmetrical internal loop. Embodiment 168. The engineered RNA structure of Embodiment 166, wherein the internal loop is an asymmetrical internal loop. Embodiment 169. The engineered RNA structure of Embodiment 166, wherein the internal loop is formed by from about 5 to about 10 nucleotides of either the candidate guide RNA or the target RNA. Embodiment 170. The engineered RNA structure of Embodiment 159, wherein the one or more structural features comprise a hairpin. Embodiment 171. The engineered RNA structure of Embodiment 170, wherein the hairpin is a non-recruitment hairpin. Embodiment 172. The engineered RNA structure of Embodiment 170, wherein the hairpin comprises a loop portion of from about 3 to about 15 nucleotides in length. Embodiment 173. The engineered RNA structure of Embodiment 159, wherein the one or more structural features comprise a mismatch. Embodiment 174. The engineered RNA structure of Embodiment 173, wherein the mismatch comprises a base in the candidate guide RNA opposite to and unpaired with a base in the target RNA. Embodiment 175. The engineered RNA structure of Embodiment 173, wherein the mismatch comprises an A/A mismatch, an A/G mismatch, an A/C mismatch, a G/G mismatch, a C/C mismatch, or a C/U mismatch. Embodiment 176. The engineered RNA structure of Embodiment 173 wherein the mismatch comprises an A/C mismatch and wherein the A is in the target RNA molecule and the C is in the candidate guide RNA. Embodiment 177. The engineered RNA structure of Embodiment 176, wherein the A in the A/C mismatch is the base of the nucleotide in the target RNA that is edited by the RNA editing entity. Embodiment 178. The engineered RNA structure of Embodiment 159, wherein the one or more structural features comprise a wobble base pair. Embodiment 179. The engineered RNA structure of Embodiment 178, wherein the wobble base pair comprises a guanine paired with a uracil. Embodiment 180. The engineered RNA structure of any one of Embodiment 145-Embodiment 179, wherein each of the one or more structural features are present in a micro-footprint of the candidate guide RNA. Embodiment 181. The engineered RNA structure of any one of Embodiment 145-Embodiment 179, wherein at least one of the one or more structural features are present in a micro-footprint of the candidate guide RNA. Embodiment 182. The engineered RNA structure of Embodiment 181, wherein at least one of the one or more structural features is present in a macro-footprint of the candidate guide RNA. Embodiment 183. The engineered RNA structure of Embodiment 182, wherein the macro-footprint comprises at least two structural features, wherein the at least two structural features comprise a first barbell and a second barbell, wherein the first barbell and the second barbell flank opposing ends of the micro-footprint. Embodiment 184. The engineered RNA structure of any one of Embodiment 145-Embodiment 179, wherein each of the one or more structural features are present in a micro-footprint and a macro-footprint of the candidate guide RNA. Embodiment 185. The engineered RNA structure of Embodiment 184, wherein the macro-footprint comprises at least two structural features, wherein the at least two structural features comprise a first barbell and a second barbell, wherein the first barbell and the second barbell flank opposing ends of the micro-footprint. Embodiment 186. The engineered RNA structure of any one of Embodiment 145-Embodiment 179, wherein the candidate guide RNA, upon hybridization to the target RNA, comprises a first 6/6 symmetrical internal loop and a second 6/6 symmetrical internal loop flanking one or more structural features selected from a bulge, internal loop, mismatch, and wobble base pair, wherein the first 6/6 symmetrical internal loop and the second 6/6 symmetrical internal loop each comprise 6 nucleotides of the candidate guide RNA and 6 nucleotides of the target RNA. Embodiment 187. The engineered RNA structure of any one of Embodiment 145-Embodiment 186, wherein at least one candidate guide RNA further comprises at least one recruitment hairpin. Embodiment 188. The engineered RNA structure of Embodiment 187, wherein the recruitment hairpin is a GluR2 hairpin. Embodiment 189. The engineered RNA structure of any one of Embodiment 145-Embodiment 188, wherein the RNA editing entity is; (a) an ADAR; (b) a variant ADAR; (c) a catalytically active deaminase domain; or (d) a fusion protein comprising any one of (a)-(c). Embodiment 190. The engineered RNA structure of any one of Embodiment 145-Embodiment 189, wherein the engineered RNA structure has a length of from about 50 nucleotides to about 500 nucleotides. Embodiment 191. The engineered RNA structure of Embodiment 190, wherein the engineered RNA structure has a length of about 100. Embodiment 192. The engineered RNA structure of Embodiment 190, wherein the engineered RNA structure has a length of about 230. Embodiment 193. A library of engineered RNA structures comprising a plurality of engineered RNA structures according to any one of Embodiment 145-Embodiment 192, where the plurality of engineered RNA structures comprises from 10 to 1×10 8 different engineered RNA structures comprising different structural features. Embodiment 194. A method for producing a vector encoding a guide RNA comprising: (a) contacting a plurality of self-annealing RNA structures with an RNA editing entity, wherein a self-annealing RNA structure of the plurality comprises: (i) a target RNA, (ii) a candidate guide RNA, and (iii) a linker that covalently attaches the target RNA and the candidate guide RNA, wherein the target RNA and candidate guide RNA form a guide-target RNA scaffold that comprises one or more structural features, wherein the linker forms a hairpin loop, and wherein the contacting occurs under conditions that allows the RNA editing entity to edit a target RNA in the plurality of self-annealing RNA structures; (b) identifying one or more self-annealing RNA structures in the plurality of self-annealing RNA structures that comprise an edited target RNA; wherein the one or more identified self-annealing RNA structures that comprise an edited target RNA each comprise a candidate guide RNA suitable for editing the target RNA; and (c) formulating the candidate guide RNA in a vector.

### EXAMPLES

Examples are provided below to illustrate the present invention. These examples are not meant to constrain the present invention to any particular application or theory of operation.

### Example 1

### High Throughput Screening (HTS) for Guide RNAs

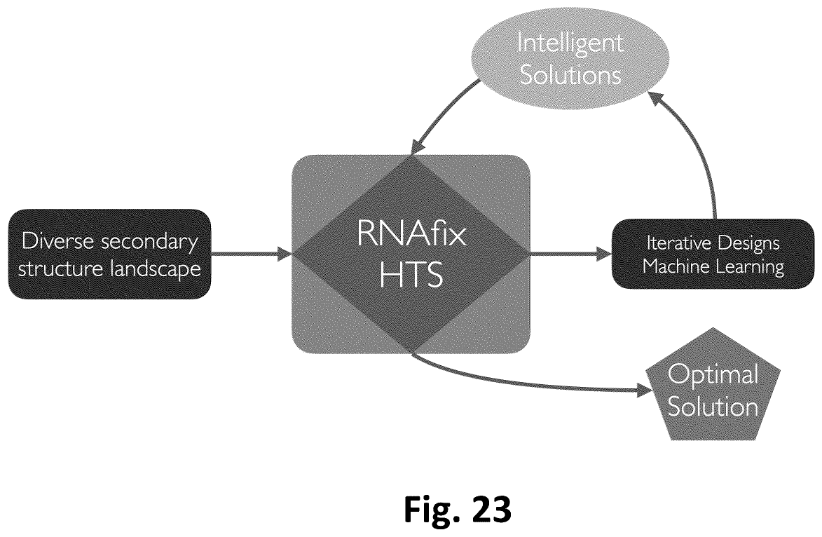

FIG. 23 shows a schematic of the high throughput screening (HTS) platform disclosed herein for discovery of guide RNAs (gRNA). As shown in the flowchart, the HTS platform of the present disclosure scans a diverse secondary structure landscape to assess gRNA efficiency and selectivity. An iterative design process along with machine learning is carried out, which can further refine gRNA design to identify an exemplary superior gRNA for any given disease target (e.g., LRRK2, ABCA4, SERPINA1, SNCA, APP).

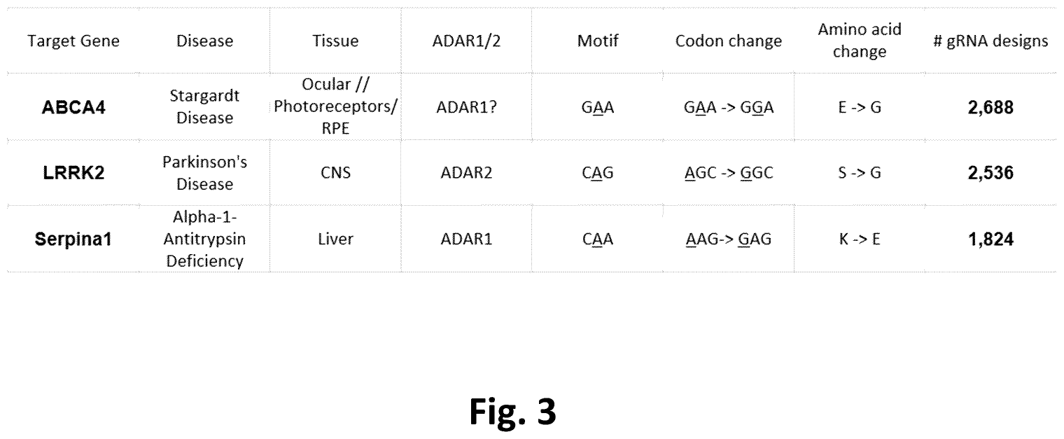

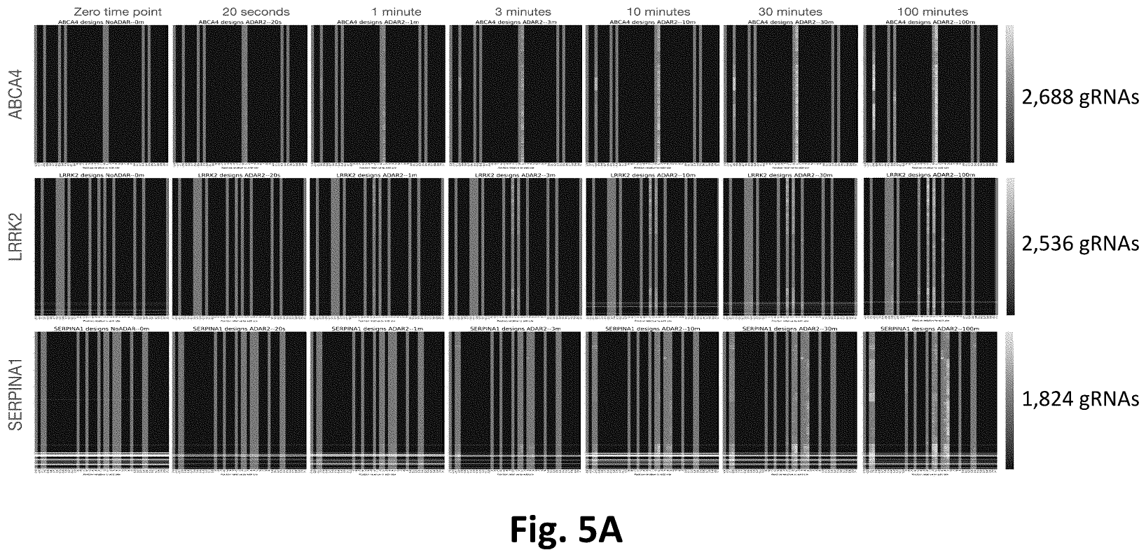

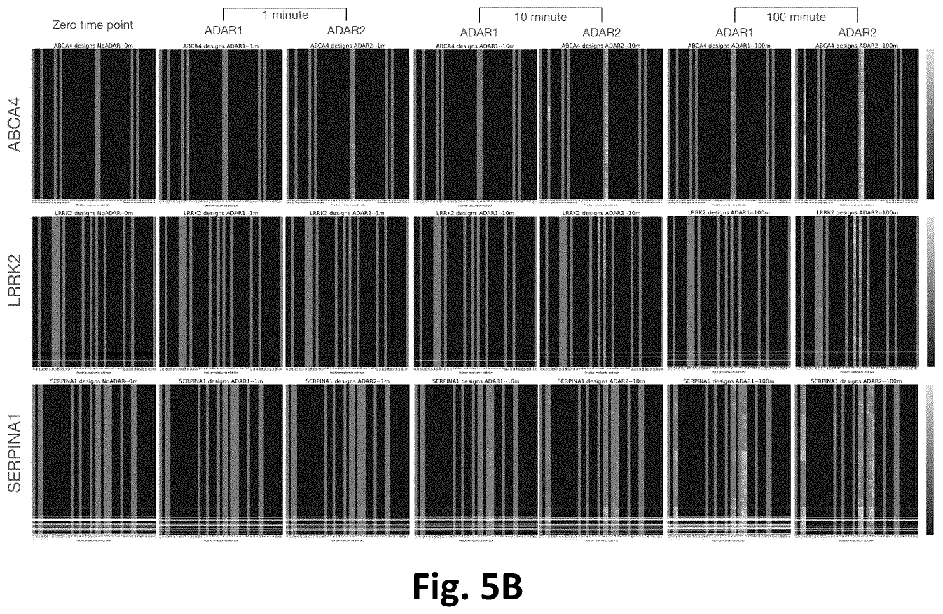

High throughput screens were performed to identify gRNAs for LRRK2, ABCA4 and SERPINA1 according to the methods and compositions described herein (See FIGS. 3 and 5). After contact with ADAR1 or ADAR2 at different time points (0, 1, 10 and 100 minutes), self-annealing RNA structures were assessed for edited target RNAs by NGS sequencing.

### Example 2

### High Throughput Screening of Engineered Guide RNAs Targeting LRRK2 for RNA Editing

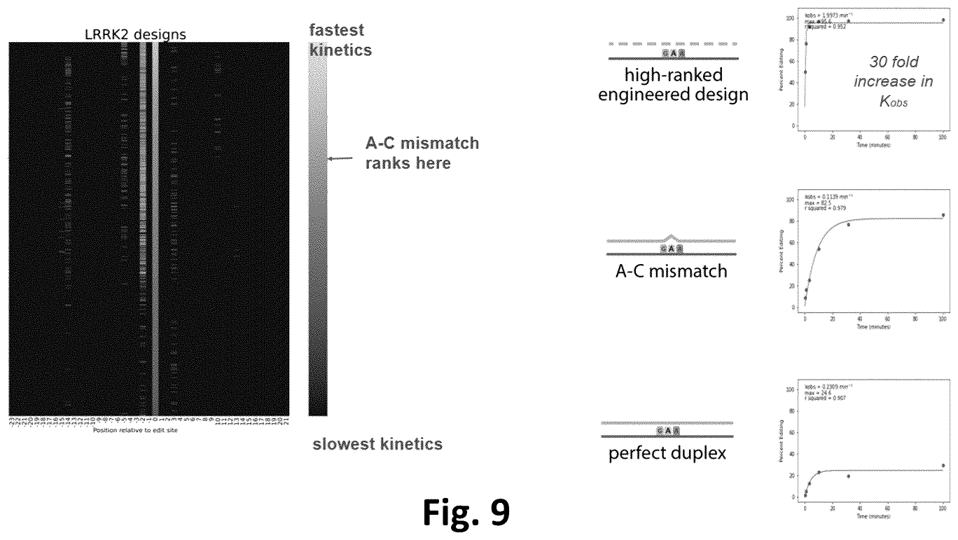

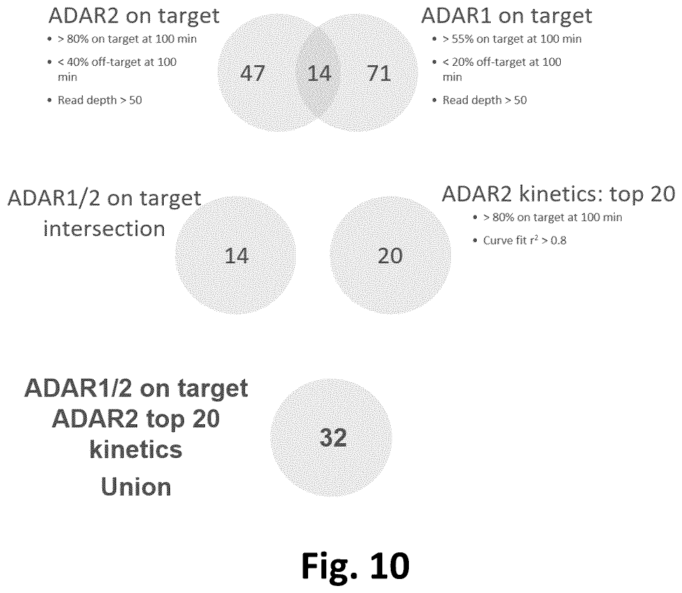

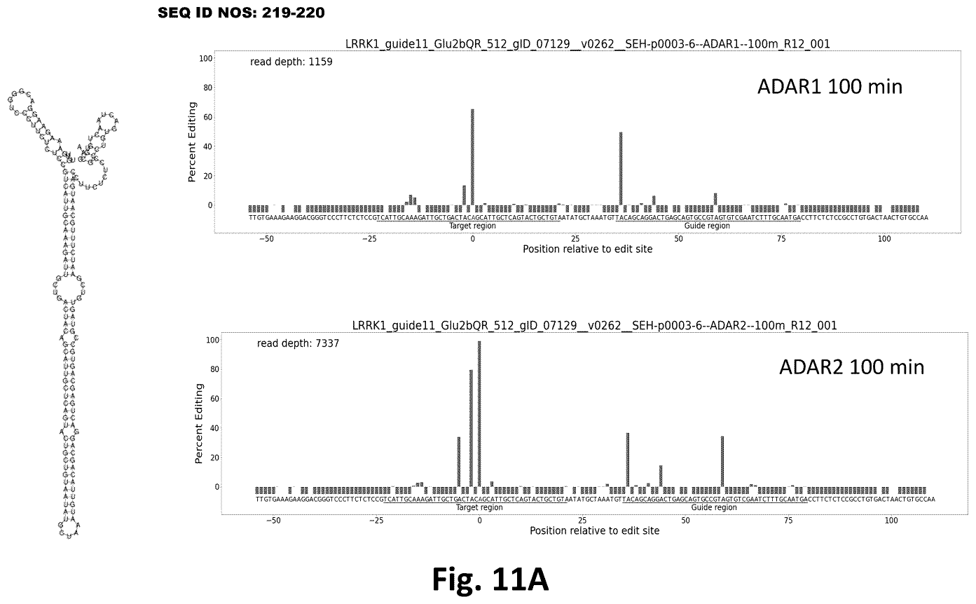

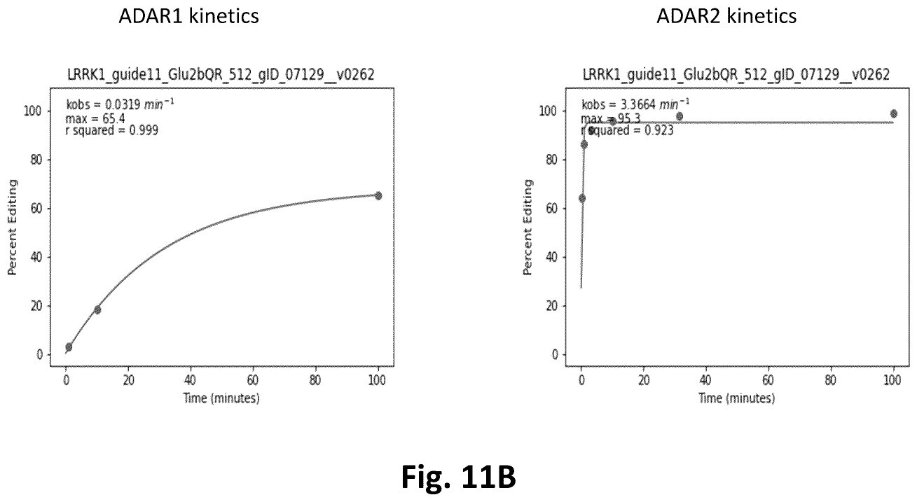

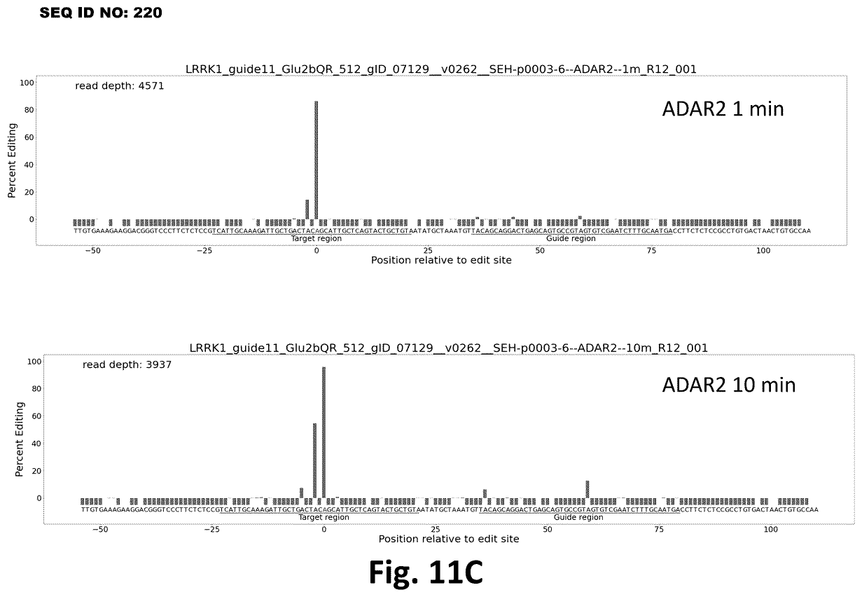

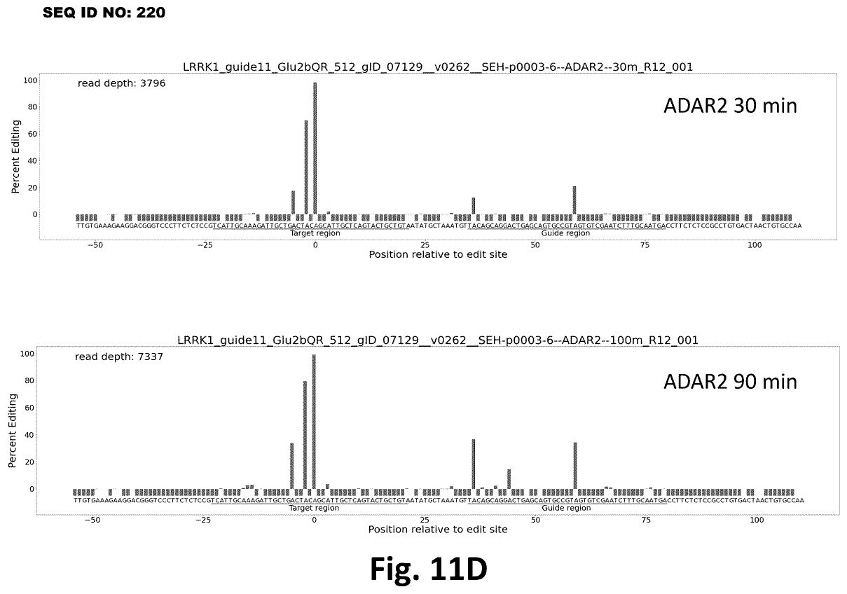

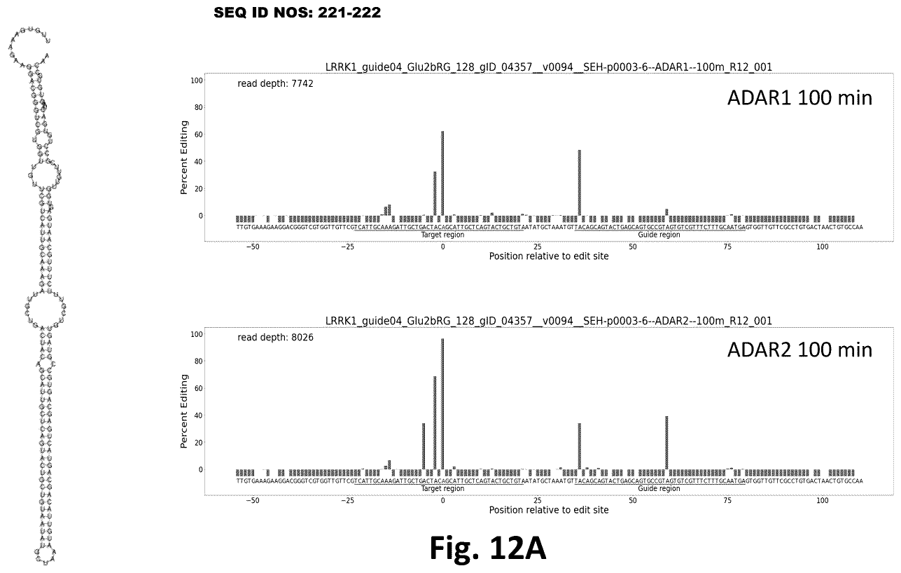

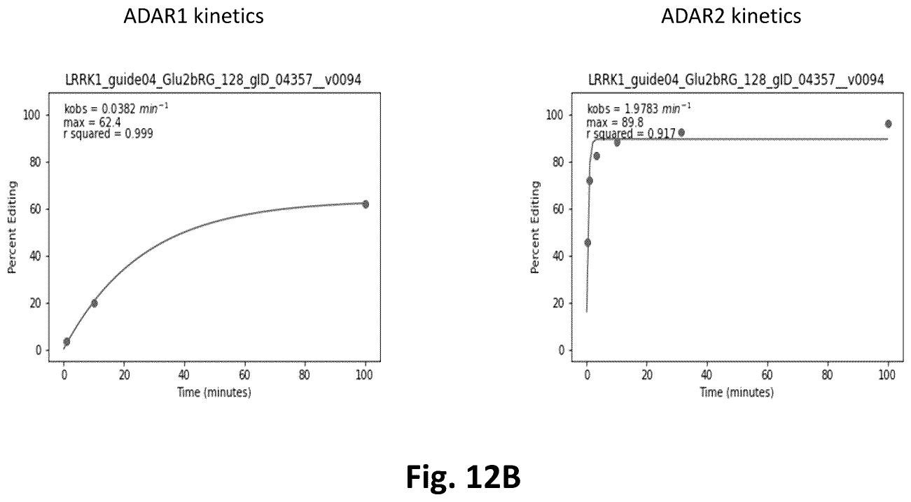

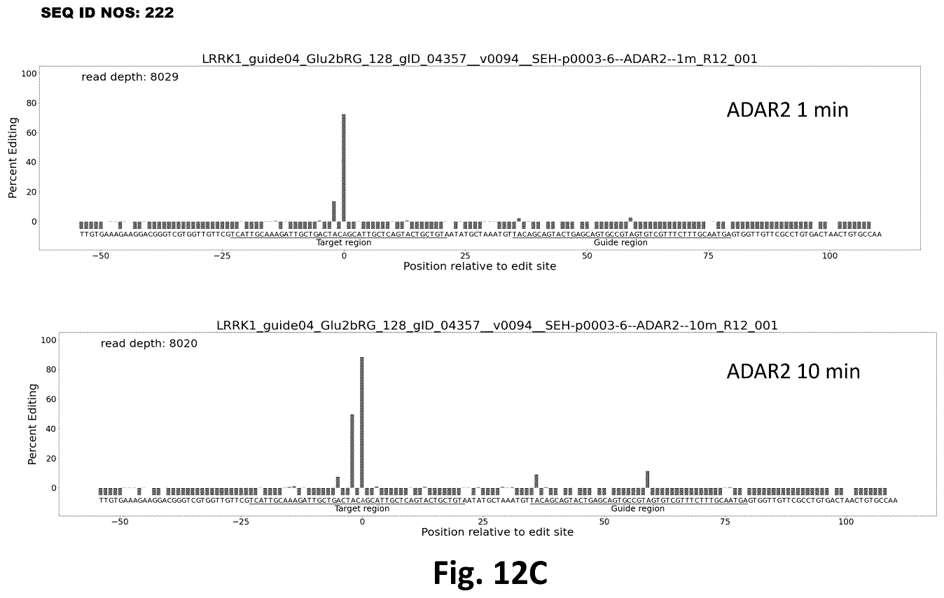

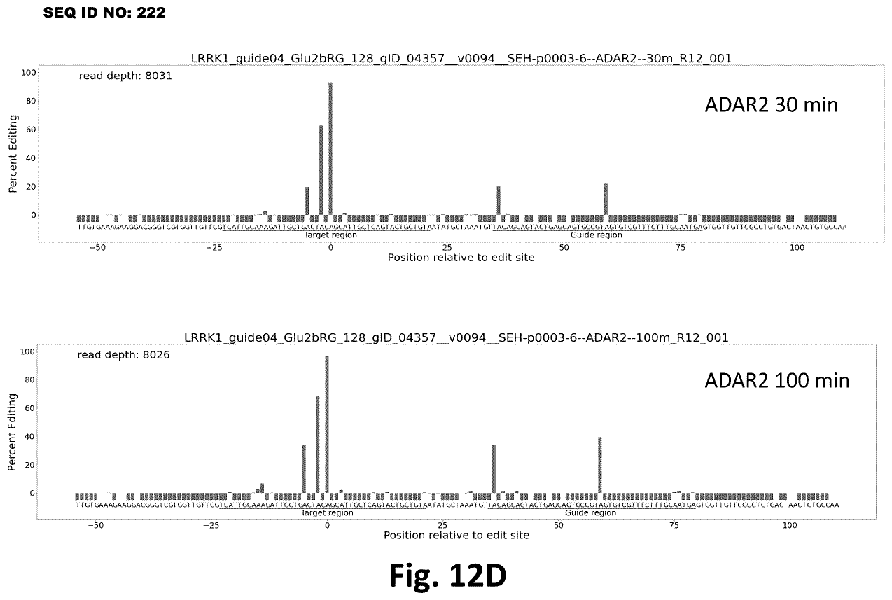

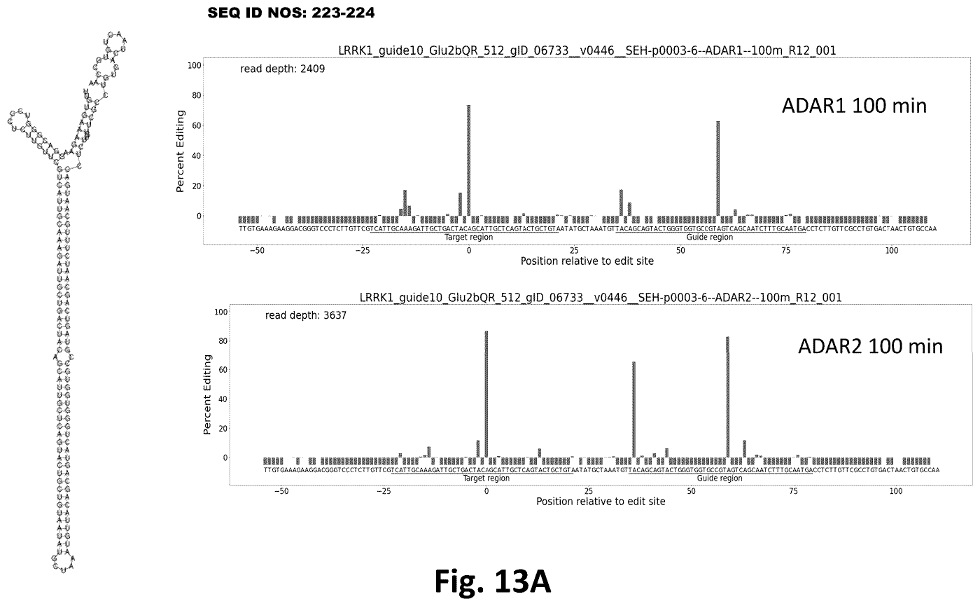

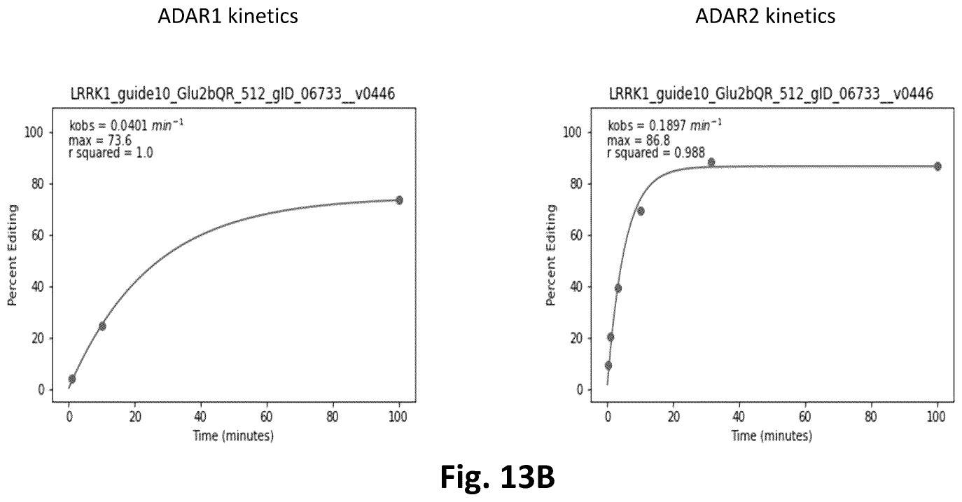

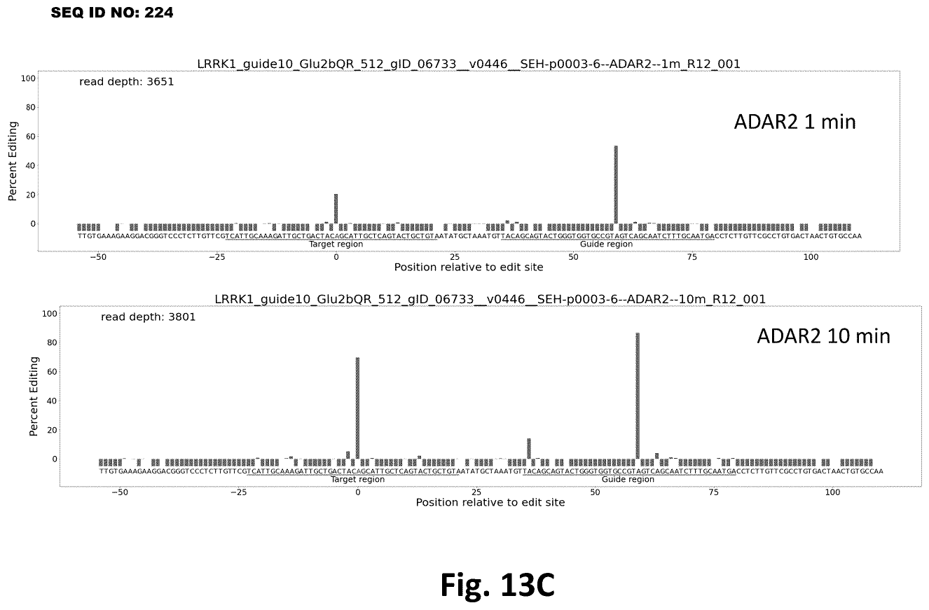

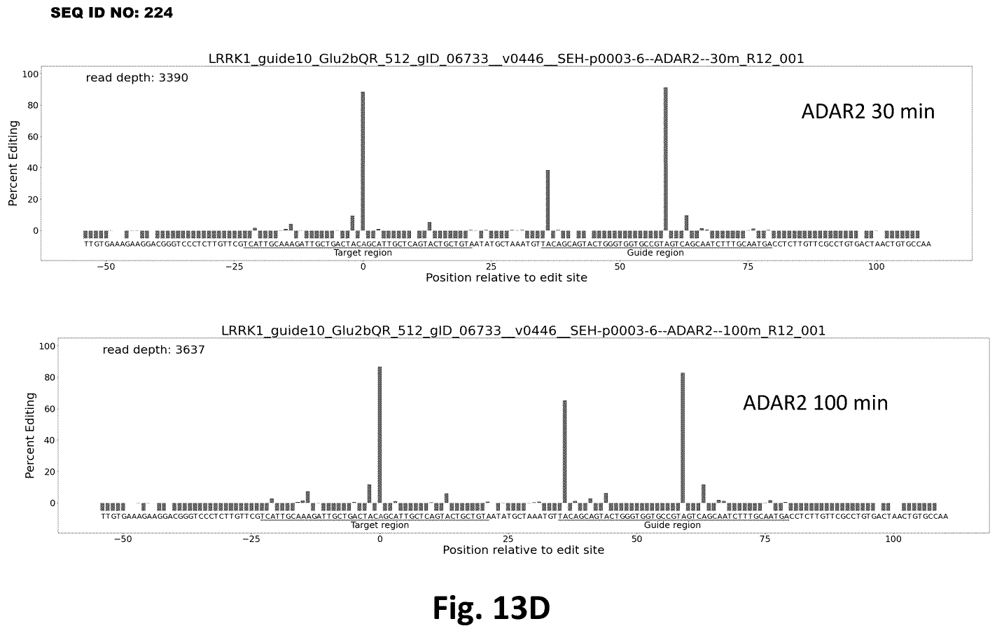

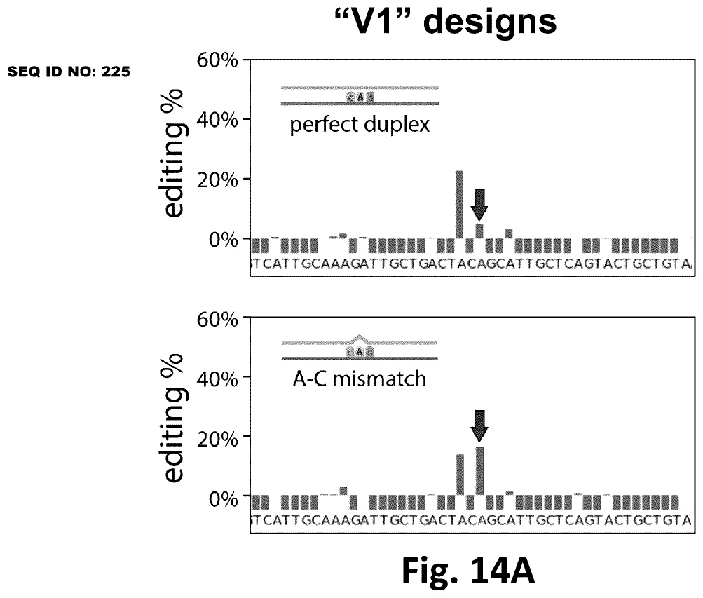

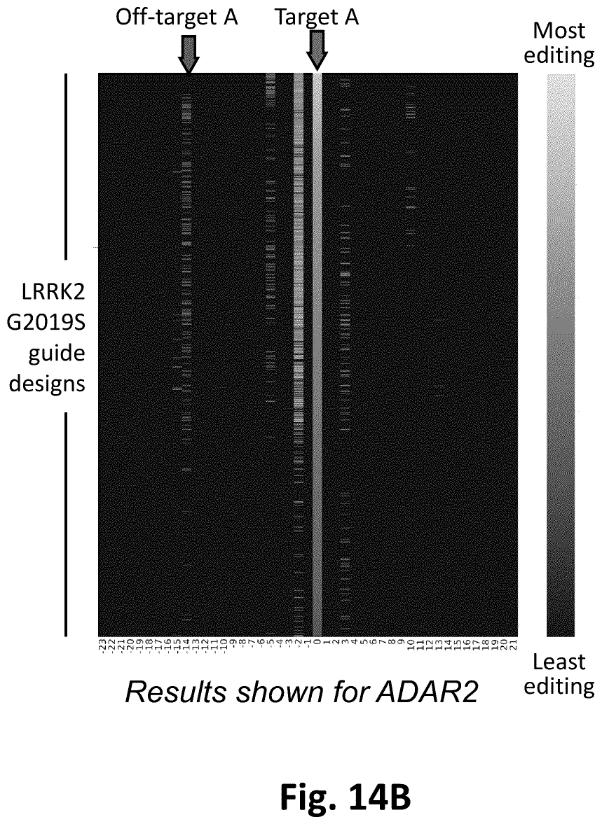

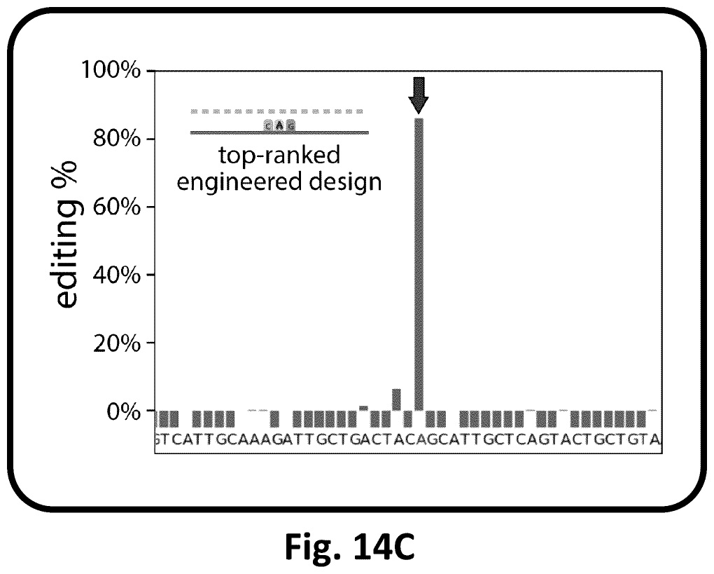

A high throughput screen was carried out to identify guide RNAs for ADAR1 and ADAR2 mediated editing of the LRRK2*G2019S mutation according to the methods described herein (see FIG. 3). The self-annealing RNA structure library screened included 2,536 different gRNAs. Candidate guide RNAs were assessed based on several metrics including: 1) target RNA editing rate kinetics; 2) on-target vs. off-target editing specificity; and 3) ADAR1 vs, ADAR2 agreement (see FIGS. 9 and 10). Exemplary self-annealing RNA structures are depicted in FIGS. 11-13. The subject screening methods identified gRNAs that exhibited superior on-target editing of the LRRK2*G2019S mutation as compared to previous gRNAs (FIGS. 14A-C). FIG. 14B is an NGS readout that shows a profile of the ADAR2 mediated edits of the LRRK2*G2019S target by the self-annealing RNA structures library, including those that mediate on-target editing. The x-axis indicates the LRRK2 target sequence with each position denoted to the target A edit site (denoted as 0). The y-axis represents the individual guides that were tested. Selected candidate gRNA designs will be further tested against LRRK2*G2019S in relevant disease model cell lines.

### Example 3

### High Throughput Screening of Engineered Guide RNAs Targeting ABCA4 for RNA Editing

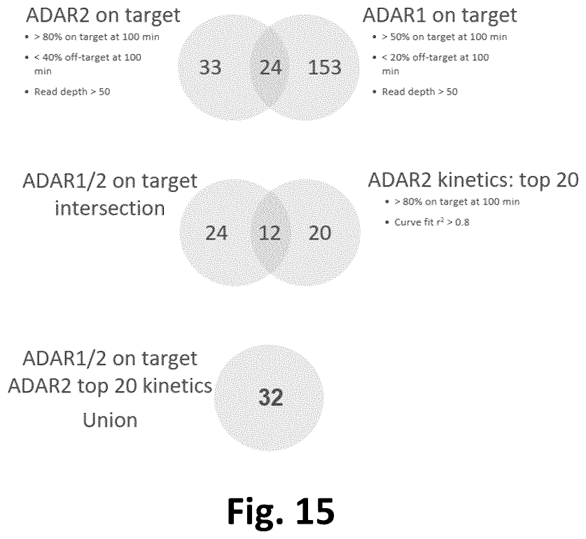

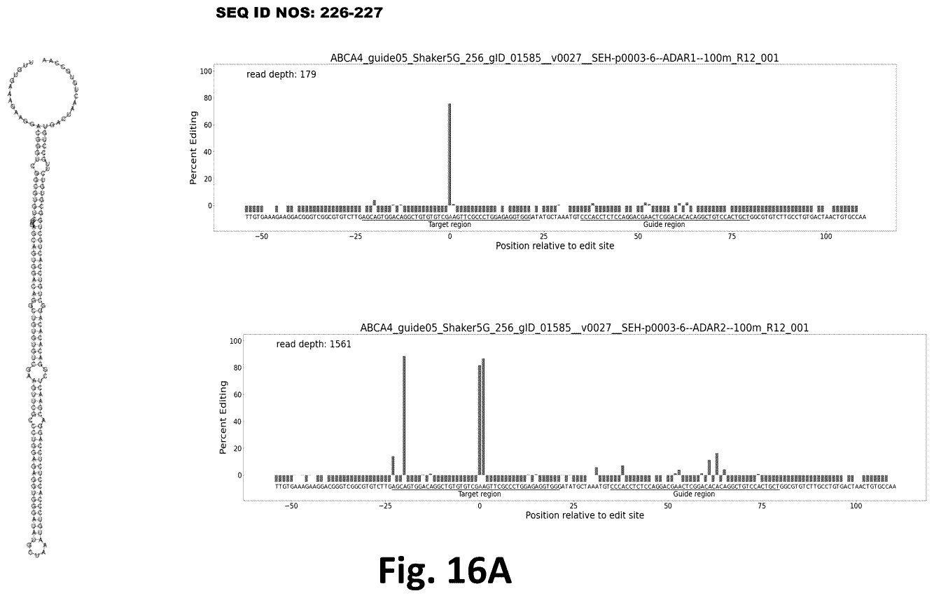

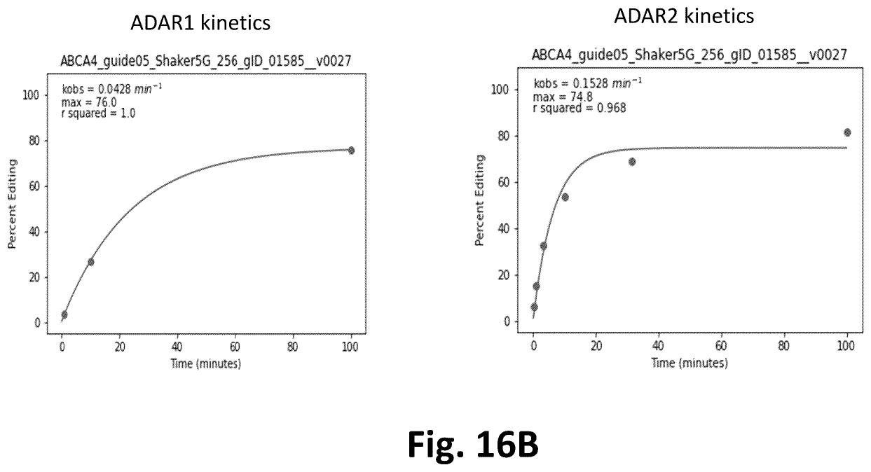

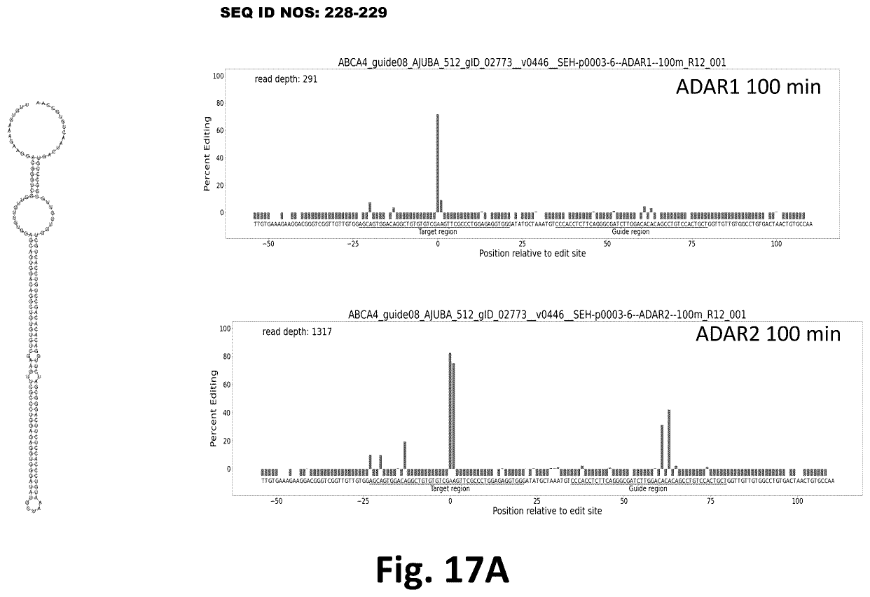

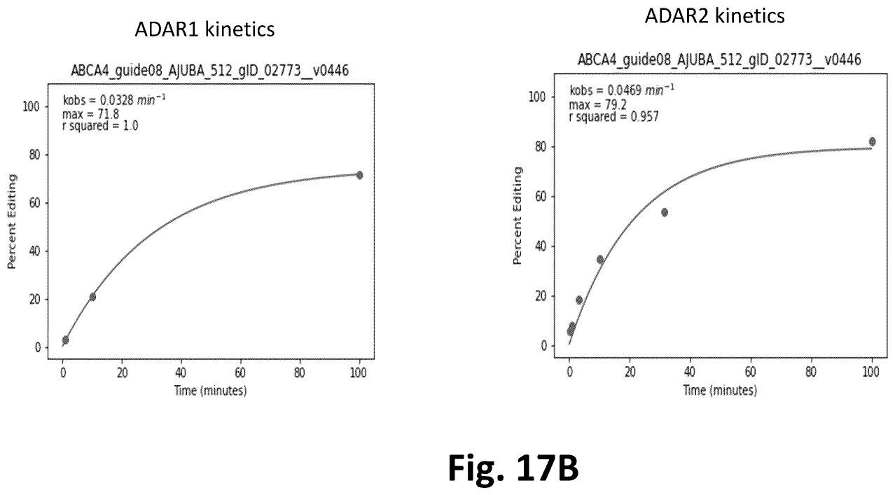

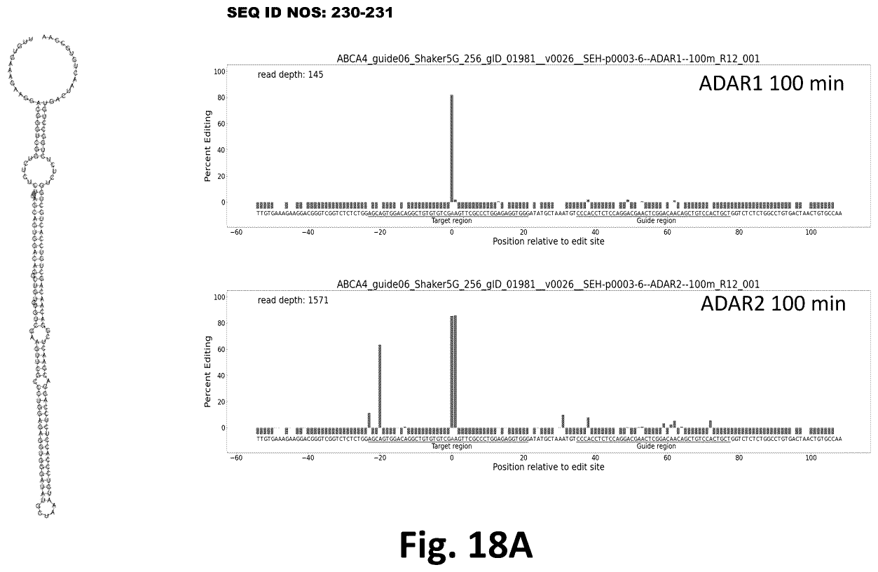

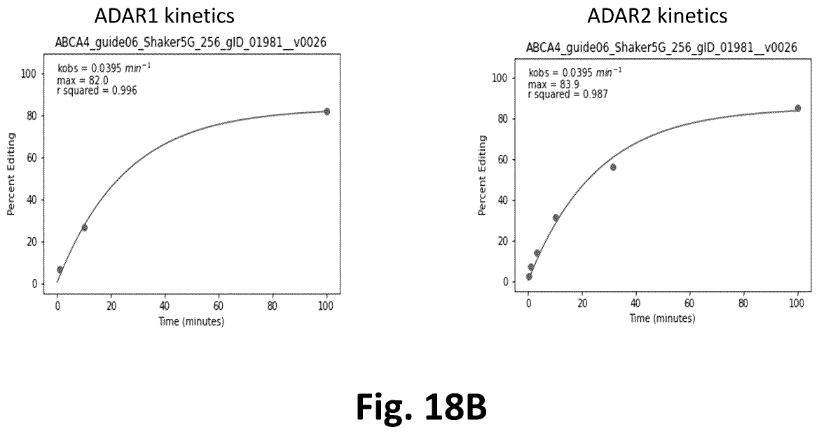

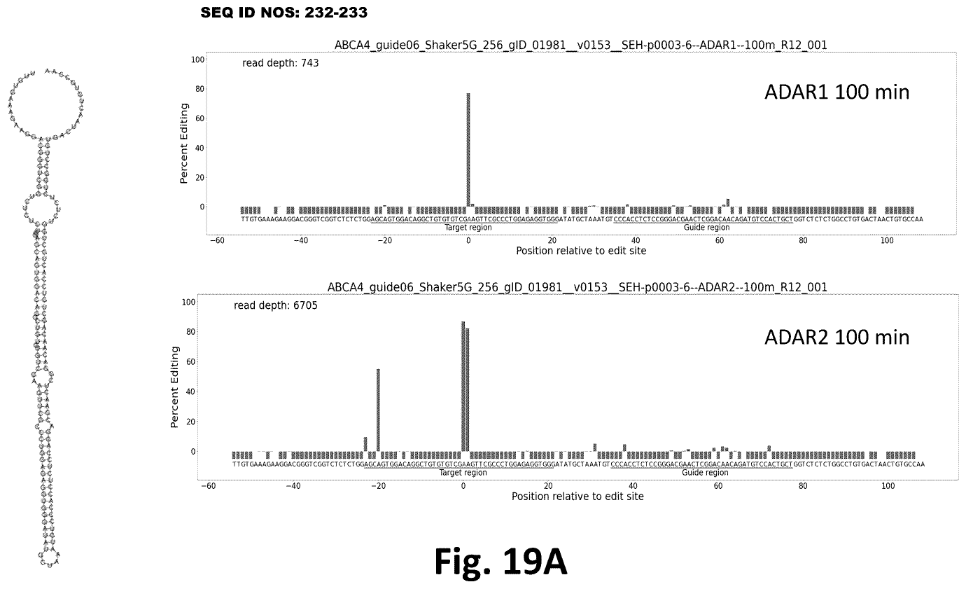

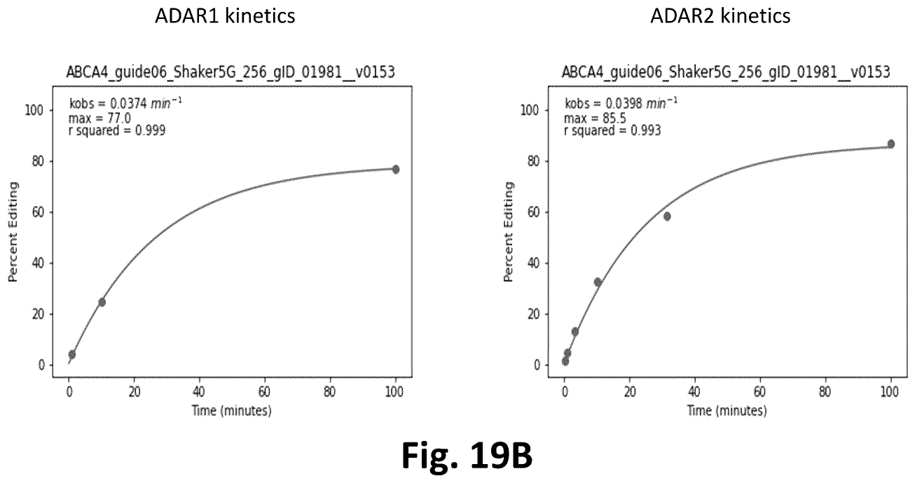

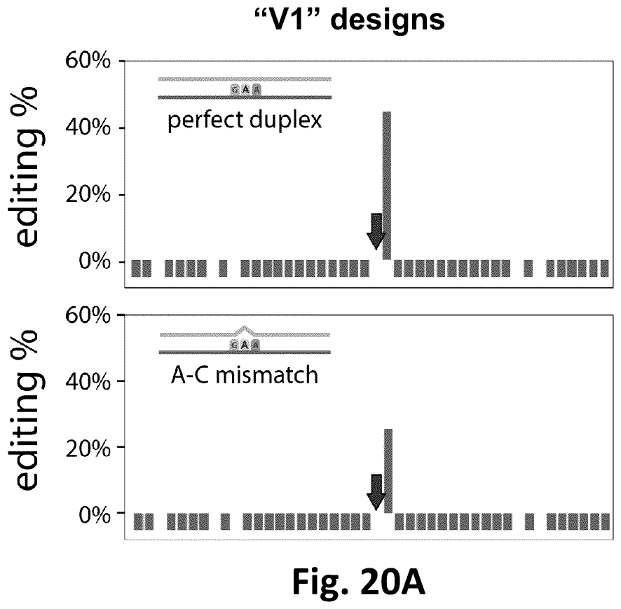

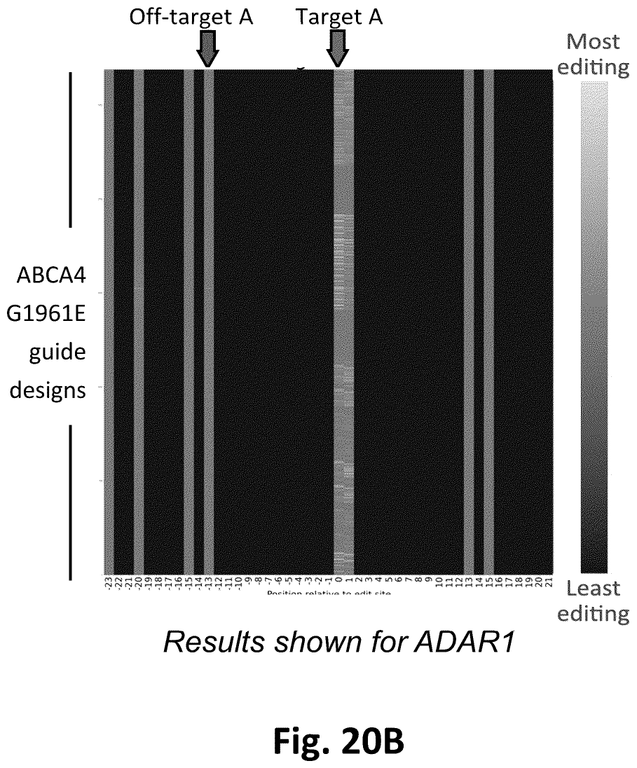

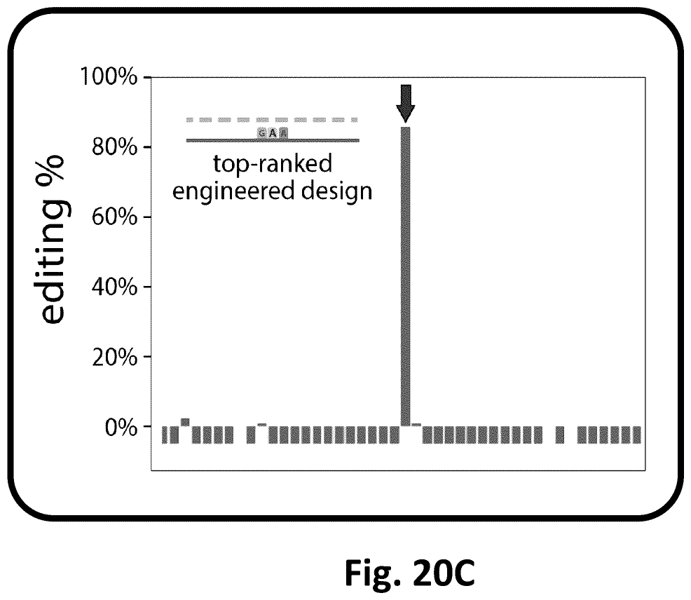

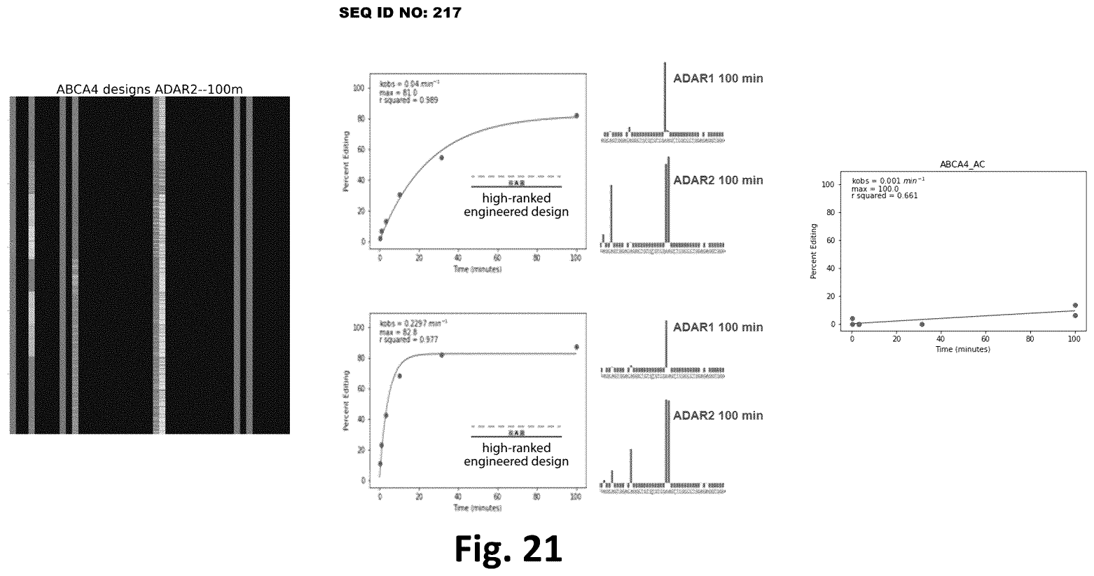

A high throughput screen was carried out to identify guide RNAs for ADAR1 and ADAR2 mediated editing of the ABCA4*G1961E mutation according to the methods described herein (see FIG. 3). The self-annealing RNA structure library screened included 2,688 different gRNAs. Candidate guide RNAs were assessed based on several metrics including: 1) target RNA editing rate kinetics; 2) on-target vs. off-target editing specificity; and 3) ADAR1 vs, ADAR2 agreement (see FIG. 15). Exemplary self-annealing RNA structures are depicted in FIGS. 16-19). The ABCA4 target mutation is in a “5′G” context that is known to be refractory to endogenous ADAR editing (see FIG. 20A). However, the subject screening methods identified gRNAs that exhibited superior editing of the ABCA4*G1961E mutation as compared to previous gRNAs (FIGS. 20B and C). FIG. 20B is an NGS readout that shows a profile of the ADAR1 mediated edits of the ABCA4*G1961E target by the self-annealing RNA structures library, including those that mediate on-target editing. The x-axis indicates the LRRK2 target sequence with each position denoted to the target A edit site (denoted as 0). The y-axis represents the individual guides that were tested. Selected candidate gRNA designs will be further tested against ABCA4*G1961E in relevant cell lines. Moreover, the lead self-annealing RNA structures identified in the screen exhibited enhanced kinetics for editing of ABCA4 as compared to previous gRNA designs (FIG. 21, middle and right). This demonstrates for the first time that endogenous ADAR can be made to edit a novel “5′G” site with the right guide sequence.

### Example 4

### High Throughput Screening of Engineered Guide RNAs Targeting SERPINA1 for RNA Editing

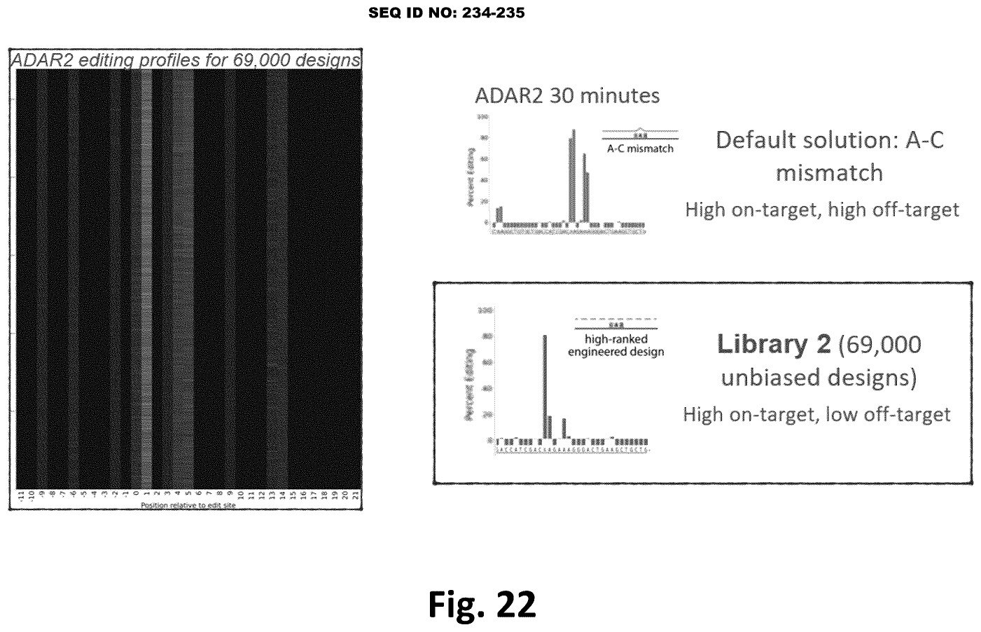

Additional screening was performed to identify gRNAs for SERPINA1 according to the methods described herein. In an initial screen performed, three self-annealing RNA structures were identified that exhibited>70% on-target editing at 100 mins and <70% ADAR2 off-target editing at 100 mins (read depth>50). Ten self-annealing RNA structures were identified that exhibited>40% on-target editing at 100 mins and <40% off-target editing at 100 mins (read depth>50). Further, in the initial ADAR2 kinetics studies, the top 20 self-annealing RNA structures identified exhibited>70% on-target editing at 100 mins (curve fit r²>0.8). A second screen was able to identify candidate RNAs that exhibit high on-target ADAR2 mediated editing for SERPINA1 (FIG. 22).

### Example 5

### High Throughput Screening of Engineered Guide RNAs Targeting the LRRK2 G2019S Mutation for RNA Editing

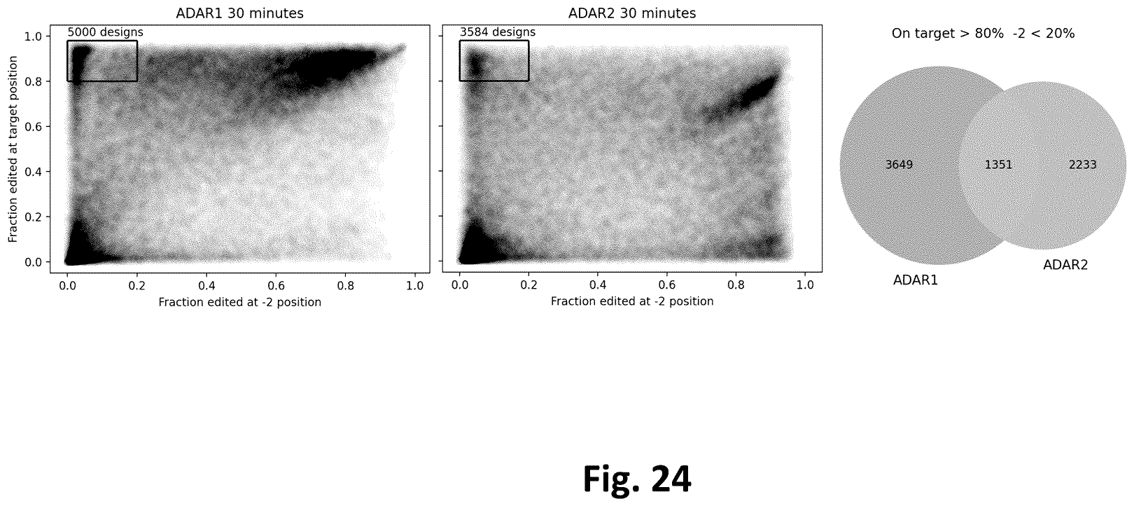

This example describes a high throughput screen designed to screen a library of over 100,000 structurally randomized guide designs targeting the LRRK2 G2019S mutation, a clinically relevant mutation resulting in an adenosine (the target adenosine), which lies two nucleotides downstream of an off-target adenosine. This mutation causes up to 5% of cases of familial Parkinson's disease. The library was incubated with ADAR1 or ADAR2 for 30 minutes. FIG. 24 shows results from the high throughput screen. The fraction edited at the −2 position (x-axis) and target position (y-axis) are depicted in the graph at left and the graph at center. The box in the top left section of each graph details the designs with >80% on target editing and <20% off-target editing at the −2 position. The Venn diagram on the right shows the overlap between the ADAR1 and ADAR2 designs that have >80% on target editing and <20% off target editing at the −2 position. Many designs had a preference for ADAR1 or ADAR2, while some remained selective for the target adenosine with both enzymes. The data demonstrates the importance of profiling both ADAR1 and ADAR2 enzymes when targeting tissues, such as brain, where both ADARs can be present.

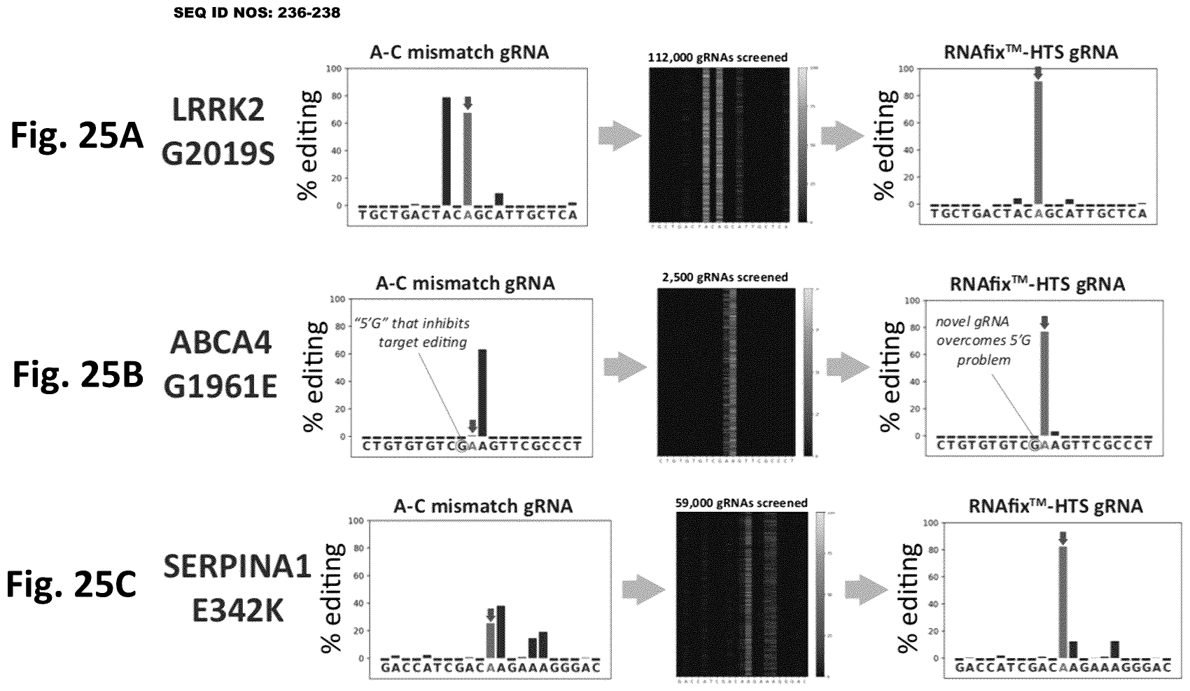

FIG. 25A shows results from the high throughput screen of over 100,000 guide designs targeting the LRRK2 G2019S G to A mutation. From left to right is: a bar graph showing percent editing at each nucleotide relative to the target A (indicated by the arrow) for a standard gRNA forming just an A/C mismatch with the target RNA, an NGS readout of the 112,000 gRNAs that were screened, and a bar graph showing percent editing at each nucleotide relative to the target A (indicated by the arrow) for the engineered gRNA discovered using the methods of high throughput screening disclosed herein. As demonstrated by the data in FIG. 25A, the methods of high throughput screening disclosed herein identify superior novel engineered guide RNAs that show enhanced on-target editing and decreased off-target editing.

### Example 6

### High Throughput Screening of Engineered Guide RNAs Targeting the G1961E ABCA4 Mutation for RNA Editing

This example describes a high throughput screen designed to screen a library of thousands of guide designs targeting the ABCA4 G1961E mutation, a clinically relevant mutation resulting in an adenosine (the target adenosine), which is adjacent to a 5′ guanosine (a local sequence context that strongly disfavors ADAR editing. This mutation causes Stargardt disease. The library was incubated with ADAR2 for 30 minutes. FIG. 25B shows results from the high throughput screen. From left to right is: a bar graph showing percent editing at each nucleotide relative to the target A (indicated by the arrow) for a standard gRNA forming just an A/C mismatch with the target RNA, an NGS readout of the 2500 gRNAs that were screened, and a bar graph showing percent editing at each nucleotide relative to the target A (indicated by the arrow) for the engineered gRNA discovered using the methods of high throughput screening disclosed herein. Unexpectedly, the methods of high throughput screening disclosed herein identified a superior guide RNA capable of mediating high (>60%) on-target editing (in contrast to the negligible on-target adenosine editing observed in the A-C mismatch gRNA at left). The methods disclosed herein, thus, yielded designs that enable both ADAR1 and ADAR2 to edit the ABCA4 G1961 target. These results are especially surprising and represent a major breakthrough as, until now, it was thought that engineered ADAR or chemically modified gRNAs would be needed for editing of target adenosines having a 5′ guanosine.

### Example 7

### High Throughput Screening of Engineered Guide RNAs Targeting the E342K SERPINA1 Mutation for RNA Editing

This example describes a high throughput screen designed to screen a library of tens of thousands of guide designs targeting the SERINA1 E342K mutation, a clinically relevant mutation resulting in an adenosine (the target adenosine), which leads to alpha-1 antitrypsin deficiency (AATD). The library was incubated with ADAR2 for 30 minutes. FIG. 25C shows results from the high throughput screen. From left to right is: a bar graph showing percent editing at each nucleotide relative to the target A (indicated by the arrow) for a standard gRNA forming just an A/C mismatch with the target RNA, an NGS readout of the ˜59,000 gRNAs that were screened, and a bar graph showing percent editing at each nucleotide relative to the target A (indicated by the arrow) for the engineered gRNA discovered using the methods of high throughput screening disclosed herein. As seen in the figure, a convention A-C mismatch gRNA can only enable low efficiency editing and specificity was poor (as evidenced by high editing at numerous off-target adenosines), potentially due to the presence of many local adenosines near the target A. As demonstrated by the data in FIG. 25C, the methods of high throughput screening disclosed herein identify superior novel engineered guide RNAs that show enhanced on-target editing and enhanced specificity (decreased off-target editing).

### Example 8

### High Throughput Screening of Engineered Guide RNAs Targeting ABCA4 for RNA Editing

Using the compositions and methods described herein, high throughput screening (HTS) of 2,500 guide designs were generated and screen for targeting the ABCA4 G1961E mutation. Experiments included incubation of engineered guide RNAs with ADAR1 for 100 min followed by an NGS readout of editing efficiency.

Self-annealing RNA structures comprising the engineered polynucleotide sequences of TABLE 1 and the sequences of the regions targeted by the guide RNAs were contacted with ADAR1 and/or ADAR2 under conditions that allow for the editing of the regions targeted by the guide RNAs (“in cis-editing”). The regions targeted by the guide RNAs were subsequently assessed for editing using next generation sequencing (NGS).

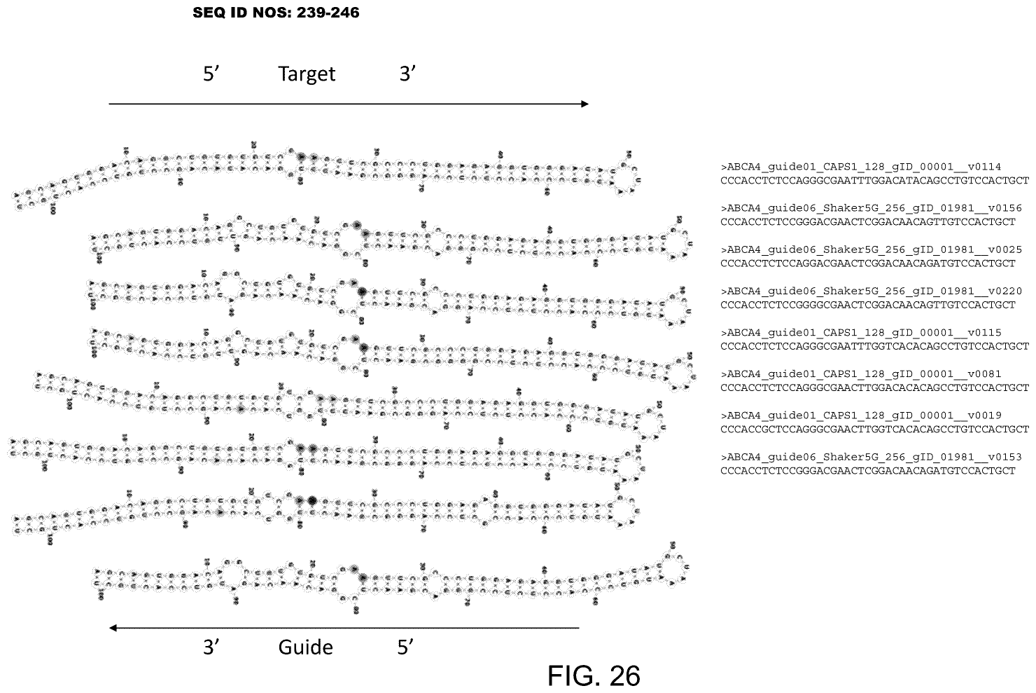

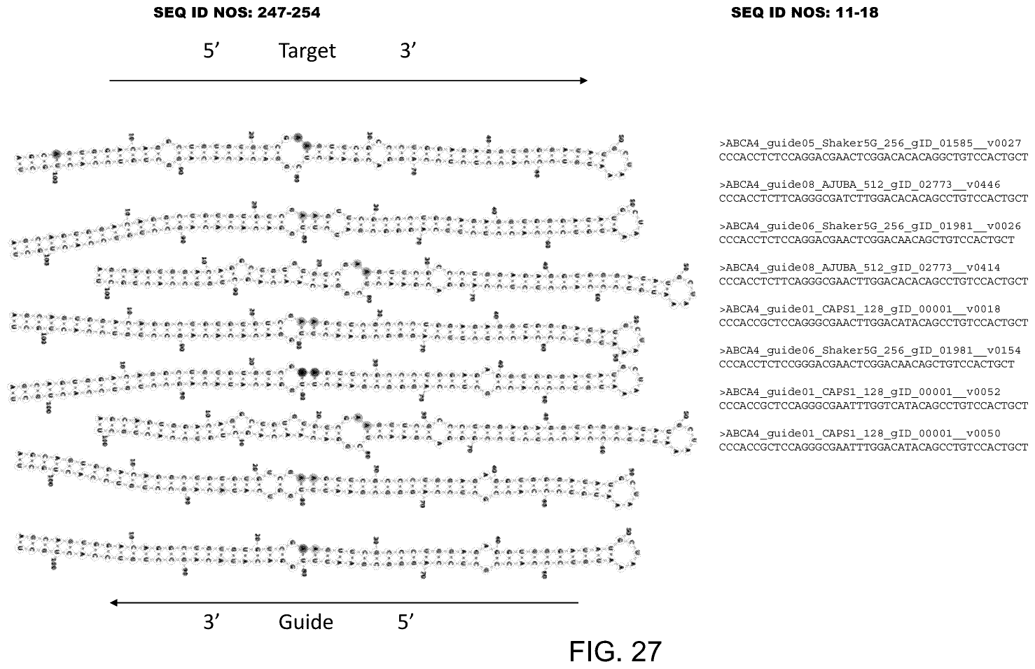

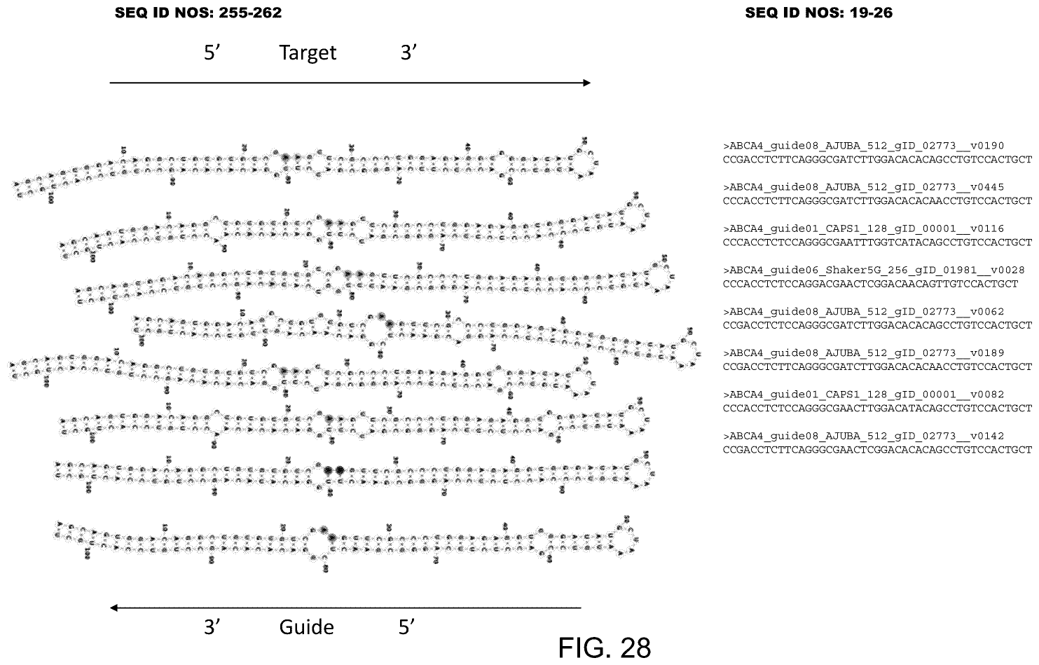

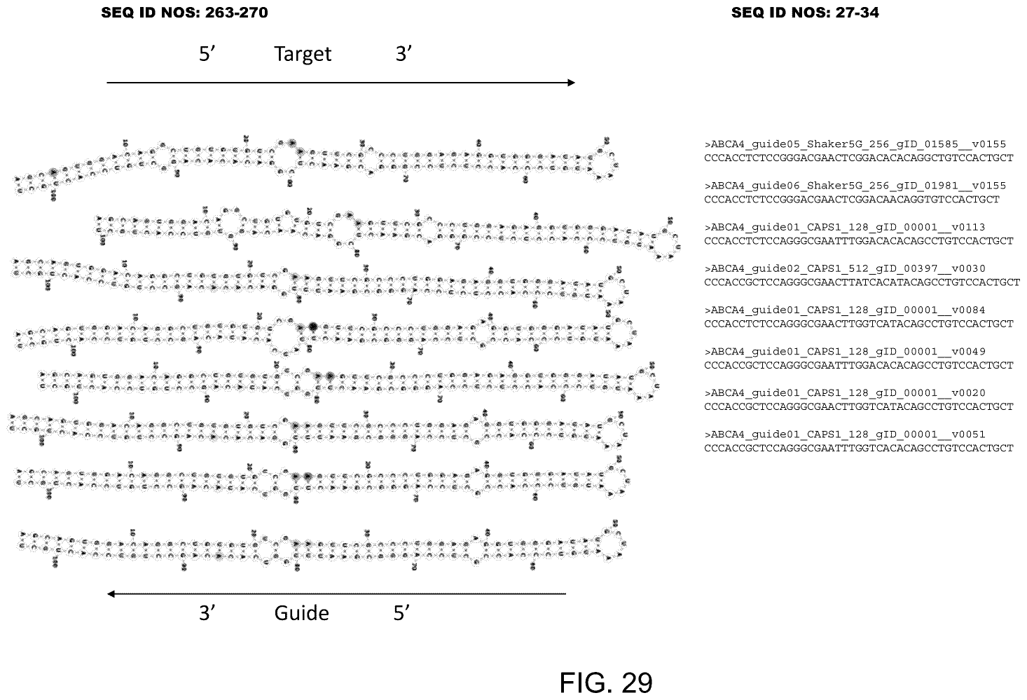

Shown in FIG. 26-FIG. 29 are structures of the self-annealing RNA structures comprising the engineered polynucleotide sequences of TABLE 1 and the sequence of the ABCA4 region targeted by the guide RNAs, highlighting the structural features summarized in TABLE 1. Controls included a perfect duplex double stranded RNA substrate between the target sequence and the guide RNA and a candidate engineered guide RNA that forms a single A/C mismatch upon hybridization to the target sequence. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 1
ABCA4 Engineered Guide RNAs, 45mers for In Cis-Editing Experiments

| SEQ | | | Structural Features | |
|---|---|---|---|---|
| ID NO | Name | Sequence | (target/guide) | Metrics |
| SEQ | Perfect | CCCACCTCTCCAG | | ADAR1 on target: 0.00% |
| ID | Duplex | GGCGAACTTCGAC | | ADAR2 on target: 27.27% |
| NO: 1 | | ACACAGCCTGTCC | | ADAR1 specificity: 1 |
| | | ACTGCT | | ADAR2 specificity: 0.97 |
| SEQ | A/C | CCCACCTCTCCAG | 1 A/C mismatch | ADAR1 on target: 33.33% |
| ID | Mismatch | GGCGAACTCCGAC | | ADAR2 on target: 13.33% |
| NO: 2 | | ACACAGCCTGTCC | | ADAR1 specificity: 1.33 |
| | | ACTGCT | | ADAR2 specificity: 1.06 |
| SEQ | >ABCA4_g | CCCACCTCTCCAG | 1 G/G mismatch | ADAR1 on target: 83.06% |
| ID | uide01_CA | GGCGAATTTGGAC | | ADAR2 on target: 88.09% |
| NO: 3 | PS1_128_gI | ATACAGCCTGTCC | | ADAR1 specificity: 1.79 |
| | D_00001__ | ACTGCT | | ADAR2 specificity: 1.46 |
| | v0114 | | | |
| SEQ | >ABCA4_g | CCCACCTCTCCGG | 1 C/A mismatch | ADAR1 on target: 68.15% |
| ID | uide06_Sha | GACGAACTCGGAC | 2/2 symmetrical bulge | ADAR2 on target: 92.00% |
| NO: 4 | ker5G_256_ | AACAGTTGTCCAC | 1/0 asymmetrical bulge | ADAR1 specificity: 1.6 |
| | gID_01981_ | TGCT | 1/0 asymmetrical bulge | ADAR2 specificity: 1.9 |
| | _v0156 | | | |
| SEQ | >ABCA4_g | CCCACCTCTCCAG | 1 C/A mismatch | ADAR1 on target: 85.34% |
| ID | uide06_Sha | GACGAACTCGGAC | 2/2 symmetrical bulge | ADAR2 on target: 81.67% |
| NO: 5 | ker5G_256_ | AACAGATGTCCAC | 1/0 asymmetrical bulge | ADAR1 specificity: 1.84 |
| | gID_01981_ | TGCT | 2/1 asymmetrical bulge | ADAR2 specificity: 1.8 |
| | _v0025 | | | |
| SEQ | >ABCA4_g | CCCACCTCTCCGG | 2/2 symmetrical bulge | ADAR1 on target: 73.27% |
| ID | uide06_Sha | GGCGAACTCGGAC | 1/0 asymmetrical bulge | ADAR2 on target: 82.46% |
| NO: 6 | ker5G_256_ | AACAGTTGTCCAC | 1/0 asymmetrical bulge | ADAR1 specificity: 1.64 |
| | gID_01981_ | TGCT | | ADAR2 specificity: 1.8 |
| | _v0220 | | | |
| SEQ | >ABCA4_g | CCCACCTCTCCAG | 1 G/G mismatch | ADAR1 on target: 76.06% |
| ID | uide01_CA | GGCGAATTTGGTC | 1 U/U mismatch | ADAR2 on target: 83.92% |
| NO: 7 | PS1_128_gI | ACACAGCCTGTCC | | ADAR1 specificity: 1.68 |
| | D_00001__ | ACTGCT | | ADAR2 specificity: 1.42 |
| | v0115 | | | |
| SEQ | >ABCA4_g | CCCACCTCTCCAG | 1 G/G mismatch | ADAR1 on target: 67.69% |
| ID | uide01_CA | GGCGAACTTGGAC | | ADAR2 on target: 81.74% |
| NO: 8 | PS1_128_gI | ACACAGCCTGTCC | | ADAR1 specificity: 1.63 |
| | D_00001__ | ACTGCT | | ADAR2 specificity: 1.51 |
| | v0081 | | | |
| SEQ | >ABCA4_g | CCCACCGCTCCAG | 1 A/G mismatch | ADAR1 on target: 38.81% |
| ID | uide01_CA | GGCGAACTTGGTC | 1 G/G mismatch | ADAR2 on target: 83.53% |
| NO: 9 | PS1_128_gI | ACACAGCCTGTCC | 1 U/U mismatch | ADAR1 specificity: 1.37 |
| | D_00001__ | ACTGCT | | ADAR2 specificity: 1.34 |
| | v0019 | | | |
| SEQ | >ABCA4_g | CCCACCTCTCCGG | 1 C/A mismatch | ADAR1 on target: 76.99% |
| ID | uide06_Sha | GACGAACTCGGAC | 2/2 symmetrical bulge | ADAR2 on target: 86.79% |
| NO: | ker5G_256_ | AACAGATGTCCAC | 1/0 asymmetrical bulge | ADAR1 specificity: 1.76 |
| 10 | gID_01981_ | TGCT | 2/1 asymmetrical bulge | ADAR2 specificity: 1.86 |
| | _v0153 | | | |
| SEQ | >ABCA4_g | CCCACCTCTCCAG | 1 C/A mismatch | ADAR1 on target: 75.98% |
| ID | uide05_Sha | GACGAACTCGGAC | 2/2 symmetrical bulge | ADAR2 on target: 81.74% |
| NO: | ker5G_256_ | ACACAGGCTGTCC | 1 G/G mismatch | ADAR1 specificity: 1.74 |
| 11 | gID_01585_ | ACTGCT | | ADAR2 specificity: 1.78 |
| | _v0027 | | | |
| SEQ | >ABCA4_g | CCCACCTCTTCAG | 1 U/U mismatch | ADAR1 on target: 71.82% |
| ID | uide08_AJU | GGCGATCTTGGAC | 1 G/G mismatch | ADAR2 on target: 82.46% |
| NO: | BA_512_gI | ACACAGCCTGTCC | | ADAR1 specificity: 1.66 |
| 12 | D_02773__ | ACTGCT | | ADAR2 specificity: 1.52 |
| | v0446 | | | |
| SEQ | >ABCA4_g | CCCACCTCTCCAG | 1 C/A mismatch | ADAR1 on target: 82.07% |
| ID | uide06_Sha | GACGAACTCGGAC | 2/2 symmetrical bulge | ADAR2 on target: 85.49% |
| NO: | ker5G_256_ | AACAGCTGTCCAC | 1/0 asymmetrical bulge | ADAR1 specificity: 1.82 |
| 13 | gID_01981_ | TGCT | 1/0 asymmetrical bulge | ADAR2 specificity: 1.84 |
| | _v0026 | | | |
| SEQ | >ABCA4_g | CCCACCTCTTCAG | 1 G/G mismatch | ADAR1 on target: 71.05% |
| ID | uide08_AJU | GGCGAACTTGGAC | | ADAR2 on target: 81.51% |
| NO: | BA_512_gI | ACACAGCCTGTCC | | ADAR1 specificity: 1.67 |
| 14 | D_02773__ | ACTGCT | | ADAR2 specificity: 01.51 |
| | v0414 | | | |
| SEQ | >ABCA4_g | CCCACCGCTCCAG | 1 G/A mismatch | ADAR1 on target: 49.09% |
| ID | uide01_CA | GGCGAACTTGGAC | 1 G/G mismatch | ADAR2 on target: 87.42% |
| NO: | PS1_128_gI | ATACAGCCTGTCC | | ADAR1 specificity: 1.46 |
| 15 | D_00001__ | ACTGCT | | ADAR2 specificity: 1.37 |
| | v0018 | | | |
| SEQ | >ABCA4_g | CCCACCTCTCCGG | 1 C/A mismatch | ADAR1 on target: 67.94% |
| ID | uide06_Sha | GACGAACTCGGAC | 2/2 symmetrical bulge | ADAR2 on target: 88.82% |
| NO: | ker5G_256_ | AACAGCTGTCCAC | 1/0 asymmetrical bulge | ADAR1 specificity: 1.68 |
| 16 | gID_01981_ | TGCT | 1/0 asymmetrical bulge | ADAR2 specificity: 1.87 |
| | _v0154 | | | |
| SEQ | >ABCA4_g | CCCACCGCTCCAG | 1 G/A mismatch | ADAR1 on target: 38.38% |
| ID | uide01_CA | GGCGAATTTGGTC | 1 G/G mismatch | ADAR2 on target: 90.74% |
| NO: | PS1_128_gI | ATACAGCCTGTCC | 1 U/U mismatch | ADAR1 specificity: 1.36 |
| 17 | D_00001__ | ACTGCT | | ADAR2 specificity: 1.35 |
| | v0052 | | | |
| SEQ | >ABCA4_g | CCCACCGCTCCAG | 1 G/A mismatch | ADAR1 on target: 53.60% |
| ID | uide01_CA | GGCGAATTTGGAC | 1 G/G mismatch | ADAR2 on target: 91.32% |
| NO: | PS1_128_gI | ATACAGCCTGTCC | | ADAR1 specificity: 1.52 |
| 18 | D_00001__ | ACTGCT | | ADAR2 specificity: 1.45 |
| | v0050 | | | |
| SEQ | >ABCA4_g | CCGACCTCTTCAG | 1 G/G mismatch | ADAR1 on target: 61.32% |
| ID | uide08_AJU | GGCGATCTTGGAC | 1 U/U mismatch | ADAR2 on target: 83.41% |
| NO: | BA_512_gI | ACACAGCCTGTCC | 1 G/G mismatch | ADAR1 specificity: 1.56 |
| 19 | D_02773__ | ACTGCT | | ADAR2 specificity: 1.54 |
| | v0190 | | | |
| SEQ | >ABCA4_g | CCCACCTCTTCAG | 1 U/U mismatch | ADAR1 on target: 75.23% |
| ID | uide08_AJU | GGCGATCTTGGAC | 1 G/G mismatch | ADAR2 on target: 81.74% |
| NO: | BA_512_gI | ACACAACCTGTCC | 1 C/A mismatch | ADAR1 specificity: 1.74 |
| 20 | D_02773__ | ACTGCT | | ADAR2 specificity: 1.67 |
| | v0445 | | | |
| SEQ | >ABCA4_g | CCCACCTCTCCAG | 1 G/G mismatch | ADAR1 on target: 71.78% |
| ID | uide01_CA | GGCGAATTTGGTC | 1 U/U mismatch | ADAR2 on target: 87.83% |
| NO: | PS1_128_gI | ATACAGCCTGTCC | | ADAR1 specificity: 1.69 |
| 21 | D_00001__ | ACTGCT | | ADAR2 specificity: 1.31 |
| | v0116 | | | |
| SEQ | >ABCA4_g | CCCACCTCTCCAG | 1 C/A mismatch | ADAR1 on target: 75.77% |
| ID | uide06_Sha | GACGAACTCGGAC | 2/2 symmetrical bulge | ADAR2 on target: 88.24% |
| NO: | ker5G_256_ | AACAGTTGTCCAC | 1/0 asymmetrical bulge | ADAR1 specificity: 1.64 |
| 22 | gID_01981_ | TGCT | 1/0 asymmetrical bulge | ADAR2 specificity: 1.86 |
| | _v0028 | | | |
| SEQ | >ABCA4_g | CCGACCTCTCCAG | 1 G/G mismatch | ADAR1 on target: 77.88% |
| ID | uide08_AJU | GGCGATCTTGGAC | 1 U/U mismatch | ADAR2 on target: 80.23% |
| NO: | BA_512_gI | ACACAGCCTGTCC | 1 G/G mismatch | ADAR1 specificity: 1.75 |
| 23 | D_02773__ | ACTGCT | | ADAR2 specificity: 1.48 |
| | v0062 | | | |
| SEQ | >ABCA4_g | CCGACCTCTTCAG | 1 G/G mismatch | ADAR1 on target: 67.61% |
| ID | uide08_AJU | GGCGATCTTGGAC | 1 U/U mismatch | ADAR2 on target: 80.01% |
| NO: | BA_512_gI | ACACAACCTGTCC | 1 G/G mismatch | ADAR1 specificity: 1.65 |
| 24 | D_02773__ | ACTGCT | 1 C/A mismatch | ADAR2 specificity: 1.67 |
| | v0189 | | | |
| SEQ | >ABCA4_g | CCCACCTCTCCAG | 1 G/G mismatch | ADAR1 on target: 78.67% |
| ID | uide01_CA | GGCGAACTTGGAC | | ADAR2 on target: 89.22% |
| NO: | PS1_128_gI | ATACAGCCTGTCC | | ADAR1 specificity: 1.75 |
| 25 | D_00001__ | ACTGCT | | ADAR2 specificity: 1.35 |
| | v0082 | | | |
| SEQ | >ABCA4_g | CCGACCTCTTCAG | 1 G/G mismatch | ADAR1 on target: 56.22% |
| ID | uide08_AJU | GGCGAACTCGGAC | 2/2 symmetrical bulge | ADAR2 on target: 81.20% |
| NO: | BA_512_gI | ACACAGCCTGTCC | | ADAR1 specificity: 1.52 |
| 26 | D_02773__ | ACTGCT | | ADAR2 specificity: 1.59 |
| | v0142 | | | |
| SEQ | >ABCA4_g | CCCACCTCTCCGG | 1 C/A mismatch | ADAR1 on target: 66.74% |
| ID | uide05_Sha | GACGAACTCGGAC | 2/2 symmetrical bulge | ADAR2 on target: 82.99% |
| NO: | ker5G_256_ | ACACAGGCTGTCC | 1 G/G mismatch | ADAR1 specificity: 1.65 |
| 27 | gID_01585_ | ACTGCT | | ADAR2 specificity: 1.77 |
| | _v0155 | | | |
| SEQ | >ABCA4_g | CCCACCTCTCCGG | 1 C/A mismatch | ADAR1 on target: 79.65% |
| ID | uide06_Sha | GACGAACTCGGAC | 2/2 symmetrical bulge | ADAR2 on target: 83.65% |
| NO: | ker5G_256_ | AACAGGTGTCCAC | 2/1 asymmetrical bulge | ADAR1 specificity: 1.8 |
| 28 | gID_01981_ | TGCT | | ADAR2 specificity: 1.83 |
| | _v0155 | | | |
| SEQ | >ABCA4_g | CCCACCTCTCCAG | 1 G/G mismatch | ADAR1 on target: 83.97% |
| ID | uide01_CA | GGCGAATTTGGAC | | ADAR2 on target: 81.88% |
| NO: | PS1_128_gI | ACACAGCCTGTCC | | ADAR1 specificity: 1.8 |
| 29 | D_00001__ | ACTGCT | | ADAR2 specificity: 1.53 |
| | v0113 | | | |
| SEQ | >ABCA4_g | CCCACCGCTCCAG | 1 A/G mismatch | ADAR1 on target: 23.40% |
| ID | uide02_CA | GGCGAACTTATCA | 2/2 symmetrical bulge | ADAR2 on target: 88.15% |
| NO: | PS1_512_gI | CATACAGCCTGTC | | ADAR1 specificity: 1.23 |
| 30 | D_00397__ | CACTGCT | | ADAR2 specificity: 1.49 |
| | v0030 | | | |
| SEQ | >ABCA4_g | CCCACCTCTCCAG | 1 G/G mismatch | ADAR1 on target: 74.79% |
| ID | uide01_CA | GGCGAACTTGGTC | 1 U/U mismatch | ADAR2 on target: 86.23% |
| NO: | PS1_128_gI | ATACAGCCTGTCC | | ADAR1 specificity: 1.72 |
| 31 | D_00001__ | ACTGCT | | ADAR2 specificity: 1.23 |
| | v0084 | | | |
| SEQ | >ABCA4_g | CCCACCGCTCCAG | 1 A/G mismatch | ADAR1 on target: 53.83% |
| ID | uide01_CA | GGCGAATTTGGAC | 1 G/G mismatch | ADAR2 on target: 86.56% |
| NO: | PS1_128_gI | ACACAGCCTGTCC | | ADAR1 specificity: 1.53 |
| 32 | D_00001__ | ACTGCT | | ADAR2 specificity: 1.59 |
| | v0049 | | | |
| SEQ | >ABCA4_g | CCCACCGCTCCAG | 1 A/G mismatch | ADAR1 on target: 36.67% |
| ID | uide01_CA | GGCGAACTTGGTC | 1 G/G mismatch | ADAR2 on target: 87.11% |
| NO: | PS1_128_gI | ATACAGCCTGTCC | 1 U/U mismatch | ADAR1 specificity: 1.34 |
| 33 | D_ 00001__ | ACTGCT | | ADAR2 specificity: 1.21 |
| | v0020 | | | |
| SEQ | >ABCA4_g | CCCACCGCTCCAG | 1 A/G mismatch | ADAR1 on target: 40.04% |
| ID | uide01_CA | GGCGAATTTGGTC | 1 G/G mismatch | ADAR2 on target: 85.77% |
| NO: | PS1_128_gI | ACACAGCCTGTCC | 1 U/U mismatch | ADAR1 specificity: 1.38 |
| 34 | D_00001__ | ACTGCT | | ADAR2 specificity: 1.48 |
| | v0051 | | | |

### Example 9

### High Throughput Screening of Engineered Guide RNAs Targeting LRRK2 for RNA Editing

Using the compositions and methods described herein, high throughput screening (HTS) of 2,540 gRNA sequences against the LRRK2*G2019S mutation identified designs with superior on-target activity and specificity. Self-annealing RNA structures comprising the engineered polynucleotide sequences of TABLE 2 (and control engineered polynucleotide sequences) and the sequences of the regions targeted by the guide RNAs were contacted with an RNA editing entity (e.g., a recombinant ADAR1 and/or ADAR2) under conditions that allow for the editing of the regions targeted by the guide RNAs. The regions targeted by the guide RNAs were subsequently assessed for editing using next generation sequencing (NGS).

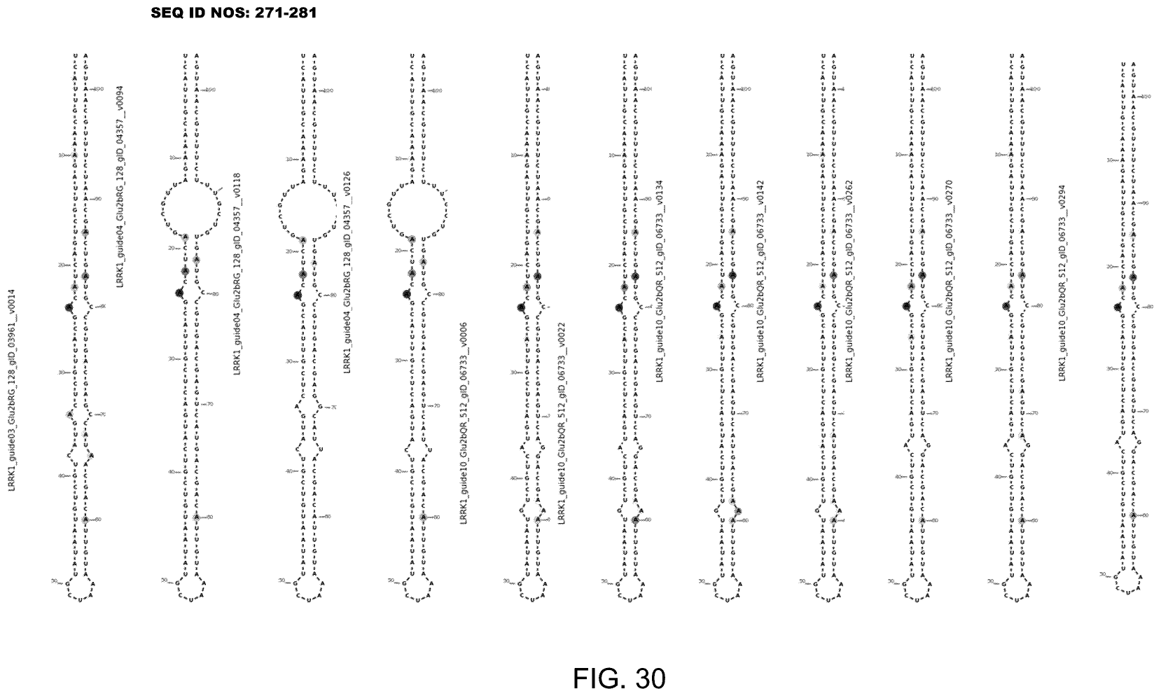

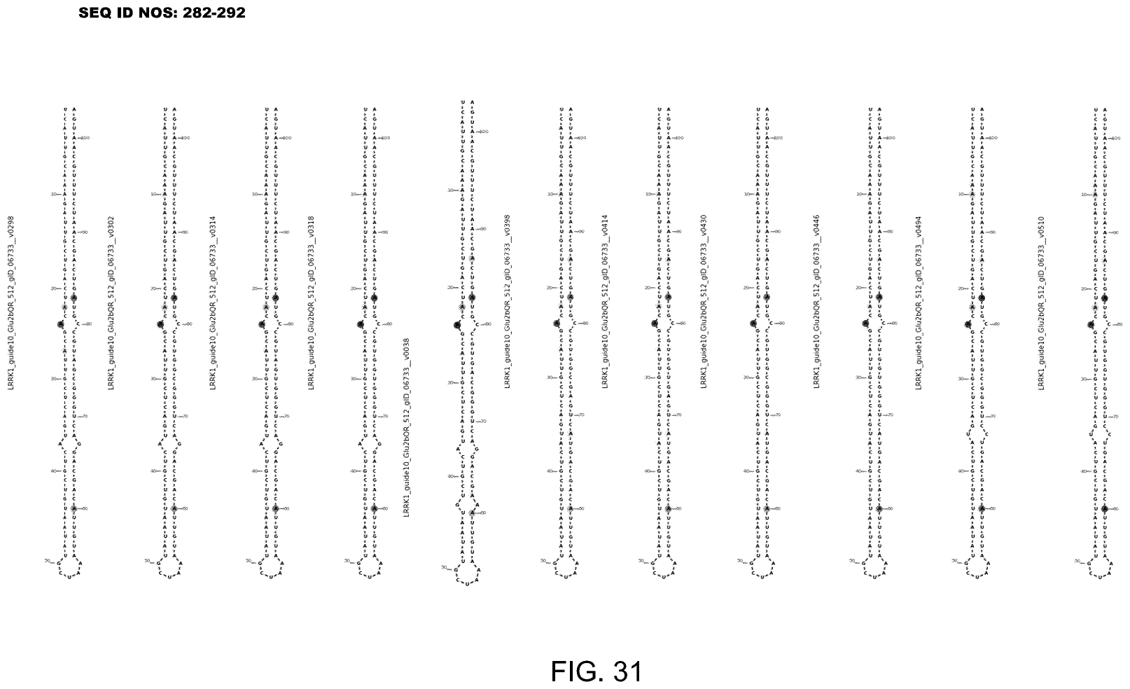

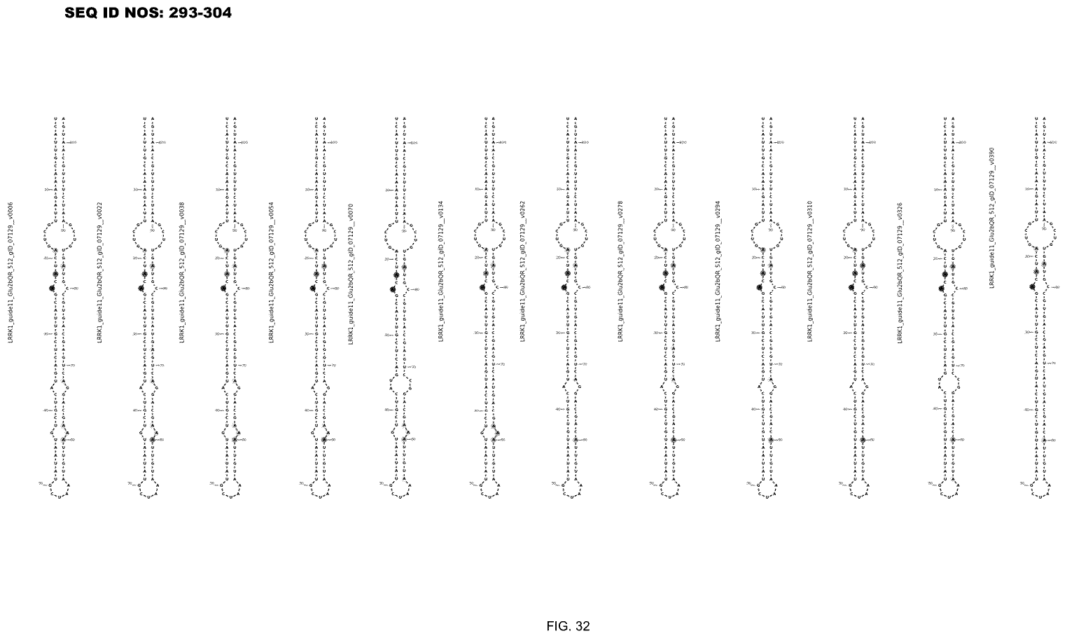

FIG. 30-FIG. 32 shows diagrams of the guide-target RNA scaffold, highlighting the various structural features provided for in the candidate engineered guide RNAs of TABLE 2. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 2
Exemplary Engineered Guide RNAs Targeting LRRK2 mRNA, 45 mers for
In Cis-Editing Experiments

| SEQ | | | | |
|---|---|---|---|---|
| ID | | | Structural Features | |
| NO | ID | Sequence | (target/guide) | Metrics |
| SEQ | LRRK1_guide | TACAGCAGTA | 1/1 A/C mismatch at 0 position | ADAR1 on |
| ID | 10_Glu2bRG_ | CTGGGTGGTG | 1/1 U/G wobble base pair at +4 | target: 73.70% |
| NO: | 512_gID_067 | CCGTAGTCAG | position | ADAR2 on |
| 35 | 33__v0446 | CAATCTTTGCA | 1/1 U/G wobble base pair at +5 | target: 86.88% |
| | | ATGA | position | ADAR1 |
| | | | 1/1 G/U wobble base pair at +6 | specificity: 1.2 |
| | | | position | ADAR2 |
| | | | 1/1 U/G wobble base pair at +8 | specificity: 1.41 |
| | | | position | |
| SEQ | LRRK1_guide | TACAGCAGGA | 4/4 symmetric bulge at −6 | ADAR1 on |
| ID | 11_Glu2bRG_ | CTGAGCAGTG | position (GCUG-GUCG) | target: 65.40% |
| NO: | 512_gID_072 | CCGTAGTGTC | 1/1 A/C mismatch at 0 position | ADAR2 on |
| 36 | 19__v0262 | GAATCTTTGCA | 1/1 U/G wobble base pair at +4 | target: 99.13% |
| | | ATGA | position | ADAR1 |
| | | | 1/1 A/G mismatch at +13 | specificity: 1.28 |
| | | | position | ADAR2 |
| | | | | specificity: 0.89 |
| SEQ | LRRK1_guide | TAAAGCAGGA | 1/1 A/C mismatch at 0 position | ADAR1 on |
| ID | 10_Glu2bQR_ | CTGAGTAGTG | 1/1 U/G wobble base pair at +4 | target: 33.17% |
| NO: | 512_gID_067 | CCGTAGTCAG | position | ADAR2 on |
| 37 | 33__v0022 | CAATCTTTGCA | 1/1 G/U wobble base pair at +6 | target: 98.42% |
| | | ATGA | position | ADAR1 |
| | | | 1/1 A/G mismatch at +13 | specificity: 1.05 |
| | | | position | ADAR2 |
| | | | | specificity: 0.99 |
| SEQ | LRRK1_guide | TACAGCAGTA | 6/6 symmetric internal loop at | ADAR1 on |
| ID | 04_Glu2bRG_ | CTGAGCAGTG | −6 position (UUGCUG-GUCGUU) | target: 62.41% |
| NO: | 128_gID_043 | CCGTAGTGTC | 1/1 A/C mismatch at 0 position | ADAR2 on |
| 38 | 57__v0094 | GTTTCTTTGCA | 1/1 U/G wobble base pair at +4 | target: 96.74% |
| | | ATGA | position | ADAR1 |
| | | | | specificity: 1.07 |
| | | | | ADAR2 |
| | | | | specificity: 0.91 |
| SEQ | LRRK1_guide | TACAGCATTA | 6/6 symmetric internal loop at | ADAR1 on |
| ID | 04_Glu2bRG_ | CTGAGCAGTG | −6 position (UUGCUG-GUCGUU) | target: 12.45% |
| NO: | 128_gID_043 | CCGTAGTGTC | 1/1 A/C mismatch at 0 position | ADAR2 on |
| 39 | 57__v0126 | GTTTCTTTGCA | 1/1 U/G wobble base pair at +4 | target: 98.98% |
| | | ATGA | position | ADAR1 |
| | | | 1/1 C/U mismatch at +14 | specificity: 0.89 |
| | | | position | ADAR2 |
| | | | | specificity: 0.9 |
| SEQ | LRRK1_guide | TACAGCAGGA | 4/4 symmetric bulge at −6 | ADAR1 on |
| ID | 11_Glu2bQR_ | CTGAGTAGTG | position (GCUG-GUCG) | target: 46.44% |
| NO: | 512_gID_071 | CCGTAGTGTC | 1/1 A/C mismatch at 0 position | ADAR2 on |
| 40 | 29__v0278 | GAATCTTTGCA | 1/1 U/G wobble base pair at +4 | target: 98.62% |
| | | ATGA | position | ADAR1 |
| | | | 1/1 G/U wobble base pair at +6 | specificity: 1.22 |
| | | | position | ADAR2 |
| | | | 1/1 A/G mismatch at +13 | specificity: 0.9 |
| | | | position | |
| SEQ | LRRK1_guide | TACAGCAGGA | 1/1 A/C mismatch at 0 position | ADAR1 on |
| ID | 10_Glu2bQR_ | CTGAGCGGTG | 1/1 U/G wobble base pair at +4 | target: 72.36% |
| NO: | 512 gID_067 | CCGTAGTCAG | position | ADAR2 on |
| 41 | 33__v0270 | CAATCTTTGCA | 1/1 U/G wobble base pair at +5 | target: 85.91% |
| | | ATGA | position | ADAR1 |
| | | | 1/1 A/G mismatch at +13 | specificity: 1.29 |
| | | | position | ADAR2 |
| | | | | specificity: 1.16 |
| SEQ | LRRK1_guide | TACAGCAGTA | 1/1 A/C mismatch at 0 position | ADAR1 on |
| ID | 10_Glu2bQR_ | CTGAGCGGTG | 1/1 U/G wobble base pair at +4 | target: 69.70% |
| NO: | 512_gID_067 | CCGTAGTCAG | position | ADAR2 on |
| 42 | 33__v0398 | CAATCTTTGCA | 1/1 U/G wobble base pair at +5 | target: 83.27% |
| | | ATGA | position | ADAR1 |
| | | | | specificity: 1.18 |
| | | | | ADAR2 |
| | | | | specificity: 1.16 |
| SEQ | LRRK1_guide | TACAGCAGGA | 1/1 A/C mismatch at 0 position | ADAR1 on |
| ID | 10_Glu2bQR_ | CTGGGTGATG | 1/1 U/G wobble base pair at +5 | target: 74.90% |
| NO: | 512_gID_067 | CCGTAGTCAG | position | ADAR2 on |
| 43 | 33__v0314 | CAATCTTTGCA | 1/1 G/U wobble base pair at +6 | target: 83.31% |
| | | ATGA | position | ADAR1 |
| | | | 1/1 U/G wobble base pair at +8 | specificity: 1.25 |
| | | | position | ADAR2 |
| | | | 1/1 A/G mismatch at +13 | specificity: 1.15 |
| | | | position | |
| SEQ | LRRK1_guide | TAAAGCAGTA | 1/1 A/C mismatch at 0 position | ADAR1 on |
| ID | 10_Glu2bQR_ | CTGAGCGGTG | 1/1 U/G wobble base pair at +4 | target: 56.32% |
| NO: | 512_gID_067 | CCGTAGTCAG | position | ADAR2 on |
| 44 | 33__v0142 | CAATCTTTGCA | 1/1 U/G wobble base pair at +5 | target: 92.45% |
| | | ATGA | position | ADAR1 |
| | | | | specificity: 1.13 |
| | | | | ADAR2 |
| | | | | specificity: 1.24 |
| SEQ | LRRK1_guide | TACAGCAGTC | 1/1 A/C mismatch at 0 position | ADAR1 on |
| ID | 10_Glu2bQR_ | CTGGGTGGTG | 1/1 U/G wobble base pair at +4 | target: 62.79% |
| NO: | 512_gID_067 | CCGTAGTCAG | position | ADAR2 on |
| 45 | 33__v0510 | CAATCTTTGCA | 1/1 U/G wobble base pair at +5 | target: 89.69% |
| | | ATGA | position | ADAR1 |
| | | | 1/1 G/U wobble base pair at +6 | specificity: 1.26 |
| | | | position | ADAR2 |
| | | | 1/1 U/G wobble base pair at +8 | specificity: 1.12 |
| | | | position | |
| | | | 1/1 U/C mismatch at +12 | |
| | | | position | |
| SEQ | LRRK1_guide | TACAGCAGGA | 4/4 symmetric bulge at −6 | ADAR1 on |
| ID | 11_Glu2bQR_ | CTGGGTAGTG | position (GCUG-GUCG) | target: 36.58% |
| NO: | 512_gID_071 | CCGTAGTGTC | 1/1 A/C mismatch at 0 position | ADAR2 on |
| 46 | 29__v0310 | GAATCTTTGCA | 1/1 U/G wobble base pair at +4 | target: 98.87% |
| | | ATGA | position | ADAR1 |
| | | | 1/1 G/U wobble base pair at +6 | specificity: 1.19 |
| | | | position | ADAR2 |
| | | | 1/1 U/G wobble base pair at +8 | specificity: 0.92 |
| | | | position | |
| | | | 1/1 A/G mismatch at +13 | |
| | | | position | |
| SEQ | LRRK1_guide | TACAGCAGGA | 1/1 A/C mismatch at 0 position | ADAR1 on |
| ID | 10_Glu2bQR_ | CTGAGCAGTG | 1/1 U/G wobble base pair at +4 | target: 73.18% |
| NO: | 512_gID_067 | CCGTAGTCAG | position | ADAR2 on |
| 47 | 3__v0262 | CAATCTTTGCA | 1/1 A/G mismatch at +13 | target: 97.93% |
| | | ATGA | position | ADAR1 |
| | | | | specificity: 1.29 |
| | | | | ADAR2 |
| | | | | specificity: 1.08 |
| SEQ | LRRK1_guide | TAAAGCAGTA | 1/1 A/C mismatch at 0 position | ADAR1 on |
| ID | 10_Glu2bQR_ | CTGAGCAGTG | 1/1 U/G wobble base pair at +4 | target: 43.41% |
| NO: | 512_gID_067 | CCGTAGTCAG | position | ADAR2 on |
| 48 | 3__v0134 | CAATCTTTGCA | | target: 97.78% |
| | | ATGA | | ADAR1 |
| | | | | specificity: 0.96 |
| | | | | ADAR2 |
| | | | | specificity: 1.21 |
| SEQ | LRRK1_guide | TAAAGCAGGC | 4/4 symmetric bulge at −6 | ADAR1 on |
| ID | 11_Glu2bQR_ | CTGAGCAGTG | position (GCUG-GUCG) | target: 33.33% |
| NO: | 512_gID_071 | CCGTAGTGTC | 1/1 A/C mismatch at 0 position | ADAR2 on |
| 49 | 29__v0070 | GAATCTTTGCA | 1/1 U/G wobble base pair at +4 | target: 98.79% |
| | | ATGA | position | ADAR1 |
| | | | 2/2 symmetric bulge at +12 | specificity: 1.15 |
| | | | position (UA-GC) | ADAR2 |
| | | | | specificity: 0.91 |
| SEQ | LRRK1_guide | TAAAGCAGGA | 4/4 symmetric bulge at −6 | ADAR1 on |
| ID | 11-Glu2bQR_ | CTGGGCAGTG | position (GCUG-GUCG) | target: 31.97% |
| NO: | 512_gID_071 | CCGTAGTGTC | 1/1 A/C mismatch at 0 position | ADAR2 on |
| 50 | 29__v0038 | GAATCTTTGCA | 1/1 U/G wobble base pair at +4 | target: 99.09% |
| | | ATGA | position | ADAR1 |
| | | | 1/1 U/G wobble base pair at +8 | specificity: 1.13 |
| | | | position | ADAR2 |
| | | | 1/1 A/G mismatch at +13 | specificity: 0.97 |
| | | | position | |
| SEQ | LRRK1_guide | TACAGCAGGA | 1/1 A/C mismatch at 0 position | ADAR1 on |
| ID | 10_Glu2bQR_ | CTGGGCGATG | 1/1 U/G wobble base pair at +5 | target: 74.08% |
| NO: | 512_gID_067 | CCGTAGTCAG | position | ADAR2 on |
| 51 | 33__v0298 | CAATCTTTGCA | 1/1 U/G wobble base pair at +8 | target: 83.71% |
| | | ATGA | position | ADAR1 |
| | | | 1 A/G mismatch at +13 position | specificity: 1.18 |
| | | | | ADAR2 |
| | | | | specificity: 1.08 |
| SEQ | LRRK1_guide | TACAGCAGGA | 1/1 A/C mismatch at 0 position | ADAR1 on |
| ID | 10_Glu2bQR_ | CTGGGCAGTG | 1/1 U/G wobble base pair at +4 | target: 63.43% |
| NO: | 512_gID_067 | CCGTAGTCAG | position | ADAR2 on |
| 52 | 33__v0294 | CAATCTTTGCA | 1/1 U/G wobble base pair at +8 | target: 96.93% |
| | | ATGA | position | ADAR1 |
| | | | 1/1 A/G mismatch at +13 | specificity: 1.2 |
| | | | position | ADAR2 |
| | | | | specificity: 1.27 |
| SEQ | LRRK1_guide | TAAAGCAGGA | 1/1 A/C mismatch at 0 position | ADAR1 on |
| ID | 10_Glu2bQR_ | CTGGGCAGTG | 1/1 U/G wobble base pair at +4 | target: 42.45% |
| NO: | 512_gID_067 | CCGTAGTCAG | position | ADAR2 on |
| 53 | 33__v0038 | CAATCTTTGCA | 1/1 U/G wobble base pair at +8 | target: 98.70% |
| | | ATGA | position | ADAR1 |
| | | | 1/1 A/G mismatch at +13 | specificity: 1.1 |
| | | | position | ADAR2 |
| | | | | specificity: 1.2 |
| SEQ | LRRK1_guide | TACAGCATTA | 6/6 symmetric internal loop at | ADAR1 on |
| ID | 04_Glu2bRG_ | CGGAGCAGTG | −6 position (UUGCUG-GUCGUU) | target: 7.65% |
| NO: | 128_gID_043 | CCGTAGTGTC | 1/1 A/C mismatch at 0 position | ADAR2 on |
| 54 | 57__v0118 | GTTTCTTTGCA | 1/1 U/G wobble base pair at +4 | target: 97.53% |
| | | ATGA | position | ADAR1 |
| | | | 1/1 A/G mismatch at +10 | specificity: 0.99 |
| | | | position | ADAR2 |
| | | | 1/1 C/U mismatch at +14 | specificity: 0.92 |
| | | | position | |
| SEQ | LRRK1_guide | TACAGCAGGC | 4/4 symmetric bulge at −6 | ADAR1 on |
| ID | 11_Glu2bQR_ | CTGAGCAGTG | position (GCUG-GUCG) | target: 56.98% |
| NO: | 512_gID_071 | CCGTAGTGTC | 1/1 A/C mismatch at 0 position | ADAR2 on |
| 55 | 29__v0326 | GAATCTTTGCA | 1/1 U/G wobble base pair at +4 | target: 98.63% |
| | | ATGA | position | ADAR1 |
| | | | 2/2 symmetric bulge at +12 | specificity: 1.28 |
| | | | position (UA-GC) | ADAR2 |
| | | | | specificity: 0.9 |
| SEQ | LRRK1_guide | TAAAGCAGGA | 4/4 symmetric bulge at −6 | ADAR1 on |
| ID | 11_Glu2bQR_ | CTGGGTAGTG | position (GCUG-GUCG) | target: 19.41% |
| NO: | 512_gID_071 | CCGTAGTGTC | 1/1 A/C mismatch at 0 position | ADAR2 on |
| 56 | 29__v0054 | GAATCTTTGCA | 1/1 U/G wobble base pair at +4 | target: 98.63% |
| | | ATGA | position | ADAR1 |
| | | | 1/1 G/U wobble base pair at +6 | specificity: 1.06 |
| | | | position | ADAR2 |
| | | | 1/1 U/G wobble base pair at +8 | specificity: 1 |
| | | | position | |
| | | | 1/1 A/G mismatch at +13 | |
| | | | position | |
| SEQ | LRRK1_guide | TACAGCAGTA | 4/4 symmetric bulge at −6 | ADAR1 on |
| ID | 11_Glu2bQR_ | CTGAGCAGTG | position (GCUG-GUCG) | target: 63.51% |
| NO: | 512_gID_071 | CCGTAGTGTC | 1/1 A/C mismatch at 0 position | ADAR2 on |
| 57 | 29__v0390 | GAATCTTTGCA | 1/1 U/G wobble base pair at +4 | target: 96.87% |
| | | ATGA | position | ADAR1 |
| | | | | specificity: 1.01 |
| | | | | ADAR2 |
| | | | | specificity: 1 |
| SEQ | LRRK1_guide | TACAGCAATA | 1/1 A/C mismatch at 0 position | ADAR1 on |
| ID | 03_Glu2bRG_ | CCGAGCAGTG | 1/1 U/G wobble base pair at +4 | target: 55.37% |
| NO: | 128_gID_039 | CCGTAGTCAG | position | ADAR2 on |
| 58 | 61__v0014 | CAATCTTTGCA | 1/1 A/G mismatch at +10 | target: 92.89% |
| | | ATGA | position | ADAR1 |
| | | | 1/1 C/A mismatch at +14 | specificity: 0.98 |
| | | | position | ADAR2 |
| | | | | specificity: 0.88 |
| SEQ | LRRK1_guide | TACAGCAGTA | 1/1 A/C mismatch at 0 position | ADAR1 on |
| ID | 10_Glu2bQR_ | CTGGGCGGTG | 1/1 U/G wobble base pair at +4 | target: 75.29% |
| NO: | 512_gID_067 | CCGTAGTCAG | position | ADAR2 on |
| 59 | 33__v0430 | CAATCTTTGCA | 1/1 U/G wobble base pair at +5 | target: 85.54% |
| | | ATGA | position | ADAR1 |
| | | | 1/1 U/G wobble base pair at +8 | specificity: 1.16 |
| | | | position | ADAR2 |
| | | | | specificity: 1.34 |
| SEQ | LRRK1_guide | TACAGCAGGA | 1/1 A/C mismatch at 0 position | ADAR1 on |
| ID | 10_Glu2bQR_ | CTGGGTGGTG | 1/1 U/G wobble base pair at +4 | target: 61.52% |
| NO: | 512_gID_067 | CCGTAGTCAG | position | ADAR2 on |
| 60 | 33__v0318 | CAATCTTTGCA | 1/1 U/G wobble base pair at +5 | target: 93.45% |
| | | ATGA | position | ADAR1 |
| | | | 1/1 G/U wobble base pair at +6 | specificity: 1.27 |
| | | | position | ADAR2 |
| | | | 1/1 U/G wobble base pair at +8 | specificity: 1.34 |
| | | | position | |
| | | | 1/1 A/G mismatch at +13 | |
| | | | position | |
| SEQ | LRRK1_guide | TAAAGCAGGA | 1/1 A/C mismatch at 0 position | ADAR1 on |
| ID | 10_Glu2bQR_ | CTGAGCAGTG | 1/1 U/G wobble base pair at +4 | target: 51.18% |
| NO: | 512_gID_067 | CCGTAGTCAG | position | ADAR2 on |
| 61 | 33__v0006 | CAATCTTTGCA | 1/1 A/G mismatch at +13 | target: 98.68% |
| | | ATGA | position | ADAR1 |
| | | | | specificity: 1.2 |
| | | | | ADAR2 |
| | | | | specificity: 1.07 |
| SEQ | LRRK1_guide | TAAAGCAGGA | 4/4 symmetric bulge at −6 | ADAR1 on |
| ID | 11_Glu2bQR_ | CTGAGTAGTG | position (GCUG-GUCG) | target: 26.50% |
| NO: | 512_gID_071 | CCGTAGTGTC | 1/1 A/C mismatch at 0 position | ADAR2 on |
| 62 | 29__v0022 | GAATCTTTGCA | 1/1 U/G wobble base pair at +4 | target: 97.89% |
| | | ATGA | position | ADAR1 |
| | | | 1/1 G/U wobble base pair at +6 | specificity: 1.13 |
| | | | position | ADAR2 |
| | | | 1/1 A/G mismatch at +13 | specificity: 0.94 |
| | | | position | |
| SEQ | LRRK1_guide | TACAGCAGTA | 1/1 A/C mismatch at 0 position | ADAR1 on |
| ID | 10_Glu2bQR_ | CTGAGTGGTG | 1/1 U/G wobble base pair at +4 | target: 76.01% |
| NO: | 512_gID_067 | CCGTAGTCAG | position | ADAR2 on |
| 63 | 33__v0414 | CAATCTTTGCA | 1/1 U/G wobble base pair at +5 | target: 86.64% |
| | | ATGA | position | ADAR1 |
| | | | 1/1 G/U wobble base pair at +6 | specificity: 1.18 |
| | | | position | ADAR2 |
| | | | | specificity: 1.14 |
| SEQ | LRRK1_guide | TACAGCAGGA | 1/1 A/C mismatch at 0 position | ADAR1 on |
| ID | 10_Glu2bQR_ | CTGGGCGGTG | 1/1 U/G wobble base pair at +4 | target: 65.42% |
| NO: | 512_gID_067 | CCGTAGTCAG | position | ADAR2 on |
| 64 | 33__v0302 | CAATCTTTGCA | 1/1 U/G wobble base pair at +5 | target: 91.92% |
| | | ATGA | position | ADAR1 |
| | | | 1/1 U/G wobble base pair at +8 | specificity: 1.26 |
| | | | position | ADAR2 |
| | | | 1/1 A/G mismatch at +13 | specificity: 1.28 |
| | | | position | |
| SEQ | LRRK1_guide | TACAGCAGTC | 1/1 A/C mismatch at 0 position | ADAR1 on |
| ID | 10_Glu2bQR_ | CTGGGCGGTG | 1/1 U/G wobble base pair at +4 | target: 67.04% |
| NO: | 512_gID_067 | CCGTAGTCAG | position | ADAR2 on |
| 65 | 33__v0494 | CAATCTTTGCA | 1/1 U/G wobble base pair at +5 | target: 89.53% |
| | | ATGA | position | ADAR1 |
| | | | 1/1 U/G wobble base pair at +8 | specificity: 1.28 |
| | | | position | ADAR2 |
| | | | 1/1 U/C mismatch at +12 | specificity: 1.02 |
| | | | position | |
| SEQ | LRRK1_guide | TAAAGCAGTA | 4/4 symmetric bulge at −6 | ADAR1 on |
| ID | 11_Glu2bQR_ | CTGAGCAGTG | position (GCUG-GUCG) | target: 38.94% |
| NO: | 512_gID_071 | CCGTAGTGTC | 1/1 A/C mismatch at 0 position | ADAR2 on |
| 66 | 29__v0134 | GAATCTTTGCA | 1/1 U/G wobble base pair at +4 | target: 97.94% |
| | | ATGA | position | ADAR1 |
| | | | | specificity: 0.95 |
| | | | | ADAR2 |
| | | | | specificity: 1.03 |
| SEQ | LRRK1_guide | TAAAGCAGGA | 4/4 symmetric bulge at −6 | ADAR1 on |
| ID | 11_Glu2bQR_ | CTGAGCAGTG | position (GCUG-GUCG) | target: 45.55% |
| NO: | 512_gID_071 | CCGTAGTGTC | 1/1 A/C mismatch at 0 position | ADAR2 on |
| 67 | 29__v0006 | GAATCTTTGCA | 1/1 U/G wobble base pair at +4 | target: 99.01% |
| | | ATGA | position | ADAR1 |
| | | | 1/1 A/G mismatch at +13 | specificity: 1.18 |
| | | | position | ADAR2 |
| | | | | specificity: 0.92 |
| SEQ | LRRK1_guide | TACAGCAGGA | 4/4 symmetric bulge at -6 | ADAR1 on |
| ID | 11_Glu2bQR_ | CTGGGCAGTG | position (GCUG-GUCG) | target: 55.62% |
| NO: | 512_gID_071 | CCGTAGTGTC | 1/1 A/C mismatch at 0 position | ADAR2 on |
| 68 | 29__v0294 | GAATCTTTGCA | 1/1 U/G wobble base pair at +4 | target: 99.09% |
| | | ATGA | position | ADAR1 |
| | | | 1/1 U/G wobble base pair at +8 | specificity: 1.25 |
| | | | position | ADAR2 |
| | | | 1/1 A/G mismatch at +13 | specificity: 0.89 |
| | | | position | |

### Example 10

### High Throughput Screening of Engineered Guide RNAs Targeting LRRK2 mRNA

Using the compositions and methods described herein, high throughput screening (HTS) of engineered guide RNAs against LRRK2. Self-annealing RNA structures comprising the guide RNA sequences of TABLE 3 and the sequences of the regions targeted by the candidate engineered guide RNAs were contacted with an RNA editing entity (e.g., a recombinant ADAR1 and/or ADAR2) under conditions that allow for the editing of the regions targeted by the guide RNAs. The regions targeted by the guide RNAs were subsequently assessed for editing using next generation sequencing (NGS).

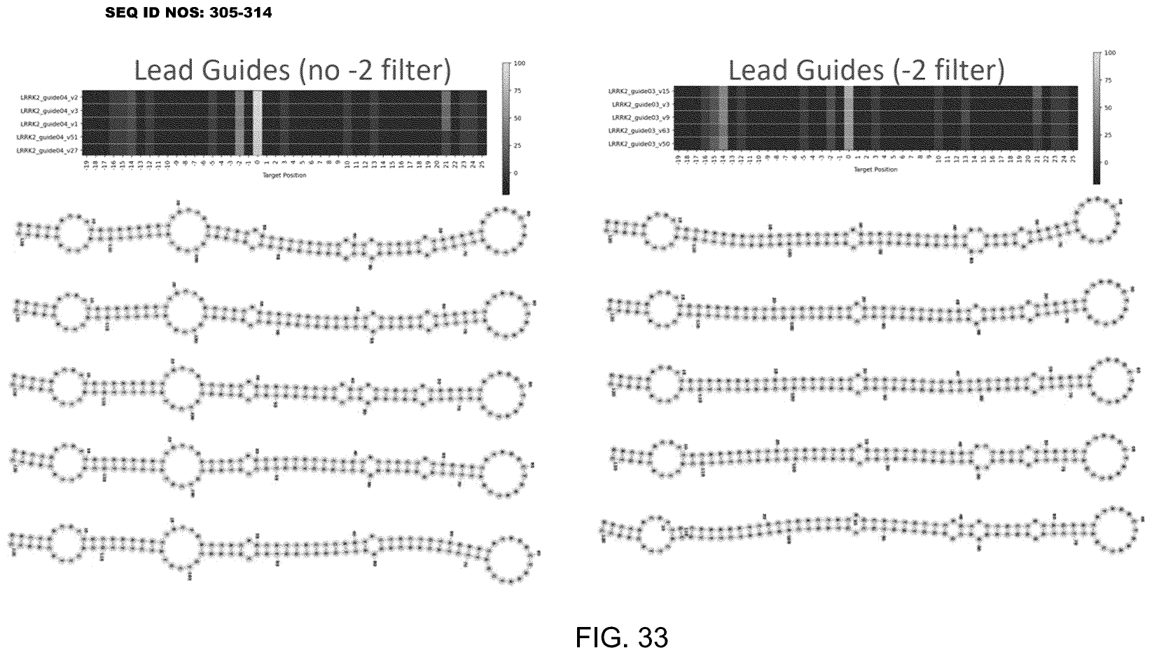

FIG. 33 shows heat maps and structures for exemplary engineered guide RNA sequences targeting a LRRK2 mRNA. The heat map provides visualization of the editing profile at the 10-minute time point. 5 engineered guide RNAs for on-target editing (with no-2 filter) are in the left graph and 5 engineered guide RNAs for on-target editing with minimal-2 editing are depicted on the right graph. The corresponding predicted secondary structures are below the heat maps.

Exemplary engineered polynucleotide sequences corresponding to FIG. 33 are shown in TABLE 3. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 3
Engineered Polynucleotide Sequences Targeting LRRK2 mRNA, 45 mers for
In Cis-Editing Experiments

| SEQ | | | | |
|---|---|---|---|---|
| ID | | | Structural Features | |
| NO | ID | Sequence | (target/guide) | Metrics |
| SEQ | LRRK2_guide04_ | GCTAAATCGATTCTAA | 4/4 symmetric bulge | ADAR2 on target: 85.80% |
| ID | v2 | AGCAGGACGGGGTGGT | 5/5 symmetric internal | ADAR2 specificity: 1.1 |
| NO: | | GCCGTAGTGTCGTATCT | loop | |
| 69 | | TTGCACTTACAGTC | 1 A/C mismatch | |
| | | | 1 A/G mismatch | |
| | | | 1 A/G mismatch | |
| | | | 1 G/A mismatch | |
| SEQ | LRRK2_guide04_ | GCTAAATCGATTCTAA | 4/4 symmetric bulge | ADAR2 on target: 87.17% |
| ID | v3 | AGCAGGACTGGGTGGT | 5/5 symmetric internal | ADAR2 specificity: 1.09 |
| NO: | | GCCGTAGTGTCGTATCT | loop | |
| 70 | | TTGCACTTACAGTC | 1 A/C mismatch | |
| | | | 1 A/G mismatch | |
| | | | 1 G/A mismatch | |
| SEQ | LRRK2_guide04_ | GCTAAATCGATTCTAA | 4/4 symmetric bulge | ADAR2 on target: 79.38% |
| ID | v1 | AGCAGGACCGGGTGGT | 5/5 symmetric internal | ADAR2 specificity: 1.05 |
| NO: | | GCCGTAGTGTCGTATCT | loop | |
| 71 | | TTGCACTTACAGTC | 1 A/C mismatch | |
| | | | 1 A/C mismatch | |
| | | | 1 A/G mismatch | |
| | | | 1 G/A mismatch | |
| SEQ | LRRK2_guide04_ | GCTAAATCGATTCTAG | 4/4 symmetric bulge | ADAR2 on target: 78.91% |
| ID | v51 | AGCAGGACTGGGTGGT | 5/5 symmetric internal | ADAR2 specificity: 1.07 |
| NO: | | GCCGTAGTGTCGTATCT | loop | |
| 72 | | TTGCACTTACAGTC | 1 A/C mismatch | |
| | | | 1 A/G mismatch | |
| | | | 1 G/A mismatch | |
| SEQ | LRRK2_guide04_ | GCTAAATCGATTCTAC | 4/4 symmetric bulge | ADAR2 on target: 79.32% |
| ID | v27 | AGCAGGACTGGGTGGT | 5/5 symmetric internal | ADAR2 specificity: 1.08 |
| NO: | | GCCGTAGTGTCGTATCT | loop | |
| 73 | | TTGCACTTACAGTC | 1 A/C mismatch | |
| | | | 1 A/G mismatch | |
| SEQ | LRRK2_guide03_ | GCTAAATCGATTCTAA | 4/4 symmetric bulge | ADAR2 on target: 74.97% |
| ID | v15 | AGCATGACTGGGTGGT | 1 A/C mismatch | ADAR2 specificity: 0.71 |
| NO: | | GCCGTAGTCAGCAATC | 2/2 symmetric bulge | |
| 74 | | TTTGCACTTACAGTC | 1 G/A mismatch | |
| SEQ | LRRK2_guide03_ | GCTAAATCGATTCTAA | 4/4 symmetric bulge | ADAR2 on target: 74.86% |
| ID | v3 | AGCAGGACTGGGTGGT | 1 A/C mismatch | ADAR2 specificity: 0.73 |
| NO: | | GCCGTAGTCAGCAATC | 1 A/G mismatch | |
| 75 | | TTTGCACTTACAGTC | 1 G/A mismatch | |
| SEQ | LRRK2_guide03_ | GCTAAATCGATTCTAA | 4/4 symmetric bulge | ADAR2 on target: 71.85% |
| ID | v9 | AGCAGTACTGGGTGGT | 1 A/C mismatch | ADAR2 specificity: 0.56 |
| NO: | | GCCGTAGTCAGCAATC | 1 G/A mismatch | |
| 76 | | TTTGCACTTACAGTC | | |
| SEQ | LRRK2_guide03_ | GCTAAATCGATTCTAG | 4/4 symmetric bulge | ADAR2 on target: 66.25% |
| ID | v63 | AGCATGACTGGGTGGT | 1 A/C mismatch | ADAR2 specificity: 0.81 |
| NO: | | GCCGTAGTCAGCAATC | 2/2 symmetric bulge | |
| 77 | | TTTGCACTTACAGTC | 1 G/A mismatch | |
| SEQ | LRRK2_guide03_ | GCTAAATCGATTCTAG | 4/4 symmetric bulge | ADAR2 on target: 69.87% |
| ID | v50 | AGCAGGACGGGGTGGT | 1 A/C mismatch | ADAR2 specificity: 0.82 |
| NO: | | GCCGTAGTCAGCAATC | 1 A/G mismatch | |
| 78 | | TTTGCACTTACAGTC | 1 A/G mismatch | |
| | | | 1 G/A mismatch | |

### Example 11

### High Throughput Screening of Engineered Guide RNAs Targeting LRRK2 mRNA

Using the compositions and methods described herein, high throughput screening (HTS) of engineered guide RNAs that target LRRK2 mRNA. Self-annealing RNA structures comprising the guide RNA sequences of TABLE 4 and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS). The guide RNAs of TABLE 4 showed specific editing of the A nucleotide at position 6055 of the mRNA encoding the LRRK2 G2019S. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 4
Exemplary Guide RNAs that target LRRK2 mRNA

| SEQ | | | | |
|---|---|---|---|---|
| ID | | | | |
| NO | ID | Sequence | Structural Features (target/guide) | Metrics |
| SEQ | LRRK2_ | ATTCTACAGC | 1/1 C/U mismatch at −4 position | ADAR1 on |
| ID | bnPCR_ | ACGACTGAGC | 1/1 A/C mismatch at 0 position | target: 55.03% |
| NO: | ID3796_ | AATGCCGTATT | 2/2 symmetric bulge at +13 position (AC- | ADAR2 on |
| 79 | count1909 | CAGCAATCTTT | CG) | target: 89.21% |
| | | GCA | | ADAR1 |
| | | | | specificity: 0.55 |
| | | | | ADAR2 |
| | | | | specificity: 1.12 |
| SEQ | LRRK2_ | ATTCTAGAGC | 1/1 C/C mismatch at −8 position | ADAR1 on |
| ID | bnPCR_ | AGTACTGAGC | 1/1 G/U wobble base pair at −6 position | target: 91.69% |
| NO: | ID47662_ | AATGCCGTGG | 1/1 U/G wobble base pair at −3 position | ADAR2 on |
| 80 | count362 | TTACCAATCTT | 1/1 A/C mismatch at 0 position | target: 89.35% |
| | | TGCA | 1/1 G/G mismatch at +19 position | ADAR1 |
| | | | | specificity: 0.61 |
| | | | | ADAR2 |
| | | | | specificity: 1.04 |
| SEQ | LRRK2_ | ATTCTACAGC | 1/1 C/A mismatch at −17 position | ADAR1 on |
| ID | bnPCR_ | AGTAGTAAGC | 1/1 G/G wobble base pair at −6 position | target: 72.09% |
| NO: | ID117434_ | TTTGCCGAAGT | 1/1 A/A wobble base pair at −2 position | ADAR2 on |
| 81 | count761 | GAGCAATCTTT | 1/1 A/C mismatch at 0 position | target: 89.77% |
| | | ACA | 2/2 symmetric bulge at +4 position (UU- | ADAR1 |
| | | | UU) | specificity: 0.75 |
| | | | 1/1 C/A mismatch at +9 position | ADAR2 |
| | | | 1/1 G/G mismatch at +11 position | specificity: 0.92 |
| SEQ | LRRK2_ | ATTCTACAGC | 1/1 U/G wobble base pair at −10 position | ADAR1 on |
| ID | bnPCR_ | ACGACTGAGC | 1/1 A/C mismatch at 0 position | target: 69.21% |
| NO: | ID70875_ | AAGGCCGTAG | 1/1 A/G mismatch at +3 position | ADAR2 on |
| 82 | count310 | TCAGCGATCTT | 2/2 symmetric bulge at +13 position (AC- | target: 90.00% |
| | | TGCA | CG) | ADAR1 |
| | | | | specificity: 0.54 |
| | | | | ADAR2 |
| | | | | specificity: 1.32 |
| SEQ | LRRK2_ | ATTCTACAGC | 1/1 U/U mismatch at −7 position | ADAR1 on |
| ID | bnPCR_ | ACTACGGGGC | 1/1 A/A mismatch at −2 position | target: 29.21% |
| NO: | ID26374_ | AATGCCGAAG | 1/1 A/C mismatch at 0 position | ADAR2 on |
| 83 | count337 | TCTGCAATCTT | 1/1 U/G wobble base pair at +8 position | target: 90.13% |
| | | TGCA | 1/1 A/G mismatch at +10 position | ADAR1 |
| | | | 1/1 C/C mismatch at +14 position | specificity: 0.46 |
| | | | | ADAR2 |
| | | | | specificity: 1.01 |
| SEQ | LRRK2_ | ATTCTACGGC | 1/1 G/U wobble base pair at −6 position | ADAR1 on |
| ID | bnPCR_ | GGTACTGACC | 1/1 A/C mismatch at 0 position | target: 93.44% |
| NO: | ID34601_ | AATCCCGTAG | 1/1 C/C mismatch at +2 position | ADAR2 on |
| 84 | count2108 | TTAGCAATCTT | 1/1 C/C mismatch at +7 position | target: 90.18% |
| | | TGCA | 1/1 U/G wobble base pair at +15 position | ADAR1 |
| | | | 1/1 U/G wobble base pair at +18 position | specificity: 0.91 |
| | | | | ADAR2 |
| | | | | specificity: 1.32 |
| SEQ | LRRK2_ | ATTCTACAGC | 1/1 G/U wobble base pair at −13 position | ADAR1 on |
| ID | bnPCR_ | GGTACTCATC | 1/1 C/U mismatch at −8 position | target: 74.58% |
| NO: | ID8689_ | AATGCCGTAG | 1/1 U/G wobble base pair at −7 position | ADAR2 on |
| 85 | count926 | TCGTCAATTTT | 1/1 A/C mismatch at 0 position | target: 90.33% |
| | | TGCA | 3/3 symmetric bulge at +7 position (CUC- | ADAR1 |
| | | | CAU) | specificity: 0.49 |
| | | | 1/1 U/G wobble base pair at +15 position | ADAR2 |
| | | | | specificity: 1.02 |
| SEQ | LRRK2_ | ATTCTAAAGC | 2/2 symmetric bulge at −7 position (CU- | ADAR1 on |
| ID | bnPCR_ | CGTACTAAGC | CC) | target: 33.31% |
| NO: | ID4669_ | AGTACCGTAG | 1/1 A/C mismatch at 0 position | ADAR2 on |
| 86 | count1929 | TCCCCAATCTT | 1/1 C/A mismatch at +2 position | target: 90.37% |
| | | TGCA | 1/1 U/G wobble base pair at +4 position | ADAR1 |
| | | | 1/1 C/A mismatch at +9 position | specificity: 0.47 |
| | | | 1/1 U/C mismatch at +15 position | ADAR2 |
| | | | 1/1 G/A mismatch at +19 position | specificity: 0.81 |
| SEQ | LRRK2_ | ATTCTAGAGC | 1/1 A/A mismatch at −2 position | ADAR1 on |
| ID | bnPCR_ | CATACTGTTCA | 1/1 A/C mismatch at 0 position | target: 13.05% |
| NO: | ID24531_ | ATGCCGAAGT | 2/2 symmetric bulge at +7 position (CU- | ADAR2 on |
| 87 | count2149 | CAGCAATCTTT | UU) | target: 90.41% |
| | | GCA | 2/2 symmetric bulge at +14 position (CU- | ADAR1 |
| | | | CA) | specificity: 0.69 |
| | | | 1/1 G/G mismatch at +19 position | ADAR2 |
| | | | | specificity: 1.31 |
| SEQ | LRRK2_ | ATTCTACAGC | 1/1 U/U mismatch at −7 position | ADAR1 on |
| ID | bnPCR_ | GGTACTGGGC | 1/1 U/G wobble base pair at -3 position | target: 79.59% |
| NO: | ID38909_ | AATGCCGTGG | 1/1 A/C mismatch at 0 position | ADAR2 on |
| 88 | count2105 | TCTGCAATCTT | 1/1 U/G wobble base pair at +8 position | target: 90.46% |
| | | TGCA | 1/1 U/G wobble base pair at +15 position | ADAR1 |
| | | | | specificity: 0.54 |
| | | | | ADAR2 |
| | | | | specificity: 1.16 |
| SEQ | LRRK2_ | ATTCTACAGCT | 1/1 C/C mismatch at −8 position | ADAR1 on |
| ID | bnPCR_ | GTACTGAGGA | 1/1 G/U wobble base pair at −6 position | target: 92.87% |
| NO: | ID13205 | ATGCCGTCTTT | 2/2 symmetric bulge at -3 position (CU- | ADAR2 on |
| 89 | count2438 | ACCAATCTTTG | CU) | target: 90.75% |
| | | CA | 1/1 A/C mismatch at 0 position | ADAR1 |
| | | | 1/1 G/G mismatch at +6 position | specificity: 0.63 |
| | | | 1/1 U/U mismatch at +15 position | ADAR2 |
| | | | | specificity: 1.36 |
| SEQ | LRRK2_ | ATTCTACAGC | 1/1 G/U wobble base pair at −6 position | ADAR1 on |
| ID | bnPCR_ | AGTACTTATCA | 1/1 U/U mismatch at −3 position | target: 88.30% |
| NO: | ID74600_ | GTGCCGTTGTT | 1/1 A/C mismatch at 0 position | ADAR2 on |
| 90 | count480 | AGCAATCTTTG | 1/1 U/G wobble base pair at +4 position | target: 91.15% |
| | | CA | 3/3 symmetric bulge at +7 position (CUC- | ADAR1 |
| | | | UAU) | specificity: 0.59 |
| | | | | ADAR2 |
| | | | | specificity: 1.2 |
| SEQ | LRRK2_ | ATTCTATAGCT | 1/1 C/U mismatch at −8 position | ADAR1 on |
| ID | bnPCR_ | GGACTGAGCT | 1/1 U/G wobble base pair at −7 position | target: 26.20% |
| NO: | ID11052_ | CTGCCGTAGTC | 1/1 A/C mismatch at 0 position | ADAR2 on |
| 91 | count1602 | GTCAATCTTTG | 2/2 symmetric bulge at +4 position (UU- | target: 91.42% |
| | | CA | UC) | ADAR1 |
| | | | 3/3 symmetric bulge at +13 position | specificity: 0.74 |
| | | | (ACU-UGG) | ADAR2 |
| | | | 1/1 G/U wobble base pair at +19 position | specificity: 1.23 |
| SEQ | LRRK2_ | ATTCTACAGC | 1/1 C/C mismatch at −8 position | ADAR1 on |
| ID | bnPCR_ | AATACTCAGC | 1/1 A/C mismatch at 0 position | target: 81.04% |
| NO: | ID22281_ | AGTGCCGTAG | 1/1 U/G wobble base pair at +4 position | ADAR2 on |
| 92 | count2661 | TCACCAATCTT | 1/1 C/C mismatch at +9 position | target: 91.91% |
| | | TGCA | 1/1 C/A mismatch at +14 position | ADAR1 |
| | | | | specificity: 0.64 |
| | | | | ADAR2 |
| | | | | specificity: 1.07 |
| SEQ | LRRK2_ | ATTCTACAAC | 1/1 U/U mismatch at −7 position | ADAR1 on |
| ID | bnPCR_ | AGTACTGCGC | 1/1 A/C mismatch at 0 position | target: 92.15% |
| NO: | ID20990_ | ACCGCCGTAG | 2/2 symmetric bulge at +3 position (AU- | ADAR2 on |
| 93 | count3129 | TCTGCAATCTT | CC) | target: 92.65% |
| | | TGCA | 1/1 U/C mismatch at +8 position | ADAR1 |
| | | | 1/1 C/A mismatch at +17 position | specificity: 0.68 |
| | | | | ADAR2 |
| | | | | specificity: 1.05 |
| SEQ | LRRK2_ | ATTCTAGAGC | 1/1 C/U mismatch at −8 position | ADAR1 on |
| ID | bnPCR_ | CGTACTGCGCT | 1/1 A/C mismatch at 0 position | target: 14.68% |
| NO: | ID27527_ | TTGCCGTAGTC | 2/2 symmetric bulge at +4 position (UU- | ADAR2 on |
| 94 | count488 | ATCAATCTTTG | UU) | target: 92.90% |
| | | CA | 1/1 U/C mismatch at +8 position | ADAR1 |
| | | | 1/1 U/C mismatch at +15 position | specificity: 0.54 |
| | | | 1/1 G/G mismatch at +19 position | ADAR2 |
| | | | | specificity: 1.15 |
| SEQ | LRRK2_ | ATTCTACAGCC | 3/3 symmetric bulge at −6 position (CUG- | ADAR1 on |
| ID | bnPCR_ | GTACTGCCCA | AAC) | target: 62.87% |
| NO: | ID18596_ | ATGCCGAAGT | 1/1 A/A mismatch at −2 position | ADAR2 on |
| 95 | count3437 | AACCAATCTTT | 1/1 A/C mismatch at 0 position | target: 93.88% |
| | | GCA | 2/2 symmetric bulge at +7 position (CU- | ADAR1 |
| | | | CC) | specificity: 0.6 |
| | | | 1/1 U/C mismatch at +15 position | ADAR2 |
| | | | | specificity: 1.14 |
| SEQ | LRRK2_ | ATTCTACCGTG | 1/1 U/U mismatch at −7 position | ADAR1 on |
| ID | bnPCR_ | GGACTGAGCA | 1/1 A/C mismatch at 0 position | target: 84.30% |
| NO: | ID5048_ | GTCCCGTAGTC | 1/1 C/C mismatch at +2 position | ADAR2 on |
| 96 | count2719 | TGCAATCTTTG | 1/1 U/G wobble base pair at +4 position | target: 94.10% |
| | | CA | 1/1 A/G mismatch at +13 position | ADAR1 |
| | | | 1/1 U/G wobble base pair at +15 position | specificity: 1.02 |
| | | | 1/1 G/U wobble base pair at +16 position | ADAR2 |
| | | | 1/1 U/C mismatch at +18 position | specificity: 1.29 |
| SEQ | LRRK2_ | ATTCTACAGC | 1/1 G/A mismatch at −1 position | ADAR1 on |
| ID | bnPCR_ | AGTACTGTTCA | 2/2 symmetric bulge at −1 position (CA- | target: 16.06% |
| NO: | ID3292_ | GTGCGCTAGT | GC) | ADAR2 on |
| 97 | count1951 | AAGCAATCTTT | 1/1 U/G wobble base pair at +4 position | target: 94.39% |
| | | GCA | 2/2 symmetric bulge at +7 position (CU- | ADAR1 |
| | | | UU) | specificity: 0.84 |
| | | | | ADAR2 |
| | | | | specificity: 1.29 |
| SEQ | LRRK2_ | ATTCTACAGGT | 1/1 U/C mismatch at −10 position | ADAR1 on |
| ID | bnPCR_ | GTACTGTTCTA | 1/1 G/U wobble base pair at −6 position | target: 71.92% |
| NO: | ID92487_ | TGCCGTTGTTA | 1/1 U/U mismatch at −3 position | ADAR2 on |
| 98 | count914 | GCCATCTTTGC | 1/1 A/C mismatch at 0 position | target: 94.50% |
| | | A | 1/1 U/U mismatch at +5 position | ADAR1 |
| | | | 2/2 symmetric bulge at +7 position (CU- | specificity: 0.72 |
| | | | UU) | ADAR2 |
| | | | 2/2 symmetric bulge at +15 position (UG- | specificity: 1.26 |
| | | | GU) | |
| SEQ | LRRK2_ | ATTCTAGGGC | 4/4 symmetric bulge at −3 position | ADAR1 on |
| ID | bnPCR_ | AGTACTGAGA | (UACA-CGAU) | target: 88.68% |
| NO: | ID12934_ | GATGCCGATG | 1/1 U/G wobble base pair at +5 position | ADAR2 on |
| 99 | count1814 | TCAGCAATCTT | 1/1 G/A mismatch at +6 position | target: 30.88% |
| | | TGCA | 1/1 U/G wobble base pair at +18 position | ADAR1 |
| | | | 1/1 G/G mismatch at +19 position | specificity: 1.21 |
| | | | | ADAR2 |
| | | | | specificity: 0.64 |
| SEQ | LRRK2_ | ATTCTACAGC | 1/1 A/C mismatch at 0 position | ADAR1 on |
| ID | bnPCR_ | AGTATTGGGC | 1/1 C/C mismatch at +2 position | target: 88.73% |
| NO: | ID22125_ | AGTCCCGTAG | 1/1 U/G wobble base pair at +4 position | ADAR2 on |
| 100 | count837 | TCAGCAATCTT | 1/1 U/G wobble base pair at +8 position | target: 78.61% |
| | | TGCA | 1/1 G/U wobble base pair at +11 position | ADAR1 |
| | | | | specificity: 1.06 |
| | | | | ADAR2 |
| | | | | specificity: 1.19 |
| SEQ | LRRK2_ | ATTCTACAGC | 1/1 C/U mismatch at −6 position | ADAR1 on |
| ID | bnPCR_ | AGGACTGGGC | 1/1 A/C mismatch at 0 position | target: 89.12% |
| NO: | ID31607_ | GATCCCGTAG | 1/1 C/C mismatch at +2 position | ADAR2 on |
| 101 | count1105 | TCATCAATCTT | 1/1 U/G wobble base pair at +5 position | target: 79.41% |
| | | TGCA | 1/1 U/G wobble base pair at +8 position | ADAR1 |
| | | | 1/1 A/G mismatch at +13 position | specificity: 1.08 |
| | | | | ADAR2 |
| | | | | specificity: 0.96 |
| SEQ | LRRK2_ | ATTCTACAGC | 1/1 C/C mismatch at −8 position | ADAR1 on |
| ID | bnPCR_ | AGTACTAAGC | 2/2 symmetric bulge at −4 position (AC- | target: 89.96% |
| NO: | ID17685_ | TATGGCGTAC | CG) | ADAR2 on |
| 102 | count2957 | GCACCAATCTT | 2/2 symmetric bulge at 0 position (AG- | target: 6.89% |
| | | TGCA | GC) | ADAR1 |
| | | | 1/1 U/U mismatch at +5 position | specificity: 0.98 |
| | | | 1/1 C/A mismatch at +9 position | ADAR2 |
| | | | | specificity: 0.34 |
| SEQ | LRRK2_ | ATTCTACAGTA | 1/1 U/G wobble base pair at −7 position | ADAR1 on |
| ID | bnPCR_ | GGACTGAGCA | 1/1 G/G mismatch at −6 position | target: 90.20% |
| NO: | ID29209_ | CTGCCGAGCT | 0/1 asymmetric bulge at −4 position (-C) | ADAR2 on |
| 103 | count871 | GGGCAATCTTT | 1/0 asymmetric bulge at −2 position (C-) | target: 88.24% |
| | | GCA | 1/1 A/C mismatch at 0 position | ADAR1 |
| | | | 1/1 U/C mismatch at +4 position | specificity: 1.33 |
| | | | 1/1 A/G mismatch at +13 position | ADAR2 |
| | | | 1/1 G/U wobble base pair at +16 position | specificity: 1.07 |
| SEQ | LRRK2_ | ATTCTACCGCA | 3/3 symmetric bulge at −6 position (CUG- | ADAR1 on |
| ID | bnPCR_ | GTACTGAGCA | AAA) | target: 90.49% |
| NO: | ID33976_ | AAGCCGAGGT | 1/1 U/G wobble base pair at −3 position | ADAR2 on |
| 104 | count681 | AAACAATCTTT | 1/1 A/A mismatch at -2 position | target: 78.40% |
| | | GCA | 1/1 A/C mismatch at 0 position | ADAR1 |
| | | | 1/1 A/A mismatch at +3 position | specificity: 1.02 |
| | | | 1/1 U/C mismatch at +18 position | ADAR2 |
| | | | | specificity: 0.74 |
| SEQ | LRRK2_ | ATTCTACAGC | 1/1 U/C mismatch at −7 position | ADAR1 on |
| ID | bnPCR_ | AGTACTGAGC | 3/3 symmetric bulge at -3 position (ACU- | target: 90.61% |
| NO: | ID39201_ | AATTCCGTTAA | UAA) | ADAR2 on |
| 105 | count637 | CCGCAATCTTT | 1/1 A/C mismatch at 0 position | target: 52.23% |
| | | GCA | 1/1 C/U mismatch at +2 position | ADAR1 |
| | | | | specificity: 1.05 |
| | | | | ADAR2 |
| | | | | specificity: 0.69 |
| SEQ | LRRK2_ | ATTCTACAGTA | 1/1 U/C mismatch at −7 position | ADAR1 on |
| ID | bnPCR_ | GTCCGGAGCT | 1/1 A/C mismatch at 0 position | target: 90.66% |
| NO: | ID87993_ | ATTCCGTAGTC | 1/1 C/U mismatch at +2 position | ADAR2 on |
| 106 | count219 | CGCAATCTTTG | 1/1 U/U mismatch at +5 position | target: 27.30% |
| | | CA | 1/1 A/G mismatch at +10 position | ADAR1 |
| | | | 1/1 U/C mismatch at +12 position | specificity: 1.04 |
| | | | 1/1 G/U wobble base pair at +16 position | ADAR2 |
| | | | | specificity: 0.45 |
| SEQ | LRRK2_ | ATTCTACAGC | 1/1 U/G wobble base pair at −7 position | ADAR1 on |
| ID | bnPCR_ | AGGACTGAGC | 2/2 symmetric bulge at 0 position (AG- | target: 90.80% |
| NO: | ID31517_ | AATGGAGTAG | GA) | ADAR2 on |
| 107 | count1083 | TCGGCAATCTT | 1/1 A/G mismatch at +13 position | target: 23.12% |
| | | TGCA | | ADAR1 |
| | | | | specificity: 1.01 |
| | | | | ADAR2 |
| | | | | specificity: 0.47 |
| SEQ | LRRK2_ | ATTCTACCGCA | 1/1 A/C mismatch at 0 position | ADAR1 on |
| ID | bnPCR_ | GTACTACCCG | 1/1 C/C mismatch at +2 position | target: 90.83% |
| NO: | ID28134_ | ATCCCGTAGTC | 1/1 U/G wobble base pair at +5 position | ADAR2 on |
| 108 | count1700 | AGCAATCTTTG | 3/3 symmetric bulge at +7 position (CUC- | target: 35.42% |
| | | CA | ACC) | ADAR1 |
| | | | 1/1 U/C mismatch at +18 position | specificity: 1.14 |
| | | | | ADAR2 |
| | | | | specificity: 0.81 |
| SEQ | LRRK2_ | ATTCTACAGG | 1/1 C/U mismatch at −8 position | ADAR1 on |
| ID | bnPCR_ | AGTTCTGAGC | 1/1 U/C mismatch at −3 position | target: 91.01% |
| NO: | ID38170_ | ATTCCCGTCGT | 1/1 A/C mismatch at 0 position | ADAR2 on |
| 109 | count773 | CATCAATCTTT | 3/3 symmetric bulge at +2 position (CAU- | target: 39.14% |
| | | GCA | UUC) | ADAR1 |
| | | | 1/1 U/U mismatch at +12 position | specificity: 1.2 |
| | | | 1/1 G/G mismatch at +16 position | ADAR2 |
| | | | | specificity: 0.69 |
| SEQ | LRRK2_ | ATTCTACAGC | 1/1 C/C mismatch at −8 position | ADAR1 on |
| ID | bnPCR_ | AGTACAGAGG | 1/1 U/G wobble base pair at −3 position | target: 91.49% |
| NO: | ID33405_ | ACTGCCGAGG | 1/1 A/A mismatch at −2 position | ADAR2 on |
| 110 | count860 | TCACCAATCTT | 1/1 A/C mismatch at 0 position | target: 81.85% |
| | | TGCA | 1/1 U/C mismatch at +4 position | ADAR1 |
| | | | 1/1 G/G mismatch at +6 position | specificity: 1.03 |
| | | | 1/1 A/A mismatch at +10 position | ADAR2 |
| | | | | specificity: 0.85 |
| SEQ | LRRK2_ | ATTCTACAGA | 1/1 A/C mismatch at 0 position | ADAR1 on |
| ID | bnPCR_ | AGGACCGAGC | 1/1 C/C mismatch at +2 position | target: 91.67% |
| NO: | ID39919_ | AGTCCCGTAG | 1/1 U/G wobble base pair at +4 position | ADAR2 on |
| 111 | count1633 | TCAGCAATCTT | 1/1 A/C mismatch at +10 position | target: 89.02% |
| | | TGCA | 1/1 A/G mismatch at +13 position | ADAR1 |
| | | | 1/1 G/A mismatch at +16 position | specificity: 0.73 |
| | | | | ADAR2 |
| | | | | specificity: 0.8 |
| SEQ | LRRK2_ | ATTCTACAGC | 3/3 symmetric bulge at −4 position (GAC- | ADAR1 on |
| ID | bnPCR_ | ATTACTGAGC | UUA) | target: 92.10% |
| NO: | ID21333_ | AATCCCGTATT | 1/1 A/C mismatch at 0 position | ADAR2 on |
| 112 | count1914 | AAGCAATCTTT | 1/1 C/C mismatch at +2 position | target: 92.36% |
| | | GCA | 1/1 C/U mismatch at +14 position | ADAR1 |
| | | | | specificity: 1.03 |
| | | | | ADAR2 |
| | | | | specificity: 0.7 |
| SEQ | LRRK2_ | ATTCTACAGC | 1/1 G/U wobble base pair at −6 position | ADAR1 on |
| ID | bnPCR_ | AGTACGAAGC | 2/2 symmetric bulge at −2 position (UA- | target: 92.46% |
| NO: | ID97672_ | AATCCCGCCG | CC) | ADAR2 on |
| 113 | count397 | TTAGCAATCTT | 1/1 A/C mismatch at 0 position | target: 20.91% |
| | | TGCA | 1/1 C/C mismatch at +2 position | ADAR1 |
| | | | 2/2 symmetric bulge at +9 position (CA- | specificity: 1.01 |
| | | | GA) | ADAR2 |
| | | | | specificity: 0.54 |
| SEQ | LRRK2_ | ATTCTACAGC | 1/1 G/A mismatch at −6 position | ADAR1 on |
| ID | bnPCR_ | AGTACTGTTCA | 6/6 symmetric internal loop at −3 | target: 93.75% |
| NO: | ID36558_ | ATTCCGATGTA | position (UACAGC-UCCGAU) | ADAR2 on |
| 114 | count1785 | AGCAATCTTTG | 2/2 symmetric bulge at +7 position (CU- | target: 56.09% |
| | | CA | UU) | ADAR1 |
| | | | | specificity: 1.4 |
| | | | | ADAR2 |
| | | | | specificity: 0.92 |
| SEQ | LRRK2_ | ATTCTACAAC | 1/1 C/A mismatch at −8 position | ADAR1 on |
| ID | bnPCR_ | AGTACTGAGC | 10/10 symmetric internal loop at −4 | target: 94.19% |
| NO: | ID16690_ | TATCCCGAATT | position (CUACAGCAUU- | ADAR2 on |
| 115 | count1976 | CAACAATCTTT | UAUCCCGAAU (SEQ ID NOS 214-215, | target: 90.41% |
| | | GCA | respectively)) | ADAR1 |
| | | | 1/1 C/A mismatch at +17 position | specificity: 1.01 |
| | | | | ADAR2 |
| | | | | specificity: 0.85 |
| SEQ | LRRK2_ | ATTCTACGGC | 1/1 C/A mismatch at −8 position | ADAR1 on |
| ID | bnPCR_ | AGTTCATAGC | 1/1 A/C mismatch at 0 position 1/1 C/C | target: 94.28% |
| NO: | ID22357_ | AATCCCGTAG | mismatch at +2 position | ADAR2 on |
| 116 | count844 | TCAACAATCTT | 2/2 symmetric bulge at +9 position (CA- | target: 12.67% |
| | | TGCC | AU) | ADAR1 |
| | | | 1/1 U/U mismatch at +12 position | specificity: 1.15 |
| | | | 1/1 U/G wobble base pair at +18 position | ADAR2 |
| | | | | specificity: 0.71 |
| SEQ | LRRK2_ | CTTCTACAGCA | 1/1 U/G wobble base pair at −3 position | ADAR1 on |
| ID | bnPCR_ | GTTCGGAGGA | 1/1 A/1 mismatch at −2 position | target: 94.31% |
| NO: | ID8030_ | ATCCCGAGGT | 1/1 A/C mismatch at 0 position | ADAR2 on |
| 117 | count919 | CAGCAATCTTT | 1/1 C/C mismatch at +2 position | target: 74.70% |
| | | GCA | 1/1 G/G mismatch at +6 position | ADAR1 |
| | | | 3/3 symmetric bulge at +10 position | specificity: 1.36 |
| | | | (AGU-UCG) | ADAR2 |
| | | | 5/0 asymmetric internal loop at +24 | specificity: 0.86 |
| | | | position (AUAUA-) | |
| | | | 1/1 G/U wobble base pair at +29 position | |
| SEQ | LRRK2_ | ATTCTACAGCT | 1/1 U/C mismatch at −7 position | ADAR1 on |
| ID | bnPCR_ | GTACTAGGCT | 1/1 A/C mismatch at 0 position | target: 95.93% |
| NO: | ID43647_ | ATCCCGTAGTC | 1/1 C/C mismatch at +2 position | ADAR2 on |
| 118 | count763 | CGCAATCTTTG | 1/1 U/U mismatch at +5 position | target: 82.09% |
| | | CA | 1/1 U/G wobble base pair at +8 position | ADAR1 |
| | | | 1/1 C/A mismatch at +9 position | specificity: 1.11 |
| | | | 1/1 U/U mismatch at +15 position | ADAR2 |
| | | | | specificity: 0.96 |
| SEQ | LRRK2_ | ATTCTACAGCC | 1/1 U/U mismatch at −7 position | ADAR1 on |
| ID | bnPCR_ | GTACTGGGAA | 1/1 A/C mismatch at 0 position | target: 90.48% |
| NO: | ID4389_ | ATCCCGTAGTC | 1/1 C/C mismatch at +2 position | ADAR2 on |
| 119 | count1135 | TGCAATCTTTG | 1/1 G/A mismatch at +6 position | target: 87.02% |
| | | CA | 1/1 U/G wobble base pair at +8 position | ADAR1 |
| | | | 1/1 U/C mismatch at +15 position | specificity: 0.94 |
| | | | | ADAR2 |
| | | | | specificity: 1.14 |
| SEQ | LRRK2_ | ATTCTACAGA | 1/1 A/C mismatch at −16 position | ADAR1 on |
| ID | bnPCR_ | AGTACAGAGA | 1/1 C/A mismatch at −8 position | target: 84.41% |
| NO: | ID38794 | CATCCCGTAGT | 1/1 G/U wobble base pair at −6 position | ADAR2 on |
| 120 | count776 | TAACAATCTTC | 5/4 asymmetric internal loop at 0 | target: 82.65% |
| | | GCA | position (AGCAU-UCCC) | ADAR1 |
| | | | 0/1 asymmetric bulge at +6 position (−A) | specificity: 1.09 |
| | | | 1/1 A/A mismatch at +10 position | ADAR2 |
| | | | 1/1 G/A mismatch at +16 position | specificity: 1.18 |
| SEQ | LRRK2_ | ATTCTACAGCC | 1/1 U/C mismatch at −7 position | ADAR1 on |
| ID | bnPCR_ | GTACTGAGCT | 1/1 A/C mismatch at 0 position | target: 87.33% |
| NO: | ID37802_ | ATCCCGTAGTC | 1/1 C/C mismatch at +2 position | ADAR2 on |
| 121 | count2433 | CGCAATCTTTG | 1/1 U/U mismatch at +5 position | target: 88.42% |
| | | CA | 1/1 U/C mismatch at +15 position | ADAR1 |
| | | | | specificity: 0.97 |
| | | | | ADAR2 |
| | | | | specificity: 1.06 |
| SEQ | LRRK2_ | ATTCTACTGCA | 2/2 symmetric bulge at −7 position (CU- | ADAR1 on |
| ID | bnPCR_ | GCACCGAGCA | CC) | target: 93.09% |
| NO: | ID489_ | ATCCCGCATTC | 7/7 symmetric internal loop at −4 | ADAR2 on |
| 122 | count3336 | CCCAATCTTTG | position (CUACAGC-CCCGCAU) | target: 84.08% |
| | | CA | 1/1 A/C mismatch at +10 position | ADAR1 |
| | | | 1/1 A/C mismatch at +13 position | specificity: 0.62 |
| | | | 1/1 U/U mismatch at +18 position | ADAR2 |
| | | | | specificity: 0.78 |
| SEQ | LRRK2_ | ATTCTACAGC | 1/1 A/C mismatch at 0 position | ADAR1 on |
| ID | bnPCR_ | AGTCCTGAGC | 1/1 C/A mismatch at +2 position | target: 85.47% |
| NO: | ID50412_ | AGTACCGTAG | 1/1 U/G wobble base pair at +4 position | ADAR2 on |
| 123 | count1318 | TCAGCAATCTT | 1/1 U/C mismatch at +12 position | target: 84.08% |
| | | TGCA | | ADAR1 |
| | | | | specificity: 0.86 |
| | | | | ADAR2 |
| | | | | specificity: 0.96 |
| SEQ | LRRK2_ | ATTCTAAGGC | 1/1 U/G wobble base pair at −7 position | ADAR1 on |
| ID | bnPCR_ | AGTACTGAGC | 1/1 A/C mismatch at 0 position | target: 85.92% |
| NO: | ID27560_ | CGCGCCGTAG | 1/1 A/C mismatch at +3 position | ADAR2 on |
| 124 | count2218 | TCGGCAATCTT | 1/1 U/G wobble base pair at +4 position | target: 82.19% |
| | | TGCA | 1/1 U/C mismatch at +5 position | ADAR1 |
| | | | 1/1 U/G wobble base pair at +18 position | specificity: 0.98 |
| | | | 1/1 G/A mismatch at +19 position | ADAR2 |
| | | | | specificity: 0.88 |

### Example 12

### High Throughput Screening of Engineered Guide RNAs Targeting SNCA mRNA

Using the compositions and methods described herein, high throughput screening (HTS) of engineered guide RNAs that target SNCA mRNA. Self-annealing RNA structures comprising the candidate engineered guide RNA sequences of TABLE 5 and the sequences of the regions targeted by the guide RNAs were contacted with an RNA editing entity (e.g., a recombinant ADAR1 and/or ADAR2) under conditions that allow for the editing of the regions targeted by the guide RNAs. The regions targeted by the guide RNAs were subsequently assessed for editing using next generation sequencing (NGS). The guide RNAs of TABLE 5 showed specific editing of the A nucleotide at translation initiation start site (TIS; the A in the ATG start coding with genomic coordinates: hg38 chr4: 89835667 strand −1) of SNCA mRNA. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 5
Exemplary guide RNAs that target SNCA mRNA

| SEQ | | | | |
|---|---|---|---|---|
| ID | | Guide RNA | | |
| NO | ID | Sequence | Structural Features (target/guide) | Metrics |
| SEQ | SNCA_guide01_ | GAAAGTACTT | 1/1 A/C mismatch at 0 position | ADAR2 on target: |
| ID | v7 | TGATGAATAC | 1/1 G/G mismatch at +14 position | 95.68% |
| NO: | | ATCCACGGCT | 1/1 G/A mismatch at +19 position | ADAR2 specificity: |
| 125 | | AATGAATTCC | | 1.95 |
| | | TTTAC | | |
| SEQ | SNCA_guide02_ | GAAAGTCCTT | 4/4 symmetric bulge at −6 position | ADAR2 on target: |
| ID | v25 | TCAAGAATAC | (UCAU-UACU) | 94.39% |
| NO: | | ATCCACGGCT | 1/1 A/C mismatch at 0 position | ADAR2 specificity: |
| 126 | | ATACTATTCC | 1/1 A/A mismatch at +12 position | 1.94 |
| | | TTTAC | | |
| SEQ | SNCA_guide01_ | GAAAGTCCTT | 1/1 A/C mismatch at 0 position | ADAR2 on target: |
| ID | v24 | TGATGCATAC | 1/1 U/C mismatch at +10 position | 95.92% |
| NO: | | ATCCACGGCT | 1/1 G/G mismatch at +14 position | ADAR2 specificity: |
| 127 | | AATGAATTCC | | 1.93 |
| | | TTTAC | | |
| SEQ | SNCA_guide02_ | GAAAGTCCTT | 4/4 symmetric bulge at −6 position | ADAR2 on target: |
| ID | v24 | TGATGCATAC | (UCAU-UACU) | 94.10% |
| NO: | | ATCCACGGCT | 1/1 A/C mismatch at 0 position | ADAR2 specificity: |
| 128 | | ATACTATTCC | 1/1 U/C mismatch at +10 position | 1.93 |
| | | TTTAC | 1/1 G/G mismatch at +14 position | |
| SEQ | SNCA_guide02_ | GAAAGTCCTT | 4/4 symmetric bulge at −6 position | ADAR2 on target: |
| ID | v22 | TGAGGCATAC | (UCAU-UACU) | 88.96% |
| NO: | | ATCCACGGCT | 1/1 A/C mismatch at 0 position | ADAR2 specificity: |
| 129 | | ATACTATTCC | 1/1 U/C mismatch at +10 position | 1.88 |
| | | TTTAC | 1/1 A/G mismatch at +12 position | |
| | | | 1/1 G/G mismatch at +14 position | |
| SEQ | SNCA_guide02_ | GAAAGTCCTT | 4/4 symmetric bulge at −6 position | ADAR2 on target: |
| ID | v21 | TGAGGAATAC | (UCAU-UACU) | 93.74% |
| NO: | | ATCCACGGCT | 1/1 A/C mismatch at 0 position | ADAR2 specificity: |
| 130 | | ATACTATTCC | 1/1 A/G mismatch at +12 position | 1.93 |
| | | TTTAC | 1/1 G/G mismatch at +14 position | |
| SEQ | SNCA_guide02_ | GAAAGTCCTT | 4/4 symmetric bulge at −6 position | ADAR2 on target: |
| ID | v18 | TGAAGCATAC | (UCAU-UACU) | 84.65% |
| NO: | | ATCCACGGCT | 1/1 A/C mismatch at 0 position | ADAR2 specificity: |
| 131 | | ATACTATTCC | 1/1 U/C mismatch at +10 position | 1.84 |
| | | TTTAC | 1/1 A/A mismatch at +12 position | |
| | | | 1/1 G/G mismatch at +14 position | |
| SEQ | SNCA_guide02_ | GAAAGTACTT | 4/4 symmetric bulge at −6 position | ADAR2 on target: |
| ID | v11 | TCACGAATAC | (UCAU-UACU) | 89.43% |
| NO: | | ATCCACGGCT | 1/1 A/C mismatch at 0 position | ADAR2 specificity: |
| 132 | | ATACTATTCC | 1/1 A/C mismatch at +12 position | 1.88 |
| | | TTTAC | 1/1 G/A mismatch at +19 position | |
| SEQ | SNCA_guide02_ | GAAAGTCCTT | 4/4 symmetric bulge at −6 position | ADAR2 on target: |
| ID | v28 | TCACGCATAC | (UCAU-UACU) | 93.20% |
| NO: | | ATCCACGGCT | 1/1 A/C mismatch at 0 position | ADAR2 specificity: |
| 133 | | ATACTATTCC | 1/1 U/C mismatch at +10 position | 1.93 |
| | | TTTAC | 1/1 A/C mismatch at +12 position | |
| SEQ | SNCA_guide02_ | GAAAGTCCTT | 4/4 symmetric bulge at −6 position | ADAR2 on target: |
| ID | v26 | TCAAGCATAC | (UCAU-UACU) | 90.62% |
| NO: | | ATCCACGGCT | 1/1 A/C mismatch at 0 position | ADAR2 specificity: |
| 134 | | ATACTATTCC | 1/1 U/C mismatch at +10 position | 1.91 |
| | | TTTAC | 1/1 A/A mismatch at +12 position | |

### Example 13

### High Throughput Screening of Engineered Guide RNAs Targeting SERPINA1 mRNA

Using the compositions and methods described herein, high throughput screening (HTS) of engineered guide RNAs that target SERPINA1 mRNA. High throughput screening (HTS) of gRNA sequences against the SERPINA1 E342K mutation identified designs with superior on-target activity and specificity.

Self-annealing RNA structures comprising the candidate engineered guide RNA sequences of TABLE 6 and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS). The guide RNAs of TABLE 6 showed specific editing of the A nucleotide. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 6
Exemplary guide RNAs that target SERPINA1 mRNA

| SEQ | | | | |
|---|---|---|---|---|
| ID | | | | |
| NO | Name | Sequence | Structural Features (target/guide) | Metrics |
| SEQ | SERP_ | GCCCCAGCA | 1/1 G/U wobble base pair at −10 position | ADAR1 on |
| ID | 006448_ | GCTTCAGTT | 2/0 asymmetric bulge at −8 position (AC-) | target: 53.40% |
| NO: | SERINA1- | CCTTAATCG | 1/1 A/C mismatch at 0 position | ADAR1 |
| 135 | 20_26_45. | TCGATGTAG | 2/2 symmetric bulge at +2 position (GA-AA) | specificity: 1.49 |
| | 62 | CACAGCC | 1/1 G/U wobble base pair at +8 position | |
| SEQ | SERP_ | ATTCCCATG | 1/1 A/C mismatch at 0 position | ADAR1 on |
| ID | 006566_ | GCCCCAGCA | 2/1 asymmetric bulge at +3 position (AA-A) | target: 45.30% |
| NO: | SERINA1- | GCTTCAGTC | 1/1 G/U wobble base pair at +6 position | ADAR1 |
| 136 | 12_34_45. | CTTACTCGT | | specificity: 1.40 |
| | 34 | CGATGGTCA | | |
| SEQ | SERP_ | CCTCTAAAA | 1/1 A/C mismatch at 0 position | ADAR1 on |
| ID | 007612_ | ACATGGCTC | 1/0 asymmetric bulge at +8 position (G−) | target: 47.50% |
| NO: | SERINA1- | CCCACAGCT | 2/2 symmetric bulge at +19 position (CU-CA) | ADAR1 |
| 137 | 6_40_45.40 | TCAGTCCTTT | 0/1 asymmetric bulge at +23 position (−U) | specificity: 1.46 |
| | | CTCGTCGA | | |
| SEQ | SERP_ | ATGGCCTGT | 1/1 U/G wobble base pair at −14 position | ADAR1 on |
| ID | 007657_ | GCAGCTTCA | 1/1 G/A mismatch at −13 position | target: 64.20% |
| NO: | SERINA1- | GTCCCTTAC | 1/1 U/G wobble base pair at −11 position | ADAR1 |
| 138 | 17_29_45. | TCGTCGATG | 0/3 asymmetric bulge at −8 position (−CCG) | specificity: 1.49 |
| | 29 | GCCGTCGGA | 1/1 A/C mismatch at 0 position | |
| | | GCTCA | 1/1 A/A mismatch at +3 position | |
| | | | 2/2 symmetric bulge at +20 position (UG-GU) | |
| | | | 1/1 G/U wobble base pair at +22 position | |
| SEQ | SERP_ | AGTAAAACA | 1/1 A/C mismatch at 0 position | ADAR1 on |
| ID | 010900_ | TGTACGCCC | 1/0 asymmetric bulge at +5 position (A-) | target: 52.60% |
| NO: | SERINA1- | CAGCCTTCA | 2/0 asymmetric bulge at +16 position (CU-) | ADAR1 |
| 139 | 9_37_45.37 | GTCCCTTCT | 0/3 asymmetric bulge at +25 position (−UAC) | specificity: 1.52 |
| | | CGTCGA | | |
| SEQ | SERP_ | AAAACAATA | 1/1 A/C mismatch at 0 position | ADAR1 on |
| ID | 010980_ | CTACCCAGC | 4/2 asymmetric bulge at +3 position (AAAG- | target: 49.90% |
| NO: | SERINA1- | AGCTTCAGT | AC) | ADAR1 |
| 140 | 12_34_45. | CCACCTCGT | 4/6 asymmetric internal loop at +24 position | specificity: 1.47 |
| | 34 | CGATGGTCA | (GCCA-AUACUA) | |
| SEQ | SERP_ | CTAATCCAT | 1/1 A/C mismatch at 0 position | ADAR1 on |
| ID | 011202_ | GGCTACCCC | 2/0 asymmetric bulge at +11 position (UG-) | target: 46.10% |
| NO: | SERINA1- | AGCAGCTTG | 0/3 asymmetric bulge at +23 position (−UAC) | ADAR1 |
| 141 | 9_37_45.37 | TCCCTTTCTC | 3/2 asymmetric bulge at +30 position (UUU- | specificity: 1.41 |
| | | GTCGATGG | UC) | |
| SEQ | SER_ | TGCTAAAAA | 1/1 U/C mismatch at −5 position | ADAR1 on |
| ID | 011563_ | CATGGTGCA | 1/1 A/C mismatch at 0 position | target: 42.00% |
| NO: | SERINA1- | GCAGCTTCA | 3/2 asymmetric bulge at +3 position (AAA- | ADAR1 |
| 142 | 9_37_45.37 | GTCCTGACT | GA) | specificity: 1.41 |
| | | CGTCGCTGG | 1/1 G/U wobble base pair at +6 position | |
| | | | 2/1 asymmetric bulge at +22 position (GG-G) | |
| | | | 1/1 G/U wobble base pair at +24 position | |
| SEQ | SERP_ | AAAAACATG | 1/1 A/C mismatch at 0 position | ADAR1 on |
| ID | 012208_ | GCCCCAGCA | 3/3 symmetric bulge at +3 position (AAA- | target: 46.40% |
| NO: | SERINA1- | GCTTCAGTC | CAA) | ADAR1 |
| 143 | 11_35_45. | CCCAACTCG | | specificity: 1.28 |
| | 35 | TCGATGGTC | | |
| SEQ | SERP_ | AAAACATGG | 1/1 A/C mismatch at 0 position | ADAR1 on |
| ID | 013687_ | CCCCAGCAG | 2/2 symmetric bulge at +3 position (AA-GA) | target: 44.90% |
| NO: | SERINA1- | CTTCAGTCC | | ADAR1 |
| 144 | 12_34_45. | CTGACTCGT | | specificity: 1.28 |
| | 34 | CGATGGTCA | | |
| SEQ | SERP_ | CTAAAAACA | 1/1 C/A mismatch at −1 position | ADAR1 on |
| ID | 016311_ | TGGCACAGC | 3/3 symmetric bulge at +2 position (GAA- | target: 47.40% |
| NO: | SERINA1- | AGCTTCAGT | CCA) | ADAR1 |
| 145 | 9_37_45.37 | CCCTCCATT | 2/1 asymmetric bulge at +22 position (GG-A) | specificity: 1.45 |
| | | ATCGATGG | | |
| SEQ | SERP_ | AACTACAAG | 0/1 asymmetric bulge at −7 position (-U) | ADAR1 on |
| ID | 101671_ | GCCCCAGCA | 1/1 A/C mismatch at 0 position | target: 48.00% |
| NO: | 1-SERINA | GCTTCATCC | 2/0 asymmetric bulge at +2 position (-A) | ADAR1 |
| 146 | 11_35_45. | CTTTACTCGT | 1/0 asymmetric bulge at +10 position (C-) | specificity: 1.44 |
| | 35 | CGATGTGG | 1/1 A/S mismatch at +27 position | |
| SEQ | SERP_ | CTAAAAACA | 1/0 asymmetric bulge at −6 position (A-) | ADAR1 on |
| ID | 017788_ | TGGCTCCCA | 0/2 asymmetric bulge at −1 position (-CU) | target: 51.30% |
| NO: | SERINA1- | GCAGCTTCA | 1/1 A/C mismatch at 0 position | ADAR1 |
| 147 | 9_37_45.37 | TATCCTTTCC | 1/1 G/U wobble base pair at +8 position | specificity: 1.51 |
| | | TGCTTCGAG | 2/2 symmetric bulge at +9 position (AC-UA) | |
| | | G | 0/1 asymmetric bulge at +23 position (−U) | |
| SEQ | SERP_ | TAAAAACAT | 0/1 asymmetric bulge at −4 position (−A) | ADAR1 on |
| ID | 019344_ | GGCCCCCAG | 1/1 C/A mismatch at −1 position | target: 45.90% |
| NO: | SERINA1- | TTCAGTCCC | 0/2 asymmetric bulge at +4 position (-AA) | ADAR1 |
| 148 | 10_36_45. | TAATTCTTAT | 1/0 asymmetric bulge at +15 position (G-) | specificity: 1.45 |
| | 36 | CGAATGGT | 2/0 asymmetric bulge at +19 position (CU-) | |
| SEQ | SERP_ | CTAAAAACG | 1/1 A/C mismatch at 0 position | ADAR1 on |
| ID | 020550_ | CCCCAGCAG | 0/3 asymmetric bulge at +5 position (−ACA) | target: 53.40% |
| NO: | SERINA1- | CTTCAGTCC | 3/0 asymmetric bulge at +26 position (CAU-) | ADAR1 |
| 149 | 9_37_45.37 | CACATTTCT | | specificity: 1.53 |
| | | CGTCGATGG | | |
| SEQ | SERP_ | GCCCCGTCA | 1/1 U/U mismatch at −16 position | ADAR1 on |
| ID | 021899_ | GTTCAGTCC | 1/1 G/U wobble base pair at −15 position | target: 51.50% |
| NO: | SERINA1- | CTGTTCTCGT | 2/2 symmetric bulge at −8 position (AC-CA) | ADAR1 |
| 150 | 20_26_45. | CGATGCACA | 1/1 A/C mismatch at 0 position | specificity: 1.48 |
| | 26 | GCATTGCC | 0/1 asymmetric bulge at +4 position (−G) | |
| | | | 1/0 asymmetric bulge at +15 position (G-) | |
| | | | 1/1 C/U mismatch at +19 position | |
| | | | 1/1 U/G wobble base pair at +20 position | |
| SEQ | SERP_ | CTAAAAACC | 1/1 A/C mismatch at 0 position | ADAR1 on |
| ID | 024066_ | CATGGCGAC | 1/1 A/C mismatch at +5 position | target: 55.80% |
| NO: | SERINA1- | AGCAGCTTC | 2/0 asymmetric bulge at +10 position (CU-) | ADAR1 |
| 151 | 9_37_45.37 | TCCCCTTCTC | 2/2 symmetric bulge at +22 position (GG-GA) | specificity: 1.49 |
| | | GTCGATGG | 0/2 asymmetric bulge at +28 position (−CC) | |
| SEQ | SERP_ | AAGCTATGG | 0/1 asymmetric bulge at −8 position (−A) | ADAR1 on |
| ID | 024794_ | CCCCAGCAG | 1/0 asymmetric bulge at −5 position (U-) | target: 42.30% |
| NO: | SERINA1- | CTTCACCCT | 1/1 A/C mismatch at 0 position | ADAR1 |
| 152 | 14_32_45. | TTCTCGTCGT | 2/0 asymmetric bulge at +9 position (AC-) | specificity: 1.41 |
| | 32 | GGATCC | 0/1 asymmetric bulge at +28 position (-U) | |
| | | | 1/1 U/G wobble base pair at +30 position | |
| SEQ | SERP_ | AAAACATGG | 2/2 symmetric bulge at −6 position (−CA/AC) | ADAR1 on |
| ID | 028982_ | CACCCCAGC | 1/1 A/C mismatch at 0 position | target: 47.00% |
| NO: | SERINA1- | AGCTTCAGC | 2/0 asymmetric bulge at +8 position (GA-) | ADAR1 |
| 153 | 12_34_45. | CTTTCTCGTC | 0/2 asymmetric bulge at +23 position (−AC) | specificity: 1.43 |
| | 34 | GAACGTCA | | |
| SEQ | SERP_ | ACATGGCCC | 0/3 asymmetric bulge at −10 position (−CUA) | ADAR1 on |
| ID | 031836_ | CAGAGCTTC | 1/1 A/C mismatch at 0 position | target: 42.20% |
| NO: | SERINA1- | AGTGCCCTT | 0/1 asymmetric bulge at +8 position (−G) | ADAR1 |
| 154 | 15_31_45. | TCTCGTCGA | 1/0 asymmetric bulge at +18 position (G-) | specificity: 1.39 |
| | 31 | TGGTCCTAA | | |
| | | GCA | | |
| SEQ | SERP_ | CTCTAAAAA | 1/1 C/A mismatch at −1 position | ADAR1 on |
| ID | 033465_ | CATGACCCC | 4/3 asymmetric bulge at +2 position (GAAA- | target: 43.60% |
| NO: | SERINA1- | AGCAGCTTC | CCG) | ADAR1 |
| 155 | 7_39_45.39 | AGTCCTCCG | 1/1 G/U wobble base pair at +6 position | specificity: 1.42 |
| | | TTATCGAT | 1/1 C/A mismatch at +25 position | |
| SEQ | SERP_ | ACATCAGGC | 1/1 A/A mismatch at −9 position | ADAR1 on |
| ID | 034669_ | CCCACAGCT | 1/1 A/C mismatch at 0 position | target: 44.70% |
| NO: | SERINA1- | TCAGTCCCT | 1/0 asymmetric bulge at +5 position (A-) | ADAR1 |
| 156 | 15_31_45. | TCTCGTCGA | 1/0 asymmetric bulge at +19 position (C-) | specificity: 1.19 |
| | 31 | TGGACAG | 0/2 asymmetric bulge at +26 position (-CA) | |
| SEQ | SERP_ | CCCCAGCGT | 1/0 asymmetric bulge at −14 position (U-) | ADAR1 on |
| ID | 034803_ | CTTCAGTCC | 1/1 C/A mismatch at −12 position | target: 49.00% |
| NO: | SERINA1- | CTTTCTCGTC | 1/1 U/G wobble base pair at −11 position | ADAR1 |
| 157 | 21_25_45. | GATGGTCGA | 1/1 A/C mismatch at 0 position | specificity: 1.18 |
| | 25 | CCAGCCT | 1/1 C/U mismatch at +16 position | |
| | | | 1/1 U/G wobble base pair at +17 position | |
| SEQ | SERP_ | GGCCCCAGC | 1/1 A/C mismatch at 0 position | ADAR1 on |
| ID | 034837_ | AGCTTCAGT | 3/3 symmetric bulge at +3 position (AAA- | target: 48.00% |
| NO: | SERINA1- | CCCAGACTC | AGA) | ADAR1 |
| 158 | 19_27_45. | GTCGATGGT | | specificity: 1.37 |
| | 27 | CAGCACAAC | | |
| SEQ | SERP_ | TAAAAAACA | 1/1 A/C mismatch at 0 position | ADAR1 on |
| ID | 035444_ | TGGCCCCAG | 0/1 asymmetric bulge at +2 position (-A) | target: 42.60% |
| NO: | SERINA1- | CAGTCAGTC | 2/0 asymmetric bulge at +13 position (AA-) | ADAR1 |
| 159 | 10_36_45. | CCTTTACTC | 1/1 G/U wobble base pair at +15 position | specificity: 1.32 |
| | 36 | GTCGATGGT | | |
| SEQ | SERP_ | GGCCAGCAG | 3/3 symmetric bulge at −6 position (CCA- | ADAR1 on |
| ID | 036632_ | CTTCACCAA | AUA) | target: 60.10% |
| NO: | SERINA1- | GTTTCTCGTC | 1/1 A/C mismatch at 0 position | ADAR1 |
| 160 | 21_25_45. | GAATATCAG | 5/5 symmetric internal loop at +6 position | specificity: 1.54 |
| | 25 | CACAGCCT | (GGGAC-CCAAG) | |
| SEQ | SERP_ | CTAAAAACA | 3/3 symmetric bulge at −1 position (CAA- | ADAR1 on |
| ID | 039699_ | TGGCCCCAG | GAU) | target: 63.60% |
| NO: | SERINA1- | CAGCTTCAG | 0/1 asymmetric bulge at +1 position (-C) | ADAR1 |
| 161 | 9_37_45.37 | TCAACTTCT | 1/2 asymmetric bulge at +7 position (G-AA) | specificity: 1.63 |
| | | CGATTCGAT | | |
| | | GG | | |
| SEQ | SERP_ | TGGCGCCCC | 0/2 asymmetric bulge at −7 position (-CU) | ADAR1 on |
| ID | 040418_ | AGCAGCTTC | 0/2 asymmetric bulge at −3 position (-GG) | target: 48.20% |
| NO: | 1SERINA- | ATCCCTTCT | 1/1 A/C mismatch at 0 position | ADAR1 |
| 162 | 18_28_45. | CGTCGGGAT | 1/0 asymmetric bulge at +5 position (A-) | specificity: 1.45 |
| | 28 | GCTGTCAGC | 1/0 asymmetric bulge at +10 position (C-) | |
| | | ACAG | 0/1 asymmetric bulge at +25 position (-C) | |

### Example 14

### High Throughput Screening of Long Engineered Guide RNAs (100Mer and Longer) Targeting LRRK2 mRNA

Using the compositions and methods described herein, high throughput screening (HTS) of long engineered guide RNAs (e.g., 100mer and longer) that target LRRK2 mRNA was performed, where said engineered guide RNAs form a micro-footprint comprised of various structural features in the guide-target RNA scaffold and form a barbell macro-footprint comprising two 6/6 internal loops near both ends of the guide-target RNA scaffold. Additionally, in this high throughput screen, self-annealing RNA structures were of a size (231 nucleotides) that allowed for screening for engineered guide RNAs that were 113 nucleotides in length, with the target adenosine to be edited positioned at the 57^{th} nucleotide. The high throughput screen was able to identify engineered guide RNAs that show high on-target adenosine editing (>60%, 30 min incubation with ADAR1 and ADAR2) and reduced to no local off-target adenosine editing (e.g., at the −2 position relative to the target adenosine to be edited, which is at position 0). Self-annealing RNA structures that formed a barbell macro-footprint in the guide-target RNA scaffold were screened to include 4 different micro-footprints (A/C mismatch (ATTCTACAGCAGTACTGAGCAATGCCGTAGTCAGCAATCTTTGCA (SEQ ID NO: 212)), 2108 (ATTCTACGGCGGTACTGACCAATCCCGTAGTTAGCAATCTTTGCA (SEQ ID NO: 84), 871 (ATTCTACAGTAGGACTGAGCACTGCCGAGCTGGGCAATCTTTGCA (SEQ ID NO: 103), and 919 (CTTCTACAGCAGTTCGGAGGAATCCCGAGGTCAGCAATCTTTGCA (SEQ ID NO: 117)), tiling the position of the barbell macro-footprint from the −22 position to the −12 position at one end of the self-annealing RNA structure and from the +12 position to the +34 position at the other end of the self-annealing RNA structure. Self-annealing RNA structures comprising 1939 distinct guide RNA sequences and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS).

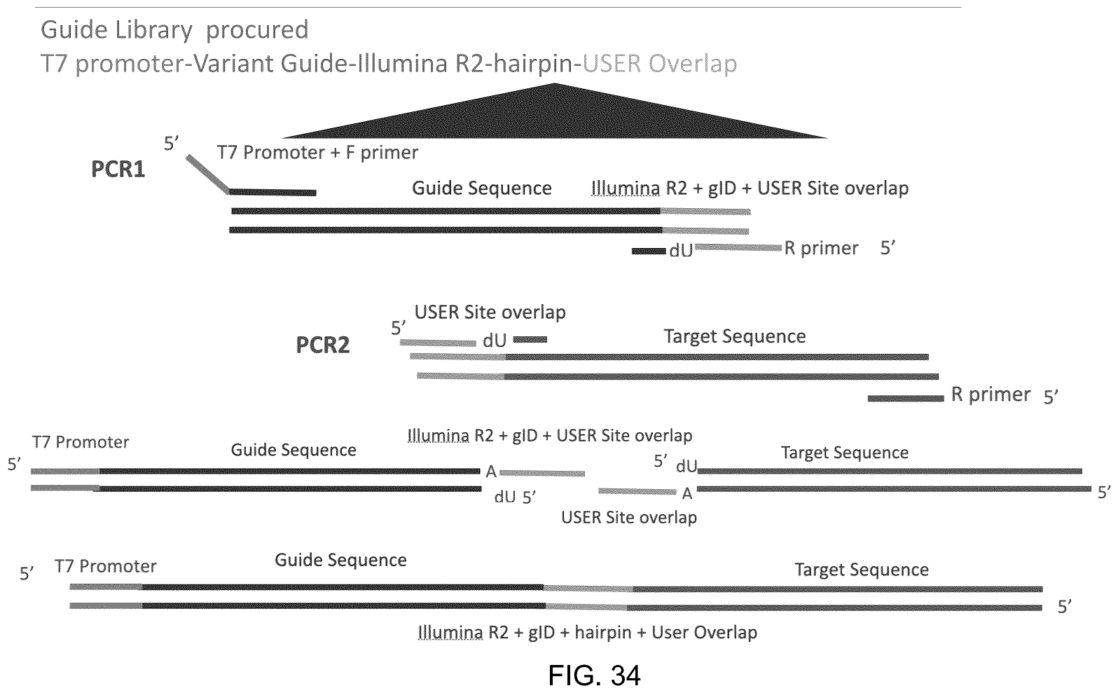

Libraries for screening of these longer engineered guides were generated as follows, and as summarized in FIG. 34: a candidate engineered guide library was procured having a constructs with a T7 promoter, followed by the candidate engineered guide RNA sequence to be tested, followed by an Illumina R2 hairpin, followed by a sequence for a USER (Uracil-specific excision reagent) site Overlap. Aspects of the reaction that are important include the USER enzyme incubation time, a 2:1 molar ratio of target to guide for longer HTS screen, and 30 cycles for the ligase cycling. This library and the target sequence were PCR amplified, incorporating a deoxy-Uridine (dU) at the 3′ end of the constructs containing the candidate engineered guide RNA sequences and at the 5′ end of the target. Next, the PCR amplified library and target are incubated with the USER enzyme, resulting in nicking at the dU positions and ligation (using Taq ligase) of a given library construct containing the candidate engineered guide RNA sequence to the target sequence.

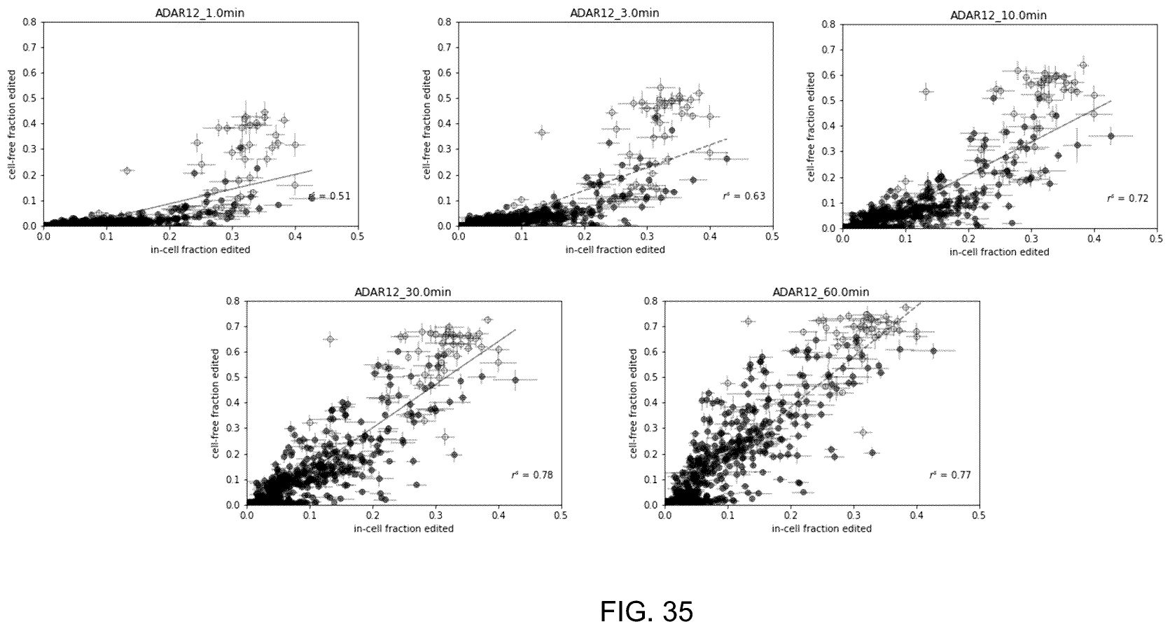

FIG. 35 shows a comparison of cell-free RNA editing using the methods and compositions described here versus in-cell RNA editing facilitated via the same engineered guide RNA sequence at various timepoints (20s, 1 min, 3 min, 10 min, 30 min and 60 min). In this experiment, 40 candidate guide RNAs were screened. 50 nM ADAR1+100 nM ADAR2 was present in each cell. The editing values for each guide and the position of the adenosine that was edited is presented in FIG. 35 as a cumulative of 6 values. The open circles represent on target adenosine editing for each guide, while the black circles represent editing of adenosines other than the on target adenosine (off target adenosines). As a whole, these data show that the cell-free high throughput screen is able to correlate well with in-cell RNA editing, in particular at certain timepoints (e.g., at 30 minutes).

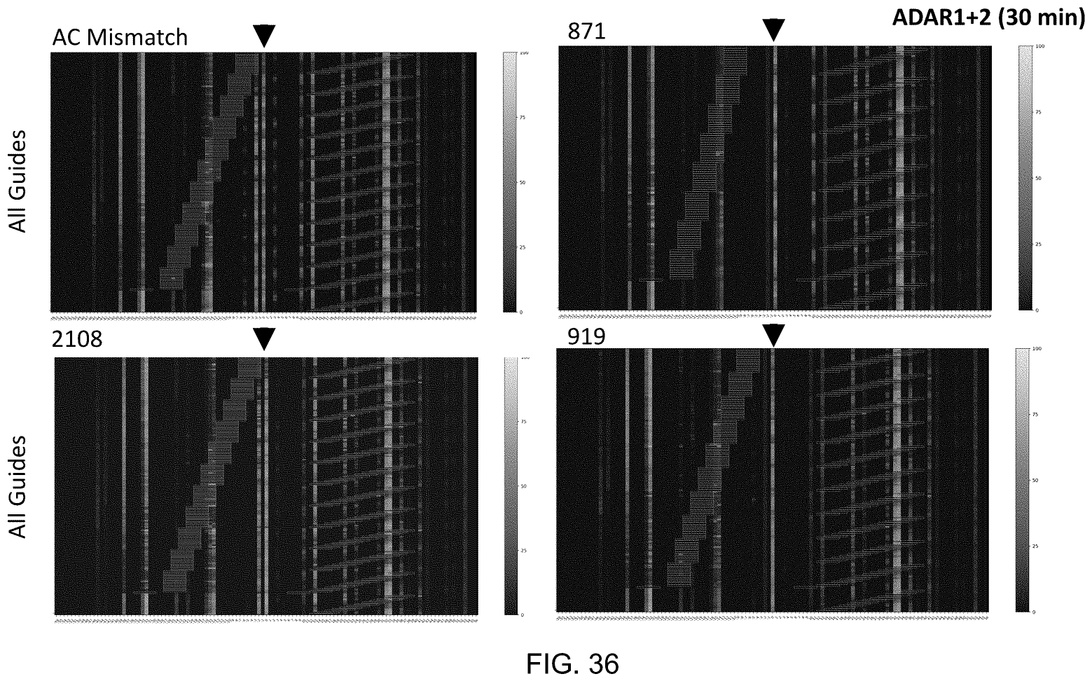

FIG. 36 shows heatmaps of all self-annealing RNA structures tested for the 4 micro-footprints described above formed within varying placement of a barbell macro-footprint. The y-axis shows all engineered guide RNAs tested and the x-axis shows the target sequence positions, with position 0 representing the target adenosine to be edited.

Exemplary engineered guide RNAs from the high throughput screen of this example are described in TABLE 7. The candidate engineered guide RNAs of TABLE 7 showed specific editing of the A nucleotide at position 6055 of the mRNA encoding the LRRK2 G2019S. Percent on-target editing is calculated by the following formula: the number of reads containing “G” at the target/the total number of reads. Specificity is calculated by the following formula: (percent on target editing+100)/(sum of off target editing percentage at selected off-targets sites+100). The addition of barbells produced specific editing patterns. In particular, the presence of barbell at position −14 and position +26 appeared to increase the specificity of ADAR editing. Thus, specificity can be improved significantly through the combination of micro-footprint structural features and macro-footprint structural features such as barbells.

TABLE 7
Exemplary Guide RNAs that target LRRK2 mRNA

| SEQ | | | | |
|---|---|---|---|---|
| ID | | | | |
| NO | ID | Sequence | Structural Features (target/guide) | Metrics |
| SEQ | LRRK2_ | CCCTGGTGTGC | 1/1 U/G wobble base pair at −3 | ADAR1 on target: 0.38% |
| ID | bnPCR_ | CCTCTGATGTT | position; | ADAR1 specificity: 0.5 |
| NO: | ID94725_ | TTTATCCCCAT | 1/1 | ADAR2 on target: 0.64% |
| 163 | count351_ | TCTACAGCGGT | A/C mismatch at 0 position; | ADAR2 specificity: 0.37 |
| | NoLoops_ | ACTGAGCAAA | 2_2/2 symmetric bulge at position | ADAR12 on target: 0.6% |
| | gID_02513 | TCCGTGGTCAG | +2 (CA-AU); | ADAR12 specificity: 0.43 |
| | | CAATCTTTGCA | 1/1 U-G wobble base pair at +15 | |
| | | ATGATGGCAG | | |
| | | CATTGGGATA | | |
| | | CAGTGTGAAG | | |
| | | AGCAGCA | | |
| SEQ | LRRK2_ | CCCTGGTGTGC | 6/6_ symmetric internal loop at −14 | ADAR1 on target: 0.32% |
| ID | bnPCR_ | CCTCTGATGTT | position (UGCAAA-AAACGU); | ADAR1 specificity: 0.77 |
| NO: | ID94725_ | TTTTAGGGGAT | 1/1 _U/G wobble base pair at −3 | ADAR2 on target: 0.7% |
| 164 | count351_- | TCTACAGCGGT | position; | ADAR2 specificity: 0.72 |
| | 14_26_ | ACTGAGCAAA | 1/1 A/C mismatch at 0 position; | ADAR12 on target: |
| | gID_08570 | TCCGTGGTCAG | 2/2 CA/AU symmetric bulge at +2 | 0.64% |
| | | CAATCAAACG | position; | ADAR12 specificity: 0.74 |
| | | TATGATGGCA | 1/1 U/G wobble at +15 position; | |
| | | GCATTGGGAT | 6/6 symmetric internal loop at +26 | |
| | | ACAGTGTGAA | position (GGGGAU-UAGGGG) | |
| | | GAGCAGCA | | |
| SEQ | LRRK2_ | CCCTGGTGTGC | 1/1 C/C mismatch at −4 position; | ADAR1 on target: 0.43% |
| ID | PbnCR_ | CCTCTGATGTT | 1/1 (U/G) wobble base pair at −3 | ADAR1 specificity: 0.54 |
| NO: | ID79791_ | TTTATCCCCAT | position; | ADAR2 on target: 0.69% |
| 165 | count610_ | TCTACATCTGT | 1/1 A/C mismatch at 0 position; | ADAR2 specificity: 0.38 |
| | NoLoops_ | AGTGAGCAAT | 1/1 C/U mismatch at +2 position; | ADAR12 on target: |
| | gID_06724 | TCCGTGCTCAG | 1/1 G/G mismatch at +11 position; | 0.66% |
| | | CAATCTTTGCA | 1/1 U/U mismatch at +15 position; | ADAR12 specificity: 0.42 |
| | | ATGATGGCAG | 1/1 C/U mismatch at +17 position | |
| | | CATTGGGATA | | |
| | | CAGTGTGAAG | | |
| | | AGCAGCA | | |
| SEQ | LRRK2_ | CCCTGGTGTGC | 6/6 symmetric internal loop at −14 | ADAR1 on target: 0.21% |
| ID | bnPCR_ | CCTCTGATGTT | position (UGCAAA-AAACGU); | ADAR1 specificity: 0.77 |
| NO: | ID79791_ | TTTTAGGGGAT | 1/1 C/C mismatch at −4 position; | ADAR2 on target: 0.66% |
| 166 | count610_- | TCTACATCTGT | 1/1 U/G wobble base pair at −3 | ADAR2 specificity: 0.71 |
| | 14_26_gID_ | AGTGAGCAAT | position; | ADAR12 on target: |
| | 09469 | TCCGTGCTCAG | 1/1 A/C mismatch at 0 position; | 0.62% |
| | | CAATCAAACG | 1/1 C/U mismatch at +2 position; | ADAR12 specificity: 0.75 |
| | | TATGATGGCA | 1/1 G/G mismatch at +11 position; | |
| | | GCATTGGGAT | 1/1 U/U mismatch +15 position; | |
| | | ACAGTGTGAA | 1/1 C/U mismatch at +17 position; | |
| | | GAGCAGCA | 6/6 symmetric internal loop at +26 | |
| | | | position (GGGGAU-UAGGGG) | |
| SEQ | LRRK2_ | CCCTGGTGTGC | 1/1 C/C mismatch at -4 position; | ADAR1 on target: 0.45% |
| ID | bnPCR_ | CCTCTGATGTT | 1/1 A/C mismatch at 0 position; | ADAR1 specificity: 0.57 |
| NO: | ID66010_ | TTTATCCCCAT | 1/1 C/C mismatch at +2 position; | ADAR2 on target: 0.67% |
| 167 | count1326_ | TCTACACCTGG | 1/1 G/A mismatch at +6 position; | ADAR2 specificity: 0.39 |
| | NoLoops_ | ACTGAGAAAT | 3/3 symmetric bulge at +13 position | ADAR12 on target: |
| | gID_09247 | CCCGTACTCAG | (ACU-UGG); | 0.65% |
| | | CAATCTTTGCA | 1/1 C/C mismatch at +17 position | ADAR12 specificity: 0.42 |
| | | ATGATGGCAG | | |
| | | CATTGGGATA | | |
| | | CAGTGTGAAG | | |
| | | AGCAGCA | | |
| SEQ | LRRK2_ | CCCTGGTGTGC | 6/6 symmetric internal loop at -14 | ADAR1 on target: 0.29% |
| ID | bnPCR_ | CCTCTGATGTT | position (UGCAAA-AAACGU); | ADAR1 specificity: 0.8 |
| NO: | ID66010_ | TTTTAGGGGAT | 1/1 C/C mismatch at -4 position; | ADAR2 on target: 0.64% |
| 168 | count1326_- | TCTACACCTGG | 1/1 A/C mismatch at 0 position; | ADAR2 specificity: 0.71 |
| | 14_26_gID_ | ACTGAGAAAT | 1/1 C/C mismatch at +2 position; | ADAR12 |
| | 11327 | CCCGTACTCAG | 1/1 G/A mismatch at +6 position; | on target: |
| | | CAATCAAACG | 3/3 symmetric bulge at +13 position | 0.62% |
| | | TATGATGGCA | (ACU-UGG); | ADAR12 specificity: 0.76 |
| | | GCATTGGGAT | 1/1 C/C mismatch at +17 position; | |
| | | ACAGTGTGAA | 6/6_symmetric internal loop at +26 | |
| | | GAGCAGCA | position (GGGGAU-UAGGGG) | |
| SEQ | LRRK2_ | CCCTGGTGTGC | 1/1 U/G wobble base pair at -3 | ADAR1 on target: 0.5% |
| ID | bnPCR_ | CCTCTGATGTT | position; | ADAR1 specificity: 0.52 |
| NO: | ID50437_ | TTTATCCCCAT | 1/1 A/C mismatch at 0 position; | ADAR2 on target: 0.7% |
| 169 | count708_ | TCTACAGCAGT | 1/1 U/G wobble base pair at +4 | ADAR2 specificity: 0.38 |
| | NoLoops_ | ACGGTGCAGT | position; | ADAR12 on target: |
| | gID_11520 | GCCGTGGTCA | 1/1 U/U mismatch at +8 position; | 0.65% |
| | | GCAATCTTTGC | 1/1 A/G mismatch at +10 position | ADAR12 specificity: 0.43 |
| | | AATGATGGCA | | |
| | | GCATTGGGAT | | |
| | | ACAGTGTGAA | | |
| | | GAGCAGCA | | |
| SEQ | LRRK2_ | CCCTGGTGTGC | 6/6 symmetric internal loop at −14 | ADAR1 on target: 0.36% |
| ID | bnPCR_ | CCTCTGATGTT | position (UGCAAA-AAACGU); | ADAR1 specificity: 0.76 |
| NO: | ID50437_ | TTTTAGGGGAT | 1/1 U/G wobble at −3 position; | ADAR2 on target: 0.68% |
| 170 | count708_- | TCTACAGCAGT | 1/1 A/C mismatch at O position; | ADAR2 specificity: 0.68 |
| | 14_26_gID_ | ACGGTGCAGT | 1/1 U/G wobble base pair at +4 | ADAR12 on target: |
| | 09857 | GCCGTGGTCA | position; | 0.69% |
| | | GCAATCAAAC | 1/1 U/U mismatch at +8 position; | ADAR12 specificity: 0.71 |
| | | GTATGATGGC | 1/1 A/G mismatch at +10 position; | |
| | | AGCATTGGGA | 6/6 symmetric internal loop at +26 | |
| | | TACAGTGTGA | position (GGGGAU-UAGGGG) | |
| | | AGAGCAGCA | | |
| SEQ | LRRK2_ | CCCTGGTGTGC | 1/1 U/G wobble base pair at −3 | ADAR1 on target: 0.4% |
| ID | bnPCR_ | CCTCTGATGTT | position; | ADAR1 specificity: 0.56 |
| NO: | ID41799_ | TTTATCCCCCT | 1/1 A/C mismatch at 0 position; | ADAR2 on target: 0.69% |
| 171 | count730_ | TCTACAGCAGT | 2/2 symmetric bulge at +4 position | ADAR2 specificity: 0.37 |
| | NoLoops_ | AGTGAGTTTTG | (UU-UU); | ADAR12 on target: |
| | gID_04947 | CCGTGGTCAG | 1/1 G/U wobble base pair at +6 | 0.66% |
| | | CAATCTTTGCA | position; | ADAR12 specificity: 0.41 |
| | | ATGATGGCAG | 1/1 G/G mismatch at +11 position; | |
| | | CATTGGGATA | 1/1 U/C mismatch at +25 position | |
| | | CAGTGTGAAG | | |
| | | AGCAGCA | | |
| SEQ | LRRK2_ | CCCTGGTGTGC | 6/6 symmetric internal loop at - | ADAR1 on target: 0.3% |
| ID | bnPCR_ | CCTCTGATGTT | 14_position (UGCAAA- | ADAR1 specificity: 0.79 |
| NO: | ID41799_ | TTTTAGGGGCT | AAACGU); | ADAR2 |
| 172 | count730_- | TCTACAGCAGT | 1/1 U/G wobble base pair at −3 | on target: |
| | 14_26_gID_ | AGTGAGTTTTG | position; | 0.68% |
| | 07755 | CCGTGGTCAG | 1/1 A/C mismatch at 0 position; | ADAR2 specificity: 0.67 |
| | | CAATCAAACG | 2/2 symmetric bulge at +4 position | ADAR12 on target: |
| | | TATGATGGCA | (UU-UU); | 0.65% |
| | | GCATTGGGAT | 1/1 G/U wobble base pair at +6 | ADAR12 specificity: 0.73 |
| | | ACAGTGTGAA | position; | |
| | | GAGCAGCA | 1/1 G/G mismatch at +11 position; | |
| | | | 7/7 symmetric internal loop at +25 | |
| | | | position (UGGGGAU- | |
| | | | UAGGGGC) | |
| SEQ | LRRK2_ | CCCTGGTGTGC | 1/1 C/A mismatch at −4 position; | ADAR1 on target: 0.45% |
| ID | bnPCR_ | CCTCTGATGTT | 1/1 U/G wobble base pair at −3 | ADAR1 specificity: 0.57 |
| NO: | ID35277_ | TTTATCCCCAT | position; | ADAR2 |
| 173 | count295_ | TCTACAGCAGT | 1/1 A/C mismatch at 0 position; | on target: 0.66% |
| | NoLoops_ | CCTGTACAGTG | 1/1 U/G wobble base pair at +4 | ADAR2 specificity: 0.39 |
| | gID_06840 | CCGTGATCAG | position; | ADAR12 on target: |
| | | CAATCTTTGCA | 2/2 symmetric bulge at +7 position | 0.64% |
| | | ATGATGGCAG | (CU-UA); | ADAR12 specificity: 0.44 |
| | | CATTGGGATA | 1/1 U/C mismatch at +12 position | |
| | | CAGTGTGAAG | | |
| | | AGCAGCA | | |
| SEQ | LRRK2_ | CCCTGGTGTGC | 6/6 symmetric internal loop at −14 | ADAR1 on target: 0.27% |
| ID | bnPCR_ | CCTCTGATGTT | position (UGCAAA-AAACGU); | ADAR1 specificity: 0.76 |
| NO: | ID35277_ | TTTTAGGGGAT | 1/1 C/A mismatch at −4 position; | ADAR2 on target: 0.68% |
| 174 | count295_- | TCTACAGCAGT | 1/1 U/G wobble base pair at −3 | ADAR2 specificity: 0.68 |
| | 14_26_gID_ | CCTGTACAGTG | position; | ADAR12 on target: |
| | 09359 | CCGTGATCAG | 1/1 A/C mismatch at 0 position; | 0.65% |
| | | CAATCAAACG | 1/1 U/G wobble base pair at +4 | ADAR12 specificity: 0.71 |
| | | TATGATGGCA | position; | |
| | | GCATTGGGAT | 2/2 symmetric bulge at +7 position | |
| | | ACAGTGTGAA | (CU-UA); | |
| | | GAGCAGCA | 1/1 U/C mismatch at +12 position; | |
| | | | 6/6 symmetric internal loop at +26 | |
| | | | position (GGGGAU-UAGGGG | |
| SEQ | LRRK2_ | CCCTGGTGTGC | 6/6 symmetric internal loop at −10 | ADAR1 on target: 0.33% |
| ID | bnPCR_ | CCTCTGATGTT | position (AAGAUU-CUAGGC); | ADAR1 specificity: 0.7 |
| NO: | ID34601_ | TTTATCCCCAT | 1/1 G/U wobble base pair at −6 | ADAR2 |
| 175 | count2108_- | TCGCCTGAGGT | position; | on target: 0.63% |
| | 10_16_gID_ | ACTGACCAAT | 1/1 A/C mismatch at 0 position; | ADAR2 specificity: 0.52 |
| | 01444 | CCCGTAGTTAG | 1/1 C/C mismatch at +2 position; | ADAR12 on target: 0.6% |
| | | CCTAGGCTGC | 1/1 C/C mismatch at +7 position; | ADAR12 specificity: 0.58 |
| | | AATGATGGCA | 1/1 U/G wobble base pair at +15 | |
| | | GCATTGGGAT | position; | |
| | | ACAGTGTGAA | 6/6 symmetric internal loop at +16 | |
| | | GAGCAGCA | position (GCUGUA-GCCUGA) | |
| SEQ | LRRK2_ | CCCTGGTGTGC | 6/6 symmetric internal loop at −22 | ADAR1 on target: 0.22% |
| ID | bnPCR_ | CCTCTGATGTT | position (GCCAUC-AACCUG); | ADAR1 specificity: 0.73 |
| NO: | ID33405_ | TTTCTACAGAT | 1/1 C/C mismatch at −8 position; | ADAR2 on target: 0.61% |
| 176 | count860_- | TCTACAGCAGT | 1/1 U/G wobble base pair at −3 | ADAR2 specificity: 0.56 |
| | 22_26_gID_ | ACAGAGGACT | position; | ADAR12 on target: |
| | 00318 | GCCGAGGTCA | 1/1 A/A mismatch at −2 position; | 0.56% |
| | | CCAATCTTTGC | 1/1 A/C mismatch at 0 position; | ADAR12 specificity: 0.63 |
| | | AATAACCTGA | 1/1 U/C mismatch at +4 position; | |
| | | GCATTGGGAT | 1/1 G/G mismatch at +6 position; | |
| | | ACAGTGTGAA | 1/1 A/A mismatch at +10 position; | |
| | | GAGCAGCA | 2/2 symmetric bulge at +26 position | |
| | | | (GG-AG); | |
| | | | 3/3 symmetric bulge at +29 position | |
| | | | (GAU-CUA) | |
| SEQ | LRRK2_ | CCCTGGTGTGC | 6/6 symmetric internal loop at −20 | ADAR1 on target: 0.22% |
| ID | bnPCR_ | CCTCTGATGTT | position (CAUCAU-UACUAC); | ADAR1 specificity: 0.73 |
| NO: | ID33405_ | TTTTAGGGGAT | 1/1 C/C mismatch at −8 position; | ADAR2 on target: 0.63% |
| 177 | count860_- | TCTACAGCAGT | 1/1 U/G wobble base pair at −3 | ADAR2 specificity: 0.58 |
| | 20_26_gID_ | ACAGAGGACT | position; | ADAR12 on target: |
| | 09192 | GCCGAGGTCA | 1/1 A/A mismatch at −2 position; | 0.58% |
| | | CCAATCTTTGC | 1/1 A/C mismatch at 0 position; | ADAR12 specificity: 0.64 |
| | | ATACTACGCA | 1/1 U/C mismatch at +4 position; | |
| | | GCATTGGGAT | 1/1 G/G mismatch at +6 position; | |
| | | ACAGTGTGAA | 1/1 A/A mismatch at +10 position; | |
| | | GAGCAGCA | 6/6 symmetric internal loop at +26 | |
| | | | position (GGGGAU-UAGGGG) | |
| SEQ | LRRK2_ | CCCTGGTGTGC | 6/6 symmetric internal loop at −16 | ADAR1 on target: 0.21% |
| ID | bnPCR_ | CCTCTGATGTT | position (AUUGCA-GUCUAG); | ADAR1 specificity: 0.76 |
| NO: | ID33405_ | TTTCGGGAGAT | 1/1 C/C mismatch at −8 position; | ADAR2 on target: 0.66% |
| 178 | count860_- | TCTACAGCAGT | 1/1 U/G wobble at −3 position; | ADAR2 specificity: 0.6 |
| | 16_26_gID_ | ACAGAGGACT | 1/1 A/A mismatch at −2 position; | ADAR12 on target: |
| | 11898 | GCCGAGGTCA | 1/1 A/C mismatch at 0 position; | 0.59% |
| | | CCAATCTTGTC | 1/1 U/C mismatch at +4 position; | ADAR12 specificity: 0.63 |
| | | TAGGATGGCA | 1/1 G/G mismatch at +6 position; | |
| | | GCATTGGGAT | 1/1 A/A mismatch at +10 position; | |
| | | ACAGTGTGAA | 6/6 symmetric internal loop at +26 | |
| | | GAGCAGCA | position (GGGGAU-CGGGAG) | |
| (SEQ | LRRK2_ | CCCTGGTGTGC | 6/6 symmetric internal loop at −12 | ADAR1 on target: 0.39% |
| ID | bnPCR_ | CCTCTGATGTA | position (CAAAGA-CGCUAA); | ADAR1 specificity: 0.75 |
| NO: | ID33405_ | TGTTCCCCCAT | 1/1 C/C mismatch at −8 position; | ADAR2 on target: 0.45% |
| 216) | count860_- | TCTACAGCAGT | 1/1 U/G wobble base pair at −3 | ADAR2 specificity: 0.55 |
| | 12_30_gID_ | ACAGAGGACT | position; | ADAR12 on target: 0.5% |
| | 05698 | GCCGAGGTCA | 1/1 A/A mismatch at −2 position; | ADAR12 specificity: 0.66 |
| | | CCAACGCTAA | 1/1 A/C mismatch at 0 position; | |
| | | CAATGATGGC | 1/1 U/C mismatch at +4 position; | |
| | | AGCATTGGGA | 1/1 G/G mismatch at +6 position; | |
| | | TACAGTGTGA | 1/1 A/A mismatch at +10 position; | |
| | | AGAGCAGCA | 4/4 symmetric bulge at +30 position | |
| | | | (AUAA-GUUC); | |
| | | | 1/1 A/A mismatch at +35 position | |
| SEQ | LRRK2_ | CCCTGGTGTGC | 6/6 symmetric internal loop at −8 | ADAR1 on target: 0.33% |
| ID | bnPCR_ | CCTCTGATGTT | position (GAUUGC-CUUUGA); | ADAR1 specificity: 0.67 |
| NO: | ID33405_ | TTTATGGAAA | 1/1 U/G wobble base pair at −3 | ADAR2 on target: 0.58% |
| 179 | count860_- | GTCTACAGCA | position; | ADAR2 specificity: 0.58 |
| | 8_24_gID_ | GTACAGAGGA | 1/1 A/A mismatch at −2 position; | ADAR12 on target: |
| | 05952 | CTGCCGAGGT | 1/1 A/C mismatch at 0 position; | 0.49% |
| | | CACTTTGATTT | 1/1 U/C mismatch at +4 position; | ADAR12 specificity: 0.64 |
| | | GCAATGATGG | 1/1 G/G mismatch at +6 position; | |
| | | CAGCATTGGG | 1/1 A/A mismatch at +10 position; | |
| | | ATACAGTGTG | 6/6 symmetric internal loop at +24 | |
| | | AAGAGCAGCA | position (AUGGGG-GGAAAG) | |
| SEQ | LRRK2_ | CCCTGGTGTGC | 6/6 symmetric internal loop at −22 | ADAR1 on target: 0.25% |
| ID | bnPCR_ | CCTCTGATGTT | position (GCCAUC-CAAAAA); | ADAR1 specificity: 0.72 |
| NO: | ID29209_ | TTTCGAAGAAT | 1/1 U/G wobble base pair at −7 | ADAR2 |
| 180 | count871_- | TCTACAGTAG | position; | on target: 0.59% |
| | 22_26_gID_ | GACTGAGCAC | 1/1 G/G mismatch at −6 position; | ADAR2 specificity: 0.61 |
| | 01244 | TGCCGAGCTG | 0/1 asymmetric bulge at −4 position | ADAR12 on target: |
| | | GGCAATCTTTG | (-C); | 0.56% |
| | | CAATCAAAAA | 1/0 asymmetric bulge at −2 position | ADAR12 specificity: 0.67 |
| | | AGCATTGGGA | (A-); | |
| | | TACAGTGTGA | 1/1 A/C mismatch at 0 position; | |
| | | AGAGCAGCA | 1/1 U/C mismatch at +4 position; | |
| | | | 1/1 A/G mismatch at +13 position; | |
| | | | 1/1 G/U wobble at +16 position; | |
| | | | 6/6 symmetric internal loop at +26 | |
| | | | position (GGGGAU-CGAAGA) | |
| SEQ | LRRK2_ | CCCTGGTGTGC | 6/6 symmetric internal loop at −20 | ADAR1 on target: 0.25% |
| ID | bnPCR_ | CCTCTGATGTT | position (CAUCAU-UACUAC); | ADAR1 specificity: 0.73 |
| NO: | ID29209_ | TTTTAGGGGAT | 1/1 U/G wobble base pair at −7 | ADAR2 on target: 0.62% |
| 181 | count871_- | TCTACAGTAG | position; | ADAR2 specificity: 0.67 |
| | 20_26_gID_ | GACTGAGCAC | 1/1 G/G mismatch at −6 position; | ADAR12 on target: 0.6% |
| | 07422 | TGCCGAGCTG | 0/1 asymmetric bulge at −4 position | ADAR12 specificity: 0.69 |
| | | GGCAATCTTTG | (-C); | |
| | | CATACTACGC | 1/0 asymmetric bulge at −2 position | |
| | | AGCATTGGGA | (A-); | |
| | | TACAGTGTGA | 1/1 A/C mismatch at 0 position; | |
| | | AGAGCAGCA | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +26 | |
| | | | position (GGGGAU-UAGGGG) | |
| SEQ | LRRK2_ | CCCTGGTGTGC | 6/6 symmetric internal loop at −16 | ADAR1 on target: 0.32% |
| ID | bnPCR_ | CCTCTGATGTT | position (AUUGCA-CAUUUG); | ADAR1 specificity: 0.77 |
| NO: | ID29209_ | TTTCTACAGAT | 1/1 U/G wobble base pair at −7 | ADAR2 on target: 0.6% |
| 182 | count871_- | TCTACAGTAG | position; | ADAR2 specificity: 0.66 |
| | 16_26_gID_ | GACTGAGCAC | 1/1 G/G mismatch at −6 position; | ADAR12 on target: |
| | 01312 | TGCCGAGCTG | 0/1 aysmmetric bulge at −4 position | 0.57% |
| | | GGCAATCTTCA | (-C); | ADAR12 specificity: 0.69 |
| | | TTTGGATGGCA | 1/0 asymmetric bulge at −2 position | |
| | | GCATTGGGAT | (A-); | |
| | | ACAGTGTGAA | 1/1 A/C mismatch at 0 position; | |
| | | GAGCAGCA | 1/1 U/C mismatch at +4 position; | |
| | | | 1/1 A/G mismatch at +13 position; | |
| | | | 1/1 G/U wobble base pair at +16 | |
| | | | position; | |
| | | | 2/2 symmetric bulge at +26 position | |
| | | | (GG-AG); | |
| | | | 3/3 symmetric bulge at +29 position | |
| | | | (GAU-CUA) | |
| SEQ | LRRK2_ | CCCTGGTGTGC | 6/6 symmetric internal loop at −16 | ADAR1 on target: 0.28% |
| ID | bnPCR_ | CCTCTGATGTT | position (AUUGCA-GAGUCG); | ADAR1 specificity: 0.81 |
| NO: | ID29209_ | TTTATGGGGTG | 1/1 U/G wobble base pair at −7 | ADAR2 on target: 0.62% |
| 183 | count871_- | TCTACAGTAG | position; | ADAR2 specificity: 0.73 |
| | 16_24_gID_ | GACTGAGCAC | 1/1 G/G mismatch at −6 position; | ADAR12 on target: |
| | 00727 | TGCCGAGCTG | 0/1 asymmetric bulge at −4 position | 0.58% |
| | | GGCAATCTTG | (-C); | ADAR12 specificity: 0.76 |
| | | AGTCGGATGG | 1/0 asymmetric bulge at −2 position | |
| | | CAGCATTGGG | (A-); | |
| | | ATACAGTGTG | 1/1 A/C mismatch at 0 position; | |
| | | AAGAGCAGCA | 1/1 U/C mismatch at +4 position; | |
| | | | 1/1 A/G mismatch at +13 position; | |
| | | | 1/1 G/U wobble at +16 position; 6/6 | |
| | | | symmetric internal loop at +24 | |
| | | | position (AUGGGG-GGGGUG) | |
| SEQ | LRRK2_ | CCCTGGTGTGC | 6/6 symmetric internal loop at −16 | ADAR1 on target: 0.2% |
| ID | bnPCR_ | CCTCTGATGTT | position (AUUGCA-CUCCCA); | ADAR1 specificity: 0.81 |
| NO: | ID29209_ | TTTATCCGAAA | 1/1 U/G wobble at −7 position; | ADAR2 on target: 0.55% |
| 184 | count871_- | CATACAGTAG | 1/1 G/G mismatch at −6 positon; | ADAR2 specificity: 0.74 |
| | 16_22_gID_ | GACTGAGCAC | 0/1 asymmetric bulge at −4 position | ADAR12 |
| | 11427 | TGCCGAGCTG | (-C); | on target: |
| | | GGCAATCTTCT | 1/0 asymmetric bulge at −2 position | 0.48% |
| | | CCCAGATGGC | (A-); | ADAR12 specificity: 0.75 |
| | | AGCATTGGGA | 1/1 A/C mismatch at 0 position; | |
| | | TACAGTGTGA | 1/1 U/C mismatch at +4 position; | |
| | | AGAGCAGCA | 1/1 A/G mismatch at +13 position; | |
| | | | 1/1 G/U wobble base pair at +16 | |
| | | | position; | |
| | | | 6/6 symmetric internal loop at +22 | |
| | | | position (GAAUGG-GAAACA) | |
| SEQ | LRRK2_ | CCCTGGTGTGC | 6/6 symmetric internal loop at −10 | ADAR1 on target: 0.45% |
| ID | bnPCR_ | CCTCTGATGTT | position (AAGAUU-CUCAGG); | ADAR1 specificity: 0.78 |
| NO: | ID29209_ | TTTCATGAGAT | 1/1 U/G wobble base pair at −7 | ADAR2 on target: 0.64% |
| 185 | count871_- | TCTACAGTAG | position; | ADAR2 specificity: 0.7 |
| | 10_26_gID_ | GACTGAGCAC | 1/1 G/G mismatch at −6 position; | ADAR12 on target: |
| | 00091 | TGCCGAGCTG | 0/1 asymmetric bulge at −4 position | 0.59% |
| | | GGCCTCAGGT | (-C); | ADAR12 specificity: 0.73 |
| | | GCAATGATGG | 1/0 asymmetric bulge at −2 position | |
| | | CAGCATTGGG | (A-); | |
| | | ATACAGTGTG | 1/1 A/C mismatch at 0 position; | |
| | | AAGAGCAGCA | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +26 | |
| | | | position (GGGGAU-CAUGAG) | |
| SEQ | LRRK2_ | CCCTGGTGTGC | 4/4 symmetric bulge at −9 position | ADAR1 on target: 0.38% |
| ID | bnPCR_ | CCTCTGATGTT | (AUUG-GUUC); | ADAR1 specificity: 0.74 |
| NO: | ID29209_ | TTTTACAGAAT | 1/1 U/G wobble base pair at −7 | ADAR2 on target: 0.57% |
| 186 | count871_- | TCTACAGTAG | position; | ADAR2 specificity: 0.65 |
| | 8_26_gID_ | GACTGAGCAC | 1/1 G/G mismatch at −6 position; | ADAR12 on target: |
| | 11298 | TGCCGAGCTG | 0/1 asymmetric bulge at −4 position | 0.53% |
| | | GGGTTCCTTTG | (-C); | ADAR12 specificity: 0.71 |
| | | CAATGATGGC | 1/0 asymmetric bulge at −2 position | |
| | | AGCATTGGGA | (A-); | |
| | | TACAGTGTGA | 1/1 A/C mismatch at 0 position; | |
| | | AGAGCAGCA | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +26 | |
| | | | position (GGGGAU-UACAGA) | |
| SEQ | LRRK2_ | CCCTGGTGTGC | 6/6 symmetric internal loop at −20 | ADAR1 on target: 0.39% |
| ID | bnPCR_ | CCTCTGATGTT | position (CAUCAU-UACUAC); | ADAR1 specificity: 0.71 |
| NO: | ID28134_ | TTTTAGGGGAT | 1/1 A/C mismatch at 0 position; | ADAR2 on target: 0.7% |
| 187 | count1700_- | TCTACCGCAGT | 1/1 C/C mismatch at +2 position; | ADAR2 specificity: 0.64 |
| | 20_26_gID_ | ACTACCCGATC | 1/1 U/G wobble base pair at +5 | ADAR12 on target: |
| | 06283 | CCGTAGTCAG | position; | 0.56% |
| | | CAATCTTTGCA | 3/3 symmetric bulge at +7 position | ADAR12 specificity: 0.7 |
| | | TACTACGCAG | (CUC-ACC); | |
| | | CATTGGGATA | 1/1 U/C mismatch at +18 position; | |
| | | CAGTGTGAAG | 6/6 symmetric internal loop at +26 | |
| | | AGCAGCA | position (GGGGAU-UAGGGG) | |
| SEQ | LRRK2_ | CCCTGGTGTGC | 1/1 U/G wobble base pair at −7 | ADAR1 on target: 0.44% |
| ID | bnPCR_ | CCTCTGATGTT | position; | ADAR1 specificity: 0.65 |
| NO: | ID24310_ | TTTATCCCCAT | 1/1 G/G mismatch at −6 position; | ADAR2 on target: 0.7% |
| 188 | count1321_ | TCTAGGGCAG | 1/1 U/G wobble base pair at −3 | ADAR2 specificity: 0.39 |
| | NoLoops_ | TAGGGTGCAC | position; | ADAR12 on target: |
| | gID_11060 | TGCCGTGGTG | 1/1 A/C mismatch at 0 position; | 0.68% |
| | | GGCAATCTTTG | 1/1 U/C mismatch at +4 position; | ADAR12 specificity: 0.46 |
| | | CAATGATGGC | 1/1 U/U mismatch at +8 position; | |
| | | AGCATTGGGA | 2/2 symmetric bulge at +10 position | |
| | | | (AG-GG); | |
| | | TACAGTGTGA | 1/1 U/G wobble base pair at +18 | |
| | | AGAGCAGCA | position; | |
| | | | 1/1 G/G mismatch at +19 position | |
| SEQ | LRRK2_ | CCCTGGTGTGC | 6/6 symmetric internal loop at −14 | ADAR1 on target: 0.15% |
| ID | bnPCR_ | CCTCTGATGTT | position (UGCAAA-AAACGU); | ADAR1 specificity: 0.82 |
| NO: | ID24310_ | TTTTAGGGGAT | 1/1 U/G wobble base pair at −7 | ADAR2 |
| 189 | count1321_- | TCTAGGGCAG | position; | on target: 0.67% |
| | 14_26_gID_ | TAGGGTGCAC | 1/1 G/G mismatch at −6 position; | ADAR2 specificity: 0.72 |
| | 02264 | TGCCGTGGTG | 1/1 U/G wobble base pair at −3 | ADAR12 on target: |
| | | GGCAATCAAA | position; | 0.64% |
| | | CGTATGATGG | 1/1 A/C mismatch at 0 position; | ADAR12 specificity: 0.75 |
| | | CAGCATTGGG | 1/1 U/C mismatch at +4 position; | |
| | | ATACAGTGTG | 1/1 U/U mismatch at +8 position; | |
| | | AAGAGCAGCA | 2/2 symmetric bulge at +10 position | |
| | | | (AG-GG); | |
| | | | 1/1 U/G wobble at +18 position; | |
| | | | 1/1 G/G mismatch at +19 position; | |
| | | | 6/6 symmetric internal loop at +26 | |
| | | | position (GGGGAU-UAGGGG) | |
| SEQ | LRRK2_ | CCCTGGTGTGC | 1/1 U/G wobble base pair at −3 | ADAR1 on target: 0.56% |
| ID | bnPCR_ | CCTCTGATGTT | position; | ADAR1 specificity: 0.59 |
| NO: | ID23393_ | TTTATCCCCAT | 1/1 A/C mismatch at 0 position; | ADAR2 on target: 0.69% |
| 190 | count2397_ | TCTACCGCTGT | 1/1 C/C mismatch at +2 position; | ADAR2 specificity: 0.41 |
| | NoLoops_ | GCTGGGCAAT | 1/1 U/G wobble base pair at +8 | ADAR12 on target: |
| | gID_09625 | CCCGTGGTCA | position; | 0.67% |
| | | GCAATCTTTGC | 1/1 U/G wobble base pair at +12 | ADAR12 specificity: 0.45 |
| | | AATGATGGCA | position; | |
| | | GCATTGGGAT | 1/1 U/U mismatch at +15 position; | |
| | | ACAGTGTGAA | 1/1 U/C mismatch at +18 position | |
| | | GAGCAGCA | | |
| SEQ | LRRK2_ | CCCTGGTGTGC | 6/6 symmetric internal loop at −20 | ADAR1 on target: 0.41% |
| ID | bnPCR_ | CCTCTGATGTT | position (CAUCAU-UACUAC); | ADAR1 specificity: 0.67 |
| NO: | ID23393_ | TTTTAGGGGAT | 1/1 U/G wobble base pair at −3 | ADAR2 on target: 0.64% |
| 191 | count2397_- | TCTACCGCTGT | position; | ADAR2 specificity: 0.7 |
| | 20_26_gID_ | GCTGGGCAAT | 1/1 A/C mismatch at 0 position; | ADAR12 on target: |
| | 06774 | CCCGTGGTCA | 1/1 C/C mismatch at +2 position; | 0.63% |
| | | GCAATCTTTGC | 1/1 U/G wobble base pair at +8 | ADAR12 specificity: 0.73 |
| | | ATACTACGCA | position; | |
| | | GCATTGGGAT | 1/1 U/G wobble base pair at +12 | |
| | | ACAGTGTGAA | position; | |
| | | GAGCAGCA | 1/1 U/U mismatch at +15 position; | |
| | | | 1/1 U/C mismatch at +18 position; | |
| | | | 6/6 symmetric internal loop at +26 | |
| | | | position (GGGGAU-UAGGGG) | |
| SEQ | LRRK2_ | CCCTGGTGTGC | 6/6 symmetric internal loop at −14 | ADAR1 on target: 0.39% |
| ID | bnPCR_ | CCTCTGATGTT | position (UGCAAA-AAACGU); | ADAR1 specificity: 0.83 |
| NO: | ID23393_ | TTTTAGGGGAT | 1/1 U/G wobble base pair at −3 | ADAR2 on target: 0.67% |
| 192 | count2397_- | TCTACCGCTGT | position; | ADAR2 specificity: 0.75 |
| | 14_26_gID_ | GCTGGGCAAT | 1/1 A/C mismatch at 0 position; | ADAR12 on target: |
| | 06456 | CCCGTGGTCA | 1/1 C/C mismatch at +2 position; | 0.62% |
| | | GCAATCAAAC | 1/1 U/G wobble base pair at +8 | ADAR12 specificity: 0.75 |
| | | GTATGATGGC | position; | |
| | | AGCATTGGGA | 1/1 U/G wobble base pair at +12 | |
| | | TACAGTGTGA | position; | |
| | | AGAGCAGCA | 1/1 U/G mismatch at +15 position; | |
| | | | 1/1 U/C mismatch at +18 position; | |
| | | | 6/6 symmetric internal loop at +26 | |
| | | | position (GGGGAU-UAGGGG) | |
| SEQ | LRRK2_ | CCCTGGTGTGC | 1/1 U/G wobble at −3 position; | ADAR1 on target: 0.48% |
| ID | bnPCR_ | CCTCTGATGTT | 1/1 A/C mismatch at 0 position; | ADAR1 specificity: 0.5 |
| NO: | ID22759_ | TTTATCCCCAT | 1/1 U/G wobble base pair at +4 | ADAR2 on target: 0.68% |
| 193 | count1590_ | TCTACAACAGT | position; | ADAR2 specificity: 0.43 |
| | NoLoops_ | ACGGTGAAGT | 5/5 symmetric internal loop at +6 | ADAR12 on target: |
| | gID_06335 | GCCGTGGTCA | position (GCUCA-GGUGA); | 0.62% |
| | | GCAATCTTTGC | 1/1 C/A mismatch at +17 position | ADAR12 specificity: 0.46 |
| | | AATGATGGCA | | |
| | | GCATTGGGAT | | |
| | | ACAGTGTGAA | | |
| | | GAGCAGCA | | |
| SEQ | LRRK2_ | CCCTGGTGTGC | 6/6 symmetric internal loop at −14 | ADAR1 on target: 0.28% |
| ID | bnPCR_ | CCTCTGATGTT | position (UGCAAA-AAACGU); | ADAR1 specificity: 0.77 |
| NO: | ID22759_ | TTTTAGGGGAT | 1/1 U/G wobble base pair at −3 | ADAR2 on target: 0.68% |
| 194 | count1590_- | TCTACAACAGT | position; | ADAR2 specificity: 0.74 |
| | 14_26_gID_ | ACGGTGAAGT | 1/1 A/C mismatch at 0 position; | ADAR12 on target: |
| | 08476 | GCCGTGGTCA | 1/1 U/G wobble base pair at +4 | 0.66% |
| | | GCAATCAAAC | position; | ADAR12 specificity: 0.77 |
| | | GTATGATGGC | 5/5 symmetric internal loop at +6 | |
| | | AGCATTGGGA | position (GCUCA-GGUGA); | |
| | | TACAGTGTGA | 1/1 C/A mismatch at +17 position; | |
| | | AGAGCAGCA | 6/6 symmetric internal loop at +26 | |
| | | | position (GGGGAU-UAGGGG) | |
| SEQ | LRRK2_ | CCCTGGTGTGC | 9/9 symmetric internal loop at −19 | ADAR1 on target: 0.43% |
| ID | bnPCR_ | CCTCTGATGTT | position (GCCAUCAUU- | ADAR1 specificity: 0.74 |
| NO: | ID22357_ | TTTCTAGATAT | CAUUAUUUG); | ADAR2 on target: 0.64% |
| 195 | count844_- | TCTACGGCAGT | 1/1 C/A mismatch at −8 position; | ADAR2 specificity: 0.5 |
| | 22_26_gID_ | TCATAGCAATC | 1/1 A/C mismatch at 0 position; | ADAR12 on target: |
| | 02673 | CCGTAGTCAA | 1/1 C/C mismatch at +2 position; | 0.52% |
| | | CAATCTTTGCC | 2/2 symmetric bulge at +9 position | ADAR12 specificity: 0.57 |
| | | ATTATTTGAGC | (CA-AU); | |
| | | ATTGGGATAC | 1/1 U/G mismatch at +12 position; | |
| | | AGTGTGAAGA | 1/1 U/G wobble base pair at +18 | |
| | | GCAGCA | position; | |
| | | | 1/1 G/U wobble base pair at +26 | |
| | | | position; | |
| | | | 5/5 symmetric internal loop at +27 | |
| | | | position (GGGAU-CUAGA) | |
| SEQ | LRRK2_ | CCCTGGTGTGC | 8/8 symmetric internal loop at −12 | ADAR1 on target: 0.4% |
| ID | bnPCR_ | CCTCTGATGTT | position (UGCAAAGA-GGGUCC | ADAR1 specificity: 0.82 |
| NO: | ID22357_ | TTTCGGAGGAT | CC); | ADAR2 on target: 0.52% |
| 196 | count844_- | TCTACGGCAGT | 1/1 C/A mismatch at −8 position; | ADAR2 specificity: 0.71 |
| | 12_26_gID_ | TCATAGCAATC | 1/1 A/C mismatch at 0 position; | ADAR12 on target: |
| | 07465 | CCGTAGTCAA | 1/1 C/C mismatch at +2 position; | 0.48% |
| | | CAAGGGTCCC | 2/2 symmetric bulge at +9 position | ADAR12 specificity: 0.74 |
| | | CATGATGGCA | (CA-AU); | |
| | | GCATTGGGAT | 1/1 U/U mismatch at +12 position; | |
| | | ACAGTGTGAA | 1/1 U/G wobble base pair at +18 | |
| | | GAGCAGCA | position; | |
| | | | 6/6 symmetric internal loop at +26 | |
| | | | position (GGGGAU-CGGAGG) | |
| SEQ | LRRK2_ | CCCTGGTGTGC | 8/8 symmetric internal loop at −12 | ADAR1 on target: 0.3% |
| ID | bnPCR_ | CCTCTGATGTT | position (UGCAAAGA-CAAUAU | ADAR1 specificity: 0.79 |
| NO: | ID22357_ | TTTATCCCCAT | CC); | ADAR2 on target: 0.68% |
| 197 | count844_- | CACCCTGCAGT | 1/1 | ADAR2 specificity: 0.67 |
| | 12_18_gID_ | TCATAGCAATC | C/A mismatch at −8 position; | ADAR12 on target: |
| | 11904 | CCGTAGTCAA | 1/1 A/C mismatch at 0 position; | 0.65% |
| | | CAACAATATC | 1/1 C/C mismatch at +2 position; | ADAR12 specificity: 0.73 |
| | | CATGATGGCA | 2/2 symmetric bulge at +9 position | |
| | | GCATTGGGAT | (CA-AU); | |
| | | ACAGTGTGAA | 1/1 U/U mismatch at +12 position; | |
| | | GAGCAGCA | 6/6 symmetric internal loop at +18 | |
| | | | position (UGUAGA-CACCCU) | |
| SEQ | LRRK2_ | CCCTGGTGTGC | 6/6 symmetric internal loop at −22 | ADAR1 on target: 0.5% |
| ID | bnPCR_ | CCTCTGATGTT | position (GCCAUC-AAGCGA); | ADAR1 specificity: 0.66 |
| NO: | ID16690_ | TTTCTGGTGAT | 1/1 C/A mismatch at -8 position; | ADAR2 on target: 0.64% |
| 198 | count1976_- | TCTACAACAGT | 10/10 symmetric internal loop at | ADAR2 specificity: 0.51 |
| | 22_26_gID_ | ACTGAGCTATC | −4→5 position (CUACAGCAUU- | ADAR12 |
| | 01893 | CCGAATTCAA | UAUCCCGAAU (SEQ ID NOS | on target: 0.6% |
| | | CAATCTTTGCA | 214-215, respectively)); | ADAR12 specificity: 0.56 |
| | | ATAAGCGAAG | 1/1 C/A mismatch at +17 position; | |
| | | CATTGGGATA | 1/1 G/G mismatch at +26 position; | |
| | | CAGTGTGAAG | 1/1 G/U wobble base pair at +27 | |
| | | AGCAGCA | position; | |
| | | | 4/4 symmetric bulge at +28 position | |
| | | | (GGAU-CUGG) | |
| SEQ | LRRK2_ | CCCTGGTGTGC | 6/6 symmetric internal loop at −22 | ADAR1 on target: 0.22% |
| ID | bnPCR_ | CCTCTGATGTT | position (GCCAUC-CAAUCA); | ADAR1 specificity: 0.7 |
| NO: | ID16690_ | TTTATCCCCAT | 1/1 C/A mismatch at −8 position; | ADAR2 on target: 0.61% |
| 199 | count1976_- | TCGAAAAAAG | 10/10 symmetrica internal loop at | ADAR2 specificity: 0.61 |
| | 22_16_gID_ | TACTGAGCTAT | −4→5 position (CUACAGCAUU- | ADAR12 on target: |
| | 06801 | CCCGAATTCA | UAUCCCGAAU (SEQ ID NOS | 0.57% |
| | | ACAATCTTTGC | 214-215, respectively)); | ADAR12 specificity: 0.61 |
| | | AATCAATCAA | 6/6 symmetric internal loop at +16 | |
| | | GCATTGGGAT | position (GCUGUA-GAAAAA) | |
| | | ACAGTGTGAA | | |
| | | GAGCAGCA | | |
| SEQ | LRRK2_ | CCCTGGTGTGC | 6/6 symmetric internal loop at −16 | ADAR1 on target: 0.22% |
| ID | bnPCR_ | CCTCTGATGTT | position (AUUGCA-CUAUUA); | ADAR1 specificity: 0.8 |
| NO: | ID16690_ | TTTATCCCCAT | 1/1 C/A mismatch at -8 position; | ADAR2 on target: 0.66% |
| 200 | count1976_- | TCGAATAAAG | 10/10 symmetric internal loop at | ADAR2 specificity: 0.67 |
| | 16_16_gID_ | TACTGAGCTAT | −4→5 position (CUACAGCAUU- | ADAR12 on target: 0.6% |
| | 10719 | CCCGAATTCA | UAUCCCGAAU (SEQ ID NOS | ADAR12 specificity: 0.7 |
| | | ACAATCTTCTA | 214-215, respectively)); | |
| | | TTAGATGGCA | 6/6 symmetric internal loop at +16 | |
| | | GCATTGGGAT | position (GCUGUA-GAAUAA) | |
| | | ACAGTGTGAA | | |
| | | GAGCAGCA | | |
| SEQ | LRRK2_ | CCCTGGTGTGC | 1/1 C/U mismatch at −8 position; | ADAR1 on target: 0.49% |
| ID | bnPCR_ | CCTCTGATGTT | 1/1 U/G wobble base pair at −3 | ADAR1 specificity: 0.54 |
| NO: | ID14524_ | TTTATCCCCAT | position; | ADAR2 on target: 0.7% |
| 201 | count2063_ | TCTACAGCAGT | 1/1 A/C mismatch at 0 position; | ADAR2 specificity: 0.38 |
| | NoLoops_ | AGTGTGCAGT | 1/1 U/g wobble base pair at +4 | ADAR12 on target: |
| | gID_01159 | GCCGTGGTCAT | position; | 0.66% |
| | | CAATCTTTGCA | 1/1 U/U mismatch at +8 position; | ADAR12 specificity: 0.43 |
| | | ATGATGGCAG | 1/1 G/G mismatch at +11 position | |
| | | CATTGGGATA | | |
| | | CAGTGTGAAG | | |
| | | AGCAGCA | | |
| SEQ | LRRK2_ | CCCTGGTGTGC | 6/6 symmetric internal loop at -14 | ADAR1 on target: 0.31% |
| ID | bnPCR_ | CCTCTGATGTT | position (UGCAAA-AAACGU); | ADAR1 specificity: 0.77 |
| NO: | ID14524_ | TTTTAGGGGAT | 1/1 C/U mismatch at −8 position; | ADAR2 on target: 0.72% |
| 202 | count2063_- | TCTACAGCAGT | 1/1 U/G wobble base pair at −3 | ADAR2 specificity: 0.69 |
| | 14_26_gID_ | AGTGTGCAGT | position; | ADAR12 on target: |
| | 05349 | GCCGTGGTCAT | 1/1 A/C mismatch at 0 position; | 0.68% |
| | | CAATCAAACG | 1/1 U/G wobble base pair at +4 | ADAR12 specificity: 0.73 |
| | | TATGATGGCA | position; | |
| | | GCATTGGGAT | 1/1 U/U mismatch at +8 position; | |
| | | ACAGTGTGAA | 1/1 G/G mismatch at +11 position; | |
| | | GAGCAGCA | 6/6 symmetric internal loop at +26 | |
| | | | position (GGGGAU-UAGGGG) | |
| SEQ | LRRK2_ | CCCTGGTGTGC | 6/6 symmetric internal loop at −22 | ADAR1 on target: 0.37% |
| ID | bnPCR_ | CCTCTGATGTT | position (GCCAUC-AAGAGA); | ADAR1 specificity: 0.68 |
| NO: | ID8030_ | TTTCAGTAGCT | 1/1 U/G wobble base pair at -3 | ADAR2 |
| 203 | count919_- | TCTACAGCAGT | position; | on target: 0.6% |
| | 22_26_gID_ | TCGGAGGAAT | 1/1 A/A mismatch at −2 position; | ADAR2 specificity: 0.61 |
| | 12156 | CCCGAGGTCA | 1/1 A/C mismatch at 0 position; | ADAR12 on target: |
| | | GCAATCTTTGC | 1/1 C/C mismatch at +2 position; | 0.56% |
| | | AATAAGAGAA | 1/1 G/G mismatch at +6 position; | ADAR12 specificity: 0.68 |
| | | GCATTGGGAT | 3/3 symmetric bulge at +10 position | |
| | | ACAGTGTGAA | (AGU-UCG); | |
| | | GAGCAGCA | 7/7 symmetric internal loop at +25 | |
| | | | position (UGGGGAU- | |
| | | | CAGUAGC) | |
| SEQ | LRRK2_ | CCCTGGTGTGC | 6/6 symmetric internal loop at −20 | ADAR1 on target: 0.37% |
| ID | bnPCR_ | CCTCTGATGTT | position (CAUCAU-UACUAC); | ADAR1 specificity: 0.72 |
| NO: | ID8030_ | TTTTAGGGGCT | 1/1 U/G wobble base pair at −3 | ADAR2 on target: 0.59% |
| 204 | count919_- | TCTACAGCAGT | position; | ADAR2 specificity: 0.67 |
| | 20_26_gID_ | TCGGAGGAAT | 1/1 A/A mismatch at −2 position; | ADAR12 on target: |
| | 00460 | CCCGAGGTCA | 1/1 A/C mismatch at 0 position; | 0.53% |
| | | GCAATCTTTGC | 1/1 C/C mismatch at +2 position; | ADAR12 specificity: 0.72 |
| | | ATACTACGCA | 1/1 G/G mismatch at +6 position; | |
| | | GCATTGGGAT | 3/3 symmetric bulge at +10 position | |
| | | ACAGTGTGAA | (AGU-UCG); | |
| | | GAGCAGCA | 7/7 symmetric internal loop at +25 | |
| | | | position (UGGGGAU- | |
| | | | UAGGGGC) | |
| SEQ | LRRK2_ | CCCTGGTGTGC | 6/6 symmetric internal loop at −18 | ADAR1 on target: 0.36% |
| ID | bnPCR_ | CCTCTGATGTT | position (UCAUUG-GUAGCU); | ADAR1 specificity: 0.77 |
| NO: | ID8030_ | TTTATGGAACG | 1/1 U/G wobble base pair at −3 | ADAR2 on target: 0.63% |
| 205 | count919_- | TCTACAGCAGT | position; | ADAR2 specificity: 0.67 |
| | 18_24_gID_ | TCGGAGGAAT | 1/1 A/A mismatch at −2 position; | ADAR12 |
| | 04981 | CCCGAGGTCA | 1/1 A/C mismatch at 0 position; | on target: |
| | | GCAATCTTTGG | 1/1 C/C mismatch at +2 position; | 0.57% |
| | | TAGCTTGGCA | 1/1 G/G mismatch at +6 position; | ADAR12 specificity: 0.7 |
| | | GCATTGGGAT | 3/3 symmetric bulge at +10 position | |
| | | ACAGTGTGAA | (AGU-UCG); | |
| | | GAGCAGCA | 6/6 symmetric internal loop at +24 | |
| | | | position (AUGGGG-GGAACG) | |
| SEQ | LRRK2_ | CCCTGGTGTGC | 6/6 symmetric internal loop at −18 | ADAR1 on target: 0.33% |
| ID | bnPCR_ | CCTCTGATGTT | position (UCAUUG-GUCUUC); | ADAR1 specificity: 0.78 |
| NO: | ID8030_ | TTTATCCAGCG | 1/1 U/G wobble base pair at −3 | ADAR2 on target: 0.62% |
| 206 | count919_- | GGTACAGCAG | position; | ADAR2 specificity: 0.69 |
| | 18_22_gID_ | TTCGGAGGAA | 1/1 A/A mismatch at −2 position; | ADAR12 on target: 0.5% |
| | 06761 | TCCCGAGGTC | 1/1 A/C mismatch at 0 position; | ADAR12 specificity: 0.73 |
| | | AGCAATCTTTG | 1/1 C/C mismatch at +2 position; | |
| | | GTCTTCTGGCA | 1/1 G/G mismatch at +6 position; | |
| | | GCATTGGGAT | 3/3 symmetric bulge at +10 position | |
| | | ACAGTGTGAA | (AGU-UCG); | |
| | | GAGCAGCA | 6/6 symmetric internal loop at +22 | |
| | | | position (GAAUGG-AGCGGG) | |
| SEQ | LRRK2_ | CCCTGGTGTGC | 6/6 symmetric internal loop at −16 | ADAR1 on target: 0.43% |
| ID | bnPCR_ | CCTCTGATGTT | position (AUUGCA-ACCCUG); | ADAR1 specificity: 0.75 |
| NO: | ID8030_ | TCAACGGCCCT | 1/1 U/G wobble base pair at −3 | ADAR2 on target: 0.56% |
| 207 | count919_- | TCTACAGCAGT | position; | ADAR2 specificity: 0.65 |
| | 16_28_gID_ | TCGGAGGAAT | 1/1 A/A mismatch at −2 position; | ADAR12 on target: |
| | 07038 | CCCGAGGTCA | 1/1 A/C mismatch at 0 position; | 0.53% |
| | | GCAATCTTACC | 1/1 C/C mismatch at +2 position; | ADAR12 specificity: 0.66 |
| | | CTGGATGGCA | 1/1 G/G mismatch at +6 position; | |
| | | GCATTGGGAT | 3/3 symmetric bulge at +10 position | |
| | | ACAGTGTGAA | (AGU-UCG); | |
| | | GAGCAGCA | 1/0 asymmetric bulge at +25 | |
| | | | position (U-); | |
| | | | 5/6 asymmetric internal loop at +29 | |
| | | | position (GAUAA-CAACGG) | |
| SEQ | LRRK2_ | CCCTGGTGTGC | 6/6 symmetric internal loop at −16 | ADAR1 on target: 0.38% |
| ID | bnPCR_ | CCTCTGATGTT | position (AUUGCA-AAAUUA); | ADAR1 specificity: 0.81 |
| NO: | ID8030_ | TTTATTGAGCC | 1/1 U/G wobble base pair at −3 | ADAR2 on target: 0.58% |
| 208 | count919_- | TCTACAGCAGT | position; | ADAR2 specificity: 0.69 |
| | 16_24_gID_ | TCGGAGGAAT | 1/1 A/A mismatch at −2 position; | ADAR12 on target: |
| | 09086 | CCCGAGGTCA | 1/1 A/C mismatch at 0 position; | 0.52% |
| | | GCAATCTTAA | 1/1 C/C mismatch at +2 position; | ADAR12 specificity: 0.72 |
| | | ATTAGATGGC | 1/1 G/G mismatch at +6 position; | |
| | | AGCATTGGGA | 3/3 symmetric bulge at +10 position | |
| | | TACAGTGTGA | (AGU-UCG); | |
| | | AGAGCAGCA | 5/5 symmetric internal loop at +24 | |
| | | | position (AUGGG-GAGCC); | |
| | | | 1/1 G/U wobble base pair at +29 | |
| | | | position | |
| SEQ | LRRK2_ | CCCTGGTGTGC | 6/6 symmetric internal loop at −14 | ADAR1 on target: 0.35% |
| ID | bnPCR_ | CCTCTGATGTT | position (UGCAAA-AAACGU); | ADAR1 specificity: 0.82 |
| NO: | ID8030_ | TTTTAGGGGCT | 1/1 U/G wobble base pair at −3 | ADAR2 on target: 0.59% |
| 209 | coun1919_- | TCTACAGCAGT | position; | ADAR2 specificity: 0.73 |
| | 14_26_gID_ | TCGGAGGAAT | 1/1 A/A mismatch at −2 position; | ADAR12 on target: |
| | 07899 | CCCGAGGTCA | 1/1 A/C mismatch at 0 position; | 0.55% |
| | | GCAATCAAAC | 1/1 C/C mismatch at +2 position; | ADAR12 specificity: 0.75 |
| | | GTATGATGGC | 1/1 G/G mismatch at +6 position; | |
| | | AGCATTGGGA | 3/3 symmetric bulge at +10 position | |
| | | TACAGTGTGA | (AGU-UCG); | |
| | | AGAGCAGCA | 7/7 symmetric internal loop at +25 | |
| | | | position (UGGGGAU- | |
| | | | UAGGGGC) | |
| SEQ | LRRK2_ | CCCTGGTGTGC | 6/6 symmetric internal loop at −12 | ADAR1 on target: 0.46% |
| ID | bnPCR_ | CCTCTGATGTT | position (CAAAGA-GACUAA); | ADAR1 specificity: 0.83 |
| NO: | ID8030_ | TTTATGAGGCG | 1/1 U/G wobble base pair at −3 | ADAR2 on target: 0.67% |
| 210 | count919_- | TCTACAGCAGT | position; | ADAR2 specificity: 0.7 |
| | 12_24_gID_ | TCGGAGGAAT | 1/1 A/A mismatch at −2 position; | ADAR12 on target: |
| | 03363 | CCCGAGGTCA | 1/1 A/C mismatch at 0 position; | 0.58% |
| | | GCAAGACTAA | 1/1 C/C mismatch at +2 position; | ADAR12 specificity: 0.76 |
| | | CAATGATGGC | 1/1 G/G mismatch at +6 position; | |
| | | AGCATTGGGA | 3/3 symmetric bulge at +10 position | |
| | | TACAGTGTGA | (AGU-UCG); | |
| | | AGAGCAGCA | 6/6 symmetric internal loop at +24 | |
| | | | position (AUGGGG-GAGGCG) | |
| SEQ | LRRK2_ | CCCTGGTGTGC | 6/6 symmetric internal loop at −10 | ADAR1 on target: 0.5% |
| ID | bnPCR_ | CCTCTGATGTT | position (AAGAUU-UUAGCC); | ADAR1 specificity: 0.84 |
| NO: | ID8030_ | TTTATTGAGCC | 1/1 U/G wobble base pair at -3 | ADAR2 on target: 0.48% |
| 211 | count919_- | TCTACAGCAGT | position; | ADAR2 specificity: 0.75 |
| | 10_24_gID_ | TCGGAGGAAT | 1/1 A/A mismatch at −2 position; | ADAR12 on target: |
| | 09672 | CCCGAGGTCA | 1/1 A/C mismatch at 0 position; | 0.59% |
| | | GCTTAGCCTGC | 1/1 C/C mismatch at +2 position; | ADAR12 specificity: 0.77 |
| | | AATGATGGCA | 1/1 G/G mismatch at +6 position; | |
| | | GCATTGGGAT | 3/3 symmetric bulge (AGU-UCG); | |
| | | ACAGTGTGAA | 5/5 symmetric internal loop at +24 | |
| | | GAGCAGCA | position (AUGGG-GAGCC); | |
| | | | 1/1 G/U wobble base pair at +29 | |
| | | | position | |

### Example 15

### Singleplex Screening of Engineered Guide RNAs In-Trans

Using the compositions and methods described herein, a singleplex screen of longer engineered guide RNAs (e.g., 100mers) in-trans was performed. FIG. 37 shows a schematic of the constructs that can be used for high throughput screening of longer engineered guide RNAs (e.g., 100mers) in-trans, which includes an RNA having the target sequence and a heterologous RNA having the candidate engineered guide RNA and where the RNA having the target sequence and the heterologous RNA having the candidate engineered guide RNA are not linked together or, in other words, are not a single contiguous sequence

Validation of using the compositions shown in FIG. 37 was carried out in a singleplex screen (one engineered guide RNA per target) for LRRK2, SNCA, and GRN. In summary, the forward (F) g-block refers to the RNA having the target RNA sequence and the reverse (R) g-block refers to the heterologous, unlinked RNA having the candidate guide RNA sequence. In some cases, the RNA having the target RNA sequence can be the F g-block and the RNA having the candidate guide RNA sequence can be the R b g-block. The compositions and methods disclosed herein were designed to imprint the gID and universal R sequences onto the RNA having the target RNA sequence in order to map a given engineered guide RNA sequence to the RNA having the edited target RNA sequence. In short the RNA having a candidate guide RNA sequence further comprises a gID sequence and a Universal R sequence. Incubation with the Bst3.0 enzyme results in writing of the complementary DNA sequence of the gID and Universal R sequence onto the RNA having the target RNA sequence, resulting in an RNA-DNA hybrid molecule. Subsequent incubation with the RT enzyme after an ADAR incubation results in amplification of a DNA sequence having the edited target the mapped gID sequence, a mapped Universal R sequence as well as a unique UMI barcode.

The in-trans singleplex assay was carried out as follows. Each F and R g-block for generating the SNCA and LRRK2 and Progranulin RNAs for the cell free “in trans” assay come as 250 ng. These g-blocks were resuspended in 25 uL for 10 ng/uL final concentration. 12.5 uL (125 ng) was used in the subsequent IVT reaction. An IVT enzyme mastermix was prepared according to the table below.

TABLE 8
IVT Enzyme Mastermix

| | | # of reactions |
|---|---|---|
| Reagent | 1x | 9 |
| NTP buffer mix | 10 | 99 |
| Water | 5.5 | 54.45 |
| T7 RNA polymerase | 2 | 19.8 |
| Template DNA | 12.5 | — |
| Total | 30 | |

12.5 uL of template DNA for each oligo and 17.5 uL of the IVT mixture was added into a tube. Reactions were placed on a PCR block and PCR amplified.

For the singleplex screen, incubations of target RNA to candidate guide RNA tested included 1:1.1; 1:5 Target/Guide. Three separate reactions were run testing an exemplary SNCA candidate guide RNA, an exemplary LRRK2 candidate guide RNA, and an exemplary GRN candidate guide RNA. Following incubation, reactions were denatured and the following mastermix was made.

TABLE 9
Bst Enzyme Mastermix

| | |
|---|---|
| 1x | 12x |

| | | | |
|---|---|---|---|
| 10x Buffer | 4 | uL | 52.8 |
| Bst3.0 | 1 | uL | 13.2 |
| 10 mM dNTP | 1 | uL | 13.2 |
| H2O | 14 | uL | 184.8 |

Denatured RNA was added to the above described master mix and allowed to incubate. Once cooled to room temperature, RNA samples were cleaned up and resuspended in H2O. An RT reaction was run to confirm that the barcode (gID+Universal R sequences) had been mapped onto the RNA having the target RNA sequence. Sanger sequencing can be performed after incubation with ADAR to evaluate editing as well as the barcode associated with the guide.

While preferred embodiments of the present disclosure have been shown and described herein, such embodiments are provided by way of example only. Numerous variations, changes, and substitutions can occur without departing from the present disclosure. It should be understood that various alternatives to the embodiments described herein may be employed.

1. A method for identifying a guide RNA suitable for editing a target RNA comprising:
(a) contacting a first self-annealing RNA structure to an RNA editing entity, wherein the first self-annealing RNA structure comprises:
(i) a target RNA,
(ii) a candidate guide RNA, and
(iii) a linker that covalently attaches the target RNA and the candidate guide RNA, wherein
the target RNA and candidate guide RNA form a guide-target RNA scaffold,
when the candidate guide RNA hybridizes to the target RNA, a hairpin loop is formed that comprises at least part of the linker, and
the contacting occurs under conditions that allow the RNA editing entity to edit a base of a nucleotide in the target RNA in the first self-annealing RNA structure; and
(b) identifying an edited target RNA; wherein the edited target RNA identifies a candidate guide RNA suitable for editing the target RNA.

2. The method of claim 1, wherein the RNA editing entity is:
(a) an adenosine deaminase acting on RNA (ADAR);
(b) an ADAR variant;
(c) a catalytically active deaminase domain; or
(d) a fusion protein comprising any one of (a)-(c).

3. The method of claim 1, wherein the RNA editing entity comprises human ADAR (hADAR).

4. The method of claim 3, wherein the hADAR is ADAR1 and/or ADAR2.

5. (canceled)

6. The method of claim 1, wherein
the method is a high throughput method of screening,
the method employs a plurality of self-annealing RNA structures,
the plurality of self-annealing RNA structures includes the first self-annealing RNA structure, and
each respective self-annealing RNA structure in at least a subset of the plurality of self-annealing RNA structures comprises (i) the target RNA, (ii) a respective candidate guide RNA, and (iii) the linker that covalently attaches the target RNA and the respective candidate guide RNA.

7-22. (canceled)

23. The method of claim 1, wherein the first self-annealing RNA structure has a mismatch that comprises a base in the candidate guide RNA that is opposite to and unpaired with a base in the target RNA.

24. The method of claim 23, wherein the mismatch comprises an A/A mismatch, an A/G mismatch, an A/C mismatch, a G/G mismatch, a C/C mismatch, or a C/U mismatch.

25. The method of claim 23, wherein the mismatch comprises an A/C mismatch and wherein the A is in the target RNA and the C is in the candidate guide RNA.

26. The method of claim 25, wherein the A in the A/C mismatch is the base of the nucleotide in the target RNA chemically modified by the RNA editing entity.

27-38. (canceled)

39. The method of claim 6, wherein the plurality of self-annealing RNA structures comprises at least about 10 to 1×10⁸ different self-annealing RNA structures.

40. The method of claim 6, wherein
the identifying comprises:
amplifying the plurality of self-annealing RNA structures to generate a plurality of amplicons; and
sequencing the plurality of amplicons,
wherein one or more of the plurality of self-annealing RNA structures, which each comprise an edited target RNA, are identified based on a sequence of each amplicon in the plurality of amplicons.

41. The method of claim 40, wherein the sequencing is next generation sequencing (NGS).

42. The method of claim 40, wherein
each self-annealing RNA structure in the plurality of self-annealing RNA structures further comprises one or more universal primer binding sites, and
the plurality of amplicons is derived by amplification of the plurality of self-annealing RNA structures using one or more universal primers that bind to the one or more universal primer binding sites.

43-52. (canceled)

53. The method claim 1, wherein the first self-annealing RNA structure comprises from 5′- to 3′-: UPBS1-the target RNA—the hairpin loop—the candidate guide RNA—UPBS2, wherein UPBS1 and UPBS2 are a first universal primer binding site and a second universal primer binding site, respectively.

54. The method of claim 1, wherein the first self-annealing RNA structure comprises a first universal primer binding site, a second universal primer binding site, a first barcode, and a second barcode.

55-56. (canceled)

57. The method of claim 1, wherein the first self-annealing RNA structure has a length of from about 50 nucleotides to about 500 nucleotides.

58. The method of claim 57, wherein the first self-annealing RNA structure has a length of about 100.

59. The method of claim 57, wherein the first self-annealing RNA structure has a length of about 230.

60-121. (canceled)

122. A method for identifying a guide RNA suitable for editing a target RNA comprising:
(a) contacting each self-annealing RNA structure in a plurality of self-annealing RNA structures to an RNA editing entity, wherein each respective self-annealing RNA structure in at least a subset of the plurality of self-annealing RNA structures comprises:
(i) a respective target RNA,
(ii) a respective candidate guide RNA, and
(iii) a respective linker that covalently attaches the respective target RNA and the respective candidate guide RNA, wherein
the respective target RNA and the respective candidate guide RNA form a corresponding guide-target RNA scaffold,
when the respective candidate guide RNA hybridizes to the respective target RNA, a respective hairpin loop is formed that comprises at least part of the respective linker, and
the contacting occurs under conditions that allow the RNA editing entity to edit a base of a nucleotide in the respective target RNA in the respective self-annealing RNA structure; and
(b) identifying an edited target RNA; wherein the edited target RNA identifies a respective candidate guide RNA in the plurality of self-annealing RNA structures suitable for editing the target RNA, wherein each respective target RNA and each respective linker in the plurality of self-annealing RNA structures is the same and at least the subset of the plurality of self-annealing RNA structures comprises a plurality of different candidate guide RNAs.

123. The method of claim 122, wherein the RNA editing entity is:
(a) an adenosine deaminase acting on RNA (ADAR);
(b) an ADAR variant;
(c) a catalytically active deaminase domain; or
(d) a fusion protein comprising any one of (a)-(c).

124. The method of claim 122, wherein the RNA editing entity comprises human ADAR (hADAR).

125. The method of claim 124, wherein the hADAR is ADAR1 and/or ADAR2.

126. The method of claim 122, wherein each respective self-annealing RNA structure in the subset of self-annealing RNA structures has a mismatch that comprises a base in the respective candidate guide RNA that is opposite to and unpaired with a base in the respective target RNA.

127. The method of claim 126, wherein the mismatch comprises an A/A mismatch, an A/G mismatch, an A/C mismatch, a G/G mismatch, a C/C mismatch, or a C/U mismatch.

128. The method of claim 126, wherein the mismatch comprises an A/C mismatch and wherein the A is in the target RNA and the C is in the candidate guide RNA.

129. The method of claim 128, wherein the A in the A/C mismatch is the base of the nucleotide in the target RNA chemically modified by the RNA editing entity.

130. The method of claim 122, where the subset of the plurality of self-annealing RNA structures comprises at least about 10 to 1×10⁸ different self-annealing RNA structures.

131. The method of claim 122, wherein
the identifying comprises:
amplifying the plurality of self-annealing RNA structures to generate a plurality of amplicons; and
sequencing the plurality of amplicons,
wherein one or more self-annealing RNA structures of the plurality of self-annealing RNA structures, which each comprise an edited target RNA, are identified based on a sequence of each amplicon in the plurality of amplicons.

132. The method of claim 131, wherein the sequencing is next generation sequencing (NGS).

133. The method of claim 131, wherein
each self-annealing RNA structure in the plurality of self-annealing RNA structures further comprises one or more universal primer binding sites, and
the plurality of amplicons is derived by amplification of the plurality of self-annealing RNA structures using one or more universal primers that bind to the one or more universal primer binding sites.

134. The method 122, wherein each self-annealing RNA structure in the subset of the plurality of self-annealing RNA structures comprises from 5′- to 3′-: UPBS1-the respective target RNA—the respective hairpin loop—the respective candidate guide RNA—UPBS2, wherein UPBS1 and UPBS2 are a first universal primer binding site and a second universal primer binding site, respectively.

135. The method of claim 122 wherein each self-annealing RNA structure in the subset of the plurality of self-annealing RNA structures comprises a first universal primer binding site, a second universal primer binding site, a first barcode, and a second barcode.

136. The method of claim 122, wherein each self-annealing RNA structure in the subset of the plurality of self-annealing RNA structures has a length of from about 50 nucleotides to about 500 nucleotides.
